SEQUENCE LISTING (529 sequences)

SEQ ID NO: 1 - protein, 9 aa, Homo sapiens
  MISC_FEATURE (5)...(5): The amino acid at this position is optionally phosphorylated
AIMRSPQMV

SEQ ID NO: 2 - protein, 11 aa, Homo sapiens
  MISC_FEATURE (4)...(4): The amino acid at this position is optionally phosphorylated
ALDSGASLLHL

SEQ ID NO: 3 - protein, 9 aa, Homo sapiens
  MISC_FEATURE (5)...(5): The amino acid at this position is optionally phosphorylated
ALGNTPPFL

SEQ ID NO: 4 - protein, 9 aa, Homo sapiens
  MISC_FEATURE (5)...(5): The amino acid at this position is optionally phosphorylated
ALMGSPQLV

SEQ ID NO: 5 - protein, 11 aa, Homo sapiens
  MISC_FEATURE (5)...(5): The amino acid at this position is optionally phosphorylated
ALMGSPQLVAA

SEQ ID NO: 6 - protein, 11 aa, Homo sapiens
  MISC_FEATURE (5)...(5): The amino acid at this position is optionally phosphorylated
AVVSPPALHNA

SEQ ID NO: 7 - protein, 9 aa, Homo sapiens
  MISC_FEATURE (6)...(6): The amino acid at this position is optionally phosphorylated
DLKRRSMSI

SEQ ID NO: 8 - protein, 9 aa, Homo sapiens
  MISC_FEATURE (5)...(5): The amino acid at this position is optionally phosphorylated
ELFSSPPAV

SEQ ID NO: 9 - protein, 9 aa, Homo sapiens
  MISC_FEATURE (4)...(4): The amino acid at this position is optionally phosphorylated
FLDTPIAKV

SEQ ID NO: 10 - protein, 9 aa, Homo sapiens
  MISC_FEATURE (4)...(4): The amino acid at this position is optionally phosphorylated
GIDSPSSSV

SEQ ID NO: 11 - protein, 9 aa, Homo sapiens
  MISC_FEATURE (4)...(4): The amino acid at this position is optionally phosphorylated
GLDSGFHSV

SEQ ID NO: 12 - protein, 9 aa, Homo sapiens
  MISC_FEATURE (4)...(4): The amino acid at this position is optionally phosphorylated
GLISPVWGA

SEQ ID NO: 13 - protein, 9 aa, Homo sapiens
  MISC_FEATURE (5)...(5): The amino acid at this position is optionally phosphorylated
GLLDSPTSI

SEQ ID NO: 14 - protein, 9 aa, Homo sapiens
  MISC_FEATURE (5)...(5): The amino acid at this position is optionally phosphorylated
IMDRTPEKL

SEQ ID NO: 15 - protein, 7 aa, Homo sapiens
  MISC_FEATURE (4)...(4): The amino acid at this position is optionally phosphorylated
KAFSPVR

SEQ ID NO: 16 - protein, 9 aa, Homo sapiens
  MISC_FEATURE (4)...(4): The amino acid at this position is optionally phosphorylated
KAFSPVRSV

SEQ ID NO: 17 - protein, 9 aa, Homo sapiens
  MISC_FEATURE (4)...(4): The amino acid at this position is optionally phosphorylated
KIASEIAQL

SEQ ID NO: 18 - protein, 9 aa, Homo sapiens
  MISC_FEATURE (4)...(4): The amino acid at this position is optionally phosphorylated
KIGSIIFQV

SEQ ID NO: 19 - protein, 10 aa, Homo sapiens
  MISC_FEATURE (4)...(4): The amino acid at this position is optionally phosphorylated
KLASPELERL

SEQ ID NO: 20 - protein, 9 aa, Homo sapiens
  MISC_FEATURE (4)...(4): The amino acid at this position is optionally phosphorylated
KLDSPRVTV

SEQ ID NO: 21 - protein, 10 aa, Homo sapiens
  MISC_FEATURE (6)...(6): The amino acid at this position is optionally phosphorylated
KLFPDTPLAL

SEQ ID NO: 22 - protein, 11 aa, Homo sapiens
  MISC_FEATURE (7)...(7): The amino acid at this position is optionally phosphorylated
KLIDIVSSQKV

SEQ ID NO: 23 - protein, 9 aa, Homo sapiens
  MISC_FEATURE (8)...(8): The amino acid at this position is optionally phosphorylated
KLIDRTESL

SEQ ID NO: 24 - protein, 9 aa, Homo sapiens
  MISC_FEATURE (4)...(4): The amino acid at this position is optionally phosphorylated
KLMSDVEDV

SEQ ID NO: 25 - protein, 11 aa, Homo sapiens
  MISC_FEATURE (4)...(4): The amino acid at this position is optionally phosphorylated
KLMSPKADVKL

SEQ ID NO: 26 - protein, 9 aa, Homo sapiens
  MISC_FEATURE (4)...(4): The amino acid at this position is optionally phosphorylated
KQDSLVINL

SEQ ID NO: 27 - protein, 9 aa, Homo sapiens
  MISC_FEATURE (4)...(4): The amino acid at this position is optionally phosphorylated
KTMSGTFLL

SEQ ID NO: 28 - protein, 9 aa, Homo sapiens
  MISC_FEATURE (4)...(4): The amino acid at this position is optionally phosphorylated
KVASLLHQV

SEQ ID NO: 29 - protein, 9 aa, Homo sapiens
  MISC_FEATURE (4)...(4): The amino acid at this position is optionally phosphorylated
LMFSPVTSL

SEQ ID NO: 30 - protein, 9 aa, Homo sapiens
  MISC_FEATURE (4)...(4): The amino acid at this position is optionally phosphorylated
RASSLSITV

SEQ ID NO: 31 - protein, 9 aa, Homo sapiens
  MISC_FEATURE (4)...(4): The amino acid at this position is optionally phosphorylated
RLASASRAL

SEQ ID NO: 32 - protein, 9 aa, Homo sapiens
  MISC_FEATURE (4)...(4): The amino acid at this position is optionally phosphorylated
RLASLQSEV

SEQ ID NO: 33 - protein, 9 aa, Homo sapiens
  MISC_FEATURE (4)...(4): The amino acid at this position is optionally phosphorylated
RLASYLDKV

SEQ ID NO: 34 - protein, 9 aa, Homo sapiens
  MISC_FEATURE (4)...(4): The amino acid at this position is optionally phosphorylated
RLASYLDRV

SEQ ID NO: 35 - protein, 7 aa, Homo sapiens
  MISC_FEATURE (4)...(4): The amino acid at this position is optionally phosphorylated
RLDSYVR

SEQ ID NO: 36 - protein, 9 aa, Homo sapiens
  MISC_FEATURE (4)...(4): The amino acid at this position is optionally phosphorylated
RLDSYVRSL

SEQ ID NO: 37 - protein, 7 aa, Homo sapiens
  MISC_FEATURE (4)...(4): The amino acid at this position is optionally phosphorylated
RLFSKEL

SEQ ID NO: 38 - protein, 8 aa, Homo sapiens
  MISC_FEATURE (4)...(4): The amino acid at this position is optionally phosphorylated
RLFSKELR

SEQ ID NO: 39 - protein, 9 aa, Homo sapiens
  MISC_FEATURE (4)...(4): The amino acid at this position is optionally phosphorylated
RLFSKELRC

SEQ ID NO: 40 - protein, 9 aa, Homo sapiens
  MISC_FEATURE (4)...(4): The amino acid at this position is optionally phosphorylated
RLLSDLEEL

SEQ ID NO: 41 - protein, 10 aa, Homo sapiens
  MISC_FEATURE (4)...(4): The amino acid at this position is optionally phosphorylated
RLLSTDAEAV

SEQ ID NO: 42 - protein, 9 aa, Homo sapiens
  MISC_FEATURE (5)...(5): The amino acid at this position is optionally phosphorylated
RLSDTPPLL

SEQ ID NO: 43 - protein, 9 aa, Homo sapiens
  MISC_FEATURE (4)...(4): The amino acid at this position is optionally phosphorylated
RLSSPLHFV

SEQ ID NO: 44 - protein, 9 aa, Homo sapiens
  MISC_FEATURE (4)...(4): The amino acid at this position is optionally phosphorylated
RMYSFDDVL

SEQ ID NO: 45 - protein, 10 aa, Homo sapiens
  MISC_FEATURE (4)...(4): The amino acid at this position is optionally phosphorylated
RQASIELPSM

SEQ ID NO: 46 - protein, 12 aa, Homo sapiens
  MISC_FEATURE (4)...(4): The amino acid at this position is optionally phosphorylated
RQASIELPSMAV

SEQ ID NO: 47 - protein, 9 aa, Homo sapiens
  MISC_FEATURE (4)...(4): The amino acid at this position is optionally phosphorylated
RQASLSISV

SEQ ID NO: 48 - protein, 11 aa, Homo sapiens
  MISC_FEATURE (4)...(4): The amino acid at this position is optionally phosphorylated
RQDSTPGKVFL

SEQ ID NO: 49 - protein, 9 aa, Homo sapiens
  MISC_FEATURE (4)...(4): The amino acid at this position is optionally phosphorylated
RQISQDVKL

SEQ ID NO: 50 - protein, 9 aa, Homo sapiens
  MISC_FEATURE (4)...(4): The amino acid at this position is optionally phosphorylated
RQLSALHRA

SEQ ID NO: 51 - protein, 10 aa, Homo sapiens
  MISC_FEATURE (4)...(4): The amino acid at this position is optionally phosphorylated
RQLSSGVSEI

SEQ ID NO: 52 - protein, 9 aa, Homo sapiens
  MISC_FEATURE (4)...(4): The amino acid at this position is optionally phosphorylated
RSLSESYEL

SEQ ID NO: 53 - protein, 10 aa, Homo sapiens
  MISC_FEATURE (4)...(4): The amino acid at this position is optionally phosphorylated
RSLSQELVGV

SEQ ID NO: 54 - protein, 9 aa, Homo sapiens
  MISC_FEATURE (4)...(4): The amino acid at this position is optionally phosphorylated
RTFSPTYGL

SEQ ID NO: 55 - protein, 9 aa, Homo sapiens
  MISC_FEATURE (4)...(4): The amino acid at this position is optionally phosphorylated
RTLSHISEA

SEQ ID NO: 56 - protein, 10 aa, Homo sapiens
  MISC_FEATURE (4)...(4): The amino acid at this position is optionally phosphorylated
RTYSGPMNKV

SEQ ID NO: 57 - protein, 9 aa, Homo sapiens
  MISC_FEATURE (4)...(4): The amino acid at this position is optionally phosphorylated
RVASPTSGV

SEQ ID NO: 58 - protein, 9 aa, Homo sapiens
  MISC_FEATURE (4)...(4): The amino acid at this position is optionally phosphorylated
SIMSPEIQL

SEQ ID NO: 59 - protein, 9 aa, Homo sapiens
  MISC_FEATURE (4)...(4): The amino acid at this position is optionally phosphorylated
SISSMEVNV

SEQ ID NO: 60 - protein, 9 aa, Homo sapiens
  MISC_FEATURE (5)...(5): The amino acid at this position is optionally phosphorylated
SISSTPPAV

SEQ ID NO: 61 - protein, 9 aa, Homo sapiens
  MISC_FEATURE (6)...(6): The amino acid at this position is optionally phosphorylated
SLFGGSVKL

SEQ ID NO: 62 - protein, 10 aa, Homo sapiens
  MISC_FEATURE (4)...(4): The amino acid at this position is optionally phosphorylated
SLFSGDEENA

SEQ ID NO: 63 - protein, 9 aa, Homo sapiens
  MISC_FEATURE (4)...(4): The amino acid at this position is optionally phosphorylated
SLFSPQNTL

SEQ ID NO: 64 - protein, 10 aa, Homo sapiens
  MISC_FEATURE (4)...(4): The amino acid at this position is optionally phosphorylated
SLFSSEESNL

SEQ ID NO: 65 - protein, 12 aa, Homo sapiens
  MISC_FEATURE (4)...(4): The amino acid at this position is optionally phosphorylated
SLFSSEESNLGA

SEQ ID NO: 66 - protein, 9 aa, Homo sapiens
  MISC_FEATURE (8)...(8): The amino acid at this position is optionally phosphorylated
SLHDIQLSL

SEQ ID NO: 67 - protein, 9 aa, Homo sapiens
  MISC_FEATURE (8)...(8): The amino acid at this position is optionally phosphorylated
SLQPRSHSV

SEQ ID NO: 68 - protein, 9 aa, Homo sapiens
  MISC_FEATURE (4)...(4): The amino acid at this position is optionally phosphorylated
SLQSLETSV

SEQ ID NO: 69 - protein, 9 aa, Homo sapiens
  MISC_FEATURE (4)...(4): The amino acid at this position is optionally phosphorylated
SMSSLSREV

SEQ ID NO: 70 - protein, 9 aa, Homo sapiens
  MISC_FEATURE (5)...(5): The amino acid at this position is optionally phosphorylated
SMTRSPPRV

SEQ ID NO: 71 - protein, 9 aa, Homo sapiens
  MISC_FEATURE (8)...(8): The amino acid at this position is optionally phosphorylated
SVKPRRTSL

SEQ ID NO: 72 - protein, 10 aa, Homo sapiens
  MISC_FEATURE (4)...(4): The amino acid at this position is optionally phosphorylated
TVFSPTLPAA

SEQ ID NO: 73 - protein, 9 aa, Homo sapiens
  MISC_FEATURE (5)...(5): The amino acid at this position is optionally phosphorylated
VLFSSPPQM

SEQ ID NO: 74 - protein, 9 aa, Homo sapiens
  MISC_FEATURE (4)...(4): The amino acid at this position is optionally phosphorylated
VLLSPVPEL

SEQ ID NO: 75 - protein, 9 aa, Homo sapiens
  MISC_FEATURE (4)...(4): The amino acid at this position is optionally phosphorylated
VLYSPQMAL

SEQ ID NO: 76 - protein, 9 aa, Homo sapiens
  MISC_FEATURE (5)...(5): The amino acid at this position is optionally phosphorylated
VMIGSPKKV

SEQ ID NO: 77 - protein, 9 aa, Homo sapiens
  MISC_FEATURE (1)...(1): The amino acid at this position is optionally phosphorylated
YLQSRYYRA

SEQ ID NO: 78 - protein, 9 aa, Homo sapiens
  MISC_FEATURE (4)...(4): The amino acid at this position is optionally phosphorylated
APDSPRAFL

SEQ ID NO: 79 - protein, 10 aa, Homo sapiens
  MISC_FEATURE (6)...(6): The amino acid at this position is optionally phosphorylated
APRKGSFSAL

SEQ ID NO: 80 - protein, 10 aa, Homo sapiens
  MISC_FEATURE (6)...(6): The amino acid at this position is optionally phosphorylated
APRNGSGVAL

SEQ ID NO: 81 - protein, 9 aa, Homo sapiens
  MISC_FEATURE (6)...(6): The amino acid at this position is optionally phosphorylated
APRRYSSSL

SEQ ID NO: 82 - protein, 10 aa, Homo sapiens
  MISC_FEATURE (4)...(4): The amino acid at this position is optionally phosphorylated
APRSPPPSRP

SEQ ID NO: 83 - protein, 10 aa, Homo sapiens
  MISC_FEATURE (9)...(9): The amino acid at this position is optionally phosphorylated
APSLFHLNTL

SEQ ID NO: 84 - protein, 11 aa, Homo sapiens
  MISC_FEATURE (8)...(8): The amino acid at this position is optionally phosphorylated
APSSARASPLL

SEQ ID NO: 85 - protein, 11 aa, Homo sapiens
  MISC_FEATURE (5)...(5): The amino acid at this position is optionally phosphorylated
FPLDSPKTLVL

SEQ ID NO: 86 - protein, 9 aa, Homo sapiens
  MISC_FEATURE (6)...(6): The amino acid at this position is optionally phosphorylated
FPRRHSVTL

SEQ ID NO: 87 - protein, 9 aa, Homo sapiens
  MISC_FEATURE (7)...(7): The amino acid at this position is optionally phosphorylated
FRGRYRSPY

SEQ ID NO: 88 - protein, 9 aa, Homo sapiens
  MISC_FEATURE (4)...(4): The amino acid at this position is optionally phosphorylated
FRKSMVEHY

SEQ ID NO: 89 - protein, 10 aa, Homo sapiens
  MISC_FEATURE (9)...(9): The amino acid at this position is optionally phosphorylated
GPPYQRRGSL

SEQ ID NO: 90 - protein, 10 aa, Homo sapiens
  MISC_FEATURE (6)...(6): The amino acid at this position is optionally phosphorylated
GPRPGSPSAL

SEQ ID NO: 91 - protein, 8 aa, Homo sapiens
  MISC_FEATURE (6)...(6): The amino acid at this position is optionally phosphorylated
GPRSASLL

SEQ ID NO: 92 - protein, 10 aa, Homo sapiens
  MISC_FEATURE (6)...(6): The amino acid at this position is optionally phosphorylated
GPRSASLLSL

SEQ ID NO: 93 - protein, 10 aa, Homo sapiens
  MISC_FEATURE (6)...(6): The amino acid at this position is optionally phosphorylated
GPRSASLLSL

SEQ ID NO: 94 - protein, 9 aa, Homo sapiens
  MISC_FEATURE (4)...(4): The amino acid at this position is optionally phosphorylated
GPRSPKAPP

SEQ ID NO: 95 - protein, 8 aa, Homo sapiens
  MISC_FEATURE (7)...(7): The amino acid at this position is optionally phosphorylated
HPKRSVSL

SEQ ID NO: 96 - protein, 9 aa, Homo sapiens
  MISC_FEATURE (4)...(4): The amino acid at this position is opti onally phosphorylated
HRYSTPHAF

SEQ ID NO: 97 - protein, 11 aa, Homo sapiens
  MISC_FEATURE (4)...(4): The amino acid at this position is optionally phosphorylated
KPASPKFIVTL

SEQ ID NO: 98 - protein, 9 aa, Homo sapiens
  MISC_FEATURE (8)...(8): The amino acid at this position is optionally phosphorylated
KPPYRSHSL

SEQ ID NO: 99 - protein, 9 aa, Homo sapiens
  MISC_FEATURE (6)...(6): The amino acid at this position is optionally phosphorylated
KPRPLSMDL

SEQ ID NO: 100 - protein, 9 aa, Homo sapiens
  MISC_FEATURE (8)...(8): The amino acid at this position is optionally phosphorylated
KPRPPPLSP

SEQ ID NO: 101 - protein, 9 aa, Homo sapiens
  MISC_FEATURE (6)...(6): The amino acid at this position is optionally phosphorylated
  MISC_FEATURE (8)...(8): The amino acid at this position is optionally phosphorylated
KPRRFSRSL

SEQ ID NO: 102 - protein, 9 aa, Homo sapiens
  MISC_FEATURE (4)...(4): The amino acid at this position is optionally phosphorylated
KPRSPFSKI

SEQ ID NO: 103 - protein, 9 aa, Homo sapiens
  MISC_FEATURE (4)...(4): The amino acid at this position is optionally phosphorylated
KPRSPPRAL

SEQ ID NO: 104 - protein, 11 aa, Homo sapiens
  MISC_FEATURE (4)...(4): The amino acid at this position is optionally phosphorylated
KPRSPPRALVL

SEQ ID NO: 105 - protein, 9 aa, Homo sapiens
  MISC_FEATURE (4)...(4): The amino acid at this position is optionally phosphorylated
KPRSPVVEL

SEQ ID NO: 106 - protein, 9 aa, Homo sapiens
  MISC_FEATURE (4)...(4): The amino acid at this position is optionally phosphorylated
KPSSPRGSL

SEQ ID NO: 107 - protein, 10 aa, Homo sapiens
  MISC_FEATURE (4)...(4): The amino acid at this position is optionally phosphorylated
KPSSPRGSLL

SEQ ID NO: 108 - protein, 10 aa, Homo sapiens
  MISC_FEATURE (4)...(4): The amino acid at this position is optionally phosphorylated
KPVSPKSGTL

SEQ ID NO: 109 - protein, 9 aa, Homo sapiens
  MISC_FEATURE (4)...(4): The amino acid at this position is optionally phosphorylated
KPYSPLASL

SEQ ID NO: 110 - protein, 10 aa, Homo sapiens
  MISC_FEATURE (4)...(4): The amino acid at this position is optionally phosphorylated
KRASGQAFEL

SEQ ID NO: 111 - protein, 9 aa, Homo sapiens
  MISC_FEATURE (4)...(4): The amino acid at this position is optionally phosphorylated
LPASPRARL

SEQ ID NO: 112 - protein, 9 aa, Homo sapiens
  MISC_FEATURE (8)...(8): The amino acid at this position is optionally phosphorylated
LPIFSRLSI

SEQ ID NO: 113 - protein, 9 aa, Homo sapiens
  MISC_FEATURE (6)...(6): The amino acid at this position is optionally phosphorylated
LPKGLSASL

SEQ ID NO: 114 - protein, 10 aa, Homo sapiens
  MISC_FEATURE (5)...(5): The amino acid at this position is optionally phosphorylated
LPRGSSPSVL

SEQ ID NO: 115 - protein, 9 aa, Homo sapiens
  MISC_FEATURE (6)...(6): The amino acid at this position is optionally phosphorylated
LPRPASPAL

SEQ ID NO: 116 - protein, 9 aa, Homo sapiens
  MISC_FEATURE (6)...(6): The amino acid at this position is optionally phosphorylated
LPRSSSMAA

SEQ ID NO: 117 - protein, 11 aa, Homo sapiens
  MISC_FEATURE (6)...(6): The amino acid at this position is optionally phosphorylated
LPRSSSMAAGL

SEQ ID NO: 118 - protein, 10 aa, Homo sapiens
  MISC_FEATURE (6)...(6): The amino acid at this position is optionally phosphorylated
MPRQPSATRL

SEQ ID NO: 119 - protein, 10 aa, Homo sapiens
  MISC_FEATURE (4)...(4): The amino acid at this position is optionally phosphorylated
QPRTPSPLVL

SEQ ID NO: 120 - protein, 10 aa, Homo sapiens
  MISC_FEATURE (6)...(6): The amino acid at this position is optionally phosphorylated
RARGISPIVF

SEQ ID NO: 121 - protein, 9 aa, Homo sapiens
  MISC_FEATURE (4)...(4): The amino acid at this position is optionally phosphorylated
RKLSVILIL

SEQ ID NO: 122 - protein, 10 aa, Homo sapiens
  MISC_FEATURE (4)...(4): The amino acid at this position is optionally phosphorylated
RLLSPQQPAL

SEQ ID NO: 123 - protein, 9 aa, Homo sapiens
  MISC_FEATURE (6)...(6): The amino acid at this position is optionally phosphorylated
RPAFFSPSL

SEQ ID NO: 124 - protein, 9 aa, Homo sapiens
  MISC_FEATURE (5)...(5): The amino acid at this position is optionally phosphorylated
RPAKSMDSL

SEQ ID NO: 125 - protein, 9 aa, Homo sapiens
  MISC_FEATURE (4)...(4): The amino acid at this position is optionally phosphorylated
RPASAGAML

SEQ ID NO: 126 - protein, 9 aa, Homo sapiens
  MISC_FEATURE (4)...(4): The amino acid at this position is optionally phosphorylated
RPASPAAKL

SEQ ID NO: 127 - protein, 9 aa, Homo sapiens
  MISC_FEATURE (4)...(4): The amino acid at this position is optionally phosphorylated
RPASPEPEL

SEQ ID NO: 128 - protein, 9 aa, Homo sapiens
  MISC_FEATURE (4)...(4): The amino acid at this position is optionally phosphorylated
RPASPGPSL

SEQ ID NO: 129 - protein, 10 aa, Homo sapiens
  MISC_FEATURE (4)...(4): The amino acid at this position is optionally phosphorylated
RPASPQRAQL

SEQ ID NO: 130 - protein, 9 aa, Homo sapiens
  MISC_FEATURE (4)...(4): The amino acid at this position is optionally phosphorylated
RPASPSLQL

SEQ ID NO: 131 - protein, 10 aa, Homo sapiens
  MISC_FEATURE (4)...(4): The amino acid at this position is optionally phosphorylated
RPASPSLQLL

SEQ ID NO: 132 - protein, 11 aa, Homo sapiens
  MISC_FEATURE (4)...(4): The amino acid at this position is optionally phosphorylated
RPASYKKKSML

SEQ ID NO: 133 - protein, 10 aa, Homo sapiens
  MISC_FEATURE (4)...(4): The amino acid at this position is optionally phosphorylated
RPDSPTRPTL

SEQ ID NO: 134 - protein, 11 aa, Homo sapiens
  MISC_FEATURE (4)...(4): The amino acid at this position is optionally phosphorylated
RPDSRLGKTEL

SEQ ID NO: 135 - protein, 10 aa, Homo sapiens
  MISC_FEATURE (9)...(9): The amino acid at this position is optionally phosphorylated
RPDVAKRLSL

SEQ ID NO: 136 - protein, 11 aa, Homo sapiens
  MISC_FEATURE (10)...(10): The amino acid at this position is optionally phosphorylated
RPFHGISTVSL

SEQ ID NO: 137 - protein, 9 aa, Homo sapiens
  MISC_FEATURE (4)...(4): The amino acid at this position is optionally phosphorylated
RPFSPREAL

SEQ ID NO: 138 - protein, 9 aa, Homo sapiens
  MISC_FEATURE (4)...(4): The amino acid at this position is optionally phosphorylated
RPGSRQAGL

SEQ ID NO: 139 - protein, 9 aa, Homo sapiens
  MISC_FEATURE (4)...(4): The amino acid at this position is optionally phosphorylated
RPISPGLSY

SEQ ID NO: 140 - protein, 9 aa, Homo sapiens
  MISC_FEATURE (4)...(4): The amino acid at this position is optionally phosphorylated
RPISPPHTY

SEQ ID NO: 141 - protein, 10 aa, Homo sapiens
  MISC_FEATURE (4)...(4): The amino acid at this position is optionally phosphorylated
RPISPRIGAL

SEQ ID NO: 142 - protein, 9 aa, Homo sapiens
  MISC_FEATURE (6)...(6): The amino acid at this position is optionally phosphorylated
RPKLSSPAL

SEQ ID NO: 143 - protein, 9 aa, Homo sapiens
  MISC_FEATURE (4)...(4): The amino acid at this position is optionally phosphorylated
RPKSNIVLL

SEQ ID NO: 144 - protein, 9 aa, Homo sapiens
  MISC_FEATURE (4)...(4): The amino acid at this position is optionally phosphorylated
RPKSPLSKM

SEQ ID NO: 145 - protein, 10 aa, Homo sapiens
  MISC_FEATURE (4)...(4): The amino acid at this position is optionally phosphorylated
RPKSVDFDSL

SEQ ID NO: 146 - protein, 9 aa, Homo sapiens
  MISC_FEATURE (4)...(4): The amino acid at this position is optionally phosphorylated
RPKTPPVVI

SEQ ID NO: 147 - protein, 9 aa, Homo sapiens
  MISC_FEATURE (4)...(4): The amino acid at this position is optionally phosphorylated
RPLSLLLAL

SEQ ID NO: 148 - protein, 9 aa, Homo sapiens
  MISC_FEATURE (4)...(4): The amino acid at this position is optionally phosphorylated
RPLSVVYVL

SEQ ID NO: 149 - protein, 9 aa, Homo sapiens
  MISC_FEATURE (4)...(4): The amino acid at this position is optionally phosphorylated
RPMSESPHM

SEQ ID NO: 150 - protein, 10 aa, Homo sapiens
  MISC_FEATURE (4)...(4): The amino acid at this position is optionally phosphorylated
RPNSPSPTAL

SEQ ID NO: 151 - protein, 9 aa, Homo sapiens
  MISC_FEATURE (4)...(4): The amino acid at this position is optionally phosphorylated
RPPSPGPVL

SEQ ID NO: 152 - protein, 10 aa, Homo sapiens
  MISC_FEATURE (6)...(6): The amino acid at this position is optionally phosphorylated
RPQRATSNVF

SEQ ID NO: 153 - protein, 8 aa, Homo sapiens
  MISC_FEATURE (6)...(6): The amino acid at this position is optionally phosphorylated
RPRAATVV

SEQ ID NO: 154 - protein, 9 aa, Homo sapiens
  MISC_FEATURE (6)...(6): The amino acid at this position is optionally phosphorylated
RPRAATVVA

SEQ ID NO: 155 - protein, 11 aa, Homo sapiens
  MISC_FEATURE (6)...(6): The amino acid at this position is optionally phosphorylated
RPRANSGGVDL

SEQ ID NO: 156 - protein, 10 aa, Homo sapiens
  MISC_FEATURE (6)...(6): The amino acid at this position is optionally phosphorylated
RPRARSVDAL

SEQ ID NO: 157 - protein, 10 aa, Homo sapiens
  MISC_FEATURE (5)...(5): The amino acid at this position is optionally phosphorylated
RPRDTRRISL

SEQ ID NO: 158 - protein, 9 aa, Homo sapiens
  MISC_FEATURE (5)...(5): The amino acid at this position is optionally phosphorylated
RPRGSESLL

SEQ ID NO: 159 - protein, 9 aa, Homo sapiens
  MISC_FEATURE (5)...(5): The amino acid at this position is optionally phosphorylated
RPRGSQSLL

SEQ ID NO: 160 - protein, 10 aa, Homo sapiens
  MISC_FEATURE (6)...(6): The amino acid at this position is optionally phosphorylated
RPRIPSPIGF

SEQ ID NO: 161 - protein, 10 aa, Homo sapiens
  MISC_FEATURE (6)...(6): The amino acid at this position is optionally phosphorylated
RPRPASSPAL

SEQ ID NO: 162 - protein, 10 aa, Homo sapiens
  MISC_FEATURE (6)...(6): The amino acid at this position is optionally phosphorylated
RPRPHSAPSL

SEQ ID NO: 163 - protein, 11 aa, Homo sapiens
  MISC_FEATURE (6)...(6): The amino acid at this position is optionally phosphorylated
RPRPSSAHVGL

SEQ ID NO: 164 - protein, 8 aa, Homo sapiens
  MISC_FEATURE (5)...(5): The amino acid at this position is optionally phosphorylated
RPRPSSVL

SEQ ID NO: 165 - protein, 11 aa, Homo sapiens
  MISC_FEATURE (5)...(5): The amino acid at this position is optionally phosphorylated
RPRPSSVLRTL

SEQ ID NO: 166 - protein, 10 aa, Homo sapiens
  MISC_FEATURE (6)...(6): The amino acid at this position is optionally phosphorylated
RPRPVSPSSL

SEQ ID NO: 167 - protein, 11 aa, Homo sapiens
  MISC_FEATURE (6)...(6): The amino acid at this position is optionally phosphorylated
RPRPVSPSSLL

SEQ ID NO: 168 - protein, 9 aa, Homo sapiens
  MISC_FEATURE (4)...(4): The amino acid at this position is optionally phosphorylated
RPRSAVEQL

SEQ ID NO: 169 - protein, 8 aa, Homo sapiens
  MISC_FEATURE (4)...(4): The amino acid at this position is optionally phosphorylated
RPRSAVLL

SEQ ID NO: 170 - protein, 10 aa, Homo sapiens
  MISC_FEATURE (4)...(4): The amino acid at this position is optionally phosphorylated
RPRSISVEEF

SEQ ID NO: 171 - protein, 9 aa, Homo sapiens
  MISC_FEATURE (4)...(4): The amino acid at this position is optionally phosphorylated
RPRSLEVTI

SEQ ID NO: 172 - protein, 12 aa, Homo sapiens
  MISC_FEATURE (6)...(6): The amino acid at this position is optionally phosphorylated
RPRSLSSPTVTL

SEQ ID NO: 173 - protein, 9 aa, Homo sapiens
  MISC_FEATURE (4)...(4): The amino acid at this position is optionally phosphorylated
RPRSMTVSA

SEQ ID NO: 174 - protein, 9 aa, Homo sapiens
  MISC_FEATURE (4)...(4): The amino acid at this position is optionally phosphorylated
RPRSMVRSF

SEQ ID NO: 175 - protein, 9 aa, Homo sapiens
  MISC_FEATURE (4)...(4): The amino acid at this position is optionally phosphorylated
RPRSPAARL

SEQ ID NO: 176 - protein, 10 aa, Homo sapiens
  MISC_FEATURE (4)...(4): The amino acid at this position is optionally phosphorylated
RPRSPNMQDL

SEQ ID NO: 177 - protein, 9 aa, Homo sapiens
  MISC_FEATURE (4)...(4): The amino acid at this position is optionally phosphorylated
RPRSPPGGP

SEQ ID NO: 178 - protein, 10 aa, Homo sapiens
  MISC_FEATURE (4)...(4): The amino acid at this position is optionally phosphorylated
RPRSPPPRAP

SEQ ID NO: 179 - protein, 9 aa, Homo sapiens
  MISC_FEATURE (4)...(4): The amino acid at this position is optionally phosphorylated
RPRSPPSSP

SEQ ID NO: 180 - protein, 10 aa, Homo sapiens
  MISC_FEATURE (4)...(4): The amino acid at this position is optionally phosphorylated
RPRSPRENSI

SEQ ID NO: 181 - protein, 9 aa, Homo sapiens
  MISC_FEATURE (4)...(4): The amino acid at this position is optionally phosphorylated
RPRSPRPPP

SEQ ID NO: 182 - protein, 10 aa, Homo sapiens
  MISC_FEATURE (4)...(4): The amino acid at this position is optionally phosphorylated
RPRSPRQNSI

SEQ ID NO: 183 - protein, 9 aa, Homo sapiens
  MISC_FEATURE (4)...(4): The amino acid at this position is optionally phosphorylated
RPRSPSPIS

SEQ ID NO: 184 - protein, 12 aa, Homo sapiens
  MISC_FEATURE (6)...(6): The amino acid at this position is optionally phosphorylated
RPRSPTGPSNSF

SEQ ID NO: 185 - protein, 13 aa, Homo sapiens
  MISC_FEATURE (4)...(4): The amino acid at this position is optionally phosphorylated
RPRSPTGPSNSFL

SEQ ID NO: 186 - protein, 9 aa, Homo sapiens
  MISC_FEATURE (4)...(4): The amino acid at this position is optionally phosphorylated
RPRSPWGKL

SEQ ID NO: 187 - protein, 10 aa, Homo sapiens
  MISC_FEATURE (4)...(4): The amino acid at this position is optionally phosphorylated
RPRSQYNTKL

SEQ ID NO: 188 - protein, 9 aa, Homo sapiens
  MISC_FEATURE (4)...(4): The amino acid at this position is optionally phosphorylated
RPRTPLRSL

SEQ ID NO: 189 - protein, 8 aa, Homo sapiens
  MISC_FEATURE (4)...(4): The amino acid at this position is optionally phosphorylated
RPSSLPDL

SEQ ID NO: 190 - protein, 9 aa, Homo sapiens
  MISC_FEATURE (4)...(4): The amino acid at this position is optionally phosphorylated
RPSSPALYF

SEQ ID NO: 191 - protein, 9 aa, Homo sapiens
  MISC_FEATURE (4)...(4): The amino acid at this position is optionally phosphorylated
RPTSFADEL

SEQ ID NO: 192 - protein, 9 aa, Homo sapiens
  MISC_FEATURE (4)...(4): The amino acid at this position is optionally phosphorylated
RPTSRLNRL

SEQ ID NO: 193 - protein, 9 aa, Homo sapiens
  MISC_FEATURE (4)...(4): The amino acid at this position is optionally phosphorylated
RPVSPFQEL

SEQ ID NO: 194 - protein, 9 aa, Homo sapiens
  MISC_FEATURE (4)...(4): The amino acid at this position is optionally phosphorylated
RPVSPGKDI

SEQ ID NO: 195 - protein, 9 aa, Homo sapiens
  MISC_FEATURE (6)...(6): The amino acid at this position is optionally phosphorylated
RPVSPSSLL

SEQ ID NO: 196 - protein, 10 aa, Homo sapiens
  MISC_FEATURE (4)...(4): The amino acid at this position is optionally phosphorylated
RPVSTDFAQY

SEQ ID NO: 197 - protein, 9 aa, Homo sapiens
  MISC_FEATURE (4)...(4): The amino acid at this position is optionally phosphorylated
RPVTPVSDL

SEQ ID NO: 198 - protein, 9 aa, Homo sapiens
  MISC_FEATURE (4)...(4): The amino acid at this position is optionally phosphorylated
RPWSNSRGL

SEQ ID NO: 199 - protein, 9 aa, Homo sapiens
  MISC_FEATURE (4)...(4): The amino acid at this position is optionally phosphorylated
RPWSPAVSA

SEQ ID NO: 200 - protein, 10 aa, Homo sapiens
  MISC_FEATURE (4)...(4): The amino acid at this position is optionally phosphorylated
RPYSPPFFSL

SEQ ID NO: 201 - protein, 9 aa, Homo sapiens
  MISC_FEATURE (4)...(4): The amino acid at this position is optionally phosphorylated
RPYSQVNVL

SEQ ID NO: 202 - protein, 9 aa, Homo sapiens
  MISC_FEATURE (4)...(4): The amino acid at this position is optionally phosphorylated
RTRSPSPTL

SEQ ID NO: 203 - protein, 10 aa, Homo sapiens
  MISC_FEATURE (7)...(7): The amino acid at this position is optionally phosphorylated
RVRKLPSTTL

SEQ ID NO: 204 - protein, 9 aa, Homo sapiens
  MISC_FEATURE (4)...(4): The amino acid at this position is optionally phosphorylated
SPASPKISL

SEQ ID NO: 205 - protein, 9 aa, Homo sapiens
  MISC_FEATURE (8)...(8): The amino acid at this position is optionally phosphorylated
SPFKRQLSL

SEQ ID NO: 206 - protein, 9 aa, Homo sapiens
  MISC_FEATURE (5)...(5): The amino acid at this position is optionally phosphorylated
SPFLSKRSL

SEQ ID NO: 207 - protein, 10 aa, Homo sapiens
  MISC_FEATURE (9)...(9): The amino acid at this position is optionally phosphorylated
SPGLARKRSL

SEQ ID NO: 208 - protein, 9 aa, Homo sapiens
  MISC_FEATURE (4)...(4): The amino acid at this position is optionally phosphorylated
SPKSPGLKA

SEQ ID NO: 209 - protein, 9 aa, Homo sapiens
  MISC_FEATURE (6)...(6): The amino acid at this position is optionally phosphorylated
SPRERSPAL

SEQ ID NO: 210 - protein, 9 aa, Homo sapiens
  MISC_FEATURE (7)...(7): The amino acid at this position is optionally phosphorylated
  MISC_FEATURE (8)...(8): The amino acid at this position is optionally phosphorylated
SPRGEASSL

SEQ ID NO: 211 - protein, 9 aa, Homo sapiens
  MISC_FEATURE (4): The amino acid at this position is optionally phosphorylated
SPRSPGRSL

SEQ ID NO: 212 - protein, 9 aa, Homo sapiens
  MISC_FEATURE (4)...(4): The amino acid at this position is optionally phosphorylated
SPRSPSGLR

SEQ ID NO: 213 - protein, 10 aa, Homo sapiens
  MISC_FEATURE (6)...(6): The amino acid at this position is optionally phosphorylated
SPRSPSTTYL

SEQ ID NO: 214 - protein, 11 aa, Homo sapiens
  MISC_FEATURE (4)...(4): The amino acid at this position is optionally phosphorylated
SPSSPSVRRQL

SEQ ID NO: 215 - protein, 9 aa, Homo sapiens
  MISC_FEATURE (8)...(8): The amino acid at this position is optionally phosphorylated
TPMKKHLSL

SEQ ID NO: 216 - protein, 9 aa, Homo sapiens
  MISC_FEATURE (4)...(4): The amino acid at this position is optionally phosphorylated
TPRSPPLGL

SEQ ID NO: 217 - protein, 10 aa, Homo sapiens
  MISC_FEATURE (4)...(4): The amino acid at this position is optionally phosphorylated
TPRSPPLGLI

SEQ ID NO: 218 - protein, 7 aa, Homo sapiens
  MISC_FEATURE (6)...(6): The amino acid at this position is optionally phosphorylated
VAKRLSL

SEQ ID NO: 219 - protein, 10 aa, Homo sapiens
  MISC_FEATURE (8)...(8): The amino acid at this position is optionally phosphorylated
VPRPERRSSL

SEQ ID NO: 220 - protein, 13 aa, Homo sapiens
  MISC_FEATURE (4)...(4): The amino acid at this position is optionally phosphorylated
VPRSPKHAHSSSL

SEQ ID NO: 221 - protein, 9 aa, Homo sapiens
  MISC_FEATURE (4)...(4): The amino acid at this position is optionally phosphorylated
VPTSPKSSL

SEQ ID NO: 222 - protein, 10 aa, Homo sapiens
  MISC_FEATURE (6)...(6): The amino acid at this position is optionally phosphorylated
YPDPHSPFAV

SEQ ID NO: 223 - protein, 9 aa, Homo sapiens
  MISC_FEATURE (7)...(7): The amino acid at this position is optionally phosphorylated
YPGGRRSSL

SEQ ID NO: 224 - protein, 10 aa, Homo sapiens
  MISC_FEATURE (6)...(6): The amino acid at this position is optionally phosphorylated
YPYEFSPVKM

SEQ ID NO: 225 - protein, 10 aa, Homo sapiens
  MISC_FEATURE (4)...(4): The amino acid at this position is optionally phosphorylated
FRRSPTKSSL

SEQ ID NO: 226 - protein, 11 aa, Homo sapiens
  MISC_FEATURE (4)...(4): The amino acid at this position is optionally phosphorylated
FRRSPTKSSLD

SEQ ID NO: 227 - protein, 12 aa, Homo sapiens
  MISC_FEATURE (4)...(4): The amino acid at this position is optionally phosphorylated
FRRSPTKSSLDY

SEQ ID NO: 228 - protein, 9 aa, Homo sapiens
  MISC_FEATURE (4)...(4): The amino acid at this position is optionally phosphorylated
GRKSPPPSF

SEQ ID NO: 229 - protein, 9 aa, Homo sapiens
  MISC_FEATURE (4)...(4): The amino acid at this position is optionally phosphorylated
GRLSPAYSL

SEQ ID NO: 230 - protein, 10 aa, Homo sapiens
  MISC_FEATURE (4)...(4): The amino acid at this position is optionally phosphorylated
GRLSPVPVPR

SEQ ID NO: 231 - protein, 9 aa, Homo sapiens
  MISC_FEATURE (4)...(4): The amino acid at this position is optionally phosphorylated
GRQSPSFKL

SEQ ID NO: 232 - protein, 9 aa, Homo sapiens
  MISC_FEATURE (3)...(3): The amino acid at this position is optionally phosphorylated
GRSSPPPGY

SEQ ID NO: 233 - protein, 9 aa, Homo sapiens
  MISC_FEATURE (4)...(4): The amino acid at this position is optionally phosphorylated
KRASYILRL

SEQ ID NO: 234 - protein, 9 aa, Homo sapiens
  MISC_FEATURE (4)...(4): The amino acid at this position is optionally phosphorylated
KRFSFKKSF

SEQ ID NO: 235 - protein, 9 aa, Homo sapiens
  MISC_FEATURE (4)...(4): The amino acid at this position is optionally phosphorylated
KRFSFKKSF

SEQ ID NO: 236 - protein, 9 aa, Homo sapiens
  MISC_FEATURE (4)...(4): The amino acid at this position is optionally phosphorylated
KRFSGTVRL

SEQ ID NO: 237 - protein, 9 aa, Homo sapiens
  MISC_FEATURE (4)...(4): The amino acid at this position is optionally phosphorylated
KRKSFTSLY

SEQ ID NO: 238 - protein, 9 aa, Homo sapiens
  MISC_FEATURE (6)...(6): The amino acid at this position is optionally phosphorylated
KRLEKSPSF

SEQ ID NO: 239 - protein, 9 aa, Homo sapiens
  MISC_FEATURE (4)...(4): The amino acid at this position is optionally phosphorylated
KRLSPAPQL

SEQ ID NO: 240 - protein, 9 aa, Homo sapiens
  MISC_FEATURE (4)...(4): The amino acid at this position is optionally phosphorylated
KRMSPKPEL

SEQ ID NO: 241 - protein, 9 aa, Homo sapiens
  MISC_FEATURE (5)...(5): The amino acid at this position is optionally phosphorylated
KRWQSPVTK

SEQ ID NO: 242 - protein, 9 aa, Homo sapiens
  MISC_FEATURE (4)...(4): The amino acid at this position is optionally phosphorylated
KRYSGNMEY

SEQ ID NO: 243 - protein, 9 aa, Homo sapiens
  MISC_FEATURE (4)...(4): The amino acid at this position is optionally phosphorylated
KRYSRALYL

SEQ ID NO: 244 - protein, 10 aa, Homo sapiens
  MISC_FEATURE (4)...(4): The amino acid at this position is optionally phosphorylated
QRLSPLSAAY

SEQ ID NO: 245 - protein, 9 aa, Homo sapiens
  MISC_FEATURE (4)...(4): The amino acid at this position is optionally phosphorylated
RRASIITKY

SEQ ID NO: 246 - protein, 10 aa, Homo sapiens
  MISC_FEATURE (4)...(4): The amino acid at this position is optionally phosphorylated
RRASLSEIGF

SEQ ID NO: 247 - protein, 9 aa, Homo sapiens
  MISC_FEATURE (4)...(4): The amino acid at this position is optionally phosphorylated
RRDSIVAEL

SEQ ID NO: 248 - protein, 10 aa, Homo sapiens
  MISC_FEATURE (4)...(4): The amino acid at this position is optionally phosphorylated
RRDSLQKPGL

SEQ ID NO: 249 - protein, 9 aa, Homo sapiens
  MISC_FEATURE (4)...(4): The amino acid at this position is optionally phosphorylated
RRFSGTAVY

SEQ ID NO: 250 - protein, 9 aa, Homo sapiens
  MISC_FEATURE (4)...(4): The amino acid at this position is optionally phosphorylated
RRFSIATLR

SEQ ID NO: 251 - protein, 9 aa, Homo sapiens
  MISC_FEATURE (4)...(4): The amino acid at this position is optionally phosphorylated
RRFSLTTLR

SEQ ID NO: 252 - protein, 9 aa, Homo sapiens
  MISC_FEATURE (4)...(4): The amino acid at this position is optionally phosphorylated
RRFSPPRRM

SEQ ID NO: 253 - protein, 9 aa, Homo sapiens
  MISC_FEATURE (4)...(4): The amino acid at this position is optionally phosphorylated
RRFSRSDEL

SEQ ID NO: 254 - protein, 9 aa, Homo sapiens
  MISC_FEATURE (4)...(4): The amino acid at this position is optionally phosphorylated
RRFSRSPIR

SEQ ID NO: 255 - protein, 9 aa, Homo sapiens
  MISC_FEATURE (6)...(6): The amino acid at this position is optionally phosphorylated
RRFSRSPIR

SEQ ID NO: 256 - protein, 10 aa, Homo sapiens
  MISC_FEATURE (4)...(4): The amino acid at this position is optionally phosphorylated
RRFSRSPIRR

SEQ ID NO: 257 - protein, 9 aa, Homo sapiens
  MISC_FEATURE (4)...(4): The amino acid at this position is optionally phosphorylated
RRGSFEVTL

SEQ ID NO: 258 - protein, 10 aa, Homo sapiens
  MISC_FEATURE (6)...(6): The amino acid at this position is optionally phosphorylated
RRIDISPSTF

SEQ ID NO: 259 - protein, 9 aa, Homo sapiens
  MISC_FEATURE (4)...(4): The amino acid at this position is optionally phosphorylated
RRISDPEVF

SEQ ID NO: 260 - protein, 9 aa, Homo sapiens
  MISC_FEATURE (4)...(4): The amino acid at this position is optionally phosphorylated
RRISDPQVF

SEQ ID NO: 261 - protein, 9 aa, Homo sapiens
  MISC_FEATURE (4)...(4): The amino acid at this position is optionally phosphorylated
RRISQIQQL

SEQ ID NO: 262 - protein, 9 aa, Homo sapiens
  MISC_FEATURE (4)...(4): The amino acid at this position is optionally phosphorylated
RRKSQVAEL

SEQ ID NO: 263 - protein, 9 aa, Homo sapiens
  MISC_FEATURE (4)...(4): The amino acid at this position is optionally phosphorylated
RRLSADIRL

SEQ ID NO: 264 - protein, 9 aa, Homo sapiens
  MISC_FEATURE (4)...(4): The amino acid at this position is optionally phosphorylated
RRLSELLRY

SEQ ID NO: 265 - protein, 10 aa, Homo sapiens
  MISC_FEATURE (4)...(4): The amino acid at this position is optionally phosphorylated
RRLSGGSHSY

SEQ ID NO: 266 - protein, 9 aa, Homo sapiens
  MISC_FEATURE (4)...(4): The amino acid at this position is optionally phosphorylated
RRLSRKLSL

SEQ ID NO: 267 - protein, 8 aa, Homo sapiens
  MISC_FEATURE (4)...(4): The amino acid at this position is optionally phosphorylated
RRMSFQKP

SEQ ID NO: 268 - protein, 9 aa, Homo sapiens
  MISC_FEATURE (4)...(4): The amino acid at this position is optionally phosphorylated
RRMSLLSVV

SEQ ID NO: 269 - protein, 9 aa, Homo sapiens
  MISC_FEATURE (4)...(4): The amino acid at this position is optionally phosphorylated
RRNSAPVSV

SEQ ID NO: 270 - protein, 9 aa, Homo sapiens
  MISC_FEATURE (4)...(4): The amino acid at this position is optionally phosphorylated
RRPSIAPVL

SEQ ID NO: 271 - protein, 9 aa, Homo sapiens
  MISC_FEATURE (4)...(4): The amino acid at this position is optionally phosphorylated
RRPSLLSEF

SEQ ID NO: 272 - protein, 9 aa, Homo sapiens
  MISC_FEATURE (4)...(4): The amino acid at this position is optionally phosphorylated
RRPSLVHGY

SEQ ID NO: 273 - protein, 9 aa, Homo sapiens
  MISC_FEATURE (4)...(4): The amino acid at this position is optionally phosphorylated
RRPSYTLGM

SEQ ID NO: 274 - protein, 9 aa, Homo sapiens
  MISC_FEATURE (4)...(4): The amino acid at this position is optionally phosphorylated
RRRSLERLL

SEQ ID NO: 275 - protein, 7 aa, Homo sapiens
  MISC_FEATURE (5)...(5): The amino acid at this position is optionally phosphorylated
RRSFSLE

SEQ ID NO: 276 - protein, 7 aa, Homo sapiens
  MISC_FEATURE (4)...(4): The amino acid at this position is optionally phosphorylated
RRSSFLQ

SEQ ID NO: 277 - protein, 9 aa, Homo sapiens
  MISC_FEATURE (4)...(4): The amino acid at this position is optionally phosphorylated
RRSSFLQVF

SEQ ID NO: 278 - protein, 9 aa, Homo sapiens
  MISC_FEATURE (4)...(4): The amino acid at this position is optionally phosphorylated
RRSSIQSTF

SEQ ID NO: 279 - protein, 9 aa, Homo sapiens
  MISC_FEATURE (4)...(4): The amino acid at this position is optionally phosphorylated
RRSSQSWSL

SEQ ID NO: 280 - protein, 9 aa, Homo sapiens
  MISC_FEATURE (8)...(8): The amino acid at this position is optionally phosphorylated
RRVVQRSSL

SEQ ID NO: 281 - protein, 9 aa, Homo sapiens
  MISC_FEATURE (4)...(4): The amino acid at this position is optionally phosphorylated
RRYSKFFDL

SEQ ID NO: 282 - protein, 9 aa, Homo sapiens
  MISC_FEATURE (4)...(4): The amino acid at this position is optionally phosphorylated
RRYSPPIQR

SEQ ID NO: 283 - protein, 8 aa, Homo sapiens
  MISC_FEATURE (4)...(4): The amino acid at this position is optionally phosphorylated
RSRSPLEL

SEQ ID NO: 284 - protein, 10 aa, Homo sapiens
  MISC_FEATURE (5)...(5): The amino acid at this position is optionally phosphorylated
SPRRSRSISL

SEQ ID NO: 285 - protein, 9 aa, Homo sapiens
  MISC_FEATURE (8)...(8): The amino acid at this position is optionally phosphorylated
SRFNRRVSV

SEQ ID NO: 286 - protein, 10 aa, Homo sapiens
  MISC_FEATURE (5)...(5): The amino acid at this position is optionally phosphorylated
AEQGSPRVSY

SEQ ID NO: 287 - protein, 12 aa, Homo sapiens
  MISC_FEATURE (2)...(2): The amino acid at this position is optionally phosphorylated
GSPHYFSPFRPY

SEQ ID NO: 288 - protein, 9 aa, Homo sapiens
  MISC_FEATURE (4)...(4): The amino acid at this position is optionally phosphorylated
ISSSMHSLY

SEQ ID NO: 289 - protein, 9 aa, Homo sapiens
  MISC_FEATURE (5)...(5): The amino acid at this position is optionally phosphorylated
ITQGTPLKY

SEQ ID NO: 290 - protein, 10 aa, Homo sapiens
  MISC_FEATURE (9)...(9): The amino acid at this position is optionally phosphorylated
LLDPSRSYSY

SEQ ID NO: 291 - protein, 10 aa, Homo sapiens
  MISC_FEATURE (4)...(4): The amino acid at this position is optionally phosphorylated
SLDSPSYVLY

SEQ ID NO: 292 - protein, 9 aa, Homo sapiens
  MISC_FEATURE (8)...(8): The amino acid at this position is optionally phosphorylated
SLYDRPASY

SEQ ID NO: 293 - protein, 10 aa, Homo sapiens
  MISC_FEATURE (4)...(4): The amino acid at this position is optionally phosphorylated
SYPSPVATSY

SEQ ID NO: 294 - protein, 10 aa, Homo sapiens
  MISC_FEATURE (4)...(4): The amino acid at this position is optionally phosphorylated
TMASPGKDNY

SEQ ID NO: 295 - protein, 8 aa, Homo sapiens
  MISC_FEATURE (3)...(3): The amino acid at this position is optionally phosphorylated
YFSPFRPY

SEQ ID NO: 296 - protein, 11 aa, Homo sapiens
  MISC_FEATURE (4)...(4): The amino acid at this position is optionally phosphorylated
YPLSPTKISQY

SEQ ID NO: 297 - protein, 10 aa, Homo sapiens
  MISC_FEATURE (6)...(6): The amino acid at this position is optionally phosphorylated
YQRPFSPSAY

SEQ ID NO: 298 - protein, 9 aa, Homo sapiens
  MISC_FEATURE (4)...(4): The amino acid at this position is optionally phosphorylated
ATYTPQAPK

SEQ ID NO: 299 - protein, 10 aa, Homo sapiens
  MISC_FEATURE (6)...(6): The amino acid at this position is optionally phosphorylated
FLIIRTVLQL

SEQ ID NO: 300 - protein, 9 aa, Homo sapiens
  MISC_FEATURE (4)...(4): The amino acid at this position is optionally phosphorylated
FRYSGKTEY

SEQ ID NO: 301 - protein, 9 aa, Homo sapiens
  MISC_FEATURE (4)...(4): The amino acid at this position is optionally phosphorylated
GIMSPLAKK

SEQ ID NO: 302 - protein, 9 aa, Homo sapiens
  MISC_FEATURE (4)...(4): The amino acid at this position is optionally phosphorylated
IISSPLTGK

SEQ ID NO: 303 - protein, 8 aa, Homo sapiens
  MISC_FEATURE (7)...(7): The amino acid at this position is optionally phosphorylated
ILKPRRSL

SEQ ID NO: 304 - protein, 9 aa, Homo sapiens
  MISC_FEATURE (4)...(4): The amino acid at this position is optionally phosphorylated
IYQYIQSRF

SEQ ID NO: 305 - protein, 9 aa, Homo sapiens
  MISC_FEATURE (5)...(5): The amino acid at this position is optionally phosphorylated
KLPDSPALA

SEQ ID NO: 306 - protein, 10 aa, Homo sapiens
  MISC_FEATURE (5)...(5): The amino acid at this position is optionally phosphorylated
KLPDSPALAK

SEQ ID NO: 307 - protein, 11 aa, Homo sapiens
  MISC_FEATURE (5)...(5): The amino acid at this position is optionally phosphorylated
KLPDSPALAKK

SEQ ID NO: 308 - protein, 10 aa, Homo sapiens
  MISC_FEATURE (4)...(4): The amino acid at this position is optionally phosphorylated
KLPSPAPARK

SEQ ID NO: 309 - protein, 9 aa, Homo sapiens
  MISC_FEATURE (4)...(4): The amino acid at this position is optionally phosphorylated
KLRSPFLQK

SEQ ID NO: 310 - protein, 10 aa, Homo sapiens
  MISC_FEATURE (5)...(5): The amino acid at this position is optionally phosphorylated
KMPTTPVKAK

SEQ ID NO: 311 - protein, 9 aa, Homo sapiens
  MISC_FEATURE (4)...(4): The amino acid at this position is optionally phosphorylated
KRASVFVKL

SEQ ID NO: 312 - protein, 10 aa, Homo sapiens
  MISC_FEATURE (5)...(5): The amino acid at this position is optionally phosphorylated
KTPTSPLKMK

SEQ ID NO: 313 - protein, 9 aa, Homo sapiens
  MISC_FEATURE (4)...(4): The amino acid at this position is optionally phosphorylated
KVQSLRRAL

SEQ ID NO: 314 - protein, 10 aa, Homo sapiens
  MISC_FEATURE (4)...(4): The amino acid at this position is optionally phosphorylated
MTRSPPRVSK

SEQ ID NO: 315 - protein, 9 aa, Homo sapiens
  MISC_FEATURE (4)...(4): The amino acid at this position is optionally phosphorylated
RAKSPISLK

SEQ ID NO: 316 - protein, 9 aa, Homo sapiens
  MISC_FEATURE (4)...(4): The amino acid at this position is optionally phosphorylated
RILSGVVTK

SEQ ID NO: 317 - protein, 7 aa, Homo sapiens
  MISC_FEATURE (5)...(5): The amino acid at this position is optionally phosphorylated
RIYQYIQ

SEQ ID NO: 318 - protein, 9 aa, Homo sapiens
  MISC_FEATURE (5)...(5): The amino acid at this position is optionally phosphorylated
RIYQYIQSR

SEQ ID NO: 319 - protein, 10 aa, Homo sapiens
  MISC_FEATURE (5)...(5): The amino acid at this position is optionally phosphorylated
RIYQYIQSRF

SEQ ID NO: 320 - protein, 9 aa, Homo sapiens
  MISC_FEATURE (6)...(6): The amino acid at this position is optionally phosphorylated
RLFVGSIPK

SEQ ID NO: 321 - protein, 12 aa, Homo sapiens
  MISC_FEATURE (8)...(8): The amino acid at this position is optionally phosphorylated
RLLDRSPSRSAK

SEQ ID NO: 322 - protein, 9 aa, Homo sapiens
  MISC_FEATURE (4)...(4): The amino acid at this position is optionally phosphorylated
RLSSPISKR

SEQ ID NO: 323 - protein, 9 aa, Homo sapiens
  MISC_FEATURE (4)...(4): The amino acid at this position is optionally phosphorylated
RLSSPVLHR

SEQ ID NO: 324 - protein, 9 aa, Homo sapiens
  MISC_FEATURE (4)...(4): The amino acid at this position is optionally phosphorylated
RSLSVEIVY

SEQ ID NO: 325 - protein, 9 aa, Homo sapiens
  MISC_FEATURE (4)...(4): The amino acid at this position is optionally phosphorylated
RSYSRSFSR

SEQ ID NO: 326 - protein, 9 aa, Homo sapiens
  MISC_FEATURE (4)...(4): The amino acid at this position is optionally phosphorylated
RSYSYPRQK

SEQ ID NO: 327 - protein, 9 aa, Homo sapiens
  MISC_FEATURE (4)...(4): The amino acid at this position is optionally phosphorylated
RTASFAVRK

SEQ ID NO: 328 - protein, 10 aa, Homo sapiens
  MISC_FEATURE (4)...(4): The amino acid at this position is optionally phosphorylated
RTASPPPPPK

SEQ ID NO: 329 - protein, 11 aa, Homo sapiens
  MISC_FEATURE (4)...(4): The amino acid at this position is optionally phosphorylated
RTRSLSSLREK

SEQ ID NO: 330 - protein, 10 aa, Homo sapiens
  MISC_FEATURE (4)...(4): The amino acid at this position is optionally phosphorylated
RVASPTSGVK

SEQ ID NO: 331 - protein, 11 aa, Homo sapiens
  MISC_FEATURE (4)...(4): The amino acid at this position is optionally phosphorylated
RVKTPTSQSYR

SEQ ID NO: 332 - protein, 9 aa, Homo sapiens
  MISC_FEATURE (4)...(4): The amino acid at this position is optionally phosphorylated
RVLSPLIIK

SEQ ID NO: 333 - protein, 10 aa, Homo sapiens
  MISC_FEATURE (5)...(5): The amino acid at this position is optionally phosphorylated
RVRQSPLATR

SEQ ID NO: 334 - protein, 9 aa, Homo sapiens
  MISC_FEATURE (4)...(4): The amino acid at this position is optionally phosphorylated
RVYSPYNHR

SEQ ID NO: 335 - protein, 9 aa, Homo sapiens
  MISC_FEATURE (4)...(4): The amino acid at this position is optionally phosphorylated
SVKSPVTVK

SEQ ID NO: 336 - protein, 9 aa, Homo sapiens
  MISC_FEATURE (5)...(5): The amino acid at this position is optionally phosphorylated
SVRRSVLMK

SEQ ID NO: 337 - protein, 6 aa, Homo sapiens
  MISC_FEATURE (1)...(1): The amino acid at this position is optionally phosphorylated
YIQSRF

SEQ ID NO: 338 - protein, 9 aa, Homo sapiens
  MISC_FEATURE (8)...(8): The amino acid at this position is optionally phosphorylated
AENARSASF

SEQ ID NO: 339 - protein, 10 aa, Homo sapiens
  MISC_FEATURE (4)...(4): The amino acid at this position is optionally phosphorylated
AENSPTRQQF

SEQ ID NO: 340 - protein, 9 aa, Homo sapiens
  MISC_FEATURE (4)...(4): The amino acid at this position is optionally phosphorylated
AENSSSREL

SEQ ID NO: 341 - protein, 9 aa, Homo sapiens
  MISC_FEATURE (2)...(2): The amino acid at this position is optionally phosphorylated
ATAGPRLGW

SEQ ID NO: 342 - protein, 9 aa, Homo sapiens
  MISC_FEATURE (4)...(4): The amino acid at this position is optionally phosphorylated
EELSPTAKF

SEQ ID NO: 343 - protein, 8 aa, Homo sapiens
  MISC_FEATURE (3)...(3): The amino acid at this position is optionally phosphorylated
FKTQPVTF

SEQ ID NO: 344 - protein, 9 aa, Homo sapiens
  MISC_FEATURE (4)...(4): The amino acid at this position is optionally phosphorylated
GEASPSHII

SEQ ID NO: 345 - protein, 9 aa, Homo sapiens
  MISC_FEATURE (4)...(4): The amino acid at this position is optionally phosphorylated
GEISPQREV

SEQ ID NO: 346 - protein, 9 aa, Homo sapiens
  MISC_FEATURE (4)...(4): The amino acid at this position is optionally phosphorylated
GETSPRTKI

SEQ ID NO: 347 - protein, 8 aa, Homo sapiens
  MISC_FEATURE (7)...(7): The amino acid at this position is optionally phosphorylated
HEKKAYSF

SEQ ID NO: 348 - protein, 9 aa, Homo sapiens
  MISC_FEATURE (4)...(4): The amino acid at this position is optionally phosphorylated
KEKSPFRET

SEQ ID NO: 349 - protein, 9 aa, Homo sapiens
  MISC_FEATURE (8)...(8): The amino acid at this position is optionally phosphorylated
KELARQISF

SEQ ID NO: 350 - protein, 9 aa, Homo sapiens
  MISC_FEATURE (4)...(4): The amino acid at this position is optionally phosphorylated
KEMSPTRQL

SEQ ID NO: 351 - protein, 11 aa, Homo sapiens
  MISC_FEATURE (4)...(4): The amino acid at this position is optionally phosphorylated
KESSPLSSRKI

SEQ ID NO: 352 - protein, 9 aa, Homo sapiens
  MISC_FEATURE (5)...(5): The amino acid at this position is optionally phosphorylated
REAPSPLMI

SEQ ID NO: 353 - protein, 9 aa, Homo sapiens
  MISC_FEATURE (4)...(4): The amino acid at this position is optionally phosphorylated
REASPAPLA

SEQ ID NO: 354 - protein, 9 aa, Homo sapiens
  MISC_FEATURE (4)...(4): The amino acid at this position is optionally phosphorylated
REASPRLRV

SEQ ID NO: 355 - protein, 10 aa, Homo sapiens
  MISC_FEATURE (4)...(4): The amino acid at this position is optionally phosphorylated
REASPSRLSV

SEQ ID NO: 356 - protein, 10 aa, Homo sapiens
  MISC_FEATURE (6)...(6): The amino acid at this position is optionally phosphorylated
REIMGTPEYL

SEQ ID NO: 357 - protein, 9 aa, Homo sapiens
  MISC_FEATURE (4)...(4): The amino acid at this position is optionally phosphorylated
REKSPGRML

SEQ ID NO: 358 - protein, 9 aa, Homo sapiens
  MISC_FEATURE (8)...(8): The amino acid at this position is optionally phosphorylated
RELARKGSL

SEQ ID NO: 359 - protein, 9 aa, Homo sapiens
  MISC_FEATURE (4)...(4): The amino acid at this position is optionally phosphorylated
RELSPLISL

SEQ ID NO: 360 - protein, 9 aa, Homo sapiens
  MISC_FEATURE (4)...(4): The amino acid at this position is optionally phosphorylated
REPSPLPEL

SEQ ID NO: 361 - protein, 9 aa, Homo sapiens
  MISC_FEATURE (4)...(4): The amino acid at this position is optionally phosphorylated
RERSPSPSF

SEQ ID NO: 362 - protein, 9 aa, Homo sapiens
  MISC_FEATURE (4)...(4): The amino acid at this position is optionally phosphorylated
RESSPTRRL

SEQ ID NO: 363 - protein, 9 aa, Homo sapiens
  MISC_FEATURE (4)...(4): The amino acid at this position is optionally phosphorylated
REVSPAPAV

SEQ ID NO: 364 - protein, 9 aa, Homo sapiens
  MISC_FEATURE (5)...(5): The amino acid at this position is optionally phosphorylated
REYGSTSSI

SEQ ID NO: 365 - protein, 9 aa, Homo sapiens
  MISC_FEATURE (4)...(4): The amino acid at this position is optionally phosphorylated
RFKTQPVTF

SEQ ID NO: 366 - protein, 9 aa, Homo sapiens
  MISC_FEATURE (4)...(4): The amino acid at this position is optionally phosphorylated
RQKSPLFQF

SEQ ID NO: 367 - protein, 9 aa, Homo sapiens
  MISC_FEATURE (8)...(8): The amino acid at this position is optionally phosphorylated
SEFKAMDSI

SEQ ID NO: 368 - protein, 9 aa, Homo sapiens
  MISC_FEATURE (4)...(4): The amino acid at this position is optionally phosphorylated
SELSPGRSV

SEQ ID NO: 369 - protein, 9 aa, Homo sapiens
  MISC_FEATURE (4)...(4): The amino acid at this position is optionally phosphorylated
TEASPESML

SEQ ID NO: 370 - protein, 8 aa, Homo sapiens
  MISC_FEATURE (4)...(4): The amino acid at this position is optionally phosphorylated
YEGSPIKV

SEQ ID NO: 371 - protein, 9 aa, Homo sapiens
  MISC_FEATURE (4)...(4): The amino acid at this position is optionally phosphorylated
FRFSGRTEY

SEQ ID NO: 372 - protein, 9 aa, Homo sapiens
  MISC_FEATURE (4)...(4): The amino acid at this position is optionally phosphorylated
KRASFAKSV

SEQ ID NO: 373 - protein, 9 aa, Homo sapiens
  MISC_FEATURE (4)...(4): The amino acid at this position is optionally phosphorylated
LSSSVIREL

SEQ ID NO: 374 - protein, 9 aa, Homo sapiens
  MISC_FEATURE (4)...(4): The amino acid at this position is optionally phosphorylated
RKPSIVTKY

SEQ ID NO: 375 - protein, 11 aa, Homo sapiens
  MISC_FEATURE (4)...(4): The amino acid at this position is optionally phosphorylated
RRHSASNLHAL

SEQ ID NO: 376 - protein, 9 aa, Homo sapiens
  MISC_FEATURE (4)...(4): The amino acid at this position is optionally phosphorylated
RRLSFLVSY

SEQ ID NO: 377 - protein, 9 aa, Homo sapiens
  MISC_FEATURE (4)...(4): The amino acid at this position is optionally phosphorylated
RRLSYVLFI

SEQ ID NO: 378 - protein, 9 aa, Homo sapiens
  MISC_FEATURE (4)...(4): The amino acid at this position is optionally phosphorylated
RRPSYRKIL

SEQ ID NO: 379 - protein, 9 aa, Homo sapiens
  MISC_FEATURE (4)...(4): The amino acid at this position is optionally phosphorylated
RSASFSRKV

SEQ ID NO: 380 - protein, 9 aa, Homo sapiens
  MISC_FEATURE (8)...(8): The amino acid at this position is optionally phosphorylated
SRSSSVLSL

SEQ ID NO: 381 - protein, 9 aa, Homo sapiens
  MISC_FEATURE (4)...(4): The amino acid at this position is optionally phosphorylated
TRKTPESFL

SEQ ID NO: 382 - protein, 9 aa, Homo sapiens
  MISC_FEATURE (4)...(4): The amino acid at this position is optionally phosphorylated
YRYSPQSFL

SEQ ID NO: 383 - protein, 8 aa, Homo sapiens
  MISC_FEATURE (4)...(4): The amino acid at this position is optionally phosphorylated
KVDSPVIF

SEQ ID NO: 384 - protein, 8 aa, Homo sapiens
  MISC_FEATURE (4)...(4): The amino acid at this position is optionally phosphorylated
RADSPVHM

SEQ ID NO: 385 - protein, 8 aa, Homo sapiens
  MISC_FEATURE (4)...(4): The amino acid at this position is optionally phosphorylated
RSDSYVEL

SEQ ID NO: 386 - protein, 9 aa, Homo sapiens
  MISC_FEATURE (4)...(4): The amino acid at this position is optionally phosphorylated
RSESPPAEL

SEQ ID NO: 387 - protein, 9 aa, Homo sapiens
  MISC_FEATURE (4)...(4): The amino acid at this position is optionally phosphorylated
RVDSPSHGL

SEQ ID NO: 388 - protein, 8 aa, Homo sapiens
  MISC_FEATURE (4)...(4): The amino acid at this position is optionally phosphorylated
SIDSPQKL

SEQ ID NO: 389 - protein, 9 aa, Homo sapiens
  MISC_FEATURE (4)...(4): The amino acid at this position is optionally phosphorylated
RYQTQPVTL

SEQ ID NO: 390 - protein, 9 aa, Homo sapiens
  MISC_FEATURE (4)...(4): The amino acid at this position is optionally phosphorylated
VYTYIQSRF

SEQ ID NO: 391 - protein, 9 aa, Homo sapiens
  MISC_FEATURE (4)...(4): The amino acid at this position is optionally phosphorylated
RTSSFTFQN

SEQ ID NO: 392 - protein, 9 aa, Homo sapiens
  MISC_FEATURE (4)...(4): The amino acid at this position is optionally phosphorylated
RAHSEPLAL

SEQ ID NO: 393 - protein, 9 aa, Homo sapiens
  MISC_FEATURE (4)...(4): The amino acid at this position is optionally phosphorylated
ADLSPEREV

SEQ ID NO: 394 - protein, 9 aa, Homo sapiens
  MISC_FEATURE (4)...(4): The amino acid at this position is optionally phosphorylated
AGDSPGSQF

SEQ ID NO: 395 - protein, 8 aa, Homo sapiens
  MISC_FEATURE (4)...(4): The amino acid at this position is optionally phosphorylated
AKLSETIS

SEQ ID NO: 396 - protein, 7 aa, Homo sapiens
  MISC_FEATURE (2)...(2): The amino acid at this position is optionally phosphorylated
ASLGFVF

SEQ ID NO: 397 - protein, 9 aa, Homo sapiens
  MISC_FEATURE (5)...(5): The amino acid at this position is optionally phosphorylated
DAKKSPLAL

SEQ ID NO: 398 - protein, 9 aa, Homo sapiens
  MISC_FEATURE (8)...(8): The amino acid at this position is optionally phosphorylated
DLKSSKASL

SEQ ID NO: 399 - protein, 9 aa, Homo sapiens
  MISC_FEATURE (4)...(4): The amino acid at this position is optionally phosphorylated
FTKSPYQEF

SEQ ID NO: 400 - protein, 9 aa, Homo sapiens
  MISC_FEATURE (4)...(4): The amino acid at this position is optionally phosphorylated
GQLSPGVQF

SEQ ID NO: 401 - protein, 9 aa, Homo sapiens
  MISC_FEATURE (2)...(2): The amino acid at this position is optionally phosphorylated
GSPHYFSPF

SEQ ID NO: 402 - protein, 10 aa, Homo sapiens
  MISC_FEATURE (4)...(4): The amino acid at this position is optionally phosphorylated
HTASPTGMMK

SEQ ID NO: 403 - protein, 9 aa, Homo sapiens
  MISC_FEATURE (4)...(4): The amino acid at this position is optionally phosphorylated
HVYTPSTTK

SEQ ID NO: 404 - protein, 10 aa, Homo sapiens
  MISC_FEATURE (4)...(4): The amino acid at this position is optionally phosphorylated
IQFSPPFPGA

SEQ ID NO: 405 - protein, 9 aa, Homo sapiens
  MISC_FEATURE (8)...(8): The amino acid at this position is optionally phosphorylated
KASPKRLSL

SEQ ID NO: 406 - protein, 9 aa, Homo sapiens
  MISC_FEATURE (4)...(4): The amino acid at this position is optionally phosphorylated
  MISC_FEATURE (8)...(8): The amino acid at this position can be cysteinylated
KAVSLFLCY

SEQ ID NO: 407 - protein, 9 aa, Homo sapiens
  MISC_FEATURE (4)...(4): The amino acid at this position is optionally phosphorylated
KIFSGVFVK

SEQ ID NO: 408 - protein, 9 aa, Homo sapiens
  MISC_FEATURE (4)...(4): The amino acid at this position is optionally phosphorylated
KIKSFEVVF

SEQ ID NO: 409 - protein, 9 aa, Homo sapiens
  MISC_FEATURE (8)...(8): The amino acid at this position is optionally phosphorylated
KLKDRLPSI

SEQ ID NO: 410 - protein, 10 aa, Homo sapiens
  MISC_FEATURE (3)...(3): The amino acid at this position is optionally phosphorylated
KLSGDQPAAR

SEQ ID NO: 411 - protein, 7 aa, Homo sapiens
  MISC_FEATURE (5)...(5): The amino acid at this position is optionally phosphorylated
KLSGLSF

SEQ ID NO: 412 - protein, 10 aa, Homo sapiens
  MISC_FEATURE (4)...(4): The amino acid at this position is optionally phosphorylated
KTMSPSQMIM

SEQ ID NO: 413 - protein, 11 aa, Homo sapiens
  MISC_FEATURE (4)...(4): The amino acid at this position is optionally phosphorylated
KVKSSPLIEKL

SEQ ID NO: 414 - protein, 9 aa, Homo sapiens
  MISC_FEATURE (4)...(4): The amino acid at this position is optionally phosphorylated
NMDSPGPML

SEQ ID NO: 415 - protein, 9 aa, Homo sapiens
  MISC_FEATURE (6)...(6): The amino acid at this position is optionally phosphorylated
PMVTLSLNL

SEQ ID NO: 416 - protein, 11 aa, Homo sapiens
  MISC_FEATURE (8)...(8): The amino acid at this position is optionally phosphorylated
PYDPALGSPSR

SEQ ID NO: 417 - protein, 9 aa, Homo sapiens
  MISC_FEATURE (4)...(4): The amino acid at this position is optionally phosphorylated
RAFSVKFEV

SEQ ID NO: 418 - protein, 8 aa, Homo sapiens
  MISC_FEATURE (7)...(7): The amino acid at this position is optionally phosphorylated
RGDGYGTF

SEQ ID NO: 419 - protein, 9 aa, Homo sapiens
  MISC_FEATURE (4)...(4): The amino acid at this position is optionally phosphorylated
RIGSPLSPK

SEQ ID NO: 420 - protein, 9 aa, Homo sapiens
  MISC_FEATURE (5)...(5): The amino acid at this position is optionally phosphorylated
RKLRSLEQL

SEQ ID NO: 421 - protein, 9 aa, Homo sapiens
  MISC_FEATURE (4)...(4): The amino acid at this position is optionally phosphorylated
RKSSIIIRM

SEQ ID NO: 422 - protein, 11 aa, Homo sapiens
  MISC_FEATURE (6)...(6): The amino acid at this position is optionally phosphorylated
  MISC_FEATURE (7)...(7): The amino acid at this position is optionally phosphorylated
RLLDPSSPLAL

SEQ ID NO: 423 - protein, 11 aa, Homo sapiens
  MISC_FEATURE (4)...(4): The amino acid at this position is optionally phosphorylated
RLSSLRASTSK

SEQ ID NO: 424 - protein, 9 aa, Homo sapiens
  MISC_FEATURE (4)...(4): The amino acid at this position is optionally phosphorylated
RMFSPMEEK

SEQ ID NO: 425 - protein, 10 aa, Homo sapiens
  MISC_FEATURE (4)...(4): The amino acid at this position is optionally phosphorylated
RMYSPIPPSL

SEQ ID NO: 426 - protein, 9 aa, Homo sapiens
  MISC_FEATURE (4)...(4): The amino acid at this position is optionally phosphorylated
RNLSSPFIF

SEQ ID NO: 427 - protein, 9 aa, Homo sapiens
  MISC_FEATURE (4)...(4): The amino acid at this position is optionally phosphorylated
RSRSPRPAL

SEQ ID NO: 428 - protein, 9 aa, Homo sapiens
  MISC_FEATURE (4)...(4): The amino acid at this position is optionally phosphorylated
RTHSLLLLL

SEQ ID NO: 429 - protein, 9 aa, Homo sapiens
  MISC_FEATURE (4)...(4): The amino acid at this position is optionally phosphorylated
RTNSPGFQK

SEQ ID NO: 430 - protein, 9 aa, Homo sapiens
  MISC_FEATURE (4)...(4): The amino acid at this position is optionally phosphorylated
RTPSDVKEL

SEQ ID NO: 431 - protein, 9 aa, Homo sapiens
  MISC_FEATURE (4)...(4): The amino acid at this position is optionally phosphorylated
RTSSFALNL

SEQ ID NO: 432 - protein, 9 aa, Homo sapiens
  MISC_FEATURE (4)...(4): The amino acid at this position is optionally phosphorylated
RTSSPLFNK

SEQ ID NO: 433 - protein, 9 aa, Homo sapiens
  MISC_FEATURE (4)...(4): The amino acid at this position is optionally phosphorylated
RTYSHGTYR

SEQ ID NO: 434 - protein, 9 aa, Homo sapiens
  MISC_FEATURE (4)...(4): The amino acid at this position is optionally phosphorylated
RYPSNLQLF

SEQ ID NO: 435 - protein, 10 aa, Homo sapiens
  MISC_FEATURE (1)...(1): The amino acid at this position is optionally phosphorylated
SDDEKMPDLE

SEQ ID NO: 436 - protein, 9 aa, Homo sapiens
  MISC_FEATURE (8)...(8): The amino acid at this position is optionally phosphorylated
SDMPRAHSF

SEQ ID NO: 437 - protein, 9 aa, Homo sapiens
  MISC_FEATURE (4)...(4): The amino acid at this position is optionally phosphorylated
SFDSGSVRL

SEQ ID NO: 438 - protein, 11 aa, Homo sapiens
  MISC_FEATURE (2)...(2): The amino acid at this position is optionally phosphorylated
SSPIMRKKVSL

SEQ ID NO: 439 - protein, 9 aa, Homo sapiens
  MISC_FEATURE (1)...(1): The amino acid at this position is optionally phosphorylated
SYIEHIFEI

SEQ ID NO: 440 - protein, 9 aa, Homo sapiens
  MISC_FEATURE (1)...(1): The amino acid at this position is optionally phosphorylated
SYQKVIELF

SEQ ID NO: 441 - protein, 9 aa, Homo sapiens
  MISC_FEATURE (5)...(5): The amino acid at this position is optionally phosphorylated
TLLASPMLK

SEQ ID NO: 442 - protein, 9 aa, Homo sapiens
  MISC_FEATURE (8)...(8): The amino acid at this position is optionally phosphorylated
TLMERTVSL

SEQ ID NO: 443 - protein, 10 aa, Homo sapiens
  MISC_FEATURE (6)...(6): The amino acid at this position is optionally phosphorylated
VLFPESPARA

SEQ ID NO: 444 - protein, 9 aa, Homo sapiens
  MISC_FEATURE (8)...(8): The amino acid at this position is optionally phosphorylated
VLIENVASL

SEQ ID NO: 445 - protein, 9 aa, Homo sapiens
  MISC_FEATURE (8)...(8): The amino acid at this position is optionally phosphorylated
VLSDVIPSI

SEQ ID NO: 446 - protein, 9 aa, Homo sapiens
  MISC_FEATURE (8)...(8): The amino acid at this position is optionally phosphorylated
VLVVDTPSI

SEQ ID NO: 447 - protein, 10 aa, Homo sapiens
  MISC_FEATURE (4)...(4): The amino acid at this position is optionally phosphorylated
VVDSPGQEVL

SEQ ID NO: 448 - protein, 9 aa, Homo sapiens
  MISC_FEATURE (4)...(4): The amino acid at this position is optionally phosphorylated
YARSVHEEF

SEQ ID NO: 449 - protein, 15 aa, Clostridium tetani
QYIKANSKFIGITEL

SEQ ID NO: 450 - protein, 16 aa, Artificial Sequence
  Artificially synthesized tetanus peptide
AQYIKANSKFIGITEL

SEQ ID NO: 451 - protein, 9 aa, Cytomegalovirus
NLVPMVATV

SEQ ID NO: 452 - protein, 9 aa, Homo sapiens
HLFGYSWYK

SEQ ID NO: 453 - protein, 9 aa, Homo sapiens
YLSGADLNL

SEQ ID NO: 454 - protein, 9 aa, Homo sapiens
EIWTHSYKV

SEQ ID NO: 455 - protein, 9 aa, Homo sapiens
ALLAVGATK

SEQ ID NO: 456 - protein, 16 aa, Homo sapiens
WNRQLYPEWTEAQRLD

SEQ ID NO: 457 - protein, 9 aa, Homo sapiens
ALNFPGSQK

SEQ ID NO: 458 - protein, 9 aa, Homo sapiens
SQNFPGSQK

SEQ ID NO: 459 - protein, 9 aa, Homo sapiens
KTWGQYWQV

SEQ ID NO: 460 - protein, 9 aa, Homo sapiens
ITDQVPFSV

SEQ ID NO: 461 - protein, 9 aa, Homo sapiens
IMDQVPFSV

SEQ ID NO: 462 - protein, 9 aa, Homo sapiens
YLEPGPVTA

SEQ ID NO: 463 - protein, 10 aa, Homo sapiens
VLYRYGSFSV

SEQ ID NO: 464 - protein, 9 aa, Homo sapiens
LIYRRRLMK

SEQ ID NO: 465 - protein, 9 aa, Homo sapiens
KIFGSLAFL

SEQ ID NO: 466 - protein, 9 aa, Homo sapiens
VLRENTSPK

SEQ ID NO: 467 - protein, 14 aa, Homo sapiens
LLKYRAREPVTKAE

SEQ ID NO: 468 - protein, 9 aa, Homo sapiens
SLFRAVITK

SEQ ID NO: 469 - protein, 9 aa, Homo sapiens
EADPTGHSY

SEQ ID NO: 470 - protein, 9 aa, Homo sapiens
EVDPIGHLY

SEQ ID NO: 471 - protein, 15 aa, Homo sapiens
TSYVKVLHHMVKISG

SEQ ID NO: 472 - protein, 9 aa, Homo sapiens
GLYDGMEHL

SEQ ID NO: 473 - protein, 9 aa, Homo sapiens
AAGIGILTV

SEQ ID NO: 474 - protein, 23 aa, Homo sapiens
RNGYRALMDKSLHVGTQCALTRR

SEQ ID NO: 475 - protein, 20 aa, Homo sapiens
  MISC_FEATURE (12)...(12): The amino acid at this position can be phosphorylated
VPNAPPAYEKLSAEQSPPPY

SEQ ID NO: 476 - protein, 12 aa, Homo sapiens
  MISC_FEATURE (11)...(11): The amino acid at this position can be phosphorylated
PNAPPAYEKLSA

SEQ ID NO: 477 - protein, 12 aa, Homo sapiens
  MISC_FEATURE (10)...(10): The amino acid at this position can be phosphorylated
NAPPAYEKLSAE

SEQ ID NO: 478 - protein, 9 aa, Homo sapiens
  MISC_FEATURE (9)...(9): The amino acid at this position can be phosphorylated
APPAYEKLS

SEQ ID NO: 479 - protein, 12 aa, Homo sapiens
  MISC_FEATURE (9)...(9): The amino acid at this position can be phosphorylated
APPAYEKLSAEQ

SEQ ID NO: 480 - protein, 15 aa, Homo sapiens
  MISC_FEATURE (9)...(9): The amino acid at this position can be phosphorylated
APPAYEKLSAEQSPP

SEQ ID NO: 481 - protein, 16 aa, Homo sapiens
  MISC_FEATURE (9)...(9): The amino acid at this position can be phosphorylated
APPAYEKLSAEQSPPP

SEQ ID NO: 482 - protein, 17 aa, Homo sapiens
  MISC_FEATURE (9)...(9): The amino acid at this position can be phosphorylated
APPAYEKLSAEQSPPPY

SEQ ID NO: 483 - protein, 9 aa, Homo sapiens
  MISC_FEATURE (8)...(8): The amino acid at this position can be phosphorylated
PPAYEKLSA

SEQ ID NO: 484 - protein, 12 aa, Homo sapiens
  MISC_FEATURE (8)...(8): The amino acid at this position can be phosphorylated
PPAYEKLSAEQS

SEQ ID NO: 485 - protein, 9 aa, Homo sapiens
  MISC_FEATURE (7)...(7): The amino acid at this position can be phosphorylated
PAYEKLSAE

SEQ ID NO: 486 - protein, 12 aa, Homo sapiens
  MISC_FEATURE (7)...(7): The amino acid at this position can be phosphorylated
PAYEKLSAEQSP

SEQ ID NO: 487 - protein, 12 aa, Homo sapiens
  MISC_FEATURE (6)...(6): The amino acid at this position can be phosphorylated
AYEKLSAEQSPP

SEQ ID NO: 488 - protein, 12 aa, Homo sapiens
  MISC_FEATURE (5)...(5): The amino acid at this position can be phosphorylated
YEKLSAEQSPPP

SEQ ID NO: 489 - protein, 9 aa, Homo sapiens
AAQERRVPR

SEQ ID NO: 490 - protein, 9 aa, Homo sapiens
LLGPGRPYR

SEQ ID NO: 491 - protein, 10 aa, Homo sapiens
ASGPGGGAPR

SEQ ID NO: 492 - protein, 16 aa, Homo sapiens
AQYIKANSKFIGITEL

SEQ ID NO: 493 - protein, 9 aa, Homo sapiens
RLSNRLLLR

SEQ ID NO: 494 - protein, 16 aa, Homo sapiens
AQNILLSNAPLGPQFP

SEQ ID NO: 495 - protein, 11 aa, Homo sapiens
SSDYVIPIGTY

SEQ ID NO: 496 - protein, 13 aa, Homo sapiens
SDAEKSDICTDEY

SEQ ID NO: 497 - protein, 9 aa, Homo sapiens
KCDICTDEY

SEQ ID NO: 498 - protein, 9 aa, Homo sapiens
YMDGTMSQV

SEQ ID NO: 499 - protein, 21 aa, Homo sapiens [sequence omitted]

SEQ ID NO: 500 - protein, 12 aa, Artificial Sequence
  Artificially synthesized PADRE peptide
  misc_feature (3)..(3): Xaa can be any naturally occurring amino acid
AKXVAAWTLKAA

SEQ ID NO: 501 - protein, 9 aa, Epstein-Barr virus
GLCTLVAML

SEQ ID NO: 502 - protein, 9 aa, Homo sapiens
  MISC_FEATURE (4)...(4): The amino acid at this position can be phosphorylated
SIPTVSGQI

SEQ ID NO: 503 - protein, 11 aa, Homo sapiens
  MISC_FEATURE (4)...(4): The amino acid at this position can be phosphorylated
YPLSPAKVNQY

SEQ ID NO: 504 - protein, 11 aa, Homo sapiens
  MISC_FEATURE (4)...(4): The amino acid at this position can be phosphorylated
YPLSPTKISEY

SEQ ID NO: 505 - protein, 9 aa, Homo sapiens
  MISC_FEATURE (8)...(8): The amino acid at this position can be phosphorylated
VPLIRKKSL

SEQ ID NO: 506 - protein, 9 aa, Homo sapiens
  MISC_FEATURE (4)...(4): The amino acid at this position can be phosphorylated
LKLSYLTWV

SEQ ID NO: 507 - protein, 9 aa, Homo sapiens
  MISC_FEATURE (4)...(4): The amino acid at this position can be phosphorylated
KRYSEPVSL

SEQ ID NO: 508 - protein, 9 aa, Homo sapiens
  MISC_FEATURE (8)...(8): The amino acid at this position can be phosphorylated
KSGELLATW

SEQ ID NO: 509 - protein, 8 aa, Homo sapiens
  MISC_FEATURE (4)...(4): The amino acid at this position can be phosphorylated
AAESPSFL

SEQ ID NO: 510 - protein, 9 aa, Homo sapiens
  MISC_FEATURE (5)...(5): The amino acid at this position can be phosphorylated
AMPGSPVEV

SEQ ID NO: 511 - protein, 9 aa, Homo sapiens
  MISC_FEATURE (6)...(6): The amino acid at this position can be phosphorylated
KVLSPTAAK

SEQ ID NO: 512 - protein, 9 aa, Homo sapiens
  MISC_FEATURE (4)...(4): The amino acid at this position can be phosphorylated
KVLSSLVTL

SEQ ID NO: 513 - protein, 9 aa, Homo sapiens
  MISC_FEATURE (4)...(4): The amino acid at this position can be phosphorylated
KVYSSSEFL

SEQ ID NO: 514 - protein, 9 aa, Homo sapiens
  MISC_FEATURE (4)...(4): The amino acid at this position can be phosphorylated
  MISC_FEATURE (8)...(8): The amino acid at this position can be phosphorylated
RASSDIVSL

SEQ ID NO: 515 - protein, 11 aa, Homo sapiens
  MISC_FEATURE (4)...(4): The amino acid at this position can be phosphorylated
RPASEARAPGL

SEQ ID NO: 516 - protein, 9 aa, Homo sapiens
  MISC_FEATURE (7)...(7): The amino acid at this position can be phosphorylated
RPQKTQSII

SEQ ID NO: 517 - protein, 11 aa, Homo sapiens
  MISC_FEATURE (6)...(6): The amino acid at this position can be phosphorylated
RPRSGSTGSSL

SEQ ID NO: 518 - protein, 7 aa, Homo sapiens
  MISC_FEATURE (3)...(3): The amino acid at this position can be phosphorylated
RPSNPQL

SEQ ID NO: 519 - protein, 9 aa, Homo sapiens
  MISC_FEATURE (4)...(4): The amino acid at this position can be phosphorylated
RPSSGFYEL

SEQ ID NO: 520 - protein, 9 aa, Homo sapiens
  MISC_FEATURE (4)...(4): The amino acid at this position can be phosphorylated
RPTSPIQIM

SEQ ID NO: 521 - protein, 9 aa, Homo sapiens
  MISC_FEATURE (4)...(4): The amino acid at this position can be phosphorylated
RTYSGPMNK

SEQ ID NO: 522 - protein, 11 aa, Homo sapiens
  MISC_FEATURE (5)...(5): The amino acid at this position can be phosphorylated
RVRRSSFLNAK

SEQ ID NO: 523 - protein, 10 aa, Homo sapiens
  MISC_FEATURE (9)...(9): The amino acid at this position can be phosphorylated
RVWEDRPSSA

SEQ ID NO: 524 - protein, 9 aa, Homo sapiens
  MISC_FEATURE (4)...(4): The amino acid at this position can be phosphorylated
SPDSSQSSL

SEQ ID NO: 525 - protein, 8 aa, Homo sapiens
  MISC_FEATURE (5)...(5): The amino acid at this position can be phosphorylated
TDKYSKMM

SEQ ID NO: 526 - protein, 9 aa, Homo sapiens
  MISC_FEATURE (4)...(4): The amino acid at this position can be phosphorylated
VLDSPASKK

SEQ ID NO: 527 - protein, 10 aa, Homo sapiens
  MISC_FEATURE (9)...(9): The amino acid at this position can be phosphorylated
VPKSGRSSSL

SEQ ID NO: 528 - protein, 9 aa, Homo sapiens
  MISC_FEATURE (4)...(4): The amino acid at this position can be phosphorylated
YPSSPRKAL

SEQ ID NO: 529 - protein, 9 aa, Homo sapiens
  MISC_FEATURE (5)...(5): The amino acid at this position can be phosphorylated
SARRTPVSY

(19) United States
(12) Patent Application Publication
(10) Pub. No.: US 2019/0374627 A1
(43) Pub. Date: Dec. 12, 2019

(54) TARGET PEPTIDES FOR CANCER THERAPY AND DIAGNOSTICS

(71) Applicants: University of Virginia Patent Foundation, Charlottesville, VA (US); The University of Birmingham, Birmingham (GB)

(72) Inventors: Donald F. Hunt, Charlottesville, VA (US); Jeffrey Shabanowitz, Charlottesville, VA (US); Paisley Trantham Myers, Charlottesville, VA (US); Mark Cobbold, Winchester, MA (US); Nico Büttner, Schallstadt (DE); Stacy Alyse Malaker, Oakland, CA (US); Sarah Penny, Birmingham (GB)

(73) Assignees: University of Virginia Patent Foundation, Charlottesville, VA (US); The University of Birmingham, Birmingham (GB)

(21) Appl. No.: 16/098,634

(22) PCT Filed: May 5, 2017
(86) PCT No.: PCT/US2017/031266

§ 371 Date: Nov. 2, 2018

Related U.S. Application Data
(60) Provisional application No. 62/332,139, filed on May 5, 2016.

(51) Int. Cl. A61K 39/00; C12N 5/0784; C12N 5/0783; C07K 16/28; A61P 35/00; A61K 35/17; A61K 39/39; G01N 33/574; C07K 14/74; C07K 14/33

(52) U.S. Cl. CPC A61K 39/001111; C12N 5/0639; C12N 5/0636; C07K 16/2833; A61P 35/00; C12N 2502/1121; A61K 39/39; G01N 33/57492; C07K 14/70539; C07K 14/33; C12N 2502/1114; A61K 35/17

(57) ABSTRACT

A set of target peptides are presented by HLA class I molecules on the surface of hepatocellular carcinoma (HCC) ceils and/or esophageal cancer cells. They are envisioned to among other things (a) stimulate an immune response to the proliferative disease, e.g., HCC and/or esophageal cancer, (b) function as immunotherapeutics in adoptive T-cell therapy or as a vaccine, (c) facilitate antibody recognition of tumor boundaries in surgical pathology samples, (d) act as biomarkers for early detection and/or diagnosis of the disease, and (e) act as targets in the generation anti-body-like molecules which recognize the target-peptide/MHC complex.

### CROSS-REFERENCE TO RELATED APPLICATION

This application claims the benefit of U.S. Provisional Application Ser. No. 62/332,139, filed May 5, 2016, the disclosure of which is incorporated herein by reference in its entirety.

### GRANT STATEMENT

This invention was made with government support under Grant No. AI033993 awarded by National Institutes of Health. The government has certain rights in the invention.

### REFERENCE TO SEQUENCE LISTING

The Sequence Listing associated with the instant disclosure has been electronically submitted to the United States Patent and Trademark Office as a 137 kilobyte ASCII text file created on May 3, 2017 and entitled “3062_13_PCT_ST25.txt”. The Sequence Listing submitted via EFS-Web is hereby incorporated by reference in its entirety.

### TECHNICAL FIELD

The presently disclosed subject matter relates to diagnostics and therapeutics. In particular, it relates to immunotherapies and diagnostics in the context of proliferative diseases such as cancer.

### BACKGROUND

The mammalian immune system has evolved a variety of mechanisms to protect the host from cancerous cells. An important component of this response is mediated by cells referred to as T cells. Cytotoxic T lymphocytes (CTL) are specialized T cells that primarily function by recognizing and killing cancerous cells or infected cells, but they can also function by secreting soluble molecules referred to as cytokines that can mediate a variety of effects on the immune system. T helper cells primarily function by recognizing antigen on specialized antigen presenting cells, and in turn secreting cytokines that activate B cells, T cells, and macrophages. A variety of evidence suggests that immunotherapy designed to stimulate a tumor-specific CTL response would be effective in controlling cancer. For example, it has been shown that human CTL recognize sarcomas (Slovin et al., 1986), renal cell carcinomas (Schendel et al., 1993), colorectal carcinomas (Jacob et al., 1997), ovarian carcinomas (Peoples et al., 1993), pancreatic carcinomas (Peiper et al., 1997), squamous tumors of the head and neck (Yasumura et al., 1993), and squamous carcinomas of the lung (Slingluff et al., 1994; Yoshino et al., 1994). The largest number of reports of human tumor-reactive CTLs, however, has concerned melanomas (Boon et al., 1994). The ability of tumor-specific CTL to mediate tumor regression, in both human (Parmiani et al., 2002; Weber, 2002) and animal models, suggests that methods directed at increasing CTL activity would likely have a beneficial effect with respect to tumor treatment.

Liver Cancer (hepatocellular carcinoma, HCC) is the sixth most common cancer in the world. Incidence and mortality are growing in Europe and most parts of the world. Chronic liver diseases predispose for the development of HCC (liver cirrhosis of any etiology, alcoholic liver disease, chronic viral infection, autoimmunehepatitis, etc.). Unfortunately, diagnosis is often made in late stages of the disease and to this day only very limited treatment options are available for HCC, especially in advanced stage disease (Llovet et al., 2012). Since HCC has been shown to be immunogenic (Wada et al., 1998; Takayama et al., 2000; Parmiani & Anichini, 2006), immunotherapy is considered to be a promising new treatment modality. The identification of novel and specific tumor antigens provides the basis for the development of an efficient anti-cancer immunotherapy. Only a few HCC-specific tumor antigens have been characterized so far (Breous & Thimme, 2011; Buonaguro et al., 2013), although it has been shown that up to 10.000 different peptides can be presented with WIC-I-molecules on the surface of tumor cells (Zarling et al., 2000).

Esophageal cancer is also a leading cause of death from cancer worldwide. The two principal types of esophageal cancer are squamous cell carcinoma and adenocarcinoma. Both are relatively uncommon in the U.S., comprising approximately 1% of all cancers. However, the incidence of adenocarcinoma is rising at a rapid rate. The 5-year survival rates for localized and all stages combined are 34% and 17%, respectively. Moreover, there is no currently reliable method for early detection or for the prediction of treatment outcome.

Barrett's esophagus (BE), high-grade dysplasia (HGD), and invasive cancer are thought to comprise a multi-step process in the development of esophageal adenocarcinoma (EAC or OEAC). HGD has been considered as the immediate precursor of invasive adenocarcinoma, and most patients with HGD develop cancer. No intervention currently exists that prevents the progression of BE or HGD to esophageal cancer. The traditional methods for diagnosing esophageal cancer include endoscopy and barium swallow, but the poor specificity and sensitivity of these methods results in their detection only at an advanced stage. Recently however, prognostic and predictive markers have been identified that aid in the diagnosis of esophageal cancer.

Alteration in the phosphorylation status of cellular signaling proteins is a hallmark of malignant transformation. This altered phosphorylation status leads to up- or downregulation of signaling pathways, which are indispensable for tumor growth. Deregulated phosphorylation can create neoantigens that bind to major histocompatibility complex (MHC) molecules and the phosphorylation affects the antigenic identity of the presented epitopes (Mohammed et al., 2008). It has been shown that phosphoproteins are processed and presented on tumor cells and that they are recognized by the immune system in a phosphorylation-dependent manner (Zarling et al., 2006). Further studies revealed that MHC class-I molecules seem to have a higher affinity towards the phosphorylated peptide in comparison to the unphosphorylated counterpart and that the phosphate group is exposed outwards in direction to the T cell receptor (TCR) in order to improve contact with the TCR (Mohammed et al., 2008, see particularly FIG. 1 therein). The phosphoproteome therefore seems to be an attractive target for cancer immunotherapy (Zarling et al., 2000; Zarling et al., 2006; Mohammed et al., 2008; Cobbold et al., 2013).

In order for CTL to kill or secrete cytokines in response to a cancer cell, the CTL must first recognize the cancer cell (Townsend & Bodmer, 1989). This process involves the interaction of the T cell receptor, located on the surface of the CTL, with what is generically referred to as an MHC-peptide complex which is located on the surface of the cancerous cell. MHC (major histocompatibility-complex)-encoded molecules have been subdivided into two types, and are referred to as class I and class II MHC-encoded molecules. In the human immune system, MHC molecules are referred to as human leukocyte antigens (HLA). Within the MHC complex, located on chromosome six, are three different loci that encode for class I MHC molecules. MHC molecules encoded at these loci are referred to as HLA-A, HLA-B, and HLA-C. The genes that can be encoded at each of these loci are extremely polymorphic, and thus, different individuals within the population express different class I MHC molecules on the surface of their cells. HLA-A1, HLA-A2, HLA-A3, HLA-B7, HLA-B14, HLA-B27, and HLA-B44 are examples of different class I MHC molecules that can be expressed from these loci.

The peptides which associate with the MHC molecules can either be derived from proteins made within the cell, in which case they typically associate with class I MHC molecules (Rock & Goldberg, 1999); or they can be derived from proteins which are acquired from outside of the cell, in which case they typically associate with class II MHC molecules (Watts, 1997). The peptides that evoke a cancer-specific CTL response most typically associate with class I MHC molecules. The peptides themselves are typically nine amino acids in length, but can vary from a minimum length of eight amino acids to a maximum of fourteen amino acids in length. Tumor antigens can also bind to class II MHC molecules on antigen presenting cells and provoke a T helper cell response. The peptides that bind to class II MHC molecules are generally twelve to nineteen amino acids in length, but can be as short as ten amino acids and as long as thirty amino acids.

The process by which intact proteins are degraded into peptides is referred to as antigen processing. Two major pathways of antigen processing occur within cells (Rock & Goldberg, 1999). One pathway, which is largely restricted to professional antigen presenting cells such as dendritic cells, macrophages, and B cells, degrades proteins that are typically phagocytosed or endocytosed into the cell. Peptides derived from this pathway can be presented on either class I or to class II MHC molecules. A second pathway of antigen processing is present in essentially all cells of the body. This second pathway primarily degrades proteins that are made within the cells, and the peptides derived from this pathway primarily bind to class I MHC molecules. Antigen processing by this latter pathway involves polypeptide synthesis and proteolysis in the cytoplasm, followed by transport of peptides to the plasma membrane for presentation. These peptides, initially being transported into the endoplasmic reticulum of the cell, become associated with newly synthesized class I MHC molecules and the resulting complexes are then transported to the cell surface. Peptides derived from membrane and secreted proteins have also been identified. In some cases these peptides correspond to the signal sequence of the proteins which is cleaved from the protein by the signal peptidase. In other cases, it is thought that some fraction of the membrane and secreted proteins are transported from the endoplasmic reticulum into the cytoplasm where processing subsequently occurs. Once bound to the class I MHC molecule, the peptides are recognized by antigen-specific receptors on CTL. Several methods have been developed to identify the peptides recognized by CTL, each method of which relies on the ability of a CTL to recognize and kill only those cells expressing the appropriate class I MHC molecule with the peptide bound to it. Mere expression of the class I MHC molecule is insufficient to trigger the CTL to kill the target cell if the antigenic peptide is not bound to the class I MHC molecule. Such peptides can be derived from a non-self source, such as a pathogen (for example, following the infection of a cell by a bacterium or a virus) or from a self-derived protein within a cell, such as a cancerous cell. The tumor antigens from which the peptides are derived can broadly be categorized as differentiation antigens, cancer/testis antigens, mutated gene products, widely expressed proteins, viral antigens and most recently, phosphopeptides derived from dysregulated signal transduction pathways. (Zarling et al., 2006).

Immunization with HCC-derived, class I or class II MHC-encoded molecule associated peptides, or with a precursor polypeptide or protein that contains the peptide, or with a gene that encodes a polypeptide or protein containing the peptide, are forms of immunotherapy that can be employed in the treatment of HCC. Identification of the immunogens is a necessary first step in the formulation of the appropriate immunotherapeutic agent or agents. Although a large number of tumor-associated peptide antigens recognized by tumor reactive CTL have been identified, there are few examples of antigens that are derived from proteins that are selectively expressed on a broad array of tumors, as well as associated with cellular proliferation and/or transformation.

Attractive candidates for this type of antigen are peptides derived from proteins that are differentially phosphorylated on serine (Ser), threonine (Thr), and tyrosine (Tyr; Zarling et al., 2000). Due to the increased and dysregulated phosphorylation of cellular proteins in transformed cells as compared to normal cells, tumors are likely to present a unique subset of phosphorylated peptides on the cell surface that are available for recognition by cytotoxic T-lymphocytes (CTL). Presently, there is no way to predict which protein phosphorylation sites in a cell will be unique to tumors, survive the antigen processing pathway, and be presented to the immune system in the context of phosphopeptides bound to class I MHC molecules.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides in some embodiments compositions comprising at least or about 1, 2, 3, 4, 5, 6, 7, 8, 9, 10, or more synthetic target peptides. In some embodiments, each synthetic target peptide is about or at least 6, 7, 8, 9, 10, 11, 12, 13, 14 or 15 amino acids long, optionally between 8 and 50 amino acids long; and comprises an amino acid sequence selected from the group consisting of SEQ ID NOs: 1-448 and 502-529, and further wherein said composition optionally has the ability to stimulate a T cell-mediated immune response to at least one of the synthetic target peptides and/or is capable of eliciting a memory T cell response to at least one of the synthetic target peptides. In some embodiments, the synthetic target peptide comprises an amino acid sequence selected from the group consisting of SEQ ID NOs: 4, 5, 10, 11, 15, 24, 32, 33, 37, 38, 41, 42, 52, 59, 63, 64, 66, 72, 75, 80, 83-89, 91, 95, 96, 106-108, 113, 115-117, 122, 123, 127, 128, 130-132, 146-149, 157, 158, 160, 161, 163-165, 167, 174, 179, 181, 185-188, 195, 198, 203, 206, 210, 212, 215, 218, 221, 222, 224, 226, 231-233, 237, 243, 245, 253, 261, 266, 270, 274, 275, 276, 281, 285-287, 292, 293, 295, 297, 299, 303-305, 317, 320, 337, 338, 340, 343-349, 351-364, 367-371, 373, 377, 379, 382, 383, 385, 386, 393-412, 414-426, 429-436, 438-448, 464, 502, and 509-529. In some embodiments, at least one of the synthetic target peptides comprises a substitution of a serine residue with a homo-serine residue. In some embodiments, at least one of the synthetic target peptides is a phosphopeptide that comprises a non-hydrolyzable phosphate group. In some embodiments, the composition is immunologically suitable for use in a hepatocellular carcinoma (HCC) patient and/or an esophageal cancer patient. In some embodiments, the composition comprises at least 2, 3, 4, or 5 different target peptides, at least 10 different target peptides, or at least 15 different target peptides.

In some embodiments, at least one of the synthetic target peptides is capable of binding to an MHC class I molecule selected from the group consisting of an HLA-A*0201 molecule, an HLA A*0101 molecule, an HLA A*0301 molecule, an HLA B*4402 molecule, an HLA B*0702 molecule, and an HLA B*2705 molecule.

In some embodiments the composition is capable of increasing the 5-year survival rate of HCC patients and/or esophageal cancer patients treated with the composition by at least 20 percent relative to average 5-year survival rates that could have been expected without treatment with the composition. In some embodiments, the composition is capable of increasing the survival rate of HCC and/or esophageal cancer patients treated with the composition by at least 20 percent relative to a survival rate that could have been expected without treatment with the composition. In some embodiments, the composition is capable of increasing the treatment response rate of HCC and/or esophageal cancer patients treated with the composition by at least 20 percent relative to a treatment rate that could have been expected without treatment with the composition. In some embodiments, the composition is capable of increasing the overall median survival of patients of HCC and/or esophageal cancer patients treated with the composition by at least two months relative to an overall median survival that could have been expected without treatment with the composition.

In some embodiments, the presently disclosed compositions further comprise at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, and TPS.

In some embodiments, the presently disclosed compositions further comprise an adjuvant selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof.

In some embodiments, the presently disclosed compositions comprise a peptide capable of binding to an MEW class I molecule selected from the group consisting of an HLA-A*0201 molecule, an HLA A*0101 molecule, an HLA A*0301 molecule, an HLA B*4402 molecule, an HLA B*0702 molecule, and an HLA B*2705 molecule.

In some embodiments of the presently disclosed compositions, at least one of the synthetic target peptides is phosphorylated on a serine residue, a threonine residue, a tyrosine residue, or any combination thereof.

In some embodiments, the presently disclosed compositions at least one of the synthetic peptides comprises an amino acid sequence selected from the group consisting of SEQ ID NOs: 1-224, 502-508, 515-520, 524, 525, 527, and 529.

In some embodiments, the presently disclosed compositions at least one of the synthetic peptides comprises an amino acid sequence selected from the group consisting of SEQ ID NOs: 4, 5, 10, 11, 15, 24, 32, 33, 37, 38, 41, 42, 52, 59, 63, 64, 66, 72, 75, 80, 83-89, 91, 95, 96, 106-108, 113, 115-117, 122, 123, 127, 128, 130-132, 146-149, 157, 158, 160, 161, 163-165, 167, 174, 179, 181, 185-188, 195, 198, 203, 206, 210, 212, 215, 218, 221, 222, 224, 226, 231-233, 237, 243, 245, 253, 261, 266, 270, 274, 275, 276, 281, 285-287, 292, 293, 295, 297, 299, 303-305, 317, 320, 337, 338, 340, 343-349, 351-364, 367-371, 373, 377, 379, 382, 383, 385, 386, 393-412, 414-426, 429-436, 438-448, 464, 502, and 509-529.

In some embodiments, the presently disclosed compositions at least one of the synthetic target peptides is a phosphopeptide or a phosphopeptide mimetic.

In some embodiments, the presently disclosed compositions at least one of the synthetic target peptides is a phosphopeptide mimetic comprising a mimetic of phosphoserine, phosphothreonine, or phosphotyrosine.

In some embodiments, the presently disclosed compositions the phosphopeptide mimetic is a synthetic molecule in which a phosphorous atom is linked to the serine, threonine, or tyrosine amino acid residue through a carbon.

In some embodiments, the presently disclosed compositions the composition further comprises a tetanus peptide. In some embodiments, the tetanus peptide comprises an amino acid sequence that is at least 90%, 95%, or 100% identical to SEQ ID NO: 449 or SEQ ID NO: 450. In some embodiments, the tetanus peptide is about or at least 10, 11, 12, 13, 14, 15, 16, 17, 18, 19, 20, 21, 22, 23, 24, or 25 natural or non-natural amino acids in length. In some embodiments, the tetanus peptide comprises an amino acid sequence that is at least 90% identical to a 10-25 amino acid subsequence of a wild type tetanus toxoid protein. In some embodiments, the tetanus peptide binds to one or more WIC Class II molecules when administered to a subject. In some embodiments, the tetanus peptide is modified so as to prevent formation of tetanus peptide secondary structures.

The presently disclosed subject matter also provides in some embodiments in vitro populations of dendritic cells comprising the presently disclosed compositions.

The presently disclosed subject matter also provides in some embodiments in vitro populations of CD8⁺ T cells capable of being activated upon being brought into contact with a population of dendritic cells, wherein the dendritic cells comprise a composition of the presently disclosed subject matter.

The presently disclosed subject matter also provides in some embodiments antibodies and antibody-like molecules that specifically bind to a complex of an MHC class I molecule and a peptide, wherein the peptide comprises an amino acid sequence selected from the group consisting of SEQ ID NOs: 1-448 and 502-529. In some embodiments, the peptide comprises an amino acid sequence selected from the group consisting of SEQ ID NOs: 4, 5, 10, 11, 15, 24, 32, 33, 37, 38, 41, 42, 52, 59, 63, 64, 66, 72, 75, 80, 83-89, 91, 95, 96, 106-108, 113, 115-117, 122, 123, 127, 128, 130-132, 146-149, 157, 158, 160, 161, 163-165, 167, 174, 179, 181, 185-188, 195, 198, 203, 206, 210, 212, 215, 218, 221, 222, 224, 226, 231-233, 237, 243, 245, 253, 261, 266, 270, 274, 275, 276, 281, 285-287, 292, 293, 295, 297, 299, 303-305, 317, 320, 337, 338, 340, 343-349, 351-364, 367-371, 373, 377, 379, 382, 383, 385, 386, 393-412, 414-426, 429-436, 438-448, 464, 502, and 509-529. In some embodiments, the antibodies or antibody-like molecules are members of the immunoglobulin superfamily. In some embodiments, the antibodies or antibody-like molecules comprise one or more binding members selected from the group consisting an Fab, Fab′, F(ab′)₂, Fv, and a single-chain antibody.

In some embodiments, the antibodies or antibody-like molecules of the presently disclosed subject matter are conjugated to a therapeutic agent selected from the group consisting of an alkylating agent, an antimetabolite, a mitotic inhibitor, a taxoid, a vinca alkaloid, and an antibiotic. In some embodiments, an antibody or antibody-like molecule of the presently disclosed subject matter is a T cell receptor, optionally conjugated to a CD3 agonist.

The presently disclosed subject matter also provides in some embodiments in vitro populations of T cells transfected with a nucleic acid, optionally an mRNA, encoding a T cell receptor of the presently disclosed subject matter.

The presently disclosed subject matter also provides in some embodiments methods for treating and/or preventing cancer comprising administering to a subject in need thereof a therapeutically effective dose of a presently disclosed composition and/or a composition comprising at least one target peptide comprising an amino acid sequence as set forth in any of SEQ ID NOs: 1-448 and 502-529. In some embodiments, the cancer is HCC, and the at least one target peptide comprises an amino acid sequence as set forth in any of SEQ ID NOs: 1-448. In some embodiments, the at least one target peptide comprises an amino acid sequence as set forth in any of SEQ ID NOs: 4, 5, 10, 11, 15, 24, 32, 33, 37, 38, 41, 42, 52, 59, 63, 64, 66, 72, 75, 80, 83-89, 91, 95, 96, 106-108, 113, 115-117, 122, 123, 127, 128, 130-132, 146-149, 157, 158, 160, 161, 163-165, 167, 174, 179, 181, 185-188, 195, 198, 203, 206, 210, 212, 215, 218, 221, 222, 224, 226, 231-233, 237, 243, 245, 253, 261, 266, 270, 274, 275, 276, 281, 285-287, 292, 293, 295, 297, 299, 303-305, 317, 320, 337, 338, 340, 343-349, 351-364, 367-371, 373, 377, 379, 382, 383, 385, 386, 393-412, 414-426, 429-436, 438-448, 464, 502, and 509-529. In some embodiments, the at least one target peptide comprises an amino acid sequence as set forth in any of SEQ ID NOs: 16, 36, 49, 54, 81, 105, 111, 137, 139, 140, 149, 156, 159, 166, 182, 191, 193, 196, 205, 216, 242, 249, 252, 257, 259, 262, 268, 269, 271, 289, 294, 296, 374, 376, 380, 381, 385, 428, and 502-508.

The presently disclosed subject matter also provides in some embodiments methods of treating and/or preventing hepatocellular carcinoma (HCC) and/or esophageal cancer comprising administering to a subject in need thereof a therapeutically effective dose of a presently disclosed composition or a composition comprising at least one target peptide in combination with a pharmaceutically acceptable carrier.

The presently disclosed subject matter also provides in some embodiments methods for treating and/or preventing cancer, optionally hepatocellular carcinoma (HCC) and/or esophageal cancer. In some embodiments, the presently disclosed methods comprise administering to a subject in need thereof a therapeutically effective dose of the presently disclosed CD8⁺ T cells in combination with a pharmaceutically acceptable carrier.

The presently disclosed subject matter also provides in some embodiments methods for treating and/or preventing cancer, optionally hepatocellular carcinoma (HCC) and/or esophageal cancer, comprising administering to a subject in need thereof a presently disclosed in vitro population of dendritic cells in combination with a pharmaceutically acceptable carrier.

The presently disclosed subject matter also provides in some embodiments methods for treating and/or preventing hepatocellular carcinoma (HCC) and/or esophageal cancer, comprising administering to a subject in need thereof a presently disclosed population of CD8⁺ T cells in combination with a pharmaceutically acceptable carrier.

The presently disclosed subject matter also provides in some embodiments methods for making a cancer vaccine, optionally a cancer vaccine for use in treating and/or preventing hepatocellular carcinoma (HCC) and/or esophageal cancer. In some embodiments, the presently disclosed methods comprise combining a presently disclosed composition with an the adjuvant selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof and a pharmaceutically acceptable carrier; and placing the composition, adjuvant, and pharmaceutical carrier into a container, optionally into a syringe.

The presently disclosed subject matter also provides in some embodiments methods for screening target peptides for inclusion in the presently disclosed immunotherapy compositions or for use in the presently disclosed methods for using the presently disclosed compositions. In some embodiments, the methods comprise administering the target peptide to a human; determining whether the target peptide is capable of inducing a target peptide-specific memory T cell response in the human; and selecting the target peptide for inclusion in an immunotherapy composition if the target peptide elicits a memory T cell response in the human.

The presently disclosed subject matter also provides in some embodiments methods for determining a prognosis of a hepatocellular carcinoma (HCC) patient and/or an esophageal cancer patient, the methods comprising administering to the patient a target peptide comprising an amino acid sequence as set forth in any of SEQ ID NOs: 1-448 and 502-529, wherein the target peptide is associated with the patient's HCC and/or esophageal cancer; determining whether the target peptide is capable of inducing a target peptide-specific memory T cell response in the patient; and determining that the patient has a better prognosis if the patient mounts a memory T cell response to the target peptide than if the patient did not mount a memory T cell response to the target peptide. In some embodiments, the target peptide comprises an amino acid sequence as set forth in any of SEQ ID NOs: 4, 5, 10, 11, 15, 24, 32, 33, 37, 38, 41, 42, 52, 59, 63, 64, 66, 72, 75, 80, 83-89, 91, 95, 96, 106-108, 113, 115-117, 122, 123, 127, 128, 130-132, 146-149, 157, 158, 160, 161, 163-165, 167, 174, 179, 181, 185-188, 195, 198, 203, 206, 210, 212, 215, 218, 221, 222, 224, 226, 231-233, 237, 243, 245, 253, 261, 266, 270, 274, 275, 276, 281, 285-287, 292, 293, 295, 297, 299, 303-305, 317, 320, 337, 338, 340, 343-349, 351-364, 367-371, 373, 377, 379, 382, 383, 385, 386, 393-412, 414-426, 429-436, 438-448, 464, and 509-529.

The presently disclosed subject matter also provides in some embodiments kits comprising at least one target peptide composition comprising at least one target peptide comprising an amino acid sequence as set forth in any of SEQ ID NOs: 1-448 and 502-529 and a cytokine and/or an adjuvant. In some embodiments, the target peptide comprises an amino acid sequence as set forth in any of SEQ ID NOs: 4, 5, 10, 11, 15, 24, 32, 33, 37, 38, 41, 42, 52, 59, 63, 64, 66, 72, 75, 80, 83-89, 91, 95, 96, 106-108, 113, 115-117, 122, 123, 127, 128, 130-132, 146-149, 157, 158, 160, 161, 163-165, 167, 174, 179, 181, 185-188, 195, 198, 203, 206, 210, 212, 215, 218, 221, 222, 224, 226, 231-233, 237, 243, 245, 253, 261, 266, 270, 274, 275, 276, 281, 285-287, 292, 293, 295, 297, 299, 303-305, 317, 320, 337, 338, 340, 343-349, 351-364, 367-371, 373, 377, 379, 382, 383, 385, 386, 393-412, 414-426, 429-436, 438-448, 464, and 509-529. In some embodiments, the presently disclosed kits comprise at least 2, 3, 4, or 5 target peptide compositions. In some embodiments, the at least one target peptide composition is one of the compositions of disclosed herein. In some embodiments, the cytokine is selected from the group consisting of a transforming growth factor (TGF), optionally TGF-alpha and/or TGF-beta; insulin-like growth factor-I; insulin-like growth factor-II; erythropoietin (EPO); an osteoinductive factor; an interferon, optionally interferon-alpha, interferon-beta, and/or interferon-gamma; and a colony stimulating factor (CSF), optionally macrophage-CSF (M-CSF), granulocyte-macrophage-CSF (GM-CSF), and/or granulocyte-CSF (G-CSF). In some embodiments, the adjuvant is selected from the group consisting of montanide ISA-51, QS-21, a tetanus helper peptide, GM-CSF, cyclophosphamide, bacillus Calmette-Guerin (BCG), corynbacterium parvum, levamisole, azimezone, isoprinisone, dinitrochlorobenezene (DNCB), a keyhole limpet hemocyanin (KLH), complete Freund's adjuvant, incomplete Freund's adjuvant, a mineral gel, aluminum hydroxide, lysolecithin, a pluronic polyol, a polyanion, an adjuvant peptide, an oil emulsion, dinitrophenol, and diphtheria toxin (DT). In some embodiments, the cytokine is selected from the group consisting of a nerve growth factor, optionally nerve growth factor (NGF) beta; a platelet-growth factor; a transforming growth factor (TGF), optionally TGF-alpha and/or TGF-beta; insulin-like growth factor-I; insulin-like growth factor-II; erythropoietin (EPO); an osteoinductive factor; an interferon, optionally interferon-α, interferon-β, and/or interferon-γ; a colony stimulating factor (CSF), optionally macrophage-CSF (M-CSF), granulocyte-macrophage-CSF (GM-CSF), and/or granulocyte-CSF (G-CSF); an interleukin (IL), optionally IL-1, IL-1a, IL-2, IL-3, IL-4, IL-5, IL-6, IL-7, IL-8, IL-9, IL-10, IL-11, IL-12; IL-13, IL-14, IL-15, IL-16, IL-17, and/or IL-18; LIF; EPO; kit-ligand; fms-related tyrosine kinase 3 (FLT-3; also called CD135); angiostatin; thrombospondin; endostatin; tumor necrosis factor; and lymphotoxin (LT).

In some embodiments, the presently disclosed kits further comprise at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, and TPS.

In some embodiments, the at least one target peptide comprises an amino acid sequence as set forth in any of SEQ ID NOs: 1-448 and 502-529. In some embodiments, the at least one target peptide is selected from the group consisting of SEQ ID NOs: 4, 5, 10, 11, 15, 24, 32, 33, 37, 38, 41, 42, 52, 59, 63, 64, 66, 72, 75, 80, 83-89, 91, 95, 96, 106-108, 113, 115-117, 122, 123, 127, 128, 130-132, 146-149, 157, 158, 160, 161, 163-165, 167, 174, 179, 181, 185-188, 195, 198, 203, 206, 210, 212, 215, 218, 221, 222, 224, 226, 231-233, 237, 243, 245, 253, 261, 266, 270, 274, 275, 276, 281, 285-287, 292, 293, 295, 297, 299, 303-305, 317, 320, 337, 338, 340, 343-349, 351-364, 367-371, 373, 377, 379, 382, 383, 385, 386, 393-412, 414-426, 429-436, 438-448, 464, 502, and 509-529. In some embodiments, the at least one target peptide is selected from the group consisting of SEQ ID NOs: 1-224, 502-508, 515-520, 524, 525, 527, and 528, and any combination thereof. In some embodiments, the at least one target peptide is selected from the group consisting of SEQ ID NOs: 502-508, and any combination thereof. In some embodiments, the at least one target peptide composition comprises one or more synthetic target peptides that specifically bind to an HLA molecule listed in Table 1 and/or that comprises an amino acid sequence at least 90% identical, optionally 100% identical, to one of the SEQ ID NOs: listed in Tables 2, 3, 5-7, and 14. In some embodiments, the kit comprises at least two synthetic target peptides, wherein the at least two synthetic target peptides are in separate containers.

TABLE 1
Anchor Residues for Different HLA Molecules

| | Residue | Residue | Residue | Residue | Residue 9 or |
|---|---|---|---|---|---|
| | 1 | 2 | 3 | 7 | Last Residue |
| HLA A*0101 | | T, S | D, E | | Y |
| HLA A*0201 | | L, M | | | V |
| HLA A*0301 | | L, M | | | K |
| HLA A*24 | | Y, W, M | | | L, F, W |
| HLA B*0702 | | P | | | L, M, V, F |
| HLA B*1508 | | P, A | | | Y |
| HLA B*2705 | R | R | | | L, F, K, R, M |
| HLA B*4402 | | E | | | F, Y, W |
| HLA C*0501 | Y | P, A | D | | F, I, L, M, V |
| HLA C*0602 | F, Y | R, Y | A, F, Y | K, Q, R | I, L, M, V |

In some embodiments, the presently disclosed kits further comprise instructions related to determining whether the at least one synthetic target peptide of the at least one synthetic target peptide composition is capable of inducing a T cell memory response that is a T cell central memory response (Tcm) when the at least one synthetic target peptide composition is administered to a patient.

In some embodiments, the presently disclosed kits further comprise a tetanus peptide. In some embodiments, the tetanus peptide comprises an amino acid sequence that is at least 90%, 95%, or 100% identical to SEQ ID NO: 449 or SEQ ID NO: 450. In some embodiments, the tetanus peptide is about or at least 10, 11, 12, 13, 14, 15, 16, 17, 18, 19, 20, 21, 22, 23, 24, or 25 natural or non-natural amino acids in length. In some embodiments, the tetanus peptide comprises an amino acid sequence that is at least 90% identical to a 10-25 amino acid subsequence of a wild type tetanus toxoid protein. In some embodiments, the tetanus peptide binds to one or more MEW Class II molecules when administered to a subject.

### BRIEF DESCRIPTION OF THE FIGURES

FIGS. 1A-1C present a summary of the characteristics of the first 250 MHC-I-pP analyzed and their presentation. FIG. 1A is a bar graph showing that more different MHC-I-pP were presented per gram of tissue during progression of liver disease. FIG. 1B is a bar graph showing that a greater diversity but not more MHC-I-pP were presented by each cell during the course of disease. FIG. 1C is a bar graph of predicted HLA-binding of the first 250 identified HCC-specific MHC-I-pP. The most common represented types are HLA-A*0201, HLA-B*0702, HLA-B*2705, and HLA-C*07. In over ninety percent of the cases, the amino acid serine (S) was phosphorylated in HCC-specific MHC-I-pP, and the phosphate moiety was most often present at amino acid position 4 of the peptides. Abbreviations—AHL: adjacent “healthy” (i.e., non-cirrhotic) liver; ACL: adjacent cirrhotic liver; HCC: hepatocellular carcinoma tissue; HepG2: HepG2 cell line; OEAC: esophageal cancer.

FIG. 2 presents Boolean combination gates calculated and plotted as column graphs in order to assess the percentage of reactive T cells. Abbreviations—HD: healthy donor; HH: hereditary hemochromatosis patient; APC: antigen-presenting cell; IFNg-PE: phycoerythrin-labeled interferon gamma; CD107a: Cluster of Differentiation antigen 107a; IFNg: interferon gamma; TNFa: tumor necrosis factor alpha.

FIGS. 3A-3C present a summary of the characteristics of phosphopeptide-specific T cells in the blood compartment from patients with chronic liver disease. FIG. 3A is a bar graph summarizing the results of the analysis of ppCTL by 7-day flow cytometry, which revealed that phosphopeptide-specific cells (pP) produced multiple cytokines and the similar amounts of cytotoxic markers in comparison to virus-specific T cells (viral). FIG. 3B is a bar graph showing that ppCTLs resided in the memory compartment as determined by surface marker expression of CD45RA and CD27. As a control, the majority of unspecific T cells in PBMCs displayed a naive phenotype. FIG. 3C is a plot showing that ppCTLs expressed higher amounts of CTLA-4, but not PD-1, on their surface in comparison to virus-specific T cells. Expanded ppCTLs recognized the phosphorylated embodiment of the peptide IMDRtPEKL (SEQ ID NO: 14 with Thr5 phosphorylated), but did not recognize the unphosphorylated counterpart IMDRTPEKL (SEQ ID NO: 14 with Thr5 non-phosphorylated), meaning that the expanded ppCTLs were phosphopeptide-specific rather than reactive toward the unphosphorylated counterpart peptide. Abbreviations—n.s.: not significant; viral: virus-specific T cell response; pP: ppCTL response; CD3: Cluster of Differentiation Antigen 3; CD107a: Cluster of Differentiation Antigen 107a; IFNg: interferon gamma; TNFa: tumor necrosis factor alpha; N: negative control (DMSO); EFF: effector T cells; MEM: memory T cells; CTLA-4: cytotoxic T-lymphocyte-associated protein 4; PD-1: programmed cell death protein 1.

FIGS. 4A and 4B are graphs showing rapid expansion of liver-derived lymphocytes. FIG. 4A is a graph showing that the rapid expansion protocol (REP) described in Dudley et al., 2003 worked independently if the lymphocyte culture was initiated from “healthy” intrahepatic lymphocytes (DDL REP; open squares), cirrhotic intrahepatic lymphocytes (Cirrhotic IHL; open circles), or cancerous tumor-infiltrating lymphocytes (HCC TIL REP; black squares) tissue. FIG. 4B is a graph showing that CD8⁺ pre-selected cultures (black squares) expanded significantly faster than unselected cultures (open circles) in the first 14 days (d).

FIGS. 5A and 5B present the results of experiments that showed that ppCTLs were lost using REP but could be restored if lymphocyte cultures were expanded antigen-specifically. FIG. 5A is a statistical summary of all positive ppCTL-responses comparing unspecific and specific expansion. No difference is observed for virus-specific T cells. FIG. 5B is a Box and Whiskers plot of the data from Table 23 calculated with GraphPad (GraphPad Software, Inc., La Jolla, Calif., United States of America) showing that ppCTLs after expansion were functional, produced multiple cytokines, and were able to degranulate. The boxes extend from the 25th to 75th percentiles. The whiskers represent min and max values. Abbreviations—pP: phosphopeptide; n.s.: not significant; CD107a: Cluster of Differentiation antigen 107a; IFNg: interferon gamma; TNFa: tumor necrosis factor alpha.

FIG. 6 is a LogoPlot depicting the residue frequency at each position of exemplary 9-mer HLA-*A02-phosphopeptides. HLA-A*2-associated phosphopeptides have unique characteristics that distinguish them from nonphosphorylated peptides. There was a strong preference for a positively charged amino acid at position 1, a leucine at position 2, the phosphopeptide at position 4, and a valine or leucine at position 9.

FIG. 7 is an example of a typical analysis and graphical representation of a phosphopeptide-specific CD8⁺ T cell response. Boolean combinatorial gates were calculated from an intracellular cytokine staining (ICS) experiment and the percentage of cytokine producing or degranulating T cells was assessed. In this case, PBMCs were reactive (>1% reactive cells) against the viral peptide NLVPMVATV (CMV, pp65; SEQ ID NO: 455) and MHC-I-pP AVVsPPALHNA (SEQ ID NO: 6) from Bromodomain-containing protein 4 (BRD4). In both cases (viral peptide and phosphopeptide), T cells responses were comparable in quantity and quality (polyfunctional cytokine production). Abbreviations—CD107a: Cluster of Differentiation Antigen 107a; IFNg: interferon gamma; TNFa: tumor necrosis factor alpha; positive: positive control (PMA/Ionomycin); negative: negative control (DMSO).

FIG. 8 is another example of a typical analysis and graphical representation of a phosphopeptide-specific CD8⁺ T cell response showing an ex vivo CD8⁺ T cell response against the phosphopeptide RVAsPTSGV (SEQ ID NO: 57) from insulin receptor substrate-2 (IRS2) after stimulation of PBMCs for 4 hours with the peptide. Abbreviations—DMSO: dimethyl sulfoxide; IRS2 (RVAsPTSGV): Insulin receptor substrate 2 phosphopeptide RVAsPTSGV (SEQ ID NO: 57); IFNg-PE: phycoerythrin-labeled interferon gamma; TNFa-PE-Cy7: TNFa labeled with phycoerythrin-Cyanin 5.1; IFNg: interferon gamma; TNFa: tumor necrosis factor alpha; RVAsPTSGV: phosphopeptide RVAsPTSGV (SEQ ID NO: 57); positive: positive control (PMA/Ionomycin); negative: negative control (DMSO).

### BRIEF DESCRIPTION OF THE SEQUENCE LISTING

A more complete understanding of the presently disclosed subject matter can be obtained by reference to the accompanying Sequence Listing, when considered in conjunction with the subsequent Detailed Description. The embodiments presented in the Sequence Listing are intended to be exemplary only and should not be construed as limiting the presently disclosed subject matter to the listed embodiments.

SEQ ID NOs: 1-448 are the amino acid sequences of exemplary MHC class I target peptides associated with HCC. Additional details with respect to optional post-translations modifications (e.g., phosphorylation) of the amino acid sequences of SEQ ID NOs: 1-448 are provided in Tables 2-13 herein below.

SEQ ID NOs: 449 and 450 are the amino acid sequences of exemplary tetanus helper peptides.

SEQ ID NO: 451 is the amino acid sequence of a peptide from the cytomegalovirus (CMV; also referred to as human herpesvirus 5) phosphoprotein 65. It corresponds to amino acids 495-503 of Accession No. YP_081531.1 in the GENBANK® biosequence database.

SEQ ID NOs: 452-499 are exemplary peptides derived from various tumor-associated antigens (TAAs).

SEQ ID NO: 500 is the amino acid sequence of a Pan DR T helper epitopes (PADRE) peptide.

SEQ ID NO: 501 is the amino acid sequence of a peptide derived from the Epstein-Barr virus (EBV; also known as human herpesvirus 4) BMLF1 protein, which corresponds to amino acids 259-267 of Accession No. YP 401660.1 in the GENBANK® biosequence database.

SEQ ID NOs: 502-508 are the amino acid sequences of exemplary MHC class I target peptides associated with esophageal cancer. Additional details with respect to optional post-translations modifications (e.g., phosphorylation) of the amino acid sequences of SEQ ID NOs: 502-508 are provided in Table 14 herein below.

SEQ ID NOs: 509-529 are the amino acid sequences of additional exemplary MHC class I target peptides associated with HCC. Additional details with respect to optional post-translations modifications (e.g., phosphorylation) of the amino acid sequences of SEQ ID NOs: 509-529 are provided in Tables 2, 3, 6, and 9 herein below.

### DETAILED DESCRIPTION

### I. General Considerations

Advanced hepatocellular carcinoma (HCC) and esophageal cancer are serious therapeutic challenges and novel approaches are urgently needed for the treatment of these conditions. The immune system can specifically identify and eliminate tumor cells on the basis of their expression of tumor-associated antigens (TAA). This process is referred to as tumor immune surveillance, whereby the immune system identifies cancerous and/or precancerous cells and eliminates them before they can cause harm (Corthay, 2014). Therefore, immunotherapy is considered a promising new treatment modality. The basis for every immunotherapeutic approach is the identification of specific targets, which distinguishes the malignant cells from healthy cells. Very few immunogenic TAA have been characterized so far in general and even less for HCC in particular, which is considered to be an immunogenic tumor (Prieto et al., 2015).

During the course of chronic liver disease, for example, several mutations and epigenetic changes accumulate in the liver cells, which finally lead to a dysregulation of major signaling pathways that are important for malignant transformation (Whittaker et al., 2010). Similar processes are likely to be occurring in cells that give rise to esophageal cancer. Therefore, deregulation of signaling pathways with altered and augmented phosphorylation of cellular proteins is a hallmark of tumorigenesis generally and malignant transformation in particular. Phosphoproteins involved in these signaling cascades can be degraded to phosphopeptides that are presented by major histocompatibility complex (MHC) class I and -II molecules and recognized by T cells (Zarling et al., 2000; Zarling et al., 2006; Cobbold et al., 2013). The contributions of phosphopeptide-specific T cells to immune surveillance in the development of liver cancer in chronic liver disease and in tumorigenesis leading to esophageal cancer are unclear.

It was hypothesized that phosphopeptides are presented by MHC molecules with increasing amounts on the surface of altered hepatocytes and esophageal cells with progression of liver disease towards HCC and tumorigenesis leading to esophageal cancer. It was further hypothesized that the immune system monitors the liver for malignant transformed hepatocytes and the esophagus for tumorigenic cells and clears those cells with the help of phosphopeptide-specific cytotoxic T lymphocytes (ppCTLs).

Therefore, MHC class I-associated phosphopeptides (MHC-I-pP) that are presented on the surface of HCC and cells involved with tumorigenesis leading to esophageal cancer were investigated using a mass spectrometry approach. In order to show the immunogenicity of these novel identified tumor antigens, the T cell responses to these newly identified phosphoantigens in healthy individuals, in patients with chronic liver diseases, and in patients with HCC were characterized. The quantity and quality of these tumor-specific T cell responses was correlated with the patients' clinical course and HCC tumor and esophageal cancer progression.

As such, disclosed herein is a set of 460 phosphopeptides presented to the immune system by class I MHC molecules derived from human hepatocellular carcinoma (HCC), some of which are also derived from esophageal cancer, and seven (7) phosphopeptides presented to the immune system by class I MHC molecules derived from esophageal cancer but not HCC. These peptides have at least the potential to (a) stimulate an immune response to the cancer; (b) function as immunotherapeutics in adoptive T-cell therapy or as vaccine; (c) function as targets for immunotherapy based on bispecific antibodies; (d) facilitate antibody recognition of the tumor boundaries in surgical pathology samples; and (e) act as biomarkers for early detection of the disease, although the presently disclosed subject matter is not limited to just these applications.

### II. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

Following long-standing patent law convention, the terms “a”, “an”, and “the” refer to “one or more” when used in this application, including the claims. Thus, in some embodiments the phrase “a peptide” refers to one or more peptides.

The term “about”, as used herein to refer to a measurable value such as an amount of weight, time, dose (e.g., therapeutic dose), etc., is meant to encompass in some embodiments variations of ±20%, in some embodiments ±10%, in some embodiments ±5%, in some embodiments ±1%, in some embodiments ±0.1%, in some embodiments ±0.5%, and in some embodiments ±0.01% from the specified amount, as such variations are appropriate to perform the disclosed methods.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience. Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

As used herein, the phrase “amino acid sequence as set forth in any of SEQ ID NOs: [A]-[B]” refers to any amino acid sequence that is disclosed in any one or more of SEQ ID NOs: A-B. In some embodiments, the amino acid sequence is any amino acid sequence that is disclosed in any of the SEQ ID NOs. that are present in the Sequence Listing. In some embodiments, the phrase refers to the full length sequence of any amino acid sequence that is disclosed in any of the SEQ ID NOs. that are present in the Sequence Listing, such that an “amino acid sequence as set forth in any of SEQ ID NOs: [A]-[B]” refers to the full length sequence of any of the sequences disclosed in the Sequence Listing. By way of example and not limitation, in some embodiments an “amino acid sequence as set forth in any of SEQ ID NOs: 1-448 and 502-529” refers to the full length amino acid sequence disclosed in any of SEQ ID NOs: 1-448 and 502-529 and not to a subsequence of any of SEQ ID NOs: 1-448 and 502-529.

The presently disclosed subject matter relates in some embodiments to post-translationally-modified immunogenic therapeutic target peptides, e.g., phosphopeptides, for use in immunotherapy and diagnostic methods of using the target peptides, as well as methods of selecting the same to make compositions for immunotherapy, e.g., in vaccines and/or in compositions useful in adaptive cell transfer.

### III. Target Peptides

The presently disclosed subject matter relates in some embodiments to immunogenic therapeutic target peptides for use in immunotherapy and diagnostic methods of using the target peptides, as well as methods of selecting the same to make compositions for immunotherapy, e.g., in vaccines and/or in compositions useful in adaptive cell transfer. In some embodiments, the target peptides of the presently disclosed subject matter are post-translationally modified by being provided with a phosphate group, (i.e., “phosphopeptides”). In some embodiments, the target peptides of the presently disclosed subject matter are modified by having an oxidized methionine.

The target peptides of the presently disclosed subject matter are in some embodiments not the entire proteins from which they are derived. They are in some embodiments from 6 to 50 contiguous amino acid residues of the native human protein. They can in some embodiments contain exactly, about, or at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. The peptides of the presently disclosed subject matter can also in some embodiments have a length that falls in the ranges of 6-10, 9-12, 10-13, 11-14, 12-15, 15-20, 20-25, 25-30, 30-35, 35-40, and 45-50 amino acids. Exactly, about, or at least 1, 2, 3, 4, 5, 6, 7, 8, 9, 10, 11, 12, 13, 14, 15, 16, 17, or more of the amino acid residues within the recited sequence of a target peptide can phosphorylated.

Target peptides can be modified and analogs (using for example, beta-amino acids, L-amino acids, N-methylated amino acids, amidated amino acids, non-natural amino acids, retro inverse peptides, peptoids, PNA, halogenated amino acids) can be synthesized that retain their ability to stimulate a particular immune response, but which also gain one or more beneficial features, such as those described below. Thus, particular target peptides can, for example, have use for treating and vaccinating against multiple cancer types.

In some embodiments, substitutions can be made in the target peptides at residues known to interact with the MHC molecule. Such substitutions can in some embodiments have the effect of increasing the binding affinity of the target peptides for the MHC molecule and can also increase the half-life of the target peptide-MHC complex, the consequence of which is that the analog is in some embodiments a more potent stimulator of an immune response than is the original peptide.

Additionally, the substitutions can in some embodiments have no effect on the immunogenicity of the target peptide per se, but rather can prolong its biological half-life or prevent it from undergoing spontaneous alterations which might otherwise negatively impact on the immunogenicity of the peptide.

The target peptides disclosed herein can in some embodiments have differing levels of immunogenicity, MHC binding and ability to elicit CTL responses against cells displaying a native target peptide, e.g., on the surface of a tumor cell.

The amino acid sequences of the target peptides can in some embodiments be modified such that immunogenicity and/or binding is enhanced. In some embodiments, the modified target peptide binds an MHC class I molecule about or at least 10%, 20%, 30%, 40%, 50%, 60%, 70%, 80%, 90%, 95%, 100%, 110%, 125%, 150%, 175%, 200%, 225%, 250%, 275%, 300%, 350%, 375%, 400%, 450%, 500%, 600%, 700%, 800%, 1000%, or more tightly than its native (unmodified) counterpart.

However, given the exquisite sensitivity of the T-cell receptor, it cannot be foreseen whether such enhanced binding and/or immunogenicity will render a modified target peptide still capable of inducing an activated CTL that will cross react with the native target peptide being displayed on the surface of a tumor. Indeed, it is disclosed herein that the binding affinity of a target peptide does not predict its functional ability to elicit a T cell response.

Target peptides of the presently disclosed subject matter can in some embodiments be mixed together to form a cocktail. The target peptides can in some embodiments be in an admixture, or they can in some embodiments be linked together in a concatamer as a single molecule. Linkers between individual target peptides can in some embodiments be used; these can, for example, in some embodiments be formed by any 10 to 20 amino acid residues. The linkers can in some embodiments be random sequences, or they can in some embodiments be optimized for degradation by dendritic cells.

In certain specified positions, a native amino acid residue in a native human protein can in some embodiments be altered to enhance the binding to the MHC class I molecule. These can occur in “anchor” positions of the target peptides, often in positions 1, 2, 3, 9, or 10. Valine (V), alanine (A), lysine (K), leucine (L), isoleucine (I), tyrosine (Y), arginine (R), phenylalanine (F), proline (P), glutamic acid (E), glutamine (Q), threonine (T), serine (S), aspartic acid (D), tryptophan (W), and methionine (M) can also be used in some embodiments as improved anchoring residues. Anchor residues for different HLA molecules are listed below. Anchor residues for HLA molecules are listed in Table 1.

In some embodiments, the immunogenicity of a target peptide is measured using transgenic mice expressing human MHC class I genes. For example, “ADD Tg mice” express an interspecies hybrid class I MHC gene, AAD, which contains the alpha-1 and alpha-2 domains of the human HLA-A2.1 gene and the alpha-3 transmembrane and cytoplasmic domains of the mouse H-2Dd gene, under the direction of the human HLA-A2.1 promoter. Immunodetection of the HLA-A2.1 recombinant transgene established that expression was at equivalent levels to endogenous mouse class I molecules. The mouse alpha-3 domain expression enhances the immune response in this system. Compared to unmodified HLA-A2.1, the chimeric HLA-A2.1/H2-Dd MHC Class I molecule mediates efficient positive selection of mouse T cells to provide a more complete T cell repertoire capable of recognizing peptides presented by HLA-A2.1 Class I molecules. The peptide epitopes presented and recognized by mouse T cells in the context of the HLA-A2.1/H2-Dd class I molecule are the same as those presented in HLA-A2.1⁺ humans. This transgenic strain facilitates the modeling of human T cell immune responses to HLA-A2 presented antigens, and identification of those antigens. This transgenic strain is a preclinical model for design and testing of vaccines for infectious diseases or cancer therapy involving optimal stimulation of CD8⁺ cytolytic T cells.

In some embodiments, the immunogenicity of a modified target peptide is determined by the degree of Interferon gamma and/or TNF-α production of T-cells from ADD Tg mice immunized with the target peptide, e.g., by immunization with target peptide pulsed bone marrow derived dendritic cells.

In some embodiments, the modified target peptides are about or at least 10%, 20%, 30%, 40%, 50%, 60%, 70%, 80%, 90%, 95%, 100%, 110%, 125%, 150%, 175%, 200%, 225%, 250%, 275%, 300%, 350%, 375%, 400%, 450%, 500%, 600%, 700%, 800%, 1000%, 1500%, 2000%, 2500%, 3000%, 4000%, 5000%, or more immunogenic, e.g., in terms of numbers of Interferon gamma and/or TNF-alpha positive (i.e., “activated”) T-cells relative to numbers elicited by native target peptides in ADD Tg mice immunized with target peptides pulsed bone marrow derived dendritic cells. In some embodiments, the modified target peptides are able to elicit CD8⁺ T cells which are cross-reactive with the modified and the native target peptide in general and when such modified and native target peptides are complexed with MEW class I molecules in particular. In some embodiments, the CD8⁺ T cells which are cross-reactive with the modified and the native target peptides are able to reduce tumor size by about or at least 10%, 20%, 30%, 40%, 50%, 60%, 70%, 80%, 90%, 95%, 97%, or 99% in a NOD/SCID/IL-2Ryc^{-/-} knock out mouse (which has been provided transgenic T cells specific form an immune competent donor) relative to IL-2 treatment without such cross-reactive CD8⁺ T cells.

The term “capable of inducing a target peptide-specific memory T cell response in a patient” as used herein relates to eliciting a response from memory T cells (also referred to as “antigen-experienced T cell”) which are a subset of infection- and cancer-fighting T cells that have previously encountered and responded to their cognate antigen. Such T cells can recognize foreign invaders, such as bacteria or viruses, as well as cancer cells. Memory T cells have become “experienced” by having encountered antigen during a prior infection, encounter with cancer, or previous vaccination. At a second encounter with the cognate antigen, e.g., by way of an initial inoculation with a target peptide of the presently disclosed subject matter, memory T cells can reproduce to mount a faster and stronger immune response than the first time the immune system responded to the invader (e.g., through the body's own consciously unperceived recognition of a target peptide being associated with diseased tissue). This behavior can be assayed in T lymphocyte proliferation assays, which can reveal exposure to specific antigens. Memory T cells comprise two subtypes: central memory T cells (T_{CM} cells) and effector memory T cells (TEM cells). Memory cells can be either CD4⁺ or CD8⁺. Memory T cells typically express the cell surface protein CD45RO. Central memory T_{CM} cells generally express L-selectin and CCR7, they secrete IL-2, but not IFNγ or IL-4. Effector memory TEM cells, however, generally do not express L-selectin or CCR7 but produce effector cytokines like IFNγ and IL-4.

A memory T cell response generally results in the proliferation of memory T cell and/or the upregulation or increased secretion of the factors such as CD45RO, L-selectin, CCR7, IL-2, IFNγ, CD45RA, CD27, and/or IL-4. In some embodiments, the target peptides of the presently disclosed subject matter are capable of inducing a T_{CM} cell response associated with L-selectin, CCR7, IL-2 (but not IFNγ or IL-4) expression and/secretion (see e.g., Hamann et al., 1997). In some embodiments, a T_{CM} cell response is associated with an at least or about 1%, 5%, 10%, 15%, 20%, 25%, 30%, 35%, 45%, 50%, 55%, 60%, 65%, 70%, 75%, 80%, 90%, 95%, 97%, 98%, 99%, 100%, 125%, 150%, 175%, 200%, 250%, 300%, 400%, 500%, 600%, 700%, 800%, 900%, 1000%, 1500%, 2000%, or more increase in T cell CD45RO/RA, L-selectin, CCR7, or IL-2 expression and/secretion.

In some embodiments, the target peptides of the presently disclosed subject matter are capable of inducing a CD8⁺ T_{CM} cell response in a patient the first time that patient is provided the composition including the selected target peptides. As such, the target peptides of the presently disclosed subject matter can in some embodiments be referred to as “neo-antigens”. Although target peptides might be considered “self” for being derived from self-tissue, they generally are only found on the surface of cells with a dysregulated metabolism, e.g., aberrant phosphorylation, they are likely never presented to immature T cells in the thymus. As such, these “self” antigens act are neo-antigens because they are nevertheless capable of eliciting an immune response.

In some embodiments, about or at least 1%, 5%, 10%, 15%, 20%, 25%, 30%, 35%, 45%, 50%, 55%, 60%, 65%, 70%, 75%, 80%, 90%, 95%, 97%, 98%, or 99% of T cells activated by particular target peptide in a particular patient sample are T_{CM} cells. In some embodiments, a patient sample is taken exactly, about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days after an initial exposure to a particular target peptide and then assayed for target peptide specific activated T cells and the proportion of T_{CM} cells thereof. In some embodiments, the compositions of the presently disclosed subject matter are able to elicit a CD8⁺ T_{CM} cell response in at least or about 1%, 5%, 10%, 15%, 20%, 25%, 30%, 35%, 45%, 50%, 55%, 60%, 65%, 70%, 75%, 80%, 90%, 95%, 97%, 98%, 99%, or 100% of patients and/or healthy volunteers. In some embodiments, the compositions of the presently disclosed subject matter are able to elicit a CD8⁺ T_{CM} cell response in a patient about or at least 1%, 5%, 10%, 15%, 20%, 25%, 30%, 35%, 45%, 50%, 55%, 60%, 65%, 70%, 75%, 80%, 90%, 95%, 97%, 98%, 99%, or 100% of patients and/or healthy volunteers specific to all or at least or about 1, 2, 3, 4, 5, 6, 7, 8, 9, or 10 target peptides in the composition. In some embodiments, the aforementioned T cell activation tests are done by ELISpot assay.

### III. Phosphopeptides

In some embodiments, the target peptides of the presently disclosed subject matter are post-translationally-modified by being provided with a phosphate group (referred to herein as “phosphopeptides”). The term “phosphopeptides” includes MHC class I-specific phosphopeptides. Exemplary MHC class I phosphopeptides of the presently disclosed subject matter that are associated in some embodiments with hepatocellular carcinoma are set forth in Tables 2-14. The amino acid sequences of these phosphopeptides are set forth in SEQ ID NOs: 1-448 and 502-529. In Tables 2-14, phosphoserine, phosphothreonine, and phosphotyrosine residues are indicated by “s”, “t”, and “y”, respectively. Oxidized methionine residues are indicated by “m”. “Gene Name” refers to the name of the Gene as set forth in the UniProt biosequence database. A lowercase “c” in a peptide sequence indicates that in some embodiments the cysteine is present in a cysteine-cysteine disulfide bond at the surface of a cell and, in some embodiments, is presented to the immune system as such.

TABLE 2
Exemplary Class I MHC Phosphopeptides on HCC that
are Specific for HLA-A*0201

| SEQ | | | | | |
|---|---|---|---|---|---|
| ID | | | | UniProt | |
| NO. | Sequence^{#} | Start | Stop | Acc. No. | Gene Name ^{%} |
| 1 | AIMRsPQMV | 187 | 195 | P35222 | CTNNB1 |
| 2 | ALDsGASLLHL | 482 | 492 | P57078 | RIPK4 |
| 3 | ALGNtPPFL | 111 | 119 | Q7Z739 | YTHDF3 |
| 4 | ALMGsPQLV | 178 | 186 | P14923 | JUP |
| 5 | ALMGsPQLVAA | 178 | 188 | P14923 | JUP |
| 6 | AVVsPPALHNA | 905 | 915 | O60885 | BRD4 |
| 7 | DLKRRsmSI | 175 | 183 | Q96N67 | DOCK7 |
| 8 | ELFSsPPAV | 953 | 960 | O94916 | NFAT5 |
| 9 | FLDtPIAKV | 320 | 328 | Q969G9 | NKD1 |
| 10 | GIDsPSSSV | 77 | 85 | Q5JSP0 | FGD3 |
| 11 | GLDsGFHSV | 297 | 305 | O75427 | LRCH4 |
| 12 | GLIsPVWGA | 50 | 58 | Q76N32 | CEP68 |
| 13 | GLLDsPTSI | 218 | 226 | Q07352 | ZFP36L1 |
| 14 | IMDRtPEKL | 126 | 134 | O75815 | BCAR3 |
| 15 | KAFsPVR | 2 | 8 | Q02363 | ID2 |
| 16 | KAFsPVRSV | 2 | 10 | Q02363 | ID2 |
| 17 | KIAsEIAQL | 541 | 549 | Q8WXE0 | CASKIN2 |
| 18 | KIGsIIFQV | 1223 | 1231 | Q460N5 | PARP14 |
| 19 | KLAsPELERL | 70 | 79 | P05412 | JUN |
| 20 | KLDsPRVTV | 38 | 46 | D3DUF1 | FAM86A |
| 21 | KLFPDtPLAL | 587 | 596 | Q12906 | ILF3 |
| 22 | KLIDIVsSQKV | 461 | 471 | O14757 | CHEK1 |
| 23 | KLIDRTEsL | 197 | 205 | P33241 | LSP1 |
| 24 | KLMsDVEDV | 1940 | 1948 | Q9NSI6 | BRWD1 |
| 25 | KLMsPKADVKL | 44 | 54 | Q86T90 | KIAA1328 |
| 26 | KQDsLVINL | 647 | 655 | Q9Y5B9 | SUPT16H |
| 27 | KTMsGTFLL | 592 | 600 | P52630 | STAT2 |
| 28 | KVAsLLHQV | 330 | 338 | Q8NFZ5 | TNIP2 |
| 29 | LMFsPVTSL | 887 | 895 | Q9C0A6 | SETD5 |
| 30 | RASsLSITV | 839 | 847 | Q6ZS17 | FAM65A |
| 31 | RLAsASRAL | ? | ? | Unknown | Unknown |
| 32 | RLAsLQSEV | ? | ? | Unknown | Unknown |
| 33 | RLAsYLDKV | 90 | 98 | P08727 | KRT19 |
| 34 | RLAsYLDRV | 90 | 98 | P05783 | KRT18 |
| 35 | RLDsYVR | 129 | 135 | Q9Y5R8 | TRAPPC1 |
| 36 | RLDsYVRSL | 129 | 137 | Q9Y5R8 | TRAPPC1 |
| 37 | RLFsKEL | 30 | 36 | Q15543 | TAF13 |
| 38 | RLFsKELR | 30 | 37 | Q15543 | TAF13 |
| 39 | RLFsKELRC | 30 | 38 | Q15543 | TAF13 |
| 40 | RLLsDLEEL | 245 | 253 | Q8IWP9 | CCDC28A |
| 41 | RLLsTDAEAV | 168 | 177 | Q15545 | TAF7 |
| 42 | RLSDtPPLL | 205 | 213 | P20337 | RAB3B |
| 43 | RLSsPLHFV | 400 | 408 | Q8NC44 | FAM134A |
| 44 | RMYsFDDVL | 802 | 810 | Q8WWI1 | LMO7 |
| 45 | RQAsIELPSM | 249 | 258 | P33241 | LSP1 |
| 46 | RQAsIELPSMAV | 249 | 260 | P33241 | LSP1 |
| 47 | RQAsLSISV | 526 | 534 | Q9BZL6 | PRKD2 |
| 48 | RQDsTPGKVFL | 61 | 71 | P13056 | NR2C1 |
| 49 | RQIsQDVKL | 165 | 173 | Q01433 | AMPD2 |
| 50 | RQLsALHRA | 31 | 39 | P61313 | RPL15 |
| 51 | RQLsSGVSEI | 79 | 88 | P04792 | HSPB1 |
| 52 | RSLsESYEL | 104 | 112 | Q6DN90 | IQSEC1 |
| 53 | RSLsQELVGV | 333 | 342 | Q5VUA4 | ZNF318 |
| 54 | RTFsPTYGL | 426 | 434 | O15061 | SYNM |
| 55 | RTLsHISEA | 450 | 458 | Q6ZS17 | FAM65A |
| 56 | RTYsGPMNKV | 53 | 62 | Q8WVV4 | POF1B |
| 57 | RVAsPTSGV | 1097 | 1105 | Q9Y4H2 | IRS2 |
| 58 | SImsPEIQL | 154 | 162 | Q96RK0 | CIC |
| 59 | SISsMEVNV | 149 | 157 | Q9BQY9 | DBNDD2 |
| 60 | SISStPPAV | 260 | 268 | Q9H8Y8 | GORASP2 |
| 61 | SLFGGsVKL | 103 | 111 | Q8WUM4 | PDCD6IP |
| 62 | SLFsGDEENA | 22 | 31 | Q53EL6 | PDCD4 |
| 63 | SLFsPQNTL | 973 | 981 | Q5VT52 | RPRD2 |
| 64 | SLFsSEESNL | 403 | 412 | P04004 | VTN |
| 65 | SLFsSEESNLGA | 30 | 38 | Q15543 | TAF13 |
| 66 | SLHDIQLsL | 694 | 702 | Q9H7U1 | CCSER2 |
| 67 | SLQPRSHsV | 448 | 456 | Q9Y2H5 | PLEKHA6 |
| 68 | SLQsLETSV | 1233 | 1241 | P23634 | ATP2B4 |
| 69 | SMSsLSREV | 2117 | 2125 | O15027 | SEC16A |
| 70 | SMTRsPPRV | 248 | 256 | Q9BRL6 | SRSF8 |
| 71 | SVKPRRTsL | 766 | 774 | P15822 | HIVEP1 |
| 72 | TVFsPTLPAA | 375 | 384 | Q7Z2W4 | ZC3HAV1 |
| 73 | VLFSsPPQM | 67 | 75 | P33991 | MCM4 |
| 74 | VLLsPVPEL | 552 | 560 | Q9H1A4 | ANAPC1 |
| 75 | VLYsPQMAL | 372 | 380 | O60502 | MGEA5 |
| 76 | VMIGsPKKV | 1437 | 1445 | Q68CZ2 | TNS3 |
| 77 | yLQSRYYRA | 359 | 367 | Q9H422 | HIPK3 |
| 510 | AMPGsPVEV | 39 | 47 | O43439 | CBFA2T2 |
| 512 | KVLsSLVTL | 17 | 25 | E7ENL8 | ARHGEF7 |
| 513 | KVYsSSEFL | 39 | 47 | V9GYV0 | MAST3 |
| 514 | RASsDIVSL | 120 | 128 | V9GZ26 | FAM110A |
| 514 | RASsDIVsL | 120 | 128 | V9GZ26 | FAM110A |
| 521 | RTYsGPMNK | 53 | 61 | Q8WVV4 | POF1B |

TABLE 3
Exemplary Class I MHC Phosphopeptides on HCC that
are Specific for HLA-B*0702

| SEQ | | | | | |
|---|---|---|---|---|---|
| ID | | | | UniProt | |
| NO. | Sequence^{#} | Start | Stop | Acc. No. | Gene Name |
| 78 | APDsPRAFL | ? | ? | Unknown | Unknown |
| 79 | APRKGsFSAL | 5 | 14 | Q13619 | CUL4A |
| 80 | APRNGsGVAL | 549 | 558 | Q7L9B9 | EEPD1 |
| 81 | APRRYsSSL | 697 | 705 | Q68EM7 | ARHGAP17 |
| 82 | APRsPPPSRP | 8 | 17 | Q9NSA8 | SOCS-1 |
| 83 | APSLFHLNtL | 1230 | 1239 | Q96QB1 | DLC1 |
| 84 | APSSARAsPLL | ? | ? | Unknown | Unknown |
| 85 | FPLDsPKTLVL | 2071 | 2081 | Q5VUA4 | ZNF318 |
| 86 | FPRRHsVTL | 49 | 57 | Q07352 | ZFP36L1 |
| 87 | FRGRYRsPY | 91 | 99 | Q14498 | RBM39 |
| 88 | FRKsMVEHY | 97 | 106 | Q14088 | RAB33A |
| 89 | GPPYQRRGsL | 359 | 368 | P41161 | ETV5 |
| 90 | GPRPGsPSAL | 266 | 275 | Q9UJJ7 | RPUSD1 |
| 91 | GPRSAsLL | 51 | 60 | Q9Y4H4 | GPSM3 |
| 92 | GPRSAsLLSL | 51 | 60 | Q9Y4H4 | GPSM3 |
| 93 | GPRSAsLLsL | 51 | 60 | Q9Y4H4 | GPSM3 |
| 94 | GPRsPKAPP | 71 | 79 | Q6PJ34 | ARHGAP4 |
| 95 | HPKRSVsL | 160 | 167 | O60238 | BNIP3L |
| 96 | HRYsTPHAF | 230 | 238 | P04049 | RAF1 |
| 97 | KPAsPKFIVTL | 512 | 522 | Q6PJT7 | ZC3H14 |
| 98 | KPPYRSHsL | 442 | 450 | Q96GE4 | CEP95 |
| 99 | KPRPLsMDL | 279 | 287 | Q9BY89 | KIAA1671 |
| 100 | KPRPPPLsP | 328 | 336 | Q8NFL1 | TRIP10 |
| 101 | KPRRFsRsL | 209 | 217 | Q7L4I2 | RSRC2 |
| 101 | KPRRFsRSL | 209 | 217 | Q7L4I2 | RSRC2 |
| 102 | KPRsPFSKI | 185 | 193 | Q9BXF6 | RAB11F1P5 |
| 103 | KPRsPPRAL | 249 | 257 | Q86TG7 | PEG10 |
| 104 | KPRsPPRALVL | 249 | 259 | Q86TG7 | PEG10 |
| 105 | KPRsPVVEL | 667 | 675 | P25098 | GRK2 |
| 106 | KPSsPRGSL | 134 | 142 | Q96IF1 | AJUBA |
| 107 | KPSsPRGSLL | 134 | 143 | Q96IF1 | AJUBA |
| 108 | KPVsPKSGTL | 246 | 255 | Q14155 | ARHGEF7 |
| 109 | KPYsPLASL | 70 | 78 | Q13469 | NFATC2 |
| 110 | KRAsGQAFEL | 13 | 22 | P16949 | STMN1 |
| 111 | LPAsPRARL | 443 | 451 | Q3KQU3 | MAP7D1 |
| 112 | LPIFSRLsI | 483 | 491 | P47974 | ZFP36L2 |
| 113 | LPKGLSAsL | 541 | 549 | Q6PKG0 | LARP1 |
| 113 | LPKGLsASL | 541 | 549 | Q6PKG0 | LARP1 |
| 114 | LPRGsSPSVL | 105 | 114 | Q9GZN2 | TGIF2 |
| 115 | LPRPAsPAL | 2247 | 2255 | P78559 | MAP1A |
| 116 | LPRSSsMAA | 361 | 369 | Q9UQB8 | BAIAP2 |
| 117 | LPRSSsMAAGL | 361 | 371 | Q9UQB8 | BAIAP2 |
| 118 | MPRQPsATRL | 134 | 143 | Q6NZ67 | MZT2B |
| 119 | QPRtPSPLVL | 172 | 181 | P33241 | LSP1 |
| 120 | RARGIsPIVF | 303 | 312 | Q96MU7 | YTHDC1 |
| 121 | RKLsVILIL | 3 | 11 | Q13433 | SLC39A6 |
| 122 | RLLsPQQPAL | 177 | 186 | Q14814 | MEF2D |
| 123 | RPAFFsPSL | 299 | 307 | Q6ICG6 | KIAA0930 |
| 124 | RPAKsMDSL | 323 | 331 | Q7Z6I6 | ARHGAP30 |
| 125 | RPAsAGAmL | 198 | 206 | Q14814 | MEF2D |
| 126 | RPAsPAAKL | 512 | 520 | Q9P2N6 | KANSL3 |
| 127 | RPAsPEPEL | ? | ? | Unknown | Unknown |
| 128 | RPAsPGPSL | 646 | 654 | Q8IY33 | MICALL2 |
| 129 | RPAsPQRAQL | ? | ? | Unknown | Unknown |
| 130 | RPAsPSLQL | 277 | 285 | Q8WUF5 | PPP1R13L |
| 131 | RPAsPSLQLL | 277 | 286 | Q8WUF5 | PPP1R13L |
| 132 | RPAsYKKKSML | 764 | 774 | P16234 | PDGFRA |
| 133 | RPDsPTRPTL | 1646 | 1655 | Q7RTP6 | MICAL3 |
| 134 | RPDsRLGKTEL | 1225 | 1235 | Q9BYW2 | SETD2 |
| 135 | RPDVAKRLsL | 282 | 291 | O75815 | BCAR3 |
| 136 | RPFHGISTVsL | 1417 | 1427 | Q5VZ89 | DENND4C |
| 137 | RPFsPREAL | 742 | 750 | Q86V48 | LUZP1 |
| 138 | RPGsRQAGL | 175 | 184 | Q96JY6 | PDLIM2 |
| 139 | RPIsPGLSY | 364 | 372 | Q16204 | CCDC6 |
| 140 | RPIsPPHTY | 1303 | 1311 | Q9Y6N7 | ROBO1 |
| 141 | RPIsPRIGAL | 93 | 102 | Q9Y6I3 | EPN1 |
| 142 | RPKLSsPAL | 15 | 23 | Q09472 | EP300 |
| 143 | RPKsNIVLL | 222 | 230 | P11836 | MS4A1 |
| 144 | RPKsPLSKM | 1576 | 1584 | Q9HCD6 | TANC2 |
| 145 | RPKsVDFDSL | 455 | 464 | Q9Y5K6 | CD2AP |
| 146 | RPKtPPVVI | 245 | 253 | Q96A49 | SYAP1 |
| 147 | RPLsLLLAL | 12 | 20 | P78504 | JAG1 |
| 148 | RPLsVVYVL | 43 | 51 | O95382 | MAP3K6 |
| 149 | RPMsESPHM | 280 | 288 | Q07352 | ZFP36L1 |
| 150 | RPNsPSPTAL | 185 | 194 | Q9UKI8 | TLK1 |
| 151 | RPPsPGPVL | 934 | 942 | Q12770 | SCAP |
| 152 | RPQRAtSNVF | 14 | 23 | P24844 | MYL9 |
| 153 | RPRAAtVV | 333 | 340 | P10644 | PRKAR1A |
| 154 | RPRAAtVVA | 333 | 341 | P10644 | PRKAR1A |
| 155 | RPRANsGGVDL | 116 | 1172 | Q92766 | RREB1 |
| 156 | RPRARsVDAL | 488 | 497 | Q86X29 | LSR |
| 157 | RPRDtRRISL | 1862 | 1871 | Q92508 | PIEZO1 |
| 158 | RPRGsESLL | ? | ? | Unknown | Unknown |
| 159 | RPRGsQSLL | 1040 | 1048 | P21860 | ERBB3 |
| 160 | RPRIPsPIGF | 582 | 591 | Q9NRA8 | EIF4ENIF1 |
| 161 | RPRPAsSPAL | 266 | 275 | A8MQ27 | NEURL1B |
| 162 | RPRPHsAPSL | 108 | 117 | Q5JXC2 | MIIP |
| 163 | RPRPSsAHVGL | 958 | 961 | Q8TF72 | SHROOM3 |
| 164 | RPRPsSVL | 192 | 199 | Q9NTK1 | DEPP |
| 165 | RPRPsSVLRTL | ? | ? | Unknown | Unknown |
| 166 | RPRPVsPSSL | 430 | 439 | P57059 | SIK1 |
| 167 | RPRPVsPSSLL | 430 | 440 | P57059 | SIK1 |
| 168 | RPRsAVEQL | 882 | 890 | Q9HAU0 | PLEKHA5 |
| 169 | RPRsAVLL | 1873 | 1880 | Q12802 | AKAP13 |
| 170 | RPRsISVEEF | 1143 | 1152 | Q7Z333 | SETX |
| 171 | RPRsLEVTI | 239 | 247 | O15553 | MEFV |
| 172 | RPRSLsSPTVTL | 443 | 454 | Q96PU5 | NEDD4L |
| 173 | RPRsMTVSA | 457 | 465 | O43312 | MTSS1 |
| 174 | RPRsMVRSF | 1628 | 1636 | Q14185 | DOCK1 |
| 175 | RPRsPAARL | 111 | 119 | Q9P2Y4 | ZNF219 |
| 176 | RPRsPNMQDL | 214 | 223 | Q6T310 | RASL11A |
| 177 | RPRsPPGGP | 573 | 581 | Q86UZ6 | ZBTB46 |
| 178 | RPRsPPPRAP | 499 | 508 | O43900 | PRICKLE3 |
| 179 | RPRsPPSSP | 41 | 49 | P27815 | PDE4A |
| 180 | RPRsPRENSI | 689 | 698 | Q99700 | ATXN2 |
| 181 | RPRsPRPPP | ? | ? | Unknown | Unknown |
| 182 | RPRsPRQNSI | 689 | 698 | Q99700 | ATXN2 |
| 183 | RPRSPsPIS | 1015 | 1023 | P41594 | GRM5 |
| 184 | RPRsPTGPSNSF | 219 | 230 | Q96I25 | RBM17 |
| 185 | RPRsPTGPSNSFL | 219 | 231 | Q96I25 | RBM17 |
| 186 | RPRsPWGKL | 104 | 112 | O43236 | SEPT4 |
| 187 | RPRsQYNTKL | 494 | 503 | Q7Z6B7 | SRGAP1 |
| 188 | RPRtPLRSL | ? | ? | Unknown | Unknown |
| 189 | RPSsLPDL | 635 | 642 | Q8NFD5 | ARID1B |
| 190 | RPSsPALYF | 261 | 269 | Q9Y3Q8 | TSC22D4 |
| 191 | RPTsFADEL | 285 | 293 | Q9Y4E1 | WASHC2C |
| 192 | RPTsRLNRL | 860 | 868 | Q15788 | NCOA1 |
| 193 | RPVsPFQEL | ? | ? | Unknown | Unknown |
| 194 | RPVsPGKDI | 2115 | 2123 | P31629 | HIVEP2 |
| 195 | RPVSPsSLL | 432 | 440 | P57059 | SIK1 |
| 196 | RPVsTDFAQY | 666 | 675 | O14639 | ABLIM1 |
| 197 | RPVtPVSDL | 63 | 71 | Q13118 | KLF10 |
| 198 | RPWsNSRGL | 71 | 79 | Q9NRR8 | CDC42SE1 |
| 199 | RPWsPAVSA | 380 | 388 | P12755 | SKI |
| 200 | RPYsPPFFSL | 187 | 196 | Q9NYF3 | FAM53C |
| 201 | RPYsQVNVL | 165 | 173 | P46939 | UTRN |
| 202 | RTRsPSPTL | 515 | 523 | Q86UU1 | PHLDB1 |
| 203 | RVRKLPsTTL | 726 | 735 | Q15418 | RPS6KA1 |
| 204 | SPAsPKISL | 493 | 501 | Q8WWM7 | ATXN2L |
| 205 | SPFKRQLsL | 288 | 296 | B7Z5W0 | N/A |
| 206 | SPFLsKRSL | 334 | 342 | Q9NYV4 | CDK12 |
| 207 | SPGLARKRsL | 851 | 860 | Q9H2Y7 | ZNF106 |
| 208 | SPKsPGLKA | 105 | 113 | Q6JBY9 | RCSD1 |
| 209 | SPRERsPAL | 243 | 251 | Q9Y2W1 | THRAP3 |
| 210 | SPRGEASsL | 167 | 175 | Q8IY57 | YAF2 |
| 210 | SPRGEAsSL | 167 | 175 | Q8IY57 | YAF2 |
| 211 | SPRsPGRSL | ? | ? | Unknown | Unknown |
| 212 | SPRsPSGLR | 1449 | 1457 | P49815 | TSC2 |
| 213 | SPRSPsTTYL | 772 | 781 | Q13111 | CHAF1A |
| 214 | SPSsPSVRRQL | 1988 | 1998 | O75179 | ANKRD17 |
| 215 | TPMKKHLsL | 423 | 431 | Q8IX58 | FAM126B |
| 216 | TPRsPPLGL | 755 | 763 | Q16584 | MAP3K11 |
| 217 | TPRsPPLGLI | 755 | 764 | Q16584 | MAP3K11 |
| 218 | VAKRLsL | 285 | 291 | O75815 | BCAR3 |
| 219 | VPRPERRsSL | 668 | 677 | Q6UWJ1 | TMCO3 |
| 220 | VPRsPKHAHSSSL | 242 | 254 | O95425 | SVIL |
| 221 | VPTsPKSSL | 1151 | 1159 | Q70E73 | RAPH1 |
| 222 | YPDPHsPFAV | 240 | 249 | P41162 | ETV3 |
| 223 | YPGGRRsSL | 1037 | 1045 | P22897 | MRC1 |
| 224 | YPYEFsPVKM | 121 | 130 | Q6BEB4 | SP5 |
| 515 | RPAsEARAPGL | 1165 | 1175 | D6W5N0 | MAGI2 |
| 516 | RPQKTQsII | 2136 | 2144 | Q7Z333 | SETX |
| 517 | RPRSGsTGSSL | 2092 | 2102 | Q5TH69 | ARFGEF3 |
| 518 | RPsNPQL | 430 | 436 | Q8IZJ1 | UNC5B |
| 519 | RPSsGFYEL | 156 | 164 | Q9NYF0 | DACT1 |
| 520 | RPTsPIQIM | 1002 | 1010 | Q7Z7B0 | FILIP1 |
| 524 | SPDsSQSSL | 105 | 113 | F8W133 | DDIT3 |
| 525 | TDKYsKMM | 220 | 227 | Q6PI26 | SHQ1 |
| 527 | VPKSGRSSsL | 1271 | 1280 | Q9C0J8 | WDR33 |
| 528 | YPSsPRKAL | 159 | 167 | A6H8W6 | SIPA1L1 |

TABLE 4
Exemplary Class I MHC Phosphopeptides on HCC that
are Specific for HLA-B*2705

| SEQ | | | | | |
|---|---|---|---|---|---|
| ID | | | | UniProt | |
| NO. | Sequence^{#} | Start | Stop | Acc. No. | Gene Name |
| 225 | FRRsPTKSSL | 624 | 633 | Q96PK6 | RBM14 |
| 226 | FRRsPTKSSLD | 624 | 634 | Q96PK6 | RBM14 |
| 227 | FRRsPTKSSLDY | 624 | 635 | Q96PK6 | RBM14 |
| 228 | GRKsPPPSF | 713 | 721 | B4DLE8 | CRYBG3 |
| 229 | GRLsPAYSL | 536 | 544 | Q86UU1 | PHLDB1 |
| 230 | GRLsPVPVPR | 132 | 141 | Q9UKM9 | RALY |
| 231 | GRQsPSFKL | 738 | 746 | Q6IN85 | PPP4R3A |
| 232 | GRsSPPPGY | 173 | 181 | Q99759 | MAP3K3 |
| 233 | KRAsYILRL | 2084 | 2092 | Q96Q15 | SMG1 |
| 234 | KRFsFKKSF | 156 | 164 | P29966 | MARCKS |
| 235 | KRFsFKKsF | 156 | 164 | P29966 | MARCKS |
| 236 | KRFsGTVRL | 47 | 55 | P62906 | RPL10A |
| 237 | KRKsFTSLY | 955 | 963 | Q5SW79 | CEP170 |
| 238 | KRLEKsPSF | 656 | 664 | Q92625 | ANKS1A |
| 239 | KRLsPAPQL | 51 | 59 | Q9UH99 | SUN2 |
| 240 | KRmsPKPEL | 17 | 25 | P41208 | CETN2 |
| 241 | KRWQsPVTK | 593 | 601 | A9Z1X7 | SRRM1 |
| 242 | KRYsGNmEY | 275 | 283 | O95835 | LATS1 |
| 243 | KRYsRALYL | 353 | 361 | Q9UJX3 | ANAPC7 |
| 244 | QRLsPLSAAY | 110 | 119 | Q14774 | HLX |
| 245 | RRAsIITKY | 906 | 914 | Q15849 | SLC14A2 |
| 246 | RRAsLSEIGF | 177 | 186 | Q00537 | CDK17 |
| 247 | RRDsIVAEL | 96 | 104 | O14579 | COPE |
| 248 | RRDsLQKPGL | 377 | 386 | Q9NRM7 | LATS2 |
| 249 | RRFsGTAVY | 652 | 660 | Q6AHZ1 | ZNF518A |
| 250 | RRFsIATLR | 128 | 136 | Q16696 | CYP2A13 |
| 251 | RRFsLTTLR | 124 | 132 | P10632 | CYP2C8 |
| 252 | RRFsPPRRm | 248 | 256 | Q15287 | RNPS1 |
| 253 | RRFsRSDEL | 347 | 355 | P18146 | EGR1 |
| 254 | RRFsRsPIR | 2026 | 2034 | P18583 | SON |
| 255 | RRFSRsPIR | 2026 | 2034 | P18583 | SON |
| 256 | RRFsRsPIRR | 2026 | 2035 | P18583 | SON |
| 257 | RRGsFEVTL | 75 | 83 | Q8IZQ5 | SELENOH |
| 258 | RRIDIsPSTF | 677 | 686 | Q9Y2W1 | THRAP3 |
| 259 | RRIsDPEVF | 788 | 796 | Q4L180 | FILIP1L |
| 260 | RRIsDPQVF | 788 | 796 | Q4L180 | FILIP1L |
| 261 | RRIsQIQQL | 413 | 421 | O60306 | AQR |
| 262 | RRKsQVAEL | 244 | 252 | Q9BYG3 | NIFK |
| 263 | RRLsADIRL | 744 | 752 | O60307 | MAST3 |
| 264 | RRLsELLRY | 449 | 457 | P08238 | HSP90AB1 |
| 265 | RRLsGGSHSY | 332 | 341 | Q13905 | RAPGEF1 |
| 266 | RRLsRKLSL | 553 | 561 | O75167 | PHACTR2 |
| 267 | RRMsFQKP | 88 | 95 | Q8N573 | OXR1 |
| 268 | RRmsLLSVV | 314 | 322 | Q9ULI2 | RIMKLB |
| 269 | RRNsAPVSV | 1175 | 1183 | Q2M1Z3 | ARHGAP31 |
| 270 | RRPsIAPVL | 687 | 695 | Q5JUK3 | KCNT1 |
| 271 | RRPsLLSEF | 67 | 75 | O75376 | NCOR1 |
| 272 | RRPsLVHGY | 31 | 39 | P14324 | FDPS |
| 273 | RRPsYTLGM | 1629 | 1637 | O43166 | SIPA1L1 |
| 274 | RRRsLERLL | 1399 | 1407 | Q96QZ7 | MAGI1 |
| 275 | RRSFsLE | 1598 | 1604 | Q12802 | AKAP13 |
| 276 | RRSsFLQ | 585 | 591 | Q15436 | SEC23A |
| 277 | RRSsFLQVF | 585 | 593 | Q15436 | SEC23A |
| 278 | RRSsIQSTF | 231 | 239 | Q92542 | NCSTN |
| 279 | RRSsQSWSL | 29 | 37 | Q9Y4E1 | WASHC2C |
| 280 | RRVVQRSsL | 1138 | 1146 | Q04637 | EIF4G1 |
| 281 | RRYsKFFDL | 43 | 51 | A1X283 | SH3PXD2B |
| 282 | RRYsPPIQR | 594 | 602 | Q8IYB3 | SRRM1 |
| 283 | RSRsPLEL | 23 | 30 | Q92466 | DDB2 |
| 284 | SPRRsRSISL | 159 | 168 | Q16629 | SRSF7 |
| 285 | SRFNRRVsV | 92 | 100 | P13861 | PRKAR2A |

TABLE 5
Exemplary Class I MHC Phosphopeptides on HCC that
are Specific for HLA-A*01

| SEQ | | | | | |
|---|---|---|---|---|---|
| ID | | | | UniProt | |
| NO. | Sequence^{#} | Start | Stop | Acc. No. | Gene Name |
| 286 | AEQGsPRVSY | 2121 | 2130 | Q01082 | SPTBN1 |
| 287 | GsPHYFSPFRPY | 210 | 221 | Q13242 | SRSF9 |
| 288 | ISSsMHSLY | 222 | 230 | P50616 | TOB1 |
| 289 | ITQGtPLKY | 1459 | 1467 | Q9Y618 | NCOR2 |
| 290 | LLDPSRSYsY | 643 | 652 | Q9H706 | GAREM1 |
| 291 | SLDsPSYVLY | 57 | 66 | P49354 | FNTA |
| 292 | SLYDRPAsY | 760 | 768 | P16234 | PDGFRA |
| 293 | SYPsPVATSY | 441 | 450 | P18146 | EGR1 |
| 294 | TMAsPGKDNY | 3 | 12 | O60684 | KPNA6 |
| 295 | YFsPFRPY | 214 | 221 | Q13242 | SRSF9 |
| 296 | YPLsPTKISQY | 1197 | 1207 | Q86Z02 | HIPK1 |
| 297 | YQRPFsPSAY | 4 | 13 | O94875 | SORBS2 |

TABLE 6
Exemplary Class I MHC Phosphopeptides on HCC that
are Specific for HLA-A*03

| SEQ | | | | | |
|---|---|---|---|---|---|
| ID | | | | UniProt | |
| NO. | Sequence^{#} | Start | Stop | Acc. No. | Gene Name |
| 298 | ATYtPQAPK | 251 | 259 | Q53GL0O | PLEKHO1 |
| 299 | FLIIRtVLQL | 218 | 227 | Q9H255 | OR51E2 |
| 300 | FRYsGKTEY | 345 | 353 | Q9HCM4 | EPB41L5 |
| 301 | GIMsPLAKK | 253 | 261 | Q03989 | ARID5A |
| 302 | IISsPLTGK | 461 | 469 | Q9P275 | USP36 |
| 303 | ILKPRRsL | 56 | 63 | O15205 | UBD |
| 304 | IYQyIQSRF | 270 | 278 | Q9Y463 | DYRK1B |
| 305 | KLPDsPALA | 571 | 579 | Q13586 | STIM1 |
| 306 | KLPDsPALAK | 571 | 580 | Q13586 | STIM1 |
| 307 | KLPDsPALAKK | 571 | 581 | Q13586 | STIM1 |
| 308 | KLPsPAPARK | 140 | 149 | Q8IY33 | MICALL2 |
| 309 | KLRsPFLQK | 280 | 288 | Q9UJU6 | DBNL |
| 310 | KMPTtPVKAK | 47 | 56 | Q8WUA7 | TBC1D22A |
| 311 | KRAsVFVKL | 153 | 161 | P50502 | ST13 |
| 312 | KTPTsPLKMK | 112 | 121 | O60264 | SMARCA5 |
| 313 | KVQsLRRAL | 185 | 193 | Q969G5 | PRKCDBP |
| 314 | MTRsPPRVSK | 249 | 258 | Q9BRL6 | SRSF8 |
| 315 | RAKsPISLK | 509 | 517 | Q9BXL7 | CARD11 |
| 316 | RILsGVVTK | 71 | 79 | P62280 | RPS11 |
| 317 | RIYQyIQ | 269 | 275 | Q9Y463 | DYRK1B |
| 318 | RIYQyIQSR | 269 | 277 | Q9Y463 | DYRK1B |
| 319 | RIYQyIQSRF | 269 | 278 | Q9Y463 | DYRK1B |
| 320 | RLFVGsIPK | 247 | 255 | O43390 | HNRNPR |
| 321 | RLLDRSPsRSAK | 301 | 312 | O76039 | CDKL5 |
| 322 | RLSsPISKR | 327 | 335 | Q99728 | BARD1 |
| 323 | RLSsPVLHR | 139 | 147 | Q16643 | DBN1 |
| 324 | RSLsVEIVY | 863 | 871 | Q9NS56 | TOPORS |
| 325 | RSYsRSFSR | 713 | 721 | Q7Z6E9 | RBBP6 |
| 326 | RSYsYPRQK | 648 | 656 | Q9H706 | GAREM1 |
| 327 | RTAsFAVRK | 240 | 248 | Q9Y512 | SAMM50 |
| 328 | RTAsPPPPPK | 586 | 595 | M0R088 | SRRM1 |
| 329 | RTRsLSSLREK | 1975 | 1985 | O94915 | FRYL |
| 330 | RVAsPTSGVK | 1097 | 1106 | Q9Y4H2 | IRS2 |
| 331 | RVKtPTSQSYR | 885 | 895 | Q9Y2X9 | ZNF281 |
| 332 | RVLsPLIIK | 400 | 408 | Q8NCN4 | RNF169 |
| 333 | RVRQsPLATR | 40 | 49 | O75381 | PEX14 |
| 334 | RVYsPYNHR | 582 | 590 | Q9NS56 | TOPORS |
| 335 | SVKsPVTVK | 329 | 337 | Q9HCS4 | TCF7L1 |
| 336 | SVRRsVLMK | 223 | 231 | Q9H2J4 | PDCL3 |
| 337 | yIQSRF | 273 | 278 | Q9Y463 | DYRK1B |
| 511 | KVLSPtAAK | 310 | 318 | Q96QCO | PPP1R10 |
| 522 | RVRRsSFLNAK | 61 | 71 | H0Y8T6 | RAPGEF2 |
| 523 | RVWEDRPSsA | 107 | 116 | H7BZU2 | NCOR2 |
| 526 | VLDsPASKK | 175 | 183 | Q8N5I9 | C12orf45 |

TABLE 7
Exemplary Class I MHC Phosphopeptides on HCC that
are Specific for HLA-B*44

| SEQ | | | | | |
|---|---|---|---|---|---|
| ID | | | | UniProt | |
| NO. | Sequence^{#} | Start | Stop | Acc. No. | Gene Name |
| 338 | AENARSAsF | 203 | 211 | Q9UQC2 | GAB2 |
| 339 | AENsPTRQQF | 93 | 102 | Q86XP3 | DDX42 |
| 340 | AENsSSREL | 567 | 575 | P29590 | PML |
| 341 | AtAGPRLGW | 621 | 629 | Q86W92 | PPFIBP1 |
| 342 | EELsPTAKF | 117 | 125 | Q99612 | KLF6 |
| 343 | FKtQPVTF | 365 | 373 | Q7Z7L8 | C11orf96 |
| 344 | GEAsPSHII | 557 | 565 | Q9ULL5 | PRR12 |
| 345 | GEIsPQREV | 1023 | 1031 | Q8WWI1 | LMO7 |
| 346 | GETsPRTKI | 458 | 466 | Q5VU08 | ADD3 |
| 347 | HEKKAYsF | 215 | 222 | Q15418 | RPS6KA1 |
| 348 | KEKsPFRET | 1300 | 1308 | Q9Y2F5 | ICE1 |
| 349 | KELARQIsF | 177 | 185 | Q9Y385 | UBE2J1 |
| 350 | KEmsPTRQL | 36 | 44 | Q4G0N7 | FAM229B |
| 351 | KESsPLSSRKI | 291 | 301 | Q14693 | LPIN1 |
| 352 | REAPsPLmI | 1060 | 1068 | O60885 | BRD4 |
| 353 | REAsPAPLA | 1199 | 1207 | Q9P1Y6 | PHRF1 |
| 354 | REAsPRLRV | 80 | 88 | O00220 | TNFRSF10A |
| 355 | REAsPSRLSV | 504 | 513 | O75122 | CLASP2 |
| 356 | REIMGtPEYL | 193 | 202 | O94768 | STK17B |
| 357 | REKsPGRmL | 1978 | 1986 | O14578 | CIT |
| 358 | RELARKGsL | 57 | 65 | Q8IW50 | FAM219A |
| 359 | RELsPLISL | 196 | 204 | P51825 | AFF1 |
| 360 | REPsPLPEL | 654 | 662 | Q13207 | TBX2 |
| 361 | RERsPSPSF | 326 | 334 | P49585 | PCYT1A |
| 362 | RESsPTRRL | 158 | 166 | Q9C0H9 | SRCIN1 |
| 363 | REVsPAPAV | 1361 | 1369 | O60292 | SIPA1L3 |
| 364 | REYGsTSSI | 204 | 212 | O43166 | SIPA1L1 |
| 365 | RFKtQPVTF | 364 | 373 | Q7Z7L8 | C11orf96 |
| 366 | RQKsPLFQF | 240 | 248 | Q8WY36 | BBX |
| 367 | SEFKAMDsI | 898 | 906 | P35221 | CTNNA1 |
| 368 | SELsPGRSV | 103 | 111 | Q8NFF5 | FLAD1 |
| 369 | TEAsPESML | 577 | 585 | Q9H0E9 | BRD8 |
| 370 | YEGsPIKV | 67 | 74 | P06748 | NPM1 |

TABLE 8
Exemplary Class I MHC Phosphopeptides on HCC that
are Specific for HLA-C*06

| SEQ | | | | | |
|---|---|---|---|---|---|
| ID | | | | UniProt | |
| NO. | Sequence^{#} | Start | Stop | Acc. No. | Gene Name |
| 371 | FRFsGRTEY | 309 | 317 | Q9NX84 | EPB41L4B |
| 372 | KRAsFAKSV | 328 | 336 | Q96J92 | WNK4 |
| 373 | LSSsVIREL | 201 | 209 | Q8NEJ9 | NGDN |
| 374 | RKPsIVTKY | 81 | 89 | P46100 | ATRX |
| 375 | RRHsASNLHAL | 54 | 64 | P47974 | ZFP36L2 |
| 376 | RRLsFLVSY | 67 | 75 | P47897 | QARS |
| 377 | RRLsYVLFI | 107 | 115 | Q9HCL2 | GPAM |
| 378 | RRPsYRKIL | 133 | 141 | Q03060 | CREM |
| 379 | RSAsFSRKV | 316 | 324 | O75161 | NPHP4 |
| 380 | SRSSSVLsL | 636 | 644 | A1L390 | PLEKHG3 |
| 381 | TRKtPESFL | 467 | 475 | Q9Y6I3 | EPN1 |
| 382 | YRYsPQSFL | 218 | 226 | Q9HCM1 | KIAA1551 |

TABLE 9
Exemplary Class I MHC Phosphopeptideson HCC that
are Specific for HLA-C*05

| SEQ | | | | | |
|---|---|---|---|---|---|
| ID | | | | UniProt | |
| NO. | Sequence^{#} | Start | Stop | Acc. No. | Gene Name |
| 383 | KVDsPVIF | 1114 | 1121 | Q7Z401 | DENND4A |
| 384 | RADsPVHM | 444 | 451 | O95402 | MED26 |
| 385 | RSDsYVEL | 10 | 17 | Q12888 | TP53BP1 |
| 386 | RSEsPPAEL | 309 | 317 | Q14669 | TRIP12 |
| 387 | RVDsPSHGL | 685 | 693 | Q9UER7 | DAXX |
| 388 | SIDsPQKL | 724 | 731 | Q12888 | TP53BP1 |
| 509 | AAEsPSFL | 97 | 104 | Q53TG4 | NCK2 |

TABLE 10
Exemplary Class I MHC Phosphopeptides on HCC that
are Specific for HLA-A*24

| SEQ | | | | | |
|---|---|---|---|---|---|
| ID | | | | UniProt | |
| NO. | Sequence^{#} | Start | Stop | Acc. No. | Gene Name |
| 389 | RYQtQPVTL | 849 | 857 | O95425 | SVIL |
| 390 | VYTyIQSRF | 261 | 269 | Q9NR20 | DYRK4 |

TABLE 11
Exemplary Class I MHC Phosphopeptide on HCC that
is Specific for HLA-A*31

| SEQ | | | | | |
|---|---|---|---|---|---|
| ID | | | | UniProt | |
| NO. | Sequence^{#} | Start | Stop | Acc. No. | Gene Name |
| 391 | RTSsFTFQN | 440 | 448 | P27540 | ARNT |

TABLE 12
Exemplary Class I MHC Phosphopeptide on HCC that
is Specific for HLA-B*15

| SEQ | | | | | |
|---|---|---|---|---|---|
| ID | | | | UniProt | |
| NO. | Sequence^{#} | Start | Stop | Acc. No. | Gene Name |
| 392 | RAHsEPLAL | 356 | 364 | Q66K64 | DCAF15 |

TABLE 13
Exemplary Class I MHC Phosphopeptides on HCC that
are Specific for Untyped Class I HLA

| SEQ | | | | | |
|---|---|---|---|---|---|
| ID | | | | UniProt | |
| NO. | Sequence^{#} | Start | Stop | Acc. No. | Gene Name |
| 393 | ADLsPEREV | 121 | 129 | Q8TAI7 | RHEBL1 |
| 394 | AGDsPGSQF | 284 | 292 | Q12778 | FOXO1 |
| 395 | AKLsETIS | 272 | 279 | Q9ULJ1 | ODF2L |
| 396 | AsLGFVF | 115 | 121 | Q8NCK7 | SLC16A11 |
| 397 | DAKKsPLAL | 83 | 91 | Q9H759 | ZNF703 |
| 398 | DLKSSKAsL | 5742 | 5750 | Q09666 | AHNAK |
| 399 | FTKsPYQEF | 261 | 269 | P15880 | RPS2 |
| 400 | GQLsPGVQF | 69 | 77 | Q07002 | CDK18 |
| 401 | GsPHYFSPF | 210 | 218 | Q13242 | SRSF9 |
| 402 | HTAsPTGMMK | 34 | 43 | 0O4855 | SEC24D |
| 403 | HVYtPSTTK | 113 | 121 | Q9H9E1 | ANKRA2 |
| 404 | IQFsPPFPGA | 1353 | 1362 | Q9Y2G9 | SBNO2 |
| 405 | KASPKRLsL | 632 | 640 | Q765P7 | MTSS1L |
| 406 | KAVsLFLCY | 4 | 12 | P09912 | IFI6 |
| 406 | KAVsLFLcY | 4 | 12 | P09912 | IFI6 |
| 407 | KIFsGVFVK | 114 | 122 | Q6DKI1 | RPL7L1 |
| 408 | KIKsFEVVF | 6 | 14 | Q9H3M7 | TXNIP |
| 409 | KLKDRLPsI | 56 | 64 | Q53QV2 | LBH |
| 410 | KLsGDQPAAR | 1348 | 1357 | Q13428 | TCOF1 |
| 411 | KLSGLsF | 99 | 105 | P49006 | MARCKSL1 |
| 412 | KTMsPSQMIM | 846 | 855 | Q9ULJ6 | ZMIZ1 |
| 413 | KVKsSPLIEKL | 79 | 89 | Q6JBY9 | RCSD1 |
| 414 | NMDsPGPML | 107 | 115 | P32519 | ELF1 |
| 415 | PmVTLsLNL | 160 | 168 | Q8NDX9 | LY6G5B |
| 416 | PYDPALGsPSR | 58 | 68 | Q6BEB4 | SPS |
| 417 | RAFsVKFEV | 113 | 121 | P00973 | OAS1 |
| 418 | RGDGYGtF | 587 | 594 | Q9NQ94 | A1CF |
| 419 | RIGsPLSPK | 337 | 345 | Q659C4 | LARP1B |
| 420 | RKLRsLEQL | 650 | 658 | Q6NSJ5 | LRRC8E |
| 421 | RKSsIIIRM | 80 | 88 | Q02325 | PLGLB1 |
| 422 | RLLDPsSPLAL | 829 | 839 | Q9UPX8 | SHANK2 |
| 422 | RLLDPSsPLAL | 829 | 839 | Q9UPX8 | SHANK2 |
| 423 | RLSsLRASTSK | 233 | 243 | P62753 | RPS6 |
| 424 | RMFsPMEEK | 691 | 699 | Q9UHB7 | AFF4 |
| 425 | RMYsPIPPSL | 475 | 484 | Q86TG7 | PEG10 |
| 426 | RNLsSPFIF | 643 | 651 | P52569 | SLC7A2 |
| 427 | RSRsPRPAL | ? | ? | Unknown | Unknown |
| 428 | RTHsLLLLL | 5 | 13 | P34096 | RNASE4 |
| 429 | RTNsPGFQK | 515 | 523 | Q5T8P6 | RBM26 |
| 430 | RTPsDVKEL | 14 | 22 | P39687 | ANP32A |
| 431 | RTSsFALNL | 3775 | 3783 | P04114 | APOB |
| 432 | RTSsPLFNK | 125 | 133 | Q9BY89 | KIAA1671 |
| 433 | RTYsHGTYR | 451 | 459 | Q6PCB5 | RSBN1L |
| 434 | RYPsNLQLF | 464 | 472 | Q99973 | TEP1 |
| 435 | sDDEKMPDLE | 151 | 160 | Q15185 | PTGES3 |
| 436 | SDmPRAHsF | 218 | 226 | P78314 | SH3BP2 |
| 437 | SFDsGSVRL | 413 | 421 | O14896 | IRF6 |
| 438 | SsPIMRKKVSL | 1171 | 1181 | O43314 | PPIP5K2 |
| 439 | sYIEHIFEI | 61 | 69 | Q15121 | PEA15 |
| 440 | sYQKVIELF | 289 | 297 | Q96KB5 | PBK |
| 441 | TLLAsPMLK | 1248 | 1256 | P17948 | FLT1 |
| 442 | TLMERTVsL | 254 | 262 | Q8IWE2 | FAM114A1 |
| 443 | VLFPEsPARA | 4817 | 4826 | O14686 | KMT2D |
| 444 | VLIENVAsL | 31 | 39 | P18283 | GPX2 |
| 445 | VLSDVIPsI | 151 | 159 | Q6PEV8 | FAM199X |
| 446 | VLVVDTPsI | 78 | 86 | Q96F15 | GIMAP5 |
| 447 | VVDsPGQEVL | 22 | 31 | Q8WUA4 | GTF3C2 |
| 448 | YARsVHEEF | 354 | 362 | Q9BRK5 | SDF4 |
| 529 | SARRtPVSY | 1480 | 1488 | O75376 | NCOR1 |

TABLE 14
Exemplary Class I MHC Phosphopeptides on
Esophageal Cancer

| | | | | | |
|---|---|---|---|---|---|
| | | | | UniProt | |
| SEQ ID NO. | Sequence^{#} | Start | Stop | Acc. No. | Gene Name |

HLA A*0201

| | | | | | |
|---|---|---|---|---|---|
| 502 | SIPtVSGQI | 15 | 23 | Q8TF68 | ZNF384 |

HLA A*0101

| | | | | | |
|---|---|---|---|---|---|
| 503 | YPLsPAKVNQY | 1185 | 1195 | Q9H2X6 | HIPK2 |
| 504 | YPLsPTKISEY | 1197 | 1207 | Q86Z02 | HIPK1 |

HLA-B*0702

| | | | | | |
|---|---|---|---|---|---|
| 505 | VPLIRKKsL | 20 | 28 | B7ZW66 | PGA5 |

Other HLA Alleles

| | | | | | |
|---|---|---|---|---|---|
| 506 | LKLsYLTWV | 561 | 569 | 043246 | SLC7A4 |
| 507 | KRYsEPVSL | 647 | 655 | Q9COD6 | FHDC1 |
| 508 | KSGELLAtW | 168 | 176 | Q9HOK1 | SIK2 |

Exemplary MHC class I phosphopeptides of the presently disclosed subject matter that are associated in some embodiments with esophageal cancer are set forth in Table 14 and as SEQ ID NOs: 502-508, for example.

In some embodiments, the phosphopeptides of the presently disclosed subject matter comprise the amino acid sequences of at least one of the MHC class I binding peptides set forth in SEQ ID NOs: 1-448 and 502-529. Moreover, in some embodiments about or at least 1, 2, 3, 4, 5, 6, 7, 8, 9, 10, 11, 12, 13, 14, 15, or more of the serine, homo-serine, threonine, or tyrosine residues within the recited sequence is phosphorylated. The phosphorylation can in some embodiments be with a natural phosphorylation (—CH₂—O—PO₃H) or with an enzyme non-degradable, modified phosphorylation, such as (—CH₂—CF₂—PO₃H or —CH₂—CH₂—PO₃H). Some phosphopeptides can contain more than one of the amino acid sequences set forth in SEQ ID NOs: 1-448 and 502-529, for example, if they are overlapping, adjacent, or nearby within the native protein from which they are derived.

In some embodiments, the target peptides comprise a phosphopeptide mimetic. In some embodiments, the phosphopeptide mimetic replaces a phosphoserine, phosphothreonine, or phosphotyrosine residue indicated in Tables 2-14. The chemical structure of a phosphopeptide mimetic appropriate for use in the presently disclosed subject matter can in some embodiments closely approximate the natural phosphorylated residue which is mimicked, and also can in some embodiments be chemically stable (e.g., resistant to dephosphorylation by phosphatase enzymes). This can be achieved with a synthetic molecule in which the phosphorous atom is linked to the amino acid residue, not through oxygen, but through carbon. In some embodiments, a CF₂ group links the amino acid to the phosphorous atom. Mimetics of several amino acids which are phosphorylated in nature can be generated by this approach. Mimetics of phosphoserine, phosphothreonine, and phosphotyrosine can be generated by placing a CF₂ linkage from the appropriate carbon to the phosphate moiety. The mimetic molecule L-2-amino-4 (diethylphosphono)-4,4-difluorobutanoic acid (F2Pab) can in some embodiments substitute for phosphoserine (Otaka et al., 1995). L-2-amino-4-phosphono-4,4difluoro-3-methylbutanoic acid (F2Pmb) can in some embodiments substitute for phosphothreonine. L-2-amino-4-phosphono (difluoromethyl) phenylalanine (F2Pmp) can in some embodiments substitute for phosphotyrosine (Smyth et al., 1992; Akamatsu et al., 1997). Alternatively, the oxygen bridge of the natural amino acid can in some embodiments be replaced with a methylene group. In some embodiments, serine and threonine residues are substituted with homo-serine and homo-threonine residues, respectively. A phosphomimetic can in some embodiments also include vanadate, pyrophosphate or fluorophosphates.

### IV. Immunosuitablity

In some embodiments, the target peptides of the presently disclosed subject matter are combined into compositions which can be used in vaccine compositions for eliciting anti-tumor immune responses or in adoptive T-cell therapy of HCC patients and/or esophageal cancer patients. Tables 2-14 provide target peptides presented on the surface of cancer cells.

The presently disclosed subject matter provides in some embodiments target peptides which are immunologically suitable for each of the foregoing HLA alleles and, in particular, HLA-A*0201, HLA-B*0702, HLA-B*2705, HLA-A*01, HLA-A*03, HLA-B*44, HLA-C*06, HLA-C*05, HLA-A*24, HLA-A*31, and HLA-B*15. “Immunologically suitable” means that a target peptide will bind at least one allele of an MHC class I molecule in a given patient. Compositions of the presently disclosed subject matter are in some embodiments immunologically suitable for a patient when at least one target peptide of the composition will bind at least one allele of an MHC class I molecule in a given patient. Compositions of multiple target peptides presented by each of the most prevalent alleles used in a cocktail, ensures coverage of the human population and to minimize the possibility that the tumor will be able to escape immune surveillance by down-regulating expression of any one class I target peptide.

The compositions of the presently disclosed subject matter can in some embodiments have at least one target peptide specific for HLA-A*0201, HLA-B*0702, HLA-B*2705, HLA-A*01, HLA-A*03, HLA-B*44, HLA-C*06, HLA-C*05, HLA-A*24, HLA-A*31, and HLA-B*15. The compositions can in some embodiments have at least one phosphopeptide specific for an HLA allele selected from the group consisting of HLA-A*0201, HLA-B*0702, HLA-B*2705, HLA-A*01, HLA-A*03, HLA-B*44, HLA-C*06, HLA-C*05, HLA-A*24, HLA-A*31, and HLA-B*15. In some embodiments, the compositions can further comprise additional phosphopeptides from other MHC class I alleles.

As such, the compositions of the presently disclosed subject matter containing various combinations of target peptides will in some embodiments be immunologically suitable for between or about 3-88%, 80-89%, 70-79%, 60-69%, 57-59%, 55-57%, 53-55% or 51-53% or 5-90%, 10-80%, 15-75%, 20-70%, 25-65%, 30-60%, 35-55%, or 40-50% of the population of a particular cancer, e.g., HCC. In some embodiments, the compositions of the presently disclosed subject matter are able to act as vaccine compositions for eliciting anti-tumor immune responses or in adoptive T-cell therapy of HCC patients, wherein the compositions are immunologically suitable for about or at least 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 20, 19, 18, 17, 16, 15, 14, 13, 12, 11, 10, 9, 8, 7, 6, 5, 4 or 3 percent of cancer, e.g., HCC, patients.

### V. Compositions

“Target peptide compositions” as used herein refers to at least one target peptide formulated for example, as a vaccine; or as a preparation for pulsing cells in a manner such that the pulsed cells, e.g., dendritic cells, will display the at least one target peptide in the composition on their surface, e.g., to T-cells in the context of adoptive T-cell therapy.

The compositions of the presently disclosed subject matter can include in some embodiments about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50-55, 55-65, 65-80, 80-120, 90-150, 100-175, or 175-250 different target peptides.

The compositions of the presently disclosed subject matter generally include MHC class I specific target peptide(s) but in some embodiments can also include one or more target peptides specific for MHC class II or other peptides associated with tumors, e.g., tumor-associated antigen (“TAA”).

Compositions comprising the presently disclosed target peptide are typically substantially free of other human proteins or peptides. They can be made synthetically or by purification from a biological source. They can be made recombinantly. In some embodiments, they are at least 90%, 92%, 93%, 94%, at least 95%, or at least 99% pure. For administration to a human body, in some embodiments they do not contain other components that might be harmful to a human recipient. The compositions are typically devoid of cells, both human and recombinant producing cells. However, as noted below, in some cases, it can be desirable to load dendritic cells with a target peptide and use those loaded dendritic cells as either an immunotherapy agent themselves, or as a reagent to stimulate a patient's T cells ex vivo. The stimulated T cells can be used as an immunotherapy agent. In some embodiments, it can be desirable to form a complex between a target peptide and an HLA molecule of the appropriate type. Such complexes can in some embodiments be formed in vitro or in vivo. Such complexes are typically tetrameric with respect to an HLA-target peptide complex. Under certain circumstances it can be desirable to add additional proteins or peptides, for example, to make a cocktail having the ability to stimulate an immune response in a number of different HLA type hosts. Alternatively, additional proteins or peptide can provide an interacting function within a single host, such as an adjuvant function or a stabilizing function. As a non-limiting example, other tumor antigens can be used in admixture with the target peptides, such that multiple different immune responses are induced in a single patient.

Administration of target peptides to a mammalian recipient can in some embodiments be accomplished using long target peptides (e.g., longer than 15 residues) or using target peptide loaded dendritic cells (see Melief, 2009). The immediate goal is to induce activation of CD8⁺ T cells. Additional components which can be administered to the same patient, either at the same time or close in time (e.g., within 21 days of each other) include TLR-ligand oligonucleotide CpG and related target peptides that have overlapping sequences of at least 6 amino acid residues. To ensure efficacy, mammalian recipients should express the appropriate human HLA molecules to bind to the target peptides. Transgenic mammals can be used as recipients, for example, if they express appropriate human HLA molecules. If a mammal's own immune system recognizes a similar target peptide then it can be used as model system directly, without introducing a transgene. Useful models and recipients can in some embodiments be at increased risk of developing metastatic cancer, such as HCC. Other useful models and recipients can be predisposed, e.g., genetically or environmentally, to develop HCC or other cancer.

V.A. Selection of Target Peptides

Disclosed herein is the finding that immune responses can be generated against phosphorylated peptides tested in healthy and diseased individuals. The T-cells associated with these immune responses, when expanded in vitro, are able to recognize and kill malignant tissue (both established cells lines and primary tumor samples). Cold-target inhibition studies reveal that these target peptide-specific T-cell lines kill primary tumor tissue in a target peptide-specific manner.

When selecting target peptides of the presently disclosed subject matter for inclusion in immunotherapy, e.g., in adaptive cell therapy or in the context of a vaccine, one can preferably pick target peptides that in some embodiments: 1) are associated with a particular cancer/tumor cell type; 2) are associated with a gene/protein involved in cell proliferation; 3) are specific for an HLA allele carried the group of patients to be treated; and/or 4) are capable of inducing a target peptide-specific memory T cell response in the patients to be treated upon a first exposure to a composition including the selected target peptides.

V.B. Target Peptide Vaccines

The antigen target peptides can also in some embodiments be used to vaccinate an individual. The antigen target peptides can be injected alone or in some embodiments can be administered in combination with an adjuvant and a pharmaceutically acceptable carrier. Vaccines are envisioned to prevent or treat certain diseases in general and cancers in particular.

The target peptides compositions of the presently disclosed subject matter can in some embodiments be used as a vaccine for cancer, and more specifically for hepatocellular carcinoma (HCC), esophageal cancer, melanoma, leukemia, ovarian, breast, colorectal, or lung squamous cancer, sarcoma, renal cell carcinoma, pancreatic carcinomas, squamous tumors of the head and neck, brain cancer, liver cancer, prostate cancer, and cervical cancer. The compositions can in some embodiments include target peptides. The vaccine compositions can in some embodiments include only the target peptides, or peptides disclosed herein, or they can include other cancer antigens that have been identified.

The vaccine compositions can in some embodiments be used prophylactically for the purposes of preventing, reducing the risk of, and/or delaying initiation of a cancer in an individual that does not currently have cancer. Alternatively, they can be used to treat an individual that already has cancer, so that recurrence or metastasis is delayed and/or prevented. Prevention relates to a process of prophylaxis in which the individual is immunized prior to the induction or onset of cancer. For example, individuals with a history of poor life style choices and at risk for developing HCC can in some embodiments be immunized prior to the onset of the disease.

Alternatively or in addition, individuals that already have cancer can be immunized with the antigens of the presently disclosed subject matter so as to stimulate an immune response that would be reactive against the cancer. A clinically relevant immune response would be one in which the cancer partially or completely regresses and/or is eliminated from the patient, and it would also include those responses in which the progression of the cancer is blocked without being eliminated. Similarly, prevention need not be total, but can in some embodiments result in a reduced risk, delayed onset, and/or delayed progression or metastasis.

The target peptide vaccines of the presently disclosed subject matter can in some embodiments be given to patients before, after, or during any of the aforementioned stages of HCC and/or esophageal cancer. In some embodiments, they are given to patients with malignant HCC and/or malignant esophageal cancer (e.g., squamous cell carcinoma and/or adenocarcinoma).

In some embodiments, the 5-year survival rate of patients treated with the vaccines of the presently disclosed subject matter is increased by a statistically significant amount, e.g., by about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more percent, relative to the average 5-year survival rates described above.

In some embodiments, the target peptide vaccine composition of the presently disclosed subject matter will increase survival rates in patients with metastatic HCC and/or malignant esophageal cancer by a statistically significant amount of time, e.g., by about or at least, 0.25, 0.5, 0.75, 1.0, 1.25, 1.5, 1.75, 2.0, 2.25, 2.5, 2.75, 3.0, 3.25, 3.5, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.50, 9.75, 10.0, 10.25, 10.5, 10.75, 11.0, 11.25, 11.5, 11.75, or 12 months or more compared to what could have been expected without vaccine treatment at the time of filing of this disclosure.

In some embodiments, the survival rate, e.g., the 1, 2, 3, 4, or 5-year survival rate, of patients treated with the vaccines of the presently disclosed subject matter is increased by a statistically significant amount, e.g., by about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent, relative to the average 5-year survival rates described above.

The target peptide vaccines of the presently disclosed subject matter are in some embodiments envisioned to illicit a T cell associated immune response, e.g., generating activated CD8⁺ T cells specific for native target peptide/MHC class I expressing cells, specific for at least or about 1, 2, 3, 4, 5, 6, 7, 8, 9, 10, or more of the target peptides in the vaccine in a patient for about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 07, 98, 99, or 100 days after providing the vaccine to the patient.

In some embodiments, the treatment response rates of patients treated with the target peptide vaccines of the presently disclosed subject matter are increased by a statistically significant amount, e.g., by about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 07, 98, 99, 100, 150, 200, 250, 300, 350, 400, 450, 500, or more percent, relative to treatment without the vaccine.

In some embodiments, overall median survival of patients treated with the target peptide vaccines of the presently disclosed subject matter is increased by a statistically significant amount, e.g., by about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, or more percent, relative to treatment without the vaccine. In some embodiments, the overall median survival of HCC patients treated the target peptide vaccines is envisioned to be about or at least 10.0, 10.25, 10.5, 10.75, 11.0, 11.25, 11.5, 11.75, 12, 12.25, 12.5, 12.75, 13, 13.25, 13.5, 13.75, 14, 14.25, 14.5, 14.75, 15, 16, 17, 18, 19, 20, 21, 22, 23, 24, 25, 26, 27, 28, 29, 30, 31, 32, 33, 34, 35, 36, or more months.

In some embodiments, tumor size of patients treated with the target peptide vaccines of the presently disclosed subject matter is decreased by a statistically significant amount, e.g., by about, or by at least, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, or more percent, relative to treatment without the vaccine.

In some embodiments, the compositions of the presently disclosed subject matter provide an clinical tumor regression by a statistically significant amount, e.g., in about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of patients treated with a composition of the presently disclosed subject matter.

In some embodiments, the compositions of the presently disclosed subject matter provide a CTL response specific for the cancer being treated (such as but not limited to HCC and/or malignant esophageal cancer) by a statistically significant amount, e.g., in about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of patients treated with a composition of the presently disclosed subject matter.

In some embodiments, the compositions of the presently disclosed subject matter provide an increase in progression free survival in the cancer being treated (e.g., HCC and/or malignant esophageal cancer), of about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 225, 250, 275, 300, 325, 350, 375, 400, 425, 450, 475, 500, or more percent compared to the progression free survival or patients not treated with the composition.

In some embodiments, progression free survival, CTL response rates, clinical tumor regression rates, tumor size, survival rates (including but not limited to overall survival rates), and/or response rates are determined, assessed, calculated, and/or estimated weekly, monthly, bi-monthly, quarterly, semi-annually, annually, and/or bi-annually over a period of about or at least 1, 2, 3, 4, 5, 6, 7, 8, 9, 10, 11, 12, 13, 14, 15 or more years or about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 225, 250, 275, 300, 325, 350, 375, 400, 425, 450, 475, 500, or more weeks.

V.C. Compositions for Priming T Cells

Adoptive cell transfer is the passive transfer of cells, in some embodiments immune-derived cells, into a recipient host with the goal of transferring the immunologic functionality and characteristics into the host. Clinically, this approach has been exploited to transfer either immune-promoting or tolergenic cells (often lymphocytes) to patients to enhance immunity against cancer. The adoptive transfer of autologous tumor infiltrating lymphocytes (TIL) or genetically re-directed peripheral blood mononuclear cells has been used to successfully treat patients with advanced solid tumors, including melanoma and ovarian carcinoma, HCC, and/or malignant esophageal cancer (e.g., squamous cell carcinoma and/or adenocarcinoma), as well as patients with CD19-expressing hematologic malignancies. In some embodiments, adoptive cell transfer (ACT) therapies achieve T-cell stimulation ex vivo by activating and expanding autologous tumor-reactive T-cell populations to large numbers of cells that are then transferred back to the patient (see e.g., Gattinoni et al., 2006).

The target peptides of the presently disclosed subject matter can in some embodiments take the form of antigen peptides formulated in a composition added to autologous dendritic cells and used to stimulate a T helper cell or CTL response in vitro. The in vitro generated T helper cells or CTL can then be infused into a patient with cancer (Yee et al., 2002), and specifically a patient with a form of cancer that expresses one or more of antigen target peptides.

Alternatively or in addition, the target peptides of the presently disclosed subject matter can be added to dendritic cells in vitro, with the loaded dendritic cells being subsequently transferred into an individual with cancer in order to stimulate an immune response. Alternatively or in addition, the loaded dendritic cells can be used to stimulate CD8⁺ T cells ex vivo with subsequent reintroduction of the stimulated T cells to the patient. Although a particular target peptide can be identified on a particular cancer cell type, it can be found on other cancer cell types.

The presently disclosed subject matter envisions treating cancer by providing a patient with cells pulsed with a composition of target peptides. The use of dendritic cells (“DCs”) pulsed with target peptide antigens allows for manipulation of the immunogen in two ways: varying the number of cells injected and varying the density of antigen presented on each cell. Exemplary methods for DC-based based treatments can be found for example in Mackensen et al., 2000.

V.D. Additional Peptides Present in Target Peptide Compositions

The target peptide compositions (or target peptide composition kits) of the presently disclosed subject matter can in some embodiments also include at least one additional peptide derived from tumor-associated antigens. Examples of tumor-associated antigens include MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS, prostatic acid phosphatase, and the like. Particular examples of additional peptides derived from tumor-associated antigens that can be employed alone or in combination with the compositions of the presently disclosed subject matter those set forth in Table 15 below.

TABLE 15
Exemplary Peptides Derived from Tumor-associated Antigens

| | | GENBANK® |
|---|---|---|
| Polypeptide Name^{a} | Amino Acid Sequence^{b} (SEQ ID NO:) | Acc. No(s).^{c} |
| CEA₆₁₋₆₉ | HLFGYSWYK (SEQ ID NO: 452) | NP_001264092.1 |
| | | XP_005278431.1 |
| CEA₆₀₄₋₆₁₂ | YLSGADLNL (SEQ ID NO: 453) | XP_005278431.1 |
| FBP/FOLR1₁₉₁₋₁₉₉ | EIWTHSYKV (SEQ ID NO: 454) | NP_000793.1 |
| gp100₁₇₋₂₅ | ALLAVGATK (SEQ ID NO: 455) | NP_001186982.1 |
| gp100₄₄₋₅₉ | WNRQLYPEWTEAQRLD | NP_008859.1 |
| | (SEQ ID NO: 456) | |
| gp100₈₇₋₉₅ | ALNFPGSQK (SEQ ID NO: 457) | NP_008859.1 |
| gp100₈₉₋₉₅ | SQNFPGSQK (SEQ ID NO: 458) | NP_008859.1 |
| gp100₁₅₄₋₁₆₂ | KTWGQYWQV (SEQ ID NO: 459) | NP_008859.1 |
| gp100₂₀₉₋₂₁₇ | ITDQVPFSV (SEQ ID NO: 460) | NP_008859.1 |
| gp100₂₀₉₋₂₁₇ | IMDQVPFSV (SEQ ID NO: 461) | NP_008859.1 |
| gp100₂₈₀₋₂₈₈ | YLEPGPVTA (SEQ ID NO: 462) | NP_008859.1 |
| gp100₄₇₆₋₄₈₅ | VLYRYGSFSV (SEQ ID NO: 463) | NP_008859.1 |
| gp100₆₁₄₋₆₂₂ | LIYRRRLMK (SEQ ID NO: 464) | NP_008859.1 |
| Her2/neu₃₆₉₋₃₇₇ | KIFGSLAFL (SEQ ID NO: 465) | NP_004439.2 |
| Her2/neu₇₅₄₋₇₆₂ | VLRENTSPK (SEQ ID NO: 466) | NP_004439.2 |
| MAGE-A1₁₁₄₋₁₂₇ | LLKYRAREPVTKAE | NP_004979.3 |
| MAGE-A2,3,6₁₂₁₋₁₃₄ | (SEQ ID NO: 467) | NP_005352.1 |
| | | NP_0053 53.1 |
| | | NP_005354.1 |
| MAGE-A1₉₆₋₁₀₄ | SLFRAVITK (SEQ ID NO: 468) | NP_004979.3 |
| MAGE-A1₁₆₁₋₁₆₉ | EADPTGHSY (SEQ ID NO: 469) | NP_004979.3 |
| MAGE-A3₁₆₈₋₁₇₆ | EVDPIGHLY (SEQ ID NO: 470) | NP_005353.1 |
| MAGE-A3₂₈₁₋₂₉₅ | TSYVKVLHHMVKISG | NP_005353.1 |
| | (SEQ ID NO: 471) | |
| MAGE-A10₂₅₄₋₂₆₂ | GLYDGMEHL (SEQ ID NO: 472) | NP_001011543.2 |
| MART-1/MelanA₂₇₋₃₅ | AAGIGILTV (SEQ ID NO: 473) | NP_005502.1 |
| MART-1/MelanA₅₁₋₇₃ | RNGYRALMDKSLHVGTQCALTRR | NP_005502.1 |
| | (SEQ ID NO: 474) | |
| MART-1/MelanA₉₇₋₁₁₆ | VPNAPPAYEKLsAEQSPPPY | NP_005502.1 |
| | (SEQ ID NO: 475) | |
| MART-1/Me1anA₉₈₋₁₀₉ | PNAPPAYEKLsA (SEQ ID NO: 476) | NP_005502.1 |
| MART-1/Me1anA₉₉₋₁₁₀ | NAPPAYEKLsAE (SEQ ID NO: 477) | NP_005502.1 |
| MART-1/MelanA₁₀₀₋₁₀₈ | APPAYEKLs (SEQ ID NO: 478) | NP_005502.1 |
| MART-1/MelanA₁₀₀₋₁₁₁ | APPAYEKLsAEQ (SEQ ID NO: 479) | NP_005502.1 |
| MART-1/MelanA₁₀₀₋₁₁₄ | APPAYEKLsAEQSPP | NP_005502.1 |
| | (SEQ ID NO: 480) | |
| MART-1/MelanA₁₀₀₋₁₁₅ | APPAYEKLsAEQSPPP | NP_005502.1 |
| | (SEQ ID NO: 481) | |
| MART-1/MelanA₁₀₀₋₁₁₆ | APPAYEKLsAEQSPPPY | NP_005502.1 |
| | (SEQ ID NO: 482) | |
| MART-1/MelanA₁₀₁₋₁₀₉ | PPAYEKLsA (SEQ ID NO: 483) | NP_005502.1 |
| MART-1/MelanA₁₀₁₋₁₁₂ | PPAYEKLsAEQS (SEQ ID NO: 484) | NP_005502.1 |
| MART-1/MelanA₁₀₂₋₁₁₀ | PAYEKLsAE (SEQ ID NO: 485) | NP_005502.1 |
| MART-1/MelanA₁₀₂₋₁₁₃ | PAYEKLsAEQSP (SEQ ID NO: 486) | NP_005502.1 |
| MART-1/MelanA₁₀₃₋₁₁₄ | AYEKLsAEQSPP (SEQ ID NO: 487) | NP_005502.1 |
| MART-1/MelanA₁₀₄₋₁₁₅ | YEKLsAEQSPPP (SEQ ID NO: 488) | NP_005502.1 |
| NY-ESO-1 | AAQERRVPR (SEQ ID NO: 489) | AAD05203.1 |
| | | CAA10193.1 |
| NY-ESO-1 | LLGPGRPYR (SEQ ID NO: 490) | NP_001913.2 |
| NY-ESO-1₅₃₋₆₂ | ASGPGGGAPR (SEQ ID NO: 491) | NP_001318.1 |
| p2₈₃₀₋₈₄₄ | AQYIKANSKFIGITEL | NP_783831.1 |
| | (SEQ ID NO: 492) | |
| TAG-1,2 | RLSNRLLLR (SEQ ID NO: 493) | |
| Tyr₅₆₋₇₀ | AQNILLSNAPLGPQFP | NP_000363.1 |
| | (SEQ ID NO: 494) | |
| Tyr₁₄₆₋₁₅₆ | SSDYVIPIGTY (SEQ ID NO: 495) | NP_000363.1 |
| Tyr₂₄₀₋₂₅₁ | SDAEKSDICTDEY | NP_000363.1 |
| | (SEQ ID NO: 496) | |
| Tyr₂₄₃₋₂₅₁ | KCDICTDEY (SEQ ID NO: 497) | NP_000363.1 |
| Tyr₃₆₉₋₃₇₇ | YMDGTMSQV (SEQ ID NO: 498) | NP_000363.1 |
| Tyr₃₈₈₋₄₀₆ | FLLHHAFVDSIFEQWLQRHRP | NP_000363.1 |
| | (SEQ ID NO: 499) | |

^{a}Numbers listed in subscript are the amino acids positions of the listed peptide sequence in the corresponding polypeptide including, but not limited to the amino acid sequences provided in the GENBANK® biosequence database.
^{b}lower case amino acids in this column are optionally phosphorylated.
^{c}GENBANK® biosequence database Accession Numbers listed here are intended to be exemplary only and should not be interpreted to limit the disclosed peptide sequences to only these polypeptides.

Such tumor specific peptides (including the MHC class I phosphopeptides disclosed in SEQ ID NOs: 1-448 and 502-529 and in Tables 2-14) can be added to the target peptide compositions in a manner, number, and/or in an amount as if they were an additional target peptide added to the target peptide compositions as described herein.

V.E. Combination Therapies

In some embodiments, the target peptide compositions (or target peptide composition kits) of the presently disclosed subject matter are administered as a vaccine or in the form of pulsed cells as first, second, third, or fourth line treatment for the cancer. In some embodiments, the compositions of the presently disclosed subject matter are administered to a patient in combination with one or more therapeutic agents, e.g., anti-CA125 (or oregovomab Mab B43.13), anti-idiotype Ab (ACA-125), anti-HER-2 (trastuzumab, pertuzumab), anti-MUC-1 idiotypic Ab (HMFG1), HER-2/neu peptide, NY-ESO-1, anti-Programed Death-1 (“PD1”) (or PD1-antagonists such as BMS-936558), anti-CTLA-4 (or CTLA-4 antagonists), vermurafenib, ipilimumab, dacarbazine, IL-2, IFN-α, IFN-γ, temozolomide, receptor tyrosine kinase inhibitors (e.g., imatinib, gefitinib, erlotinib, sunitinib, tyrphostins, telatinib), sipileucel-T, tumor cells transfected with GM-CSF, a platinum-based agent, a taxane, an alkylating agent, an antimetabolite and/or a vinca alkaloid or combinations thereof. In an embodiment, the cancer is sensitive to or refractory, relapsed or resistant to one or more chemotherapeutic agents, e.g., a platinum-based agent, a taxane, an alkylating agent, an anthracycline (e.g., doxorubicin (e.g., liposomal doxorubicin)), an antimetabolite and/or a vinca alkaloid. In some embodiments, the cancer is, e.g., HCC, and the HCCis refractory, relapsed, or resistant to a platinum-based agent (e.g., carboplatin, cisplatin, oxaliplatin), a taxane (e.g., paclitaxel, docetaxel, larotaxel, cabazitaxel) and/or an anthracycline (e.g., doxorubicin (e.g., liposomal doxorubicin)). In some embodiments, the cancer is, e.g., HCC, and the HCC is refractory, relapsed, or resistant to an antimetabolite (e.g., an antifolate (e.g., pemetrexed, floxuridine, raltitrexed) and a pyrimidine analogue (e.g., capecitabine, cytrarabine, gemcitabine, 5FU)) and/or a platinum-based agent (e.g., carboplatin, cisplatin, oxaliplatin). In some embodiments, the cancer is, e.g., lung cancer, and the cancer is refractory, relapsed or resistant to a taxane (e.g., paclitaxel, docetaxel, larotaxel, cabazitaxel), a platinum-based agent (e.g., carboplatin, cisplatin, oxaliplatin), a vinca alkaloid (e.g., vinblastine, vincristine, vindesine, vinorelbine), a vascular endothelial growth factor (VEGF) pathway inhibitor, an epidermal growth factor (EGF) pathway inhibitor) and/or an antimetabolite (e.g., an antifolate (e.g., pemetrexed, floxuridine, raltitrexed) and a pyrimidine analogue (e.g., capecitabine, cytrarabine, gemcitabine, 5FU)). In some embodiments, the cancer is, e.g., breast cancer, and the cancer is refractory, relapsed or resistant to a taxane (e.g., paclitaxel, docetaxel, larotaxel, cabazitaxel), a vascular endothelial growth factor (VEGF) pathway inhibitor, an anthracycline (e.g., daunorubicin, doxorubicin (e.g., liposomal doxorubicin), epirubicin, valrubicin, idarubicin), a platinum-based agent (e.g., carboplatin, cisplatin, oxaliplatin), and/or an antimetabolite (e.g., an antifolate (e.g., pemetrexed, floxuridine, raltitrexed) and a pyrimidine analogue (e.g., capecitabine, cytrarabine, gemcitabine, 5FU)). In some embodiments, the cancer is, e.g., gastric cancer, and the cancer is refractory, relapsed or resistant to an antimetabolite (e.g., an antifolate (e.g., pemetrexed, floxuridine, raltitrexed) and a pyrimidine analogue (e.g., capecitabine, cytrarabine, gemcitabine, 5FU)) and/or a platinum-based agent (e.g., carboplatin, cisplatin, oxaliplatin).

In some embodiments, the target peptide compositions (or target peptide composition kits) of the presently disclosed subject matter are associated with agents that inhibit T cell apoptosis or anergy thus potentiating a T cell response (“T cell potentiator”). Such agents include B7RP1 agonists, B7-H3 antagonists, B7-H4 antagonists, HVEM antagonists, HVEM antagonists, GALS antagonists or alternatively CD27 agonists, OX40 agonists, CD137 agonists, BTLA agonists, ICOS agonists CD28 agonists, or soluble versions of PDL1, PDL2, CD80, CD96, B7RP1, CD137L, OX40 or CD70. See Pardoll, National Reviews of Cancer, Focus on Tumor Immunology & Immunotherapy, 254, April 2012, Volume 12.

In some embodiments, the T cell potentiator is a PD1 antagonist. Programmed death 1 (PD-1) is a key immune checkpoint receptor expressed by activated T cells, and it mediates immunosuppression. PD-1 functions primarily in peripheral tissues, where T cells can encounter the immunosuppressive PD-1 ligands PD-L1 (B7-H1) and PD-L2 (B7-DC), which are expressed by tumor cells, stromal cells, or both. In some embodiments, the anti-PD-1 monoclonal antibody BMS-936558 (also known as MDX-1106 and ONO-4538) is used. In some embodiments, the T cell potentiator, e.g., PD1 antagonist, is administered as an intravenous infusion at least or about every 1, 1.5, 2, 2.5, 3, 3.5, or 4 weeks of each 4, 5, 6, 7, 8, 9, or 10-week treatment cycle of about for at least 1, 2, 3, 4, 5, 6, 7, 8, 9, 10, 11, 12, 13, 14, 15, 16, 17, 18, 19, 20, or more cycles. Exemplary, non-limiting doses of the PD1 antagonists are envisioned to be exactly, about, or at least 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, or more mg/kg (see Brahmer et al., 2012).

The exemplary therapeutic agents disclosed herein above are envisioned to be administered at a concentration of, e.g., about 1 to 100 mg/m², about 10 to 80 mg/m², about 40 to 60 mg/m², e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more mg/mm². Alternatively, the exemplary therapeutic agents disclosed herein above are envisioned to be administered at a concentration of, e.g., about or at least 0.001 to 100 mg/kg or 0.1 to 1 mg/kg. In some embodiments, the exemplary therapeutic agents disclosed herein above are envisioned to be administered at a concentration of, e.g., about or at least from 0.01 to 10 mg/kg.

The target peptide compositions (or target peptide composition kits) of the presently disclosed subject matter can in some embodiments also be provided with administration of cytokines such as lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha-beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand or FLT-3, angiostatin, thrombospondin, endostatin, tumor necrosis factor and LT. As used herein, the term cytokine includes proteins from natural sources or from recombinant cell culture and biologically active equivalents of the native sequence cytokines.

The target peptide compositions of the presently disclosed subject matter can in some embodiments be provided with administration of cytokines around the time, (e.g., about or at least 1, 2, 3, or 4 weeks or days before or after) of the initial dose of a target peptide composition.

Exemplary, non-limiting doses of a cytokine would be about or at least 1-100, 10-80, 20-70, 30-60, 40-50, or 1, 2, 3, 4, 5, 6, 7, 8, 9, or 10 Mu/m²/day over about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. The cytokine can in some embodiments be delivered at least or about once every 1, 2, 3, 4, 5, 6, 7, 8, 9, 10, 11, 12, 13, 14, 15, 16, 17, 18, 19, 20, 21, 22, 23, or 24 hours. Cytokine treatment can in some embodiments be provided in at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 cycles of at least or about 1, 2, 3, 4, 5, 6, 7, 8, 9, or 10 weeks, wherein each cycle has at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 cytokine doses. Cytokine treatment can be on the same schedule as administration of the target peptide compositions or on a different (but in some embodiments overlapping) schedule.

In some embodiments, the cytokine is IL-2 and is dosed in an amount of about or at least 100,000 to 1,000,000; 200,000-900,000; 300,000-800,000; 450,000-750,000; 600,000-800,000; or 700,000-800,000; or 720,000 units (IU)/kg administered, e.g., as a bolus, every 1, 2, 3, 4, 5, 6, 7, 8, 9, 10, 11, 12, 13, 14, 15, 16, 17, 18, 19, or 20 hours for 1, 2, 3, 4, 5, 6, 7, 8, 9, 10, 11, 12, 13, or 14 days, in a cycle, for example.

### VI Types of Proliferative Disease

The compositions of the presently disclosed subject matter are envisioned to useful in the treatment of benign and malignant proliferative diseases. Excessive proliferation of cells and turnover of cellular matrix can contribute significantly to the pathogenesis of several diseases, including but not limited to cancer, atherosclerosis, rheumatoid arthritis, psoriasis, idiopathic pulmonary fibrosis, scleroderma and cirrhosis of the liver, ductal hyperplasia, lobular hyperplasia, papillomas, and others.

In some embodiments, the proliferative disease is cancer, which in some embodiments is selected from the group consisting of HCC, esophageal cancer, breast cancer, colorectal cancer, squamous carcinoma of the lung, sarcoma, renal cell carcinoma, pancreatic carcinomas, squamous tumors of the head and neck, leukemia, brain cancer, liver cancer, prostate cancer, ovarian cancer, and cervical cancer. In some embodiments, the compositions of the presently disclosed subject matter are used to treat HCC, esophageal cancer, colorectal cancer, acute myelogenous leukemia (AML), acute lyphocytic leukemia (ALL), chronic lymphocytic lymphoma (CLL), chronic myelogenous leukemia (CIVIL), breast cancer, renal cancer, pancreatic cancer, and/or ovarian cancer.

In some embodiments, the cancer is a cancer of the bladder (including accelerated and metastatic bladder cancer), breast (e.g., estrogen receptor positive breast cancer, estrogen receptor negative breast cancer, HER-2 positive breast cancer, HER-2 negative breast cancer, triple negative breast cancer, inflammatory breast cancer), colon (including colorectal cancer), kidney (e.g., renal cell carcinoma), liver, lung (including small cell lung cancer and non-small cell lung cancer (including adenocarcinoma, squamous cell carcinoma, bronchoalveolar carcinoma and large cell carcinoma)), genitourinary tract, e.g., ovary (including fallopian, endometrial and peritoneal cancers), cervix, prostate and testes, lymphatic system, rectum, larynx, pancreas (including exocrine pancreatic carcinoma), stomach (e.g., gastroesophageal, upper gastric or lower gastric cancer), gastrointestinal cancer (e.g., anal cancer), gall bladder, thyroid, lymphoma (e.g., Burkitt's, Hodgkin's, or non-Hodgkin's lymphoma), leukemia (e.g., acute myeloid leukemia), Ewing's sarcoma, nasoesophageal cancer, nasopharyngeal cancer, neural and glial cell cancers (e.g., glioblastoma multiforme), and head and neck. Exemplary cancers include but are not limited to HCC, esophageal cancer (including Barrett's esophagus (BE), high-grade dysplasia (HGD), and invasive cancer including but not limited to squamous cell carcinoma and adenocarcinoma), melanoma, breast cancer (e.g., metastatic or locally advanced breast cancer), prostate cancer (e.g., hormone refractory prostate cancer), renal cell carcinoma, lung cancer (e.g., small cell lung cancer and non-small cell lung cancer (including adenocarcinoma, squamous cell carcinoma, bronchoalveolar carcinoma and large cell carcinoma)), pancreatic cancer, gastric cancer (e.g., gastroesophageal, upper gastric or lower gastric cancer), colorectal cancer, squamous cell cancer of the head and neck, ovarian cancer (e.g., advanced ovarian cancer, platinum-based agent resistant or relapsed ovarian cancer), lymphoma (e.g., Burkitt's, Hodgkin's, or non-Hodgkin's lymphoma), leukemia (e.g., acute myeloid leukemia), and gastrointestinal cancer.

### VII. Administration of Vaccine Compositions

VII.A. Routes of Administration

The target peptide compositions of the presently disclosed subject matter can in some embodiments be administered parenterally, systemically, and/or topically. By way of example and not limitation, composition injection can be performed by intravenous (i.v). injection, sub-cutaneous (s.c). injection, intradermal (i.d). injection, intraperitoneal (i.p). injection, and/or intramuscular (i.m). injection. One or more such routes can be employed. Parenteral administration can be, for example, by bolus injection or by gradual perfusion over time. Alternatively or concurrently, administration can be by the oral route.

In some embodiments, intradermal (i.d). injection is employed. The target peptide compositions of the presently disclosed subject matter are suitable for administration of the peptides by any acceptable route such as oral (enteral), nasal, ophthal, or transdermal. In some embodiments, the administration is subcutaneous and can be administered by an infusion pump.

VII.B. Formulation

Pharmaceutical carriers, diluents, and excipients are generally added to the target peptide compositions or (target peptide compositions kits) that are compatible with the active ingredients and acceptable for pharmaceutical use. Examples of such carriers include, but are not limited to, water, saline solutions, dextrose, and/or glycerol. Combinations of carriers can also be used. The vaccine compositions can further incorporate additional substances to stabilize pH and/or to function as adjuvants, wetting agents, and/or emulsifying agents, which can serve to improve the effectiveness of the vaccine.

The target peptide compositions can include one or more adjuvants such but not limited to montanide ISA-51 (Seppic, Inc., Fairfield, N.J., United States of America); QS-21 STIMULON® brand adjuvant (Agenus Inc., Lexington, Mass., United States of America); ARLACEL® A brand mannide monooleate; oeleic acid; tetanus helper peptides (e.g., QYIKANSKFIGITEL (SEQ ID NO: 449) or AQYIKANSKFIGITEL (SEQ ID NO: 450); GM-CSF; cyclophosamide; bacillus Calmette-Guerin (BCG); corynbacterium parvum; levamisole, azimezone; isoprinisone; dinitrochlorobenezene (DNCB); keyhole limpet hemocyanins (KLH) including Freunds adjuvant (complete and incomplete); mineral gels; aluminum hydroxide (Alum); lysolecithin; pluronic polyols; polyanions; peptides; oil emulsions; nucleic acids (e.g., dsRNA) dinitrophenol; diphtheria toxin (DT); toll-like receptor (TLR, e.g., TLR3, TLR4, TLR7, TLR8 or TLR9) agonists (e.g, endotoxins such as lipopolysaccharide (LPS); monophosphoryl lipid A (MPL); polyinosinic-polycytidylic acid (poly-ICLC/HILTONOL®; Oncovir, Inc., Washington, D.C., United States of America); IMO-2055; glucopyranosyl lipid A (GLA); QS-21—a saponin extracted from the bark of the Quillaja saponaria tree, also known as the soap bark tree or Soapbark; resiquimod (TLR7/8 agonist), CDX-1401—a fusion protein consisting of a fully human monoclonal antibody with specificity for the dendritic cell receptor DEC-205 linked to the NY-ESO-1 tumor antigen; Juvaris' Cationic Lipid-DNA Complex; Vaxfectin; and combinations thereof.

Polyinosinic-Polycytidylic acid (Poly IC) is a double-stranded RNA (dsRNA) that acts as a TLR3 agonist. To increase half-life, it has been stabilized with polylysine and carboxymethylcellulose as poly-ICLC. It has been used to induce interferon in cancer patients, with intravenous doses up to 300 μg/kg. Like poly-IC, poly-ICLC is a TLR3 agonist. TLR3 is expressed in the early endosome of myeloid DC; thus poly ICLC preferentially activates myeloid dendritic cells, thus favoring a Th1 cytotoxic T-cell response. Poly ICLC activates natural killer (NK) cells, induces cytolytic potential, and induces IFN-gamma from myeloid DC.

In some embodiments, the adjuvant is provided at about or at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 micrograms per dose or per kg in each dose. In some embodiments, the adjuvant is provided at least or about 0.1, 0.2, 0.3, 0.40, 0.50, 0.60, 0.70, 0.80, 0.90, 0.100, 1.10, 1.20, 1.30, 1.40, 1.50, 1.60, 1.70, 1.80, 1.90, 2.00, 2.10, 2.20, 2.30, 2.40, 2.50, 2.60, 2.70, 2.80, 2.90, 3.00, 3.10, 3.20, 3.30, 3.40, 3.50, 3.60, 3.70, 3.80, 3.90, 4.00, 4.10, 4.20, 4.30, 4.40, 4.50, 4.60, 4.70, 4.80, 4.90, 5.00, 5.10, 5.20, 5.30, 5.40, 5.50, 5.60, 5.70, 5.80, 5.90, 6.00, 6.10, 6.20, 6.30, 6.40, 6.50, 6.60, 6.70, 6.80, 6.90, 7.00, 7.10, 7.20, 7.30, 7.40, 7.50, 7.60, 7.70, 7.80, 7.90, 8.00, 8.10, 8.20, 8.30, 8.40, 8.50, 8.60, 8.70, 8.80, 8.90, 9.00, 9.10, 9.20, 9.30, 9.40, 9.50, 9.60, 9.70, 9.80, or 9.90 grams per dose or per kg in each dose. In some embodiments, the adjuvant is given at about or at least 10, 15, 20, 25, 50, 75, 100, 125, 150, 175, 150, 175, 200, 225, 250, 275, 300, 325, 350, 375, 400, 425, 450, 500, 525, 550, 575, 600, 625, 675, 700, 725, 750, 775, 800, 900, 1000, 1100, 1200, 1300, 1400, 1500, 1600, 1700, 1800, 1900, or 2000 endotoxin units (“EU”) per dose.

The target peptide compositions of the presently disclosed subject matter can in some embodiments be provided with an administration of cyclophosamide around the time, (e.g., about or at least 1, 2, 3, or 4 weeks or days before or after) the initial dose of a target peptide composition. An exemplary dose of cyclophosamide would in some embodiments be about or at least 100, 200, 300, 400, 500, 600, 700, 800, 900, or 1000 mg/m²/day over about or at least 1, 2, 3, 4, 5, 6, 7, 8, 9, or 10 days.

The compositions of the presently disclosed subject matter can in some embodiments comprise the presently disclosed target peptides in the free form and/or in the form of a pharmaceutically acceptable salt.

As used herein, “a pharmaceutically acceptable salt” refers to a derivative of the disclosed target peptides wherein the target peptide is modified by making acid or base salts of the target peptide. For example, acid salts are prepared from the free base (typically wherein the neutral form of the drug has a neutral—NH₂ group) involving reaction with a suitable acid. Suitable acids for preparing acid salts include both organic acids such as but not limited to acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids such as but not limited to hydrochloric acid, hydrobromic acid, sulfuric acid, nitric acid, phosphoric acid, and the like. Conversely, basic salts of acid moieties which can be present on a target peptide are prepared using a pharmaceutically acceptable base such as sodium hydroxide, potassium hydroxide, ammonium hydroxide, calcium hydroxide, trimmethylamine or the like. By way of example and not limitation, the compositions can in some embodiments comprise the target peptides as salts of acetic acid (acetates), ammonium, or hydrochloric acid (chlorides).

In some embodiments, a composition can include one or more sugars, sugar alcohols, amino acids such a glycine, arginine, glutaminic acid, and others as framework former. The sugars can be mono-, di- or trisaccharide. These sugars can be used alone, as well as in combination with sugar alcohols. Examples of sugars include glucose, mannose, galactose, fructose or sorbose as monosaccharides, sucrose, lactose, maltose or trehalose as disaccharides and raffinose as a trisaccharide. A sugar alcohol can be, for example, mannitose. In some embodiments, the composition comprises sucrose, lactose, maltose, trehalose, mannitol and/or sorbitol. In some embodiments, the composition comprises mannitol.

Furthermore, in some embodiments the presently disclosed compositions can include physiological well-tolerated excipients (see e.g., the Rowe et al., 2006), such as antioxidants like ascorbic acid or glutathione, preserving agents such as phenol, m-cresole, methyl- or propylparabene, chlorobutanol, thiomersal or benzalkoniumchloride, stabilizer, framework former such as sucrose, lactose, maltose, trehalose, mannitose, mannitol and/or sorbitol, mannitol and/or lactose and solubilizer such as polyethyleneglycols (PEG), i.e. PEG 3000, 3350, 4000, or 6000, or cyclodextrines, i.e. hydroxypropyle-β-cyclodextrine, sulfobutylethyl-β-cyclodextrine or γ-cyclodextrine, or dextranes or poloxaomers, i.e. poloxaomer 407, poloxamer 188, or TWEEN™ 20, TWEEN™ 80. In some embodiments, one or more well tolerated excipients can be included, selected from the group consisting of antioxidants, framework formers, and stabilizers.

In some embodiments, the pH for intravenous and intramuscular administration is selected from pH 2 to pH 12, while the pH for subcutaneous administration is selected from pH 2.7 to pH 9.0 as the rate of in vivo dilution is reduced resulting in more potential for irradiation at the injection site. (Strickley, 2004).

VII.C. Dosage

It is understood that a suitable dosage of a target peptide composition vaccine immunogen will depend upon the age, sex, health, and weight of the recipient, the kind of concurrent treatment, if any, the frequency of treatment, and the nature of the effect desired. However, a desired dosage can be tailored to the individual subject, as determined by the researcher or clinician. The total dose employed for any given treatment can typically be determined with respect to a standard reference dose based on the experience of the researcher or clinician, such dose being administered either in a single treatment or in a series of doses, the success of which can depend on the production of a desired immunological result (i.e., successful production of a T helper cell and/or CTL-mediated response to the target peptide immunogen composition, which response gives rise to the prevention and/or treatment desired). Thus, in some embodiments the overall administration schedule can be considered in determining the success of a course of treatment and not whether a single dose, given in isolation, would or would not produce the desired immunologically therapeutic result or effect. As such, a therapeutically effective amount (i.e., that producing the desired T helper cell and/or CTL-mediated response) can in some embodiments depend on the antigenic composition of the vaccine used, the nature of the disease condition, the severity of the disease condition, the extent of any need to prevent such a condition where it has not already been detected, the manner of administration dictated by the situation requiring such administration, the weight and state of health of the individual receiving such administration, and/or the sound judgment of the clinician or researcher. Needless to say, the efficacy of administering additional doses and of increasing or decreasing the interval can be re-evaluated on a continuing basis, in view of the recipient's immunocompetence (for example, the level of T helper cell and/or CTL activity with respect to tumor-associated or tumor-specific antigens).

The concentration of the T helper or CTL stimulatory target peptides of the presently disclosed subject matter in pharmaceutical formulations are subject to wide variation, including anywhere from less than 0.01% by weight to as much as 50% or more. Factors such as volume and viscosity of the resulting composition can also be considered. The solvents, or diluents, used for such compositions can include one or more of water, phosphate buffered saline (PBS), saline itself, and/or other possible carriers and/or excipients. The immunogens of the presently disclosed subject matter can in some embodiments also be contained in artificially created structures such as liposomes, which structures can in some embodiments contain additional molecules, such as proteins or polysaccharides, inserted in the outer membranes of the structures and having the effect of targeting the liposomes to particular areas of the body, or to particular cells within a given organ or tissue. Such targeting molecules can in some embodiments be some type of immunoglobulin. Antibodies can work particularly well for targeting the liposomes to tumor cells.

Single i.d., i.m., s.c., i.p., and/or i.v. doses of e.g., about 1 to 50 μg, 1 to 100 μg, 1 to 500 μg, 1 to 1000 μg, or about 1 to 50 mg, 1 to 100 mg, 1 to 500 mg, or 1 to 1000 mg of a target peptide composition of the presently disclosed subject matter can in some embodiments be given and in some embodiments can depend from the respective compositions of target peptides with respect to total amount for all target peptides in the composition or alternatively for each individual target peptide in the composition. A single dose of a target peptide vaccine composition of the presently disclosed subject matter can in some embodiments have a target peptide amount (e.g., total amount for all target peptides in the composition or alternatively for each individual target peptide in the composition) of about or at least 1, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 950 μg. Alternatively, a single dose of a target peptide composition of the presently disclosed subject matter can in some embodiments have a total target peptide amount (e.g., total amount for all target peptides in the composition or alternatively for each individual target peptide in the composition) of about or at least 1, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 950 mg. In some embodiments, the target peptides of a composition of the presently disclosed subject matter are present in equal amounts of about 100 micrograms per dose in combination with an adjuvant peptide present in an amount of about 200 micrograms per dose.

In a single dose of the target peptide composition of the presently disclosed subject matter, the amount of each target peptide in the composition is in some embodiments equal or is in some embodiments substantially equal. Alternatively, the ratio of the target peptides present in the least amount relative to the target peptide present in the greatest amount is in some embodiments about or at least 1:1.25, 1:1.5, 1:1.75, 1:2.0, 1:2.25, 1:2.5, 1:2.75, 1:3, 1:4, 1:5, 1:6, 1:7, 1:8, 1:9, 1:10, 1:20, 1:30; 1:40, 1:50, 1:100, 1:200, 1:500, 1:1000, 1:5000; 1:10,000; or 1:100,000. Alternatively, the ratio of the target peptides present in the least amount relative to the target peptide present in the greatest amount is in some embodiments about or at least 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15 to 20; 20 to 25; 1 to 40; 1 to 30; 1 to 20; 1 to 15; 10 to 40; 10 to 30; 10 to 20; 10 to 15; 20 to 40; 20 to 30; or 20 to 25; 1 to 100; 25 to 100; 50 to 100; 75 to 100; 25 to 75, 25 to 50, or 50 to 75; 25 to 40; 25 to 50; 30 to 50; 30 to 40; or 30 to 75.

Single dosages can in some embodiments be given to a patient about or at least 1, 2, 3, 4, or 5 times per day. Single dosages can in some embodiments be given to a patient about or at least 1, 2, 3, 4, 5, 6, 7, 8, 9, 10, 11, 12, 13, 14, 15, 16, 18, 19, 20, 21, 22, 23, 24, 36, 48, 60, or 72 hours subsequent to a previous dose.

Single dosages can in some embodiments be given to a patient about or at least 1, 2, 3, 4, 5, 6, or 7 times per week or every other, third, fourth, or fifth day. Single doses can in some embodiments also be given every week, every other week, or only during 1, 2, or 3 weeks per month. A course of treatment can in some embodiments last about or at least 1, 2, 3, 4, 5, 6, 7, 8, 9, 10, 11, or 12 months.

In some embodiments, single dosages of the compositions of the presently disclosed subject matter are provided to a patient in at least two phases, e.g., during an initial phase and then a subsequent phase. An initial phase can in some embodiments be about or at least 1, 2, 3, 4, 5, or 6 weeks in length. The subsequent phase can in some embodiments last at least or about 1, 2, 3, 4, 5, 6, 7, or 8 times as long as the initial phase. The initial phase can in some embodiments be separated from the subsequent phase by about or at least 1, 2, 3, 4, 5, 6, 7, 8, 9, 10, 11, or 12 weeks or months.

The target peptide composition dosage during the subsequent phase can in some embodiments be at least 1, 2, 3, 4, 5, 6, 7, 8, 9, 10, 15, 20, 30, 40, 50, 60, 70, 80, 90, 100, 200, 300, 400, 500, 600, 700, 800, 900, or 1000 times greater than during the initial phase. The target peptide composition dosage during the subsequent phase can in some embodiments be at least 1, 2, 3, 4, 5, 6, 7, 8, 9, 10, 15, 20, 30, 40, 50, 60, 70, 80, 90, 100, 200, 300, 400, 500, 600, 700, 800, 900, or 1000 times lower than during the initial phase.

In some embodiments, the initial phase is about three weeks and the second phase is about 9 weeks. In some embodiments, the target peptide compositions would be administered to the patient on or about days 1, 8, 15, 36, 57, and 78.

VII.D. Kits and Storage

In some embodiments, the presently disclosed subject matter provides a kit. In some embodiments the kit comprises (a) a container that contains at least one target peptide composition as described above in solution or in lyophilized form; (b) optionally, a second container containing a diluent or reconstituting solution for the lyophilized formulation; and (c) also optionally, instructions for (i) use of the solution; and/or (ii) reconstitution and/or use of the lyophilized formulation. The kit can in some embodiments further comprise one or more of (iii) a buffer, (iv) a diluent, (v) a filter, (vi) a needle, and/or (v) a syringe. In some embodiments, the container is selected from the group consisting of a bottle, a vial, a syringe, a test tube, and a multi-use container. In some embodiments, the target peptide composition is lyophilized.

The kits can in some embodiments contain exactly, about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, or more target peptide-containing compositions. Each composition in the kit can in some embodiments be administered at the same time or at different times to a subject.

In some embodiments, the kits can comprise a lyophilized formulation of the presently disclosed compositions and/or vaccines in a suitable container and instructions for its reconstitution and/or use. Suitable containers include, for example, bottles, vials (e.g. dual chamber vials), syringes (such as dual chamber syringes), and test tubes. The container can in some embodiments be formed from a variety of materials such as glass or plastic. In some embodiments, the kit and/or container include instructions on or associated with the container that indicate directions for reconstitution and/or use. For example, the label can in some embodiments indicate that the lyophilized formulation is to be reconstituted to target peptide concentrations as described above. The label can in some embodiments further indicate that the formulation is useful or intended for subcutaneous administration. Lyophilized and liquid formulations are in some embodiments stored at −20° C. to −80° C.

The container holding the target peptide composition(s) can in some embodiments be a multi-use vial, which allows for repeat administrations (e.g., from 2-6 administrations) of the reconstituted formulation. The kit can in some embodiments further comprise a second container comprising a suitable diluent such as, but not limited to a sodium bicarbonate solution.

In some embodiments, upon mixing of the diluent and the lyophilized formulation, the final peptide concentration in the reconstituted formulation is at least or about 0.15, 0.20, 0.25, 0.5, 0.75, 1.0, 1.25, 1.5, 1.75, 2.0, 2.25, 2.5, 2.75, 3.0, 3.25, 3.50, 3.75, 4.0, 4.25, 4.5, 4.75, 5.0, 6.0, 7.0, 8.0, 9.0, or 10 mg/mL/target peptide. In some embodiments, upon mixing of the diluent and the lyophilized formulation, the final peptide concentration in the reconstituted formulation is at least or about 0.15, 0.20, 0.25, 0.5, 0.75, 1.0, 1.25, 1.5, 1.75, 2.0, 2.25, 2.5, 2.75, 3.0, 3.25, 3.50, 3.75, 4.0, 4.25, 4.5, 4.75, 5.0, 6.0, 7.0, 8.0, 9.0 or 10 μg/mL/target peptide.

The kit can in some embodiments further comprise other materials desirable from a commercial and user standpoint, including but not limited to other buffers, diluents, filters, needles, syringes, and/or package inserts with instructions for use.

The kits can in some embodiments have a single container that comprises the formulation of the target peptide compositions with or without other components (e.g., other compounds or compositions of these other compounds) or can in some embodiments have a distinct container for each component.

Additionally, the kits can in some embodiments comprise a formulation of the presently disclosed target peptide compositions and/or vaccines packaged for use in combination with the co-administration of a second compound such as but not limited to adjuvants (e.g. imiquimod), a chemotherapeutic agent, a natural product, a hormone or antagonist, an anti-angiogenesis agent or inhibitor, an apoptosis-inducing agent, or a chelator or a composition thereof. The components of the kit can in some embodiments be pre-complexed or each component can in some embodiments be in a separate distinct container prior to administration to a patient. The components of the kit can in some embodiments be provided in one or more liquid solutions. In some embodiments, the liquid solution is an aqueous solution. In some embodiments, the liquid solution is a sterile aqueous solution. The components of the kit can in some embodiments also be provided as solids, which in some embodiments are converted into liquids by addition of suitable solvents, which can in some embodiments be provided in another distinct container.

The container of a therapeutic kit can in some embodiments be a vial, a test tube, a flask, a bottle, a syringe, or any other article suitable to enclose a solid or liquid. In some embodiments, when there is more than one component, the kit can contain a second vial and/or other container, which allows for separate dosing. The kit can in some embodiments also contain another container for a pharmaceutically acceptable liquid. In some embodiments, a therapeutic kit contains an apparatus (e.g., one or more needles, syringes, eye droppers, pipette, etc.) that facilitates administration of the agents of the disclosure that are components of the present kit.

VII.E. Markers for Efficacy

When administered to a patient, the vaccine compositions of the presently disclosed subject matter are envisioned to have certain physiological effects, including but not limited to the induction of a T cell mediated immune response.

VII.E.1 Immunohistochemistry, Immunofluorescence, Western Blots, and Flow Cytometry

Validation and testing of antibodies for characterization of cellular and molecular features of lymphoid neogenesis has been performed. Commercially available antibodies for use in immunohistochemistry (IHC), immunofluorescence (IF), flow cytometry (FC), and western blot (WB) can in some embodiments be employed. In some embodiments, such techniques can be employed to analyze patient samples, e.g., formalin-fixed, paraffin-embedded tissue samples, for CD1a, S100, CD83, DC-LAMP, CD3, CD4, CD8, CD20, CD45, CD79a, PNAd, TNFalpha, LIGHT, CCL19, CCL21, CXCL12, TLR4, TLR7, FoxP3, PD-1 and Ki67 expression. In some embodiments, flow cytometry is used to determine CD3, CD4, CD8, CD13, CD14, CD16, CD19, CD45RA, CD45RO, CD56, CD62L, CD27, CD28, CCR7, FoxP3 (intracellular), and MHC-peptide tetramers for I MHC associated (phospho)-peptides. In some embodiments, positive control tissue selected from among normal human peripheral blood lymphocytes (PBL), PBL activated with CD3/CD28 beads (activated PBL), human lymph node tissue from non-HCC patients (LN), and inflamed human tissue from a surgical specimen of Crohn's disease (Crohn's) can be employed.

VII.E.2. ELISpot Assay

In some embodiments, vaccination site infiltrating lymphocytes and lymphocytes from the sentinel immunized nod (SIN) and vaccine site can be evaluated by ELISpot. ELISpot permits the direct counting of T-cells reacting to antigen by production of INFγ. Peripheral blood lymphocytes can be evaluated by ELISpot assay for the number of peptide-reactive T-cells. Vaccine site infiltrating lymphocytes and SIN lymphocytes can be compared to those in peripheral blood. It is envisioned that positive results of the ELISpot assay correlate with increased patient progression free survival. Progression free survival is in some embodiments defined as the time from start of treatment until death from any cause or date of last follow up.

VII.E.3. Tetramer Assay

Peripheral blood lymphocytes and lymphocytes from the SIN and vaccine site can be evaluated by flow cytometry after incubation with MHC-peptide tetramers for the number of peptide-reactive T-cells.

VII.E.4. Proliferation Assay/Cytokine Analysis

Peripheral blood mononuclear cells (PBMC), vaccine-site inflammatory cells, and lymphocytes from the SIN from patients can in some embodiments be evaluated for CD4 T cell reactivity to, e.g., tetanus helper peptide mixture, using a ³H-thymidine uptake assay. Additionally, Th1 (IL-2, IFN-gamma, TNFa), Th2 (IL-4, IL-5, IL-10), Th17 (IL-17, and IL23), and T-reg (TGF-beta) cytokines in media from 48 hours in that proliferation assay can be employed to determine if the microenvironment supports generation of Th1, Th2, Th17, and/or T-reg responses. In some embodiments, two peptides are used as negative controls: a tetanus peptide and the Pan DR T helper epitopes (PADRE) peptide (AK(X)VAAWTLKAA; SEQ ID NO: 500).

VII.E.5. Evaluation of Tumors

In some embodiments tumor tissue collected prior to treatment or at the time of progression can be evaluated by routine histology and immunohistochemistry. Alternatively or in addition, in vitro evaluations of tumor tissue and tumor infiltrating lymphocytes can be completed.

VII.E.6. Studies of Homing Receptor Expression

Patient samples can in some embodiments be studied for T cell homing receptors induced by vaccination the compositions of the presently disclosed subject matter. These include, but are not limited to, integrins (including alphaE-beta7, alpha1-beta1, alpha4-beta1), chemokine receptors (including CXCR3), and selectin ligands (including CLA, PSL) on lymphocytes, and their ligands in the vaccine sites and SIN. These can be assayed by immunohistochemistry, flow cytometry or other techniques.

VII.E.7. Studies of Gene and Protein Expression

Differences in gene expression and/or for differences in panels of proteins can in some embodiments be assayed by high-throughput screening assays (e.g. nucleic acid chips, protein arrays, etc.) in the vaccine sites and sentinel immunized nodes.

### VIII. Antibodies Including Antibody-Like Molecules

In some embodiments, the present disclosure provides antibodies and antibody-like molecules (e.g. T cell receptors) that specifically bind to the target peptides (e.g., phosphopeptides) disclosed herein, or to complexes of an MHC molecule (e.g., a class I MHC fmolecule) and the peptides disclosed herein. In some embodiments, the antibodies and antibody-like molecules (e.g. T cell receptors) specifically bind to complexes of phosphopeptides and corresponding MHC alleles as set forth in Tables 2-14.

Antibodies and antibody-like molecules (e.g. T cell receptors) specific for target peptides or target peptide/MHC complexes are, for example, useful, inter alia, for analyzing tissue to determine the pathological nature of tumor margins and/or can be employed in some embodiments as therapeutics. Alternatively, such molecules can in some embodiments be employed as therapeutics targeting cells, e.g., tumor cells, which display target peptides on their surface. In some embodiments, the antibodies and antibody-like molecules bind the target peptides or target peptide-MHC complex specifically and do not substantially cross react with non-phosphorylated native peptides.

As used herein, “antibody” and “antibody peptide(s)” refer to intact antibodies, antibody-like molecules, and binding fragments thereof that compete with intact antibodies for specific binding. Binding fragments are in some embodiments produced by recombinant DNA techniques or in some embodiments by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody in some embodiments substantially inhibits adhesion of a receptor to a counterreceptor when an excess of antibody reduces the quantity of receptor bound to counterreceptor by at least about 20%, 40%, 60%, 80%, 85%, 90%, 91%, 92%, 93%, 94%, 95%, 96%, 97%, 98%, 99%, or greater than 99% as measured, for example, in an in vitro competitive binding assay.

The term “MHC” as used herein refers to the Major Histocompability Complex, which is defined as a set of gene loci specifying major histocompatibility antigens. The term “HLA” as used herein refers to Human Leukocyte Antigens, which are defined as the histocompatibility antigens found in humans. As used herein, “HLA” is the human form of “MHC”.

The terms “MHC light chain” and “MHC heavy chain” as used herein refer to portions of MHC molecules. Structurally, class I molecules are heterodimers comprised of two non-covalently bound polypeptide chains, a larger “heavy” chain (a) and a smaller “light” chain (β-2-microglobulin or β2m). The polymorphic, polygenic heavy chain (45 kDa), encoded within the MHC on chromosome six, is subdivided into three extracellular domains (designated 1, 2, and 3), one intracellular domain, and one transmembrane domain. The two outermost extracellular domains, 1 and 2, together form the groove that binds antigenic peptide. Thus, interaction with the TCR occurs at this region of the protein. The 3 domain of the molecule contains the recognition site for the CD8 protein on the CTL; this interaction serves to stabilize the contact between the T cell and the APC. The invariant light chain (12 kDa), encoded outside the MHC on chromosome 15, consists of a single, extracellular polypeptide. The terms “MHC light chain”, “β2-microglobulin”, and “β2m” are used interchangeably herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody or antibody like molecule is said to “specifically” bind an antigen when the dissociation constant is in some embodiments less than 1 μM, in some embodiments less than 100 nM, and in some embodiments less than 10 nM.

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)2 and Fv), as well as “antibody-like molecules” so long as they exhibit the desired biological activity. Antibodies (Abs) and immunoglobulins (Igs) are glycoproteins having the same structural characteristics. The term is also meant to encompass “antibody like molecules” and other members of the immunoglobulin superfamily, e.g., T-cell receptors, WIC molecules, containing e.g., an antigen-binding regions and/or variable regions, e.g., complementary determining regions (CDRs) which specifically bind the target peptides disclosed herein.

In some embodiments, antibodies and antibody-like molecules bind to the target peptides of the presently disclosed subject matter but do not substantially and/or specifically cross react with the same peptide in a modified form. See e.g., U.S. Patent Application Publication No. 2009/0226474, which is incorporated by reference.

The presently disclosed subject matter also includes antibodies that recognize target peptides associated with a tumorigenic or disease state, wherein the peptides are displayed in the context of HLA molecules. These antibodies typically mimic the specificity of a T cell receptor (TCR) but can in some embodiments have higher binding affinity such that the molecules can be employed as therapeutic, diagnostic, and/or research reagents. Methods of producing a T-cell receptor mimic of the presently disclosed subject matter include identifying a target peptide of interest (e.g., a phosphopeptide), wherein the target peptide of interest comprises an amino acid sequence as set forth in any of SEQ ID NOs: 1-448 and 502-529 (e.g., a phosphopeptide as set forth in Tables 2-14 herein). Then, an immunogen comprising at least one target peptide/WIC complex is formed. An effective amount of the immunogen is then administered to a host for eliciting an immune response, and serum collected from the host is assayed to determine if desired antibodies that recognize a three-dimensional presentation of the target peptide in the binding groove of the MHC molecule are being produced. The desired antibodies can differentiate the target peptide/MHC complex from the MHC molecule alone, the target peptide alone, and a complex of MHC and irrelevant target peptide. Finally, in some embodiments the desired antibodies are isolated.

The term “antibody” also encompasses soluble T cell receptors (TCR) which are stable at low concentrations and which can recognize MHC-peptide complexes. See e.g., U.S. Patent Application Publication No. 2002/0119149, which is incorporated by reference. Such soluble TCRs might for example be conjugated to immunostimulatory peptides and/or proteins or moieties, such as CD3 agonists (anti-CD3 antibody), for example. The CD3 antigen is present on mature human T cells, thymocytes, and a subset of natural killer cells. It is associated with the TCR and is responsible for the signal transduction of the TCR.

Antibodies specific for the human CD3 antigen are well-known. One such antibody is the murine monoclonal antibody OKT3 which was the first monoclonal antibody approved by the FDA. OKT3 is reported to be a potent T cell mitogen (see e.g., Van Wauve, 1980; U.S. Pat. No. 4,361,539) and a potent T cell killer (Wong, 1990. Other antibodies specific for the CD3 antigen have also been reported (see e.g., PCT International Patent Application Publication No. WO 2004/0106380; U.S. Patent Application Publication No. 2004/0202657; U.S. Pat. Nos. 6,750,325; 6,706,265; GB 2249310A; Clark et al., 1989; U.S. Pat. No. 5,968,509; and U.S. Patent Application Publication No. 2009/0117102). ImmTACs (Immunocore Limited, Milton Park, Abington, Oxon, United Kingdom) are innovative bifunctional proteins that combine high-affinity monoclonal T cell receptor (mTCR) targeting technology with a clinically-validated, highly potent therapeutic mechanism of action (Anti-CD3 scFv).

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond. The number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Chothia et al., 1985; Novotny & Haber, 1985).

An “isolated” antibody is one which has been separated, identified, and/or recovered from a component of the environment in which it was produced. Contaminant components of its production environment are materials which would interfere with diagnostic or therapeutic uses for the antibody, and can include enzymes, hormones, and other proteinaceous or nonproteinaceous solutes. In some embodiments, the antibody is purified as measurable by at least one of the following three different methods: 1) to in some embodiments greater than 50% by weight of antibody as determined by the Lowry method, such as but not limited to in some embodiments greater than 75% by weight, in some embodiments greater than 85% by weight, in some embodiments greater than 95% by weight, in some embodiments greater than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, in some embodiments, silver stain. Isolated antibodies include the antibody in situ within recombinant cells since at least one component of the antibody's natural environment is not present. In some embodiments, however, isolated antibodies are prepared by a method that includes at least one purification step.

The terms “antibody mutant”, “antibody variant”, and “antibody derivative” refer to an amino acid sequence variant of an antibody wherein one or more of the amino acid residues of a reference antibody has been modified (e.g., substituted, deleted, chemically modified, etc.). Such mutants necessarily have less than 100% sequence identity or similarity with the amino acid sequence of either the heavy or light chain variable domain of the reference antibody. The resultant sequence identity or similarity between the modified antibody and the reference antibody is thus in some embodiments at least 80%, in some embodiments at least 85%, in some embodiments at least 90%, in some embodiments at least 95%, in some embodiments at least 97%, and in some embodiments at least 99%.

The term “variable” in the context of variable domain of antibodies, refers to the fact that certain portions of the variable domains differ extensively in sequence among antibodies and are used in the binding and specificity of each particular antibody for its particular antigen(s). However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (Kabat et al., 1987); and (2) an approach based on crystallographic studies of antigen-antibody complexes (Chothia et al., 1989). The more highly conserved portions of variable domains are called the framework (FR) regions. The variable domains of native heavy and light chains each comprise four FR regions, largely adopting a β-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies (see Kabat et al., 1987). The constant domains are not involved directly in binding an antibody to an antigen, but exhibit various effector function, such as participation of the antibody in antibody-dependent cellular toxicity.

The term “antibody fragment” refers to a portion of a full-length antibody, generally the antigen binding or variable region. Examples of antibody fragments include Fab, Fab′, F(ab′)2 and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)2 fragment that has two antigen binding fragments which are capable of cross-linking antigen, and a residual other fragment (which is termed pFc′). As used herein, “functional fragment” with respect to antibodies, refers to Fv, F(ab) and F(ab′)2 fragments.

An “Fv” fragment is the minimum antibody fragment which contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)2 pepsin digestion product. Additional chemical couplings of antibody fragments are known to those of ordinary skill in the art.

The light chains of antibodies (immunoglobulin) from any vertebrate species can be assigned to one of two clearly distinct types, called kappa and lambda, based on the amino sequences of their constant domain.

Depending on the amino acid sequences of the constant domain of their heavy chains, immunoglobulins can be assigned to different classes. There are at least five (5) major classes of immunoglobulins: IgA, IgD, IgE, IgG and IgM, and several of these can be further divided into subclasses (isotypes), e.g., IgG₁, IgG₂, IgG₃, and IgG₄; IgA₁ and IgA₂. The heavy chains constant domains that correspond to the different classes of immunoglobulins are called alpha (α), delta (Δ), epsilon (ε), gamma (γ), and mu (μ), respectively. The subunit structures and three-dimensional configurations of different classes of immunoglobulins are well-known.

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, monoclonal antibodies can be advantageous in that they can be synthesized in hybridoma culture, uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter can in some embodiments be made by the hybridoma method first described by Kohler & Milstein, 1975, or can in some embodiments be made by recombinant methods, e.g., as described in U.S. Pat. No. 4,816,567. The monoclonal antibodies for use with the presently disclosed subject matter can in some embodiments also be isolated from phage antibody libraries using the techniques described in Clackson et al., 1991 or in Marks et al., 1991.

Utilization of the monoclonal antibodies of the presently disclosed subject matter can in some embodiments require administration of such or similar monoclonal antibody to a subject, such as a human. However, when the monoclonal antibodies are produced in a non-human animal, such as a rodent, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed subject matter can be “humanized”: that is, the antibodies can be engineered such that antigenic portions thereof are removed and like portions of a human antibody are substituted therefor, while the antibodies' affinity for specific peptide/MHC complexes is retained. This engineering can in some embodiments only involve a few amino acids, or can in some embodiments include entire framework regions of the antibody, leaving only the complementarity determining regions of the antibody intact. Several methods for humanizing antibodies are known in the art and are disclosed, for example, in U.S. Pat. Nos. 4,816,567; 5,712,120; 5,861,155; 5,869,619; 6,054,927; and 6,180,370; the entire content of each of which is hereby expressly incorporated herein by reference in its entirety.

Humanized forms of antibodies are chimeric immunoglobulins, immunoglobulin chains, or fragments thereof (such as Fv, Fab, Fab′, F(ab′)2 or other antigen-binding subsequences of antibodies) that are principally comprised of the sequence of a human immunoglobulin, and contain minimal sequence derived from a non-human immunoglobulin. In some embodiments, humanization can be performed following the method of Winter and co-workers (see e.g., Jones et al., 1986; Riechmann et al., 1988; Verhoeyen et al., 1988) by substituting rodent CDRs or CDR sequences for the corresponding sequences of a human antibody. See also U.S. Pat. No. 5,225,539. In some embodiments, Fᵥ framework residues of a human immunoglobulin are replaced by corresponding non-human residues.

Humanized antibodies can also comprise residues which are found neither in the recipient antibody nor in the imported CDR or framework sequences. In general, a humanized antibody comprises substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally can in some embodiments also comprise at least a portion of an immunoglobulin constant region (Fc), typically that of a human immunoglobulin. See e.g., Jones et al., 1986; Riechmann et al., 1988; Presta, 1992.

Many articles relating to the generation or use of humanized antibodies teach useful examples of protocols that can be utilized with the presently disclosed subject matter, such as but not limited to Shinkura et al., 1998; Yenari et al., 1998; Richards et al., 1999; Morales et al., 2000; Mihara et al., 2001; Sandborn et al., 2001; and Yenari et al., 2001, all of which are expressly incorporated in their entireties by reference. For example, a treatment protocol that can be utilized in such a method includes a single dose, generally administered intravenously, of 10-20 mg of humanized mAb per kg (Sandborn, et al., 2001). In some embodiments, alternative dosing patterns can be appropriate, such as but not limited to the use of three infusions, administered once every two weeks, of 800 to 1600 mg or even higher amounts of humanized mAb (Richards et al., 1999, op. cit.). However, it is to be understood that the presently disclosed subject matter is not limited to the treatment protocols described above, and other treatment protocols that are known to a person of ordinary skill in the art can be utilized in the methods of the presently disclosed subject matter.

The presently disclosed and claimed subject matter further includes in some embodiments fully human monoclonal antibodies against specific target peptide/MHC complexes. Fully human antibodies essentially relate to antibody molecules in which the entire sequence of both the light chain and the heavy chain, including the CDRs, arise from human genes. Such antibodies are referred to herein as “human antibodies” or “fully human antibodies”. Human monoclonal antibodies can be prepared by the trioma technique; the human B-cell hybridoma technique (see Kozbor et al., 1983), and the EBV hybridoma technique to produce human monoclonal antibodies (see Cole et al., 1985). Human monoclonal antibodies can in some embodiments be utilized in the practice of the presently disclosed subject matter and can in some embodiments be produced by using human hybridomas (see Cote et al., 1983)) or by transforming human B-cells with Epstein Barr Virus in vitro (see Cole et al., 1985).

In addition, human antibodies can also be produced using additional techniques, including but not limited to phage display libraries (Hoogenboom et al., 1991; Marks et al., 1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016; and in Marks et al., 1992; Lonberg et al., 1994; Lonberg & Huszar, 1995; Fishwild et al., 1996; Neuberger, 1996.

Human antibodies can in some embodiments additionally be produced using transgenic nonhuman animals which are modified so as to produce fully human antibodies rather than the animal's endogenous antibodies in response to challenge by an antigen. See PCT International Patent Application Publication No. WO 1994/02602). Typically, the endogenous genes encoding the heavy and light immunoglobulin chains in the non-human host are incapacitated, and active loci encoding human heavy and light chain immunoglobulins are inserted into the host's genome. The human genes are incorporated, for example, using yeast artificial chromosomes containing the requisite human DNA segments. An animal that provides all the desired modifications is then obtained as progeny by crossbreeding intermediate transgenic animals containing fewer than the full complement of the modifications.

A non-limiting example of such a nonhuman animal is a mouse, and is termed the XENOMOUSE™ as disclosed in PCT International Patent Application Publication Nos. WO 1996/33735 and WO 1996/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a non-human host, exemplified as a mouse, lacking expression of an endogenous immunoglobulin heavy chain is disclosed in U.S. Pat. No. 5,939,598, incorporated herein by reference). It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

An exemplary method for producing an antibody of interest, such as a human antibody, is disclosed in U.S. Pat. No. 5,916,771 incorporated herein by reference). It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

The antigen target peptides are known to be expressed on a variety of cancer cell types. Thus, antibodies and antibody-like molecules can be used where appropriate, in treating, diagnosing, vaccinating, preventing, retarding, and/or attenuating HCC, melanoma, ovarian cancer, breast cancer, colorectal cancer, squamous carcinoma of the lung, sarcoma, renal cell carcinoma, pancreatic carcinomas, squamous tumors of the head and neck, leukemia, brain cancer, liver cancer, prostate cancer, ovarian cancer, and cervical cancer.

Antibodies generated with specificity for the antigen target peptides can be used to detect the corresponding target peptides in biological samples. The biological sample could come from an individual who is suspected of having cancer and thus detection would serve to diagnose the cancer. Alternatively, the biological sample can in some embodiments come from an individual known to have cancer, and detection of the antigen target peptides would serve as an indicator of disease prognosis, cancer characterization, or treatment efficacy. Appropriate immunoassays are well-known in the art and include, but are not limited to, immunohistochemistry, flow cytometry, radioimmunoassay, western blotting, and ELISA. Biological samples suitable for such testing include, but are not limited to, cells, tissue biopsy specimens, whole blood, plasma, serum, sputum, cerebrospinal fluid, pleural fluid, and urine. Antigens recognized by T cells, whether helper T lymphocytes or CTL, are not recognized as intact proteins, but rather as small peptides that associate with class I or class II MHC proteins on the surface of cells. During the course of a naturally occurring immune response antigens that are recognized in association with class II MHC molecules on antigen presenting cells are acquired from outside the cell, internalized, and processed into small peptides that associate with the class II MHC molecules. Conversely, the antigens that give rise to proteins that are recognized in association with class I MHC molecules are generally proteins made within the cells, and these antigens are processed and associate with class I MHC molecules. It is now well-known that the peptides that associate with a given class I or class II MHC molecule are characterized as having a common binding motif, and the binding motifs for a large number of different class I and II MHC molecules have been determined. It is also well-known that synthetic peptides can be made which correspond to the sequence of a given antigen and which contain the binding motif for a given class I or II MHC molecule. These peptides can then be added to appropriate antigen presenting cells, and the antigen presenting cells can be used to stimulate a T helper cell or CTL response either in vitro or in vivo. The binding motifs, methods for synthesizing the peptides, and methods for stimulating a T helper cell or CTL response are all well-known and readily available.

As used herein, the terms “T cell receptor” and “TCR” are used interchangeably and refer to full length heterodimeric αβ or γδ TCRs, antigen-binding fragments of TCRs, or molecules comprising TCR CDRs or variable regions. Examples of TCRs include, but are not limited to, full-length TCRs, antigen-binding fragments of TCRs, soluble TCRs lacking transmembrane and cytoplasmic regions, single-chain TCRs containing variable regions of TCRs attached by a flexible linker, TCR chains linked by an engineered disulfide bond, monospecific TCRs, multi-specific TCRs (including bispecific TCRs), TCR fusions, human TCRs, humanized TCRs, chimeric TCRs, recombinantly produced TCRs, and synthetic TCRs. The term encompasses wild-type TCRs and genetically engineered TCRs (e.g., a chimeric TCR comprising a chimeric TCR chain which includes a first portion from a TCR of a first species and a second portion from a TCR of a second species).

As used herein, the term “TCR variable region” is understood to encompass amino acids of a given TCR which are not included within the non-variable region as encoded by the TRAC gene for TCR α chains and either the TRBC1 or TRBC2 genes for TCR β chains. In some embodiments, a TCR variable region encompasses all amino acids of a given TCR which are encoded by a TRAV gene or a TRAJ gene for a TCR α chain or a TRBV gene, a TRBD gene, or a TRBJ gene for a TCR β chain (see e.g., LeFranc & LeFranc, 2001, which is incorporated by reference herein in its entirety).

As used herein, the term “constant region” with respect to a TCR refers to the extracellular portion of a TCR that is encoded by the TRAC gene for TCR α chains and either the TRBC1 or TRBC2 genes for TCR β chains. The term constant region does not include a TCR variable region encoded by a TRAV gene or a TRAJ gene for a TCR α chain or a TRBV gene, a TRBD gene, or a TRBJ gene for a TCR β chain (see e.g., LeFranc & LeFranc, 2001, which is incorporated by reference herein in its entirety).

Kits can in some embodiments be composed for help in diagnosis, monitoring, and/or prognosis. The kits are to facilitate the detecting and/or measuring of cancer-specific target peptides or proteins. Such kits can in some embodiments contain in a single or divided container, a molecule comprising an antigen-binding region. Such molecules can in some embodiments be antibodies and/or antibody-like molecules. Additional components that can be included in the kit include, for example, solid supports, detection reagents, secondary antibodies, instructions for practicing, vessels for running assays, gels, control samples, and the like. The antibody and/or antibody-like molecules can in some embodiments be directly or indirectly labeled, as an option.

Alternatively or in addition, the antibody or antibody-like molecules specific for target peptides and/or target peptide/MHC complexes can in some embodiments be conjugated to therapeutic agents. Exemplary therapeutic agents include:

Alkylating Agents:

Alkylating agents are drugs that directly interact with genomic DNA to prevent cells from proliferating. This category of chemotherapeutic drugs represents agents that affect all phases of the cell cycle, that is, they are not phase-specific. An alkylating agent can in some embodiments include, but is not limited to, a nitrogen mustard, an ethylenimene, a methylmelamine, an alkyl sulfonate, a nitrosourea or a triazines. They include but are not limited to busulfan, chlorambucil, cisplatin, cyclophosphamide (cytoxan), dacarbazine, ifosfamide, mechlorethamine (mustargen), and melphalan.

Antimetabolites:

Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to 5-fluorouracil (5-FU), cytarabine (Ara-C), fludarabine, gemcitabine, and methotrexate.

Natural Products:

Natural products generally refer to compounds originally isolated from a natural source, and identified as having a pharmacological activity. Such compounds, as well as analogs and derivatives thereof, can in some embodiments be isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Taxoids are a class of related compounds isolated from the bark of the ash tree, Taxus brevifolia. Taxoids include, but are not limited to, compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. They include such compounds as vinblastine (VLB) and vincristine.

Antibiotics:

Certain antibiotics have both antimicrobial and cytotoxic activity. These drugs can also interfere with DNA by chemically inhibiting enzymes and mitosis or altering cellular membranes. These agents are typically not phase-specific so they work in all phases of the cell cycle. Examples of cytotoxic antibiotics include but are not limited to bleomycin, dactinomycin, daunorubicin, doxorubicin (Adriamycin), plicamycin (mithramycin), and idarubicin.

Miscellaneous Agents:

Miscellaneous cytotoxic agents that do not fall into the previous categories include but are not limited to platinum coordination complexes, anthracenediones, substituted ureas, methyl hydrazine derivatives, amsacrine, L-asparaginase, and tretinoin. Platinum coordination complexes include such compounds as carboplatin and cisplatin (cis-DDP). An exemplary anthracenedione is mitoxantrone. An exemplary substituted urea is hydroxyurea. An exemplary methyl hydrazine derivative is procarbazine (N-methylhydrazine, MIH). These examples are not limiting and it is contemplated that any known cytotoxic, cytostatic, and/or cytocidal agent can be conjugated or otherwise attached to targeting peptides and administered to a targeted organ, tissue, and/or cell type within the scope of the presently disclosed subject matter.

Chemotherapeutic (cytotoxic) agents include but are not limited to 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raioxifene, tamoxifen, taxol, temazolomide (an aqueous form of DTIC), transplatinum, vinblastine and methotrexate, vincristine, or any analog or derivative variant of the foregoing. Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog or derivative variant thereof.

The peptides identified and tested thus far in peptide-based vaccine approaches have generally fallen into one of three categories: 1) mutated on individual tumors, and thus not displayed on a broad cross section of tumors from different patients; 2) derived from unmutated tissue-specific proteins, and thus compromised by mechanisms of self-tolerance; and 3) expressed in subsets of cancer cells and normal testes.

Antigens linked to transformation or oncogenic processes are of primary interest for immunotherapeutic development based on the hypothesis that tumor escape through mutation of these proteins can be more difficult without compromising tumor growth or metastatic potential.

The target peptides of the presently disclosed subject matter are unique in that the identified target peptides are modified by intracellular modification. This modification is of particular relevance because it is associated with a variety of cellular control processes, some of which are dysregulated in cancer cells. For example, the source proteins for class I MHC-associated phosphopeptides are often known phosphoproteins, supporting the idea that the phosphopeptides are processed from folded proteins participating in signaling pathways.

Although not wishing to be bound by any particular theory, it is envisioned that the target peptides of the presently disclosed subject matter are unexpectedly superior to known tumor-associated antigen-derived peptides for use in immunotherapy because: 1) they only displayed on the surface of cells in which intracellular phosphorylation is dysregulated, i.e., cancer cells, and not normal thymus cells, and thus they are not are not compromised by self-tolerance (as opposed to TAA which are associated with overexpression or otherwise expressed on non-mutated cells); and/or 2) they identify a cell displaying them on their surface as having dysregulated phosphorylation. Thus, post-translationally-modified phosphopeptides that are differentially displayed on cancer cells and derived from source proteins objectively linked to cellular transformation and metastasis allow for more extensive anti-tumor responses to be elicited following vaccination. Target peptides are, therefore, better immunogens in peptide-based vaccines, as target peptides are derived from proteins involved with cellular growth control, survival, or metastasis and alterations in these proteins as a mechanism of immune escape can interfere with the malignant phenotype of tumors.

As such, the presently disclosed subject matter also relates in some embodiments to methods for identifying target peptides for use in immunotherapy which are displayed on transformed cells but are not substantially expressed on normal tissue in general or in the thymus in particular. In some embodiments, target peptides bind the MHC class I molecule more tightly than their non-phosphorylated native counterparts. Moreover, such target peptides can in some embodiments have additional binding strength by having amino acid substitutions at certain anchor positions. In some embodiments, such modified target peptides can remain cross-reactive with TCRs specific for native target peptide MHC complexes. Additionally, it is envisioned that the target peptides associated with proteins involved in intracellular signaling cascades or cycle regulation are of particular interest for use in immunotherapy. In some cases, the TCR binding can specifically react with the phosphate groups on the target peptide being displayed on an MHC class I molecule.

In some embodiments, the method of screening target peptides for use in immunotherapy, e.g., in adaptive cell therapy or in a vaccine, involves determining whether the candidate target peptides are capable of inducing a memory T cell response. The contemplated screening methods can include providing target peptides, e.g., those disclosed herein or those to be identified in the future, to a healthy volunteer and determining the extent to which a target peptide-specific T cell response is observed. In some embodiments, the extent to which the T cell response is a memory T cell response is also determined. In some embodiments, it is determined the extent to which a T_{CM} response is elicited, e.g., relative to other T cell types. In some embodiments, those target peptides which are capable of inducing a memory T cell response in health and/or diseased patients are selected for inclusion in the therapeutic compositions of the presently disclosed subject matter.

In some embodiments, the presently disclosed subject matter provides methods for inducing a target peptide-specific memory T cell response (e.g., T_{CM}) response in a patient by providing the patient with a composition comprising the target peptides disclosed herein. In some embodiments, the compositions are those disclosed herein and are provided in a dosing regimen disclosed herein.

In some embodiments, the presently disclosed subject matter relates to methods for determining a cancer disease prognosis. These methods involve providing a patient with target peptide compositions and determining the extent to which the patient is able to mount a target peptide specific T cell response. In some embodiments, the target peptide composition contains target peptides selected in the same substantially the same manner that one would select target peptides for inclusion in a therapeutic composition. If a patient is able to mount a significant target peptide-specific T cell response, then the patient is likely to have a better prognosis than a patient with the similar disease and therapeutic regimen that is not able to mount a target peptide-specific T cell response. In some embodiments, the methods involve determining whether the target peptide specific T cell response is a T_{CM} response. In some embodiments, the presence of a target peptide-specific T cell response as a result of the presently disclosed diagnostic methods correlates with an at least or about 5, 10, 15, 20, 25, 30, 35, 40, 45, 50, 55, 60, 65, 70, 75, 80, 85, 90, 95, 100, 125, 150, 175, 200, 250, 300, 400, 500, or more percent increase in progression free survival over standard of care.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Identification of MHC Class I-Associated Phosphopeptides (MHC-I-pP) as Novel Tumor-Specific Antigens for HCC

Several methods exist for identification of tumor antigens on the surface of cancer cells. In the past, most often a “reverse immunology” approach was used, in which the peptide sequences of the tumor antigens were predicted in silico. MEW presented peptides with low binding affinities or those carrying posttranslational modifications cannot be predicted with this approach.

Using an approach involving direct isolation of MHC-peptide-complexes from the surface of the tumor cells, which is particularly useful for identification of post-translationally modified peptides, MEW class I-bound phosphopeptides (MHC-I-pP) were identified using the following general approach. Briefly, HCC tumors were removed and lysates were prepared from tumor tissue and adjacent (distal; normal) tissue. MHC-I-pP-complexes were immunoprecipitated from the HCC and adjacent (distal; normal) liver tissue lysates and affinity purified with the help of a MHC class I-specific antibody (W6/32; see Brodsky et al., 1979). MHC-I-pP were separated and enriched from other MHC-bound peptides in several steps including elution and purification with a 10 kDa cut-off filter and IMAC chromatography before the MHC-I-pP were characterized and sequenced by HPLC-ESI-MS/MS in a high-resolution mass spectrometer as described in Abelin et al., 2015. Phosphopeptide sequences were manually assigned and comparisons were made between health and cancerous tissues.

As disclosed herein, 460 HCC-associated MHC-I-pP were identified. These data were acquired from four (4) different HCC samples and the corresponding adjacent cirrhotic or non-cirrhotic liver tissue and from a hepatoblastoma cell line (HepG2). In total, 21 HCC samples with the corresponding adjacent liver tissue were processed. Sequence data were derived from mass spectrometry analysis. Table 16 summarizes patient characteristics from the examined cohort.

TABLE 16
Patient Characteristics of the Cohort used for MHC-I-pP
Identification on HCC Tumors and Adjacent Liver Tissue*

| | | | | | | | | |
|---|---|---|---|---|---|---|---|---|
| | | | | | | | | AFP |
| ID# | Age | HLA | Aetiology | C | CTP | BCLC | Treatment Received | [kU/L] |

SAMPLES FROM FEMALES

| | | | | | | | | |
|---|---|---|---|---|---|---|---|---|
| 3081 | 67 | A*03 | ALD | 1 | B | A | RFA 9 May 2011 and | 16 |
| | | B*07 | | | | | 24 Apr. 2013 | |
| | | B*35 | | | | | | |
| 4164 | 39 | A*02 | Adenoma | 0 | | A | Left hemihepatectomy | 195 |
| | | A*03 | → HCC | | | | | |
| | | B*15 | | | | | | |
| 4233 | 74 | A*01 | DD ALD | 0 | | A | Left hemihepatectomy | 2414 |
| | | A*02 | | | | | | |
| | | B*08 | | | | | | |
| | | C*07 | | | | | | |
| 4857 | 77 | A*02 | Adenoma | 0 | | A | Resection | 35850 |
| | | A*03 | → HCC | | | | | |
| | | B*07 | | | | | | |
| | | B*44 | | | | | | |
| | | C*05 | | | | | | |
| | | C*07 | | | | | | |
| 4922 | 53 | A*03 | HBV | 1 | B | A | OLTx | 6 |
| | | A*24 | | | | | | |
| | | B*07 | | | | | | |
| | | B*53 | | | | | | |
| | | C*07 | | | | | | |
| | | C*14 | | | | | | |
| 5176 | 52 | A*01 | FNH −> | 0 | | A | Left lateral resection | — |
| | | A*24 | HCC | | | | | |
| | | B*08 | | | | | | |
| | | B*44 | | | | | | |
| | | C*05 | | | | | | |
| | | C*07 | | | | | | |
| 5549 | 64 | A*24 | cryptogenic | 0 | | A | Extended right | — |
| | | A*29 | | | | | hemihepatectomy | |
| | | B*15 | | | | | | |
| | | B*44 | | | | | | |
| | | C*03 | | | | | | |
| | | C*16 | | | | | | |

SAMPLES FROM MALES

| | | | | | | | | |
|---|---|---|---|---|---|---|---|---|
| 370 | 45 | A*02 | cryptogenic | 0 | | 0 | Resection | neg |
| | | B*08 | (Fibrolamellar | | | | | |
| | | B*18 | HCC) | | | | | |
| | | C*07 | | | | | | |
| 981 | 81 | A*01 | ? | 1 | A | 0 | Resection + RFTA in | 1 |
| | | A*02 | | | | | February 2010. Relapse −> PEI | |
| | | B*27 | | | | | November 2011, December 2011, | |
| | | B*37 | | | | | January 2011. | |
| | | C*02 | | | | | Metastasis spine −> | |
| | | C*06 | | | | | surgery October 2012 | |
| 1515 | 60 | A*02 | ALD | 1 | A | A | OLTx | 14 |
| | | A*03 | | | | | | |
| | | B*18 | | | | | | |
| | | C*05 | | | | | | |
| 3907 | 53 | A*02 | DD ASH | 1 | B | 0 | Right hemihepatectomy | 219 |
| | | A*26 | | | | | | |
| | | B*08 | | | | | | |
| | | B*49 | | | | | | |
| | | C*07 | | | | | | |
| | | C*07 | | | | | | |
| 4028 | 48 | A2+ | HCV | 1 | A | 0 | OLTx | 2 |
| | | B7− | | | | | | |
| 4908 | 80 | A*01 | A₁ATD | 0 | | A | Resection | 124 |
| | | A*24 | | | | | | |
| | | B*08 | | | | | | |
| | | B*15 | | | | | | |
| | | C*03 | | | | | | |
| | | C*07 | | | | | | |
| 5437 | 58 | A*02 | HBV | 1 | A | A | OLTx | 3 |
| | | A*03 | | | | | | |
| | | B*15 | | | | | | |
| | | B*40 | | | | | | |
| | | C*03 | | | | | | |
| | | C*04 | | | | | | |
| 5487 | 79 | A*03 | HH | 0 | | A | Caudate lobe resection | 1 |
| 5493 | 65 | A*03 | NASH DD | 1 | A | A | TACEx3 in February 2012, | 1 |
| | | | ALD | | | | July 2012, October 2012. | |
| | | | | | | | OLTx in January 2015. | |
| TIL12 | 64 | A*02 | ALD | 1 | B | A | OLTx | 9 |
| | | A*30 | | | | | | |
| | | B*18 | | | | | | |
| | | B*35 | | | | | | |
| | | C*04 | | | | | | |
| | | C*05 | | | | | | |
| 5573 | 54 | A*01 | HCV | 1 | B | A-B | OLTx | 45 |
| | | A*03 | | | | | | |
| 5721 | 57 | B*08 | HCV | 1 | A | A | Resection | — |
| 5725 | 58 | A*03 | ALD | 1 | A | A | OLTx | 16 |

*C: liver cirrhosis; CTP: Child-Turcotte-Pugh stadium; BCLC: Barcelona Clinic Liver Cancer Staging; AFP: α-fetoprotein; OLTx: Orthotopic liver transplantation.

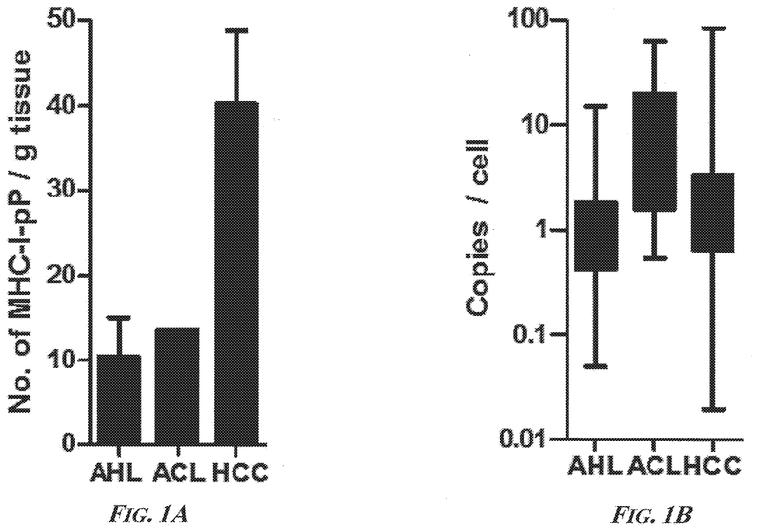

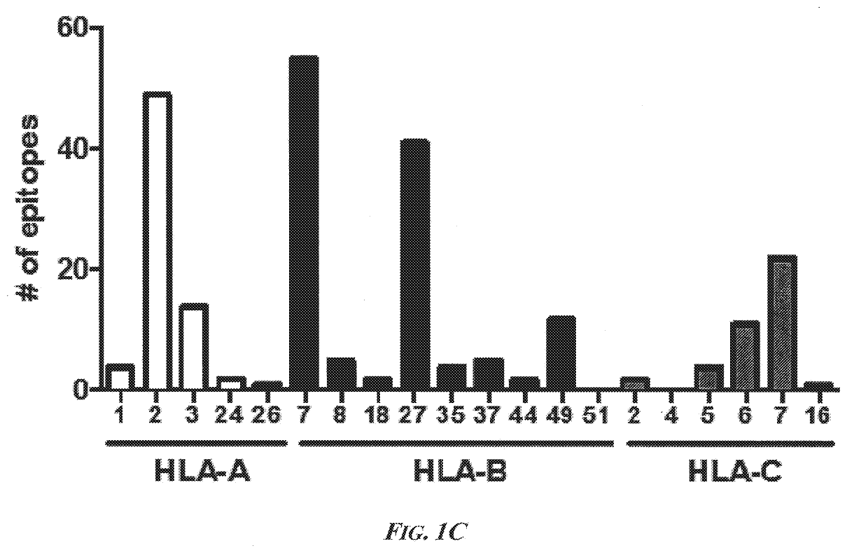

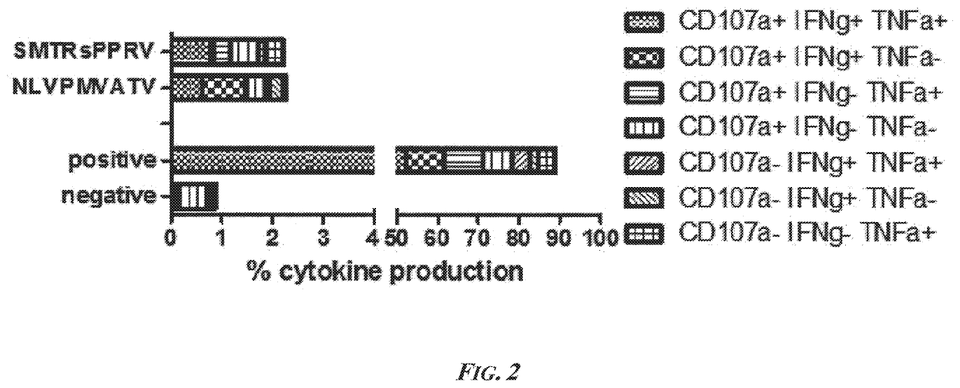

Greater varieties of MHC-I-pP and on an average more MHC-I-pP were presented on tumor tissue than on premalignant liver cirrhosis or on non-cirrhotic liver tissue. Approximately 40 different MHC-I-pP were found per gram of tumor tissue and only around 10 MHC-I-pP were found per gram of non-cirrhotic liver tissue (see FIG. 1A). The presentation of each MHC-I-pP per cell varied widely from statistically <1 copy/cell for most of the peptides up to 83 copies/cell. No differences with progression of the liver disease were observed (see FIG. 1B). This might have been due to the fact that after the development of a cancer many bystander-mutations accumulate in the cancerous cells, which can lead to the presentation of a plethora of different MHC-I-pP on each cell.

29 out of the first 250 MHC-I-pP identified were discovered on healthy tissue, but most of them were found additionally on HCC tissue (n=213), cirrhotic liver tissue (n=19), and/or an HepG2 cells (n=37). Most of the underlying proteins have not been previously associated with HCC. Some of the identified MHC-I-pP were found on other malignancies, e.g., colorectal cancer (n=109), esophageal cancers (n=25; see e.g., Tables 14 and 29), melanoma (n=29), ovarian cancer (n=38), hematological malignancies including leukemia (n=75; see also Cobbold et al., 2013), and breast cancer (n=48), further highlighting the importance of this novel class of tumor antigens for cancer growths.

Overall, peptides restricted by several different MHC class I alleles have been identified. MHC-I-pP were predicted to bind most commonly to HLA-B*0702, HLA-B*2705, HLA-A*0201, and HLA-C*07. These data were potentially biased as 5 out of 5 of the analyzed samples were HLA-A*0201 positive, 3 out of 5 samples were HLA-A*C07 positive, but only one patient was HLA-B*0702 positive (see FIG. 1C). Using a vaccination containing ˜30 MHC-I-pP, it is possible that over ninety percent of the Caucasian population would be expected to recognize on average about 3 different MHC-I-pP (see Bui et al., 2006).

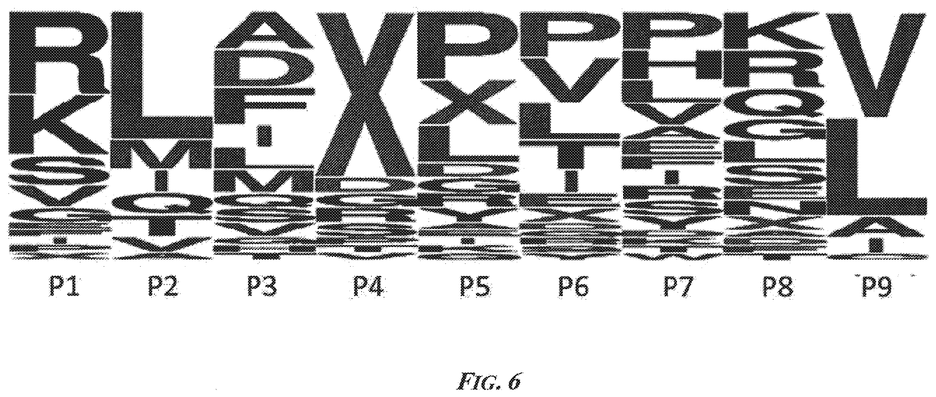

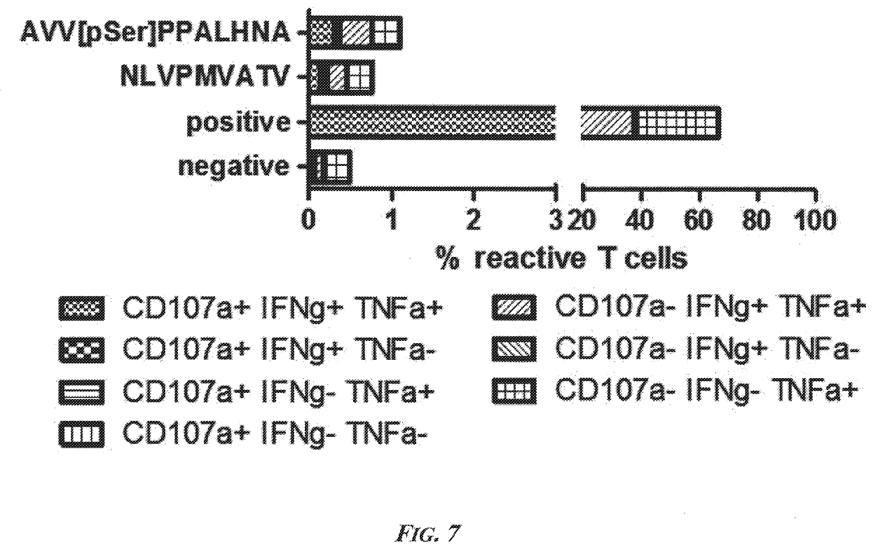

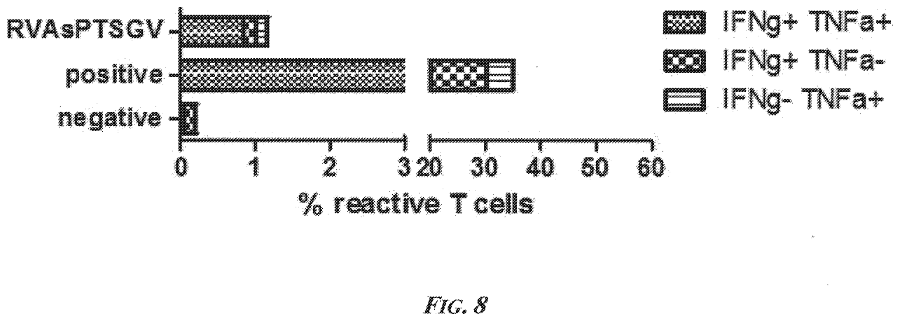

The characteristics of HCC-specific HLA-A*0201-bound phosphopeptides was also investigated, which were similar to those previously reported for HLA-A*0201-bound phosphopeptides (Mohammed et al., 2008). Briefly, each of the phosphopeptides was 7-13 amino acids in lengths and of 77 HLA-A*0201-restricted phosphopeptides, 70 contained a phosphoserine, 6 of the 77 contained a phosphothreonine, and 1 of the 77 contained a phosphotyrosine (see Table 2 and FIG. 6). The phosphate was found at position 4 in 73% of HLA-A*0201 phosphopeptides (see Table 2 and FIG. 6).

It has been reported that binding affinities of phosphopeptides are in general significantly greater than those of their non-phosphorylated counterparts and that this effect is most pronounced if the peptides are phosphorylated at P4 (see Mohammed et al., 2008). Additionally, 55% of the phosphopeptides contained a positively charged amino acid (Arg or Lys) at P1, which seems to enhance the stability of the phosphopeptide-MHC association. HLA-A*0201-restricted phosphopeptides showed a strong preference for leucine at P2 and leucine/valine at P9 corresponding to the HLA-A*0201-supertype binding motif with a hydrophobic, aliphatic amino acid [L, I, V, M, A, T, Q] at position 2 and the C-terminal end (Sette & Sidney, 1999; Sidney et al., 2008). Taken together, most HLA-A*0201-restricted phosphopeptides shared a common structure with a positively charged amino acid at position 1, a strong preference for leucine/valine at positions 2 and 9, and the phosphate moiety at position 4, which was oriented upwards, solvent oriented, and available for direct contact with the TCR (Mohammed et al., 2008; see FIG. 6).

### Example 2

### Characterization of Immune Responses Against MHC-I-pP

Previous data had indicated that T cell responses against phosphoproteins can be found in healthy individuals and to a lesser extent in patients with malignant diseases (see e.g., U.S. Patent Application Publication No. 2005/0277161; PCT International Patent Application Publication No. WO 2011/149909). These results suggested that individuals with a functional immune system create T cell responses against aberrantly phosphorylated peptides in order to eliminate those cells with signs of transformation. This may prevent further alterations and malignant transformation of the cells. A major goal of this project was to investigate if patients with chronic liver disease, HCC, and/or esophageal cancer are able to mount an efficient anti-phosphopeptide immune response during the course of disease.

From this, CD8⁺ T cell responses against newly identified MHC class I-associated phosphopeptides in healthy individuals and patients with chronic liver disease were investigated. Twenty-one of the newly identified HCC-associated HLA-A*0201-restricted phosphopeptides were selected (see Table 17) for further immunological testing in HLA-A*0201 positive patients. MHC-I-pP-specific cytotoxic CD8⁺ T cell responses (ppCTL) were assessed using intracellular cytokine staining (ICS) and several cytokines and surface markers were assessed in parallel. After 7 days of stimulation with the respective MHC-I-pP and no other cytokines, CD3- and CD8-expressing T cells were stained for at least two different cytokines (IFN-γ, TNF-α) and when required CD107a expression as a marker for their cytotoxic potential.

First, peripheral blood mononuclear cells (PBMCs) from healthy donors and patients with hereditary hemochromatosis (HH) were analyzed. HH is a chronic liver disease characterized by excessive intestinal absorption of dietary iron resulting in a pathological deposition of iron in the liver. PBMCs or lymphocytes from liver tissue were extracted and specifically stimulated with (phospho-) peptides for 7 days before intracellular cytokine staining (ICS). Doublets and dead cells, using a fixable viability dye, were excluded. Lymphocytes were gated on CD3⁺ and CD8⁺ double positive cells and were analysed for expression of IFNγ, TNFα-, and CD107a.

Phosphopeptide-specific T cell responses were not found in healthy and young donors (HD) with a mean age of 26 years, although ppCTL-responses have been identified in healthy individuals—especially in middle-aged persons—by the instant co-inventors previously. Interestingly, ppCTL-responses were found in the peripheral blood of patients with chronic liver disease in the HH cohort in around 65% of cases (see Table 18). The patients in that cohort were significantly older with a mean age of 57 years. All of the HH patients were treated with phlebotomy and therefore liver disease was well controlled. None of the patients had abnormal liver function tests or abnormal ferritin values at the time of venesection (see Table 19). There was no correlation of immune responses against MHC-I-pP with the grade of liver injury, e.g., steatosis, fibrosis, or cirrhosis.

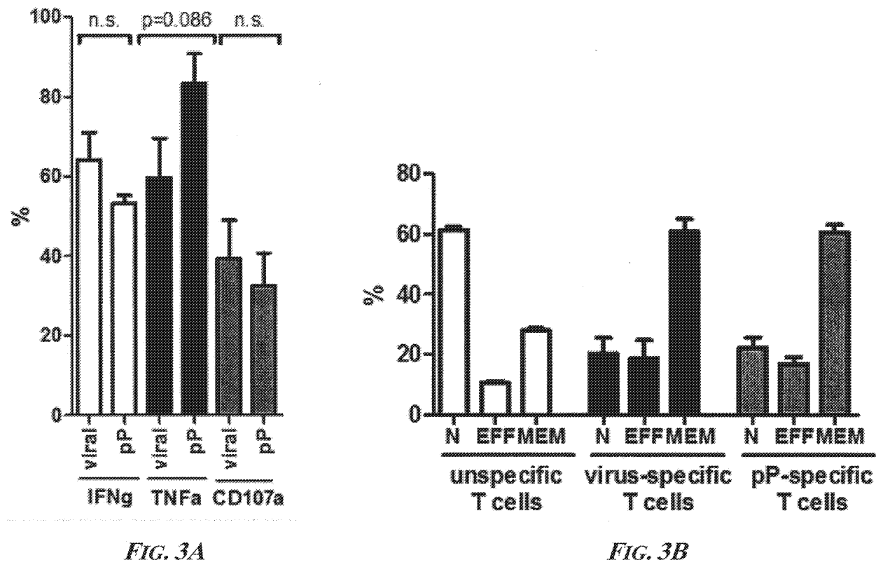

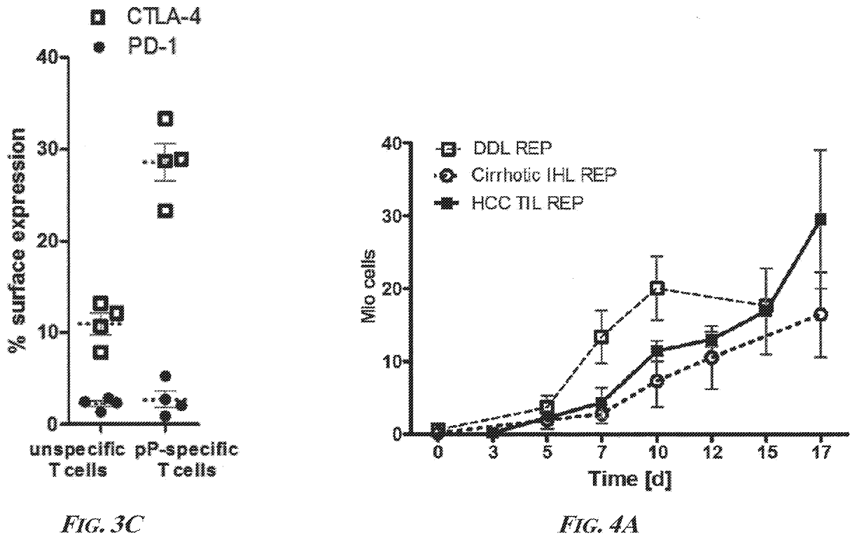

ppCTL-responses were compared with responses to immunodominant viral epitopes from cytomegalovirus (NLVPMVATV; SEQ ID NO: 451) and Epstein-Barr virus (GLCTLVAML; SEQ ID NO: 501). In most cases, T cell responses against MHC-I-pP were comparable in quantity and quality to viral immune responses (see FIGS. 2 and 3A; see also Table 18). This is in contrast to the “classic” TAA, where immune responses are often nearly not detectable (<0.1% of CD8⁺ T cells) and often show signs of exhaustion (Flecken et al., 2014). In the instant analysis, only responses with a minimum of 0.25% of reactive CD8⁺ T cells were considered positive. ppCTLs produce multiple cytokines, mainly IFNγ and TNFα (see Error! Reference source not found.3A), but also low amounts of IL-2. The production of multiple cytokines (IFNγ, TNFα and IL-2) by T lymphocytes, including the capacity to degranulate (measured by the surface expression of CD107a) is in general associated with better disease control (Almeida et al., 2007; Harari et al., 2007).

Approximately one-third of the ppCTLs were positive for the degranulation marker CD107a, indicating their ability to kill cancer cells. There was a slight tendency of ppCTLs to produce larger amounts of TNFα in comparison to virus-specific CD8⁺ T cells, which did not turn out to be significant. This suggested that TNFα was a more sensitive marker for detecting ppCTLs than IFNγ or CD107a.

ppCTLs are mainly CD27⁺ and CD45RA⁻ and therefore most-likely reside in the memory compartment (see Error! Reference source not found.3B). This suggested that only individuals that had been previously exposed to the MHC-I-pP established an immunological memory against these antigens. If healthy donors were too young, like in the instantly described healthy control group (mean age ˜26 years), they likely did not yet have the chance to be exposed to MHC-I-pP tumor antigens. However, if patients had an underlying chronic disease which predisposed them to the development of a cancer, such as like in the instant HH cohort, then phosphopeptide immune responses were measurable in over 60% of cases.

Exhausted TAA-specific T cells in the cancer microenvironment express high levels of inhibitory receptors, including PD-1 and CTLA-4, and show impaired effector cytokine/molecule production, such as IL-2, TNF-α, IFN-γ, and CD107a. PD-1- and CTLA-4 expression was measured on the surface of ppCTLs-derived from PBMCs of patients with chronic liver disease. ppCTLs expressed more CTLA-4 on their surface than virus-specific T cells from the same patients (see Error! Reference source not found., 7, and 8). PD-1 expression did not seem to be increased on the surface of ppCTLs. PD-1 expression is usually upregulated on tumor-infiltrating CD8⁺ T cells and correlates with reduced cytokine production in hepatocellular carcinoma (Bui et al., 2006) and other cancer patients. PD-1⁺ and CTLA-4⁺ double positive CD8⁺ TILs are even more severely exhausted in proliferation and cytokine production and dual blockade with monoclonal antibodies enhances T cell function in cancer (Takayama et al., 2000). The mixed pattern described herein suggested that ppCTLs were in an intermediate stage and not yet fully exhausted, at least in the peripheral blood. This favored a CTLA-4 monoclonal antibody therapy for restoring immunity against phosphopeptide tumor antigens in patients with chronic liver disease.

Specific ppCTL-lines were enriched from PBMCs with multiple rounds of stimulation against the respective phosphopeptides. A ppCTL-line against the protein serine/arginine-rich splicing factor 8 (SRSF8) secreted IFNγ, TNFα and expressed CD107a in response to stimulation only with the phosphorylated peptide IMDRtPEKL (SEQ ID NO: 14), but not to stimulation with unphosphorylated IMDRTPEKL (SEQ ID NO: 14) peptide, suggesting that recognition of MHC-I-pP in patients with chronic liver disease could be exclusively phosphate-dependent. In one HH patient, a response against the MHC-I-pP RVAsPTSGV (SEQ ID NO: 57) from the protein insulin receptor substrate 2 (Irs2) was even evident ex vivo in an ICS from PBMCs of a patient with hereditary hemochromatosis. The observation that it was possible to detect ex vivo T cell responses against MHC-I-pP was important because in vitro stimulation resulted in quantitative and functional changes of T cell responses.

### Example 3

### Initiation and Expansion of Phosphopeptide-Specific CD8⁺ T Cells for Adoptive T Cell Transfer (ACT) Therapy

It has been shown that adoptive cell transfer (ACT) of TILs can mediate cancer regression in patients with metastatic melanoma (Rosenberg & Restifo, 2015). In ACT, autologous immune cells from a patient are removed, altered and/or expanded in vitro, and then transferred back into the patient in order to kill cancer cells. It is still unclear, however, whether this approach can be applied to primary liver cancer or for targeting phosphopeptide tumor antigens.

It is a widely accepted hypothesis that a greater concentration of tumor-reactive lymphocytes can be found at tumor sites in comparison to the peripheral blood. Therefore, whether anti-phosphopeptide immune responses could be found in tumor-infiltrating lymphocytes (TILs) from HCC or in the liver compartment in general was investigated. Different protocols for intrahepatic lymphocyte (IHL) and tumor-infiltrating lymphocyte (TIL) isolation and purification exist (Morsy et al., 2005). Resected tissue specimen are either digested into a single-cell suspension (enzymatic digestion, ED) or divided into multiple tumor fragments that are individually grown in IL-2 (Dudley et al., 2003). It was a goal to understand which technique works best for liver tissue and from which compartment ppCTLs had to be extracted in order to expand ppCTLs for ACT.

In addition, several methods for expanding tumor reactive TILs have been described. Late successes in clinical trials using ACT for melanoma and epithelial cancers ACT used a technique for expanding TILs called rapid expansion protocol (REP) described in Dudley et al., 2003. With this technique, cultures are rapidly expanded in the presence of excess irradiated feeder lymphocytes, anti-CD3-antibody, and high-dose IL-2. So far, it is unclear if expansion of ppCTLs with REP has been successful for liver-derived lymphocytes and ppCTLs.

To test the feasibility of ACT with ppCTLs for patients with advanced HCC, different published extraction protocols described in Morsy et al., 2005 were tested and the proliferative potential, phenotype, and antigen specificity of expanded liver-derived ppCTLs were assessed.

A total of 41 liver specimens from explanted livers after orthotopic liver transplantation (OLTx) or from resection or from deceased donor livers (DDL) that were rejected for transplantation. In total, specimens were obtained from 6 DDLs, 5 from end-stage liver cirrhosis, and 17 from HCC patients. In each case attempts were made to obtain both tumor and adjacent tissue. Clinical parameters of the patients are summarized in Table 20. Most of the specimens came from explanted organs after transplantation and consequently most livers were severely cirrhotic.

Initiation of TIL microcultures from tissue fragments (TF) and by enzymatic digestion (ED) from tumor samples were compared. 14 out of 17 HCC tumors were minced into fragments and 10 out of 17 samples were processed into single cell suspensions by ED. 6 smaller tumors were only minced into fragments (Table 21). Initiation of lymphocyte cultures worked both for TF and ED with tumor tissue, but for adjacent tissue (distal liver tissue, 2 cm or more away from the tumor), ED was the preferred method. Initiation of microcultures from TF from HCC led to viable cell numbers in around sixty percent of cases. This is in accordance with published results from generation of TILs from gastrointestinal-tract cancer liver metastases (Turcotte et al., 2013). T cell cultures initiated by TF from liver specimens distal to the tumor often failed to induce viable T cell cultures. In contrast, initiation of cultures by ED was possible in 70-80% of cases for both tumor and distal tissue.

Lymphocyte populations from TF reached a confluent lymphocytic carpet, which was countable, after ˜14 days of culture. Until that time, cultures derived by ED had already nearly doubled. Growth of lymphocytes derived by ED in most cases outperformed cultures initiated from TF in the first 2-4 weeks.

To further characterize the cultures, cultures were analyzed by flow cytometry including multiple markers (CD3, CD4, CD8, CCR7, CD45RO, CD25, FoxP3) between weeks 5-7. Interestingly, significant differences were observed in the composition of the cultures derived by TF or ED. Cultures derived by ED yielded higher number of CD8⁺ T cells in comparison to cultures initiated with TF. In cultures from TF, CD4⁺ T cells were the predominant population. No major differences were observed in terms of CD8 T cell marker expression or CD4 markers (Table 22) and were comparable to results published for other cancers (Turcotte et al., 2014).

These results suggested that obtaining lymphocytes from TF, which was extensively used in the past for ACT in malignant melanoma and other cancers, did not seem to be the optimal method for patients with HCC. In >90 of cases, the tissue adjacent to the HCC was severely cirrhotic and this seemed to prevent exit of lymphocytes out of the tissue into the culture. Therefore, approaches in which help is given to the lymphocytes by mechanical and enzymatic disaggregation of the cirrhotic tissue seem to be preferable.

A problem that arises from ED is that larger tissue samples are needed in order to get a sufficient number of lymphocytes to start a culture. That would mean that patients with HCC would need to have surgery before ACT in order to acquire enough tumor tissue. But that is not practical considering the expected symptoms from liver cirrhosis, which would be expected to be exacerbated by surgery. A possible approach to obtain liver tissue before immunotherapy could thus be liver biopsy.

After initial outgrowth of the hepatic lymphocyte cultures, whether TILs or IHLs could be expanded in large quantities using a standard 14-day rapid expansion protocol (REP) with irradiated PBMC feeders, soluble anti-CD3 antibody, and high-dose IL-2 was tested. For all the cultures tested, an expansion of the T cells up to 1×10⁹ cells was achieved within the first 14-21 days. No differences were observed in the potential to expand lymphocytes derived from healthy liver tissue, cirrhotic liver tissue, or HCCs (see Error! Reference source not found.4A). A further expansion was also possible but not investigated.

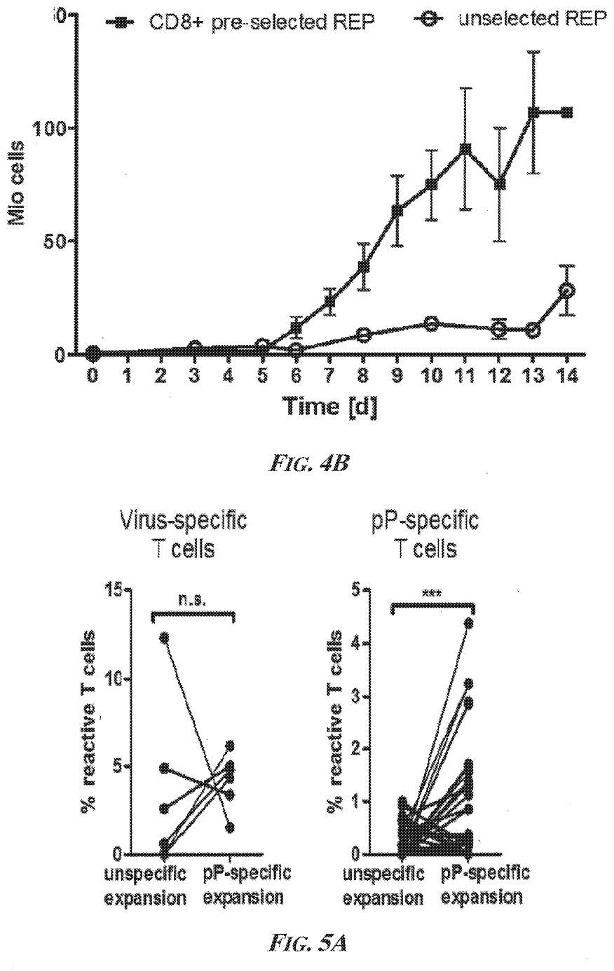

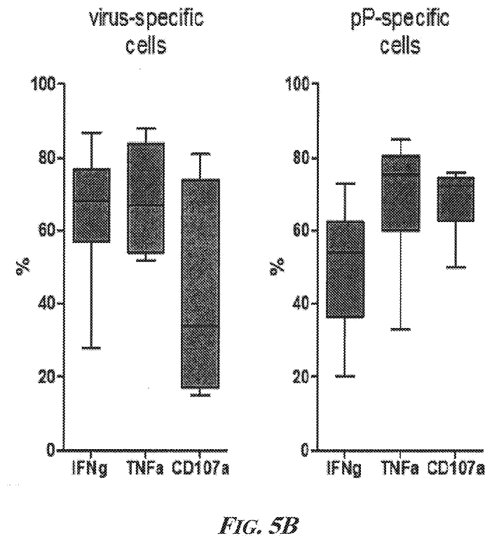

Positive selection of CD8⁺ TILs prior to REP was performed with magnetic beads in seven of the samples. Growth was accelerated in the first 14 days with CD8⁺ pre-selected T cell cultures (see FIG. 4B). It has previously been reported that a clinical grade expansion of TILs in melanoma and GI tract cancers was identical for unselected and CD8 pre-selected cultures (Prieto et al., 2010; Turcotte et al., 2014). Again, expanded cultures were further classified and phenotyped, and no major differences of the examined markers were observed pre- or post-expansion.

Taken together, expansion of liver-derived lymphocytes was easily accomplished with the REP protocol and was not dependent on the origin of the lymphocytes. Next, the expanded lymphocyte cultures were screen for MHC-I-pP reactivity. The expanded T cell cultures were stimulated with the respective phosphopeptides and analyzed 7 days later with ICS in the same way as described herein above for PBMCs.

Interestingly, only very few and minor responses were detectable in all of the cultures. Background cytokine production was much higher in expanded lymphocyte cultures and therefore often genuine T cell responses were difficult to distinguish from background. Responses, which were demonstrated in the unexpanded cultures, were completely absent in the expanded T cell cultures (see Table 23 and FIG. 5A). Interestingly virus-specific T cell reactivity was not lost during expansion of T cells. A Box and Whiskers plot (see FIG. 5B) of the data from Table 23 calculated with Graph Pad showed that ppCTLs after expansion were functional, produced multiple cytokines, and were able to degranulate.

This indicated that if expansion of T cells happened in a large scale and in an undirected way, virus-specific T cells and tumor-unspecific T cells overgrew tumor-specific T cells. This might be one reason why ACT with T cells failed to induce clinical responses on a regular basis. Overgrowth of virus-specific cells could be due to the fact that these cells were less exhausted and expressed lower amounts of inhibiting receptors Therefore, lymphocyte cultures were repeatedly stimulated before and during the expansion reaction with a phosphopeptide-pool (see Table 3). With this phosphopeptide-specific expansion, lost immune responses against phosphopeptides could be restored and could be clearly identified from the background (ssee Table 23 and FIG. 5A) in most cases. Depending on the reaction, not every clone was expanded every time, but the strongest immune responses were conserved before and after expansion. Again ppCTLs after expansion were able to produce multiple cytokines and the degranulation marker CD107a, indicating that expanded cells are fully functional after REP for ACT.

Finally, if ppCTLs could be found in TILs or the liver compartment in general was investigated. Because T cell numbers were small after initiation of the cultures, it was necessary to look in expanded T cell cultures. Therefore, all available HLA-A*0201 positive cultures derived by ED from our 3 cohorts were expanded with our new phosphopeptide-specific expansion protocol. The expanded T cells were individually stimulated with the 21 HLA-A*0201 restricted MHC-I-pP (see Table 3) for another 7 days before ICS.

Interestingly, most of the responses against MHC-I-pP were found in the cultures derived from “healthy” deceased donor livers. Only a few responses could be found in cultures derived from end-stage liver cirrhosis, although one of the responses was very strong and consisted of greater than 15% of the whole CD8⁺ T cell population. In the HCC livers, no ppCTLs could be found or expanded, neither in the tumor itself nor in the adjacent tissue. These results were consistent with observations reported for leukemia-associated phosphopeptides (Cobbold et al., 2013), where only in very few patients with cancer ppCTLs could be found.

These results suggested that tumor outgrowth was accompanied by immunosuppressive mechanisms in the tumor microenvironment and T cell exhaustion, which led to the disappearance of anti-phosphopeptide immunity during the course of disease.

Table 24 provides a summary of all ppCTL responses from pP-specifically expanded cultures from “healthy” livers, cirrhotic livers. and HCCs.

### DISCUSSION OF THE EXAMPLES

HCC develops normally after several years of chronic liver inflammation and most of the time after the development of liver cirrhosis. In the course of chronic liver diseases several mutations and epigenetic changes accumulate in the liver cells which finally lead to a dysregulation of major signaling pathways that are important for malignant transformation (Whittaker et al., 2010). Other current studies suggest that HCC can be derived from cancer stem cells (CSCs) in preneoplastic regions of altered hepatocytes (He et al., 2013). Taken together, HCC is considered to be a slowly developing malignancy to evolve from premalignant lesions in chronically damaged livers.

Therefore, it was hypothesized that phosphopeptides are presented increasingly on the surface of altered hepatocytes with progression of the disease. Young and healthy individuals are likely to clear altered, premalignant hepatocytes with the help of phosphopeptide-specific cytotoxic lymphocytes. As liver disease progresses and liver damage increases the immune system is not able to clear all the cancer progenitors and defensive mechanisms of the early tumors against the immune system gain the upper hand. Therefore, a loss of immune responses against phosphopeptides during disease progression could be a predictor for poor outcomes in patients with HCC.

Disclosed herein are 460 phosphopeptides presented to the immune system by MHC molecules derived from human hepatocellular carcinoma and/or esophageal carcinoma. It is noted that there are hundreds of different HLA alleles in the human population, and each individual expresses 3 to 6 different alleles. With respect to Caucasians, for example, most carry at least one of the following six alleles: HLA A*0201 (50%); HLA A*0101 (29%); HLA A*0301 (21%); HLA B*4402 (27%); HLA B*0702 (30%); and HLA B*2705 (7%). Since disclosed herein are phosphopeptides presented by all of these HLA alleles, it should be possible to treat heptatocellular carcinoma in approximately 90% percent of the Caucasian population using the compositions comprising the phosphopeptides disclosed herein.

Many of the underlying proteins from the identified respective MHC-I-pP can be directly linked to important HCC-characteristic, malignant signaling pathways (Whittaker et al., 2010), which highlight their importance as potential new immunotherapeutic targets. Functional annotation clustering of all identified MHC-I-pP with respect to their biological processes (GO term BP analysis) using the Database for Annotation, Visualization and Integrated Discovery v6.7 (DAVID; Huang da et al., 2009) yielded several enriched clusters of proteins involved in transcriptional regulation, cell cycle regulation, regulation of metabolic processes, apoptosis, cell death, cell migration, and many other biological processes, which have been associated with “hallmarks of cancer” (Hanahan & Weinberg, 2011; see also Table 25). Biocarta and KEGG signaling pathway mapping of all identified MHC-I-pP revealed that HCC-specific MHC-I-pP are significantly enriched in mitogen-activated protein kinase (MAPK) pathways and the Neurotrophin pathway (see Table 26). Several studies also indicate a major role of the MAPK/RAF/MEK/ERK pathways in the tumorigenesis of HCC3. This is in contrast to the “classical” TAA and cancer-associated HLA-lingandome, for which cluster formation is not observed and associations to biological processes or overrepresented pathways cannot be found (Kowalewski et al., 2015). However, due to the incomplete data set, small sample size, and low enrichment scores of these clusters, the data does not represent a complete picture of the involvement of phosphopeptides into important biological functions and their pathways yet.

Further highlighting the central position in major cancer associated pathways is the fact that some of the underlying proteins are covered by several MHC-I-pP. Those were found on different HCC and/or esophageal cancer samples and were presented by different HLA molecules. This might indicate that key proteins for malignant transformation are presented as phosphopeptides by the immune system across “HLA-borders”. For example, two MHC-I-pPs, KRYsGNmEY (SEQ ID NO: 242) and RRDsLQKPGL (SEQ ID NO: 248), were identified for the serine/threonine protein kinases LATS1 and LATS2 (see Table 27), predicted to bind to HLA-C*07 and HLA-B*2705. LATS1 and LATS2 have been shown to be negative regulators of YAP1 in the Hippo signalling pathway (Hao et al., 2008). Two different MHC-I-pP were identified for the Mitogen-activated protein kinase kinase kinase 3 and 11 (MAP3K3/11), which play a key role in the MAPK/ERK/JUN-signalling cascade and activation of B-Raf, ERK and cell proliferation (Tibbles et al., 1996; Ellinger-Ziegelbauer et al., 1997; Chadee & Kyriakis, 2004). Both peptides are predicted to bind to different MEW molecules, HLA-B*2705 and HLA-B*0702, respectively, and additionally were found on different cancers too.

Several of the phosphopeptides were identified on more than one type of cancer. These are summarized in Table 28.

While not wishing to be bound by any particular theory of operation, chronic liver diseases such as chronic hepatitis B or C infection (HBV/HCV), alcohol, non-alcoholic steatohepatitis (NASH), or autoimmunehepatitis (AIH) can lead to chronic inflammation of the liver with subsequent multiple changes in signaling pathways, oncogenes, and tumor suppressor genes (see e.g., Whittaker et al., 2010). Most of these processes are mediated with the help of kinases and phosphorylation of signaling pathways. HCC-specific phosphopeptides appear to be presented with increasing amounts on the surface of altered hepatocytes during disease progression. This can leads to an immune response against the hepatocytes showing signs of malignant transformation. During progression of liver disease towards HCC, immunosuppressive mechanisms can gain the upper hand and phosphopeptide-specific immunity can be lost.

Taken together, it was observed that phosphopeptide neoantigens could be identified on human primary liver cancers and/or esophageal cancers that were immunogenic in certain cohorts of patients. In total, 460 MHC class-I restricted phosphopeptides were identified, and it was demonstrated that more antigens were presented during the course of chronic liver disease towards development of HCC. Many of the HCC-specific MHC-I-pP were derived from genes directly linked to important functions for tumorigenesis, making these particularly interesting as immunotherapeutic targets. MHC-I-pP seemed to be the target of a pre-existing immunity, ppCTLs were functional and most likely were able to kill cancer cells. Interestingly, it seemed that patients with chronic liver disease did lose the ability to destroy cancer cells with the help of ppCTLs during the course of the disease towards end-stage liver disease. Thus, enhancing immunity against these tumor-associated antigens should provide a cancer immunotherapeutic strategy.

Adoptive T cell transfer therapy for HCC has been performed in very few clinical trials to date (Rosenberg et al., 1985; Takayama et al., 2000; Hui et al., 2009; Shimizu et al., 2014), and in all of these trials cells have been expanded using different methodologies. Disclosed herein is demonstrated that it was possible to grow and expand ppCTLs in a large scale for ACT using a directed and improved rapid expansion protocol. It is further disclosed herein that these cells remained functional and specific after expansion.

The results of the presently disclosed experiments implied that the main challenge to develop an effective adoptive T cell therapy for HCC and/or esophageal cancer using patient-derived lymphocytes might not be the in vitro proliferative capacity of the lymphocytes, but rather the selection and enrichment of tumor-reactive and in particular of phosphopeptide-specific T cells.

Furthermore, survival of these tumor-reactive T cells over a long period of time during expansion should be guaranteed in order to treat patients with “useful” anti-cancer lymphocytes. Lately, the poor therapeutic efficacy of autologous T cells against the tumor antigens gp100 and MART-1 raised significant concerns targeting this class of tumor antigens (Chandran et al., 2015). This and the fact that phosphopeptides are especially immunogenic make this class of tumor-associated antigens particularly interesting for use in immunotherapy.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Abelin et al. (2015) Nature Protocols 10:1308-1318.
Akamatsu et al. (1997) Bioorg Med Chem 5:157-163.
Almeida et al. (2007) J Exp Med 204:2473-2485.
Altman et al. (1996) Science 274:94-96.
Arentz-Hansen et al. (2000) J Exp Med 191:603-612.
Arozarena et al. (2011) Oncogene 30:4531-4543.
Bachmann et al. (2005) Clin Cancer Res 11:8606-8614.
Baron et al. (2005) J Clin Oncol 23:1993-2003.
Bertoletti et al. (1994) Nature 369:407-410.
Berzofsky et al. (1988) Immunol Rev 106:5-31.
Boon et al. (1994) Annu Rev Immunol 12:337-365.
Brahmer et al. (2012) N Engl J Med 366:2455-2465.
Breous & Thimme (2011) J Hepatol 54:830-834.
Brodsky et al. (1979) Immunol Rev 47:3-61.
Bui et al. (2006) BMC Bioinformatics 7:153.
Bullock et al. (2000) J Immunol 164:2354-2361.
Buonaguro et al. (2013) J Hepatol 59:897-903.
Büttner (2014) Verlangerungsantrag für das Stipendium “The phosphoproteome as a new target for for immunotherapy against hepatocelluar cancer” der Firtz Thyssen Stiftung. Vol. 70 000 16 (Fritz Thyssen Stiftung, University of Birmingham).
Chadee & Kyriakis (2004) Nature Cell Biol 6:770-776.
Chandran et al. (2015) Clin Cancer Res 21:534-543.
Chi (2011) Nature 471:537-538.
Chien et al. (2009) Proc Natl Acad Sci USA 106:1193-1198.
Chothia et al. (1985) J Mot Biol 186:651-666.
Chothia et al. (1989) Nature 342:877-883.
Clackson et al. (1991) Nature 352:624-628.
Clark et al. (1989) Eur J Immunol 19:381-388.
Cobbold et al. (2005) J Exp Med 202:379-386.
Cobbold et al. (2013) Sci Transl Med 5:203ra125.
Cole et al. (1985) Proc Natl Acad Sci USA 82:859.
Corthay (2014) Front Immunol 5:197.
Cote et al. (1983) Proc Natl Acad Sci USA 80:2026.
Crawford et al. (1999) Oncogene 18:2883-2891.
Demunter et al. (2002) Modern Pathol 15:454-461.
Dephoure et al. (2008) Proc Natl Acad Sci USA 105:10762-10767.
Depontieu et al. (2009) Proc Natl Acad Sci USA 106:12073-12078.
Dudley et al. (2003) J Immunother 26:332-342.
Dudley et al. (2008)J Clin Oncol 26:5233-5239.
DuPage et al. (2012) Nature 482:405-409.
Ellinger-Ziegelbauer et al. (1997) J Biol Chem 272:2668-2674.
Finn (2003) J Exp Med 198:1623-1626.
Fiol et al. (1988) Arch Biochem Biophys 267:797-802.
Fiol et al. (1990) J Biol Chem 265:6061-6065.
Fischbein et al. (2000) J Immunol 165:7316-7322.
Fishwild et al. (1996) Nature Biotechnol 14:845.
Flecken et al. (2014) Hepatology 59:1415-1426.
Gale et al. (1994) Ann Intern Med 120:646-652.
Gattinoni et al. (2006) Nature Rev Immunol 6:383-393.
Gerdes et al. (1999) Digestion 60:544-548.
Girbal-Neuhauser et al. (1999) J Immunol 162:585-594.
Goldman & DeFrancesco (2009) Nat Biotech 27:129-139.
Great Britain Patent Publication GB 2249310A.
Hall et al. (2010) Mol Cell Proteomics 9:2853-2863.
Haluska et al. (2006) Clin Cancer Res 12:2301s-2307s.
Hamann et al. (1997) J Exp Med 186:1407-1418.
Hanahan & Weinberg (2011) Cell 144:646-674.
Hao et al. (2008) J Biol Chem 283:5496-5509.
Harari et al. (2007) Proc Natl Acad Sci USA 104:16233-16238.
He et al. (1998) Science 281:1509-1512.
He et al. (2013) Cell 155:384-396.
Hirohashi et al. (2009) Cancer Sci 100:798-806.
Ho et al. (2006) J Immunol Methods 310:40-52.
Hoek et al. (2006) Pigment Cell Res 19:290-302.
Hogan et al. (1998) Cancer Res 58:5144-5150.
Homfray et al. (1998) Human Mutation 11:114-120.
Hoogenboom et al. (1991) Nucleic Acids Res 19:4133.
Horowitz et al. (1990) Blood 75:555-562.
Huang da et al. (2009) Nature Protocols 4:44-57.
Hui et al. (2009) Dig Liver Dis 41:36-41.
Hulsken et al. (1994) J Cell Biol 127:2061-2069.
Ilyas et al. (1997) Proc Natl Acad Sci USA 94:10330-10334.
Isakoff et al. (2005) Cancer Res 65:10992-11000.
Jacob et al. (1997) Int J Cancer 71:325-332.
Jimbow et al. (1975) J Cell Biol 66:663-670.
Jones et al. (1986) Nature 321:522-525.
Jones et al. (2008) Science 321:1801-1806.
Kabat et al. (1987) Sequences of Proteins of Immunological Interest, National Institute of Health, Bethesda, Md., United States of America.
Kageshita et al. (2001) Br J Dermatol 145:210-216.
Kantoff et al. (2010) N Engl J Med 363:411-422.
Kielhorn et al. (2003) Int J Cancer 103:652-656.
Kim et al. (2000) Pathol Intl 50:725-730.
Kimelman & Xu (2006) Oncogene 25:7482-7491.
Klenerman et al. (1994) Nature 369:403-407.
Kohler & Milstein (1975) Nature 256:495.
Kolb (2008) Blood 112:4371-4383.
Kolb et al. (1990) Blood 76:2462-2465.
Kowalewski et al. (2015) Proc Natl Acad Sci USA 112:E166-175.
Kozbor et al. (1983) Hybridoma, 2:7.
Krengel et al. (2004) J Cutaneous Pathol 31:1-7.
Kroger et al. (2005) Bone Marrow Transplant 35:37-43.
LeFranc & LeFranc (2001) The T Cell Receptor Factsbook, ISBN 0-12-441352-8, Academic Press, San Diego, Calif., United States of America.
Ley et al. (2008) Nature 456:66-72.
Liu et al. (2002) Cell 108:837-847.
Llovet et al. (2012) J Hepatol 56:908-943.
Lonberg & Huszar (1995) Intl Rev Immunol 13:65.
Lonberg et al. (1994) Nature 368:856.
Lucas & Coulie (2008) Sem Immunol 20:301-307.
Mackensen et al. (2000) Int J Cancer 86:385-392.
Maelandsmo et al. (2003) Clin Cancer Res 9:3383-3388.
Mamula et al. (1999) J Biol Chem 274:22321-22327.
Marafioti et al. (2004) Haematologica 89:957-964.
Marks et al. (1991) J Mot Biol 222:581-597.
Marks et al. (1992) J Biol Chem 267:16007.
Matsushita et al. (2012) Nature 482:400-404.
Melief (2009) J Med Sci 2:43-45.
Meyer et al. (2009) J Proteome Res 8:3666-3674.
Mihara et al. (2001) Clin Immunol 98:319.
Miyake et al. (2001) Pathol Intl 51:680-685.
Mohammed et al. (2008) Nat Immunol 9:1236-1243.
Molina et al. (2007) Proc Natl Acad Sci USA 104:2199-2204.
Morales et al. (2000) Nucl Med Biol 27:199.
Morin et al. (1997) Science 275:1787-1790.
Morsy et al. (2005) Lab Invest 85:285-296.
Neuberger (1996) Nature Biotechnol 14:826.
Newberg et al. (1992) J Immunol 149:136-142.
Niedermann et al. (1995) Immunity 2:289-299.
Novotny & Haber (1985) Proc Natl Acad Sci USA 82:4592-4596.
Nunes et al. (2011) Cancer Res 71:5435-5444.
Offringa (2009) Curr Opin Immunol 21:190-199.
Ogasawara et al. (2006) Histopathology 49:612-621.
Ohgaki et al. (2004) Cancer Lett 207:197-203.
Oliva et al. (2006) J Pathol 208:708-713.
Olmeda et al. (2003)Mol Biol Cell 14:2844-2860.
Omholt et al. (2001) Int J Cancer 92:839-842.
Otaka et al. (1995) Tetrahedron Lett 36:927-930.
Parmiani & Anichini (2006) J Hepatol 45:178-181.
Parmiani et al. (2002) J Natl Cancer Inst 94:805-818.
Parsons et al. (2011) Science 331:435-439.
Pavletic et al. (2007) Leukemia 21:2452-2455.
PCT International Patent Application Publication No. WO 1994/02602; WO 1996/33735; WO 1996/34096; WO 2004/0106380; WO 2011/0149909.
Pecina-Slaus et al. (2007) J Cutaneous Pathol 34:239-246.
Peiper et al. (1997) Eur J Immunol 27:1115-1123.
Peoples et al. (1993) Surgery 114:227-234.
Petersen et al. (2009) Proc Natl Acad Sci USA 106:2776-2781.
Pollock & Hayward (2002) Melanoma Res 12:183-186.
Presta (1992) Proc Natl Acad Sci USA 89:4285-4289.
Preudhomme et al. (2010) N Engl J Med 363:2511-2521.
Prieto et al. (2010) J Immunother 33:547-556.
Prieto et al. (2015) Nat Rev Gastroenterol Hepatol 12:681-700.
Rappsilber et al. (2007) Nature Protocols 2:1896-1906.
Restifo et al. (1993) J Exp Med 177:265-272.
Richards et al. (1999) Cancer Res 59:2096.
Riechmann et al. (1988) Nature 332:323-327.
Rimm et al. (1999) Am J Pathol 154:325-329.
Robila et al. (2008) J Immunol 181:7843-7852.
Rock & Goldberg (1999) Annu Rev Immunol 17:739-779.
Rosenberg & Dudley (2009) Curr Opin Immunol 21:233-240.
Rosenberg & Restifo (2015) Science 348:62-68.
Rosenberg et al. (1985) N Engl J Med 313:1485-1492.
Rosenberg et al. (1986) Science 233:1318-1321.
Rosenberg et al. (2004) Nat Med 10:909-915.
Rowe et al. (eds.) (2006) Handbook of Pharmaceutical Excipients, 5^{th} ed. Pharmaceutical Press, London, United Kingdom.
Ruppert et al. (1993) Cell 74:929-937.
Sadot et al. (2002) J Cell Sci 115:2771-2780.
Sandborn et al. (2001) Gastroenterology 120:1330-1338.
Sanders et al. (1999) Mol Pathol 52:151-157.
Schendel et al. (1993) J Immunol 151:4209-4220.
Schreiber et al. (2011) Science 331:1565-1570.
Seidensticker & Behrens (2000) Biochim Biophys Acta 1495:168-182.
Sette & Sidney (1999) Immunogenetics 50:201-212.
Sette et al. (1994) J Immunol 153:5586-5592.
Shimizu et al. (2014) Hum Vaccin Immunother 10:970-976.
Shinkura et al. (1998) Anticancer Res 18:1217.
Sidney et al. (2008) BMC Immunol 9:1.
Slawson et al. (2005) J Biol Chem 280:32944-32956.
Slawson et al. (2008) Mol Biol Cell 19:4130-4140.
Slingluff et al. (1994) Cancer Res 54:2731-2737.
Slingluff et al. (2000) Cancer Immunol Immunother 48:661-672.
Slovin et al. (1986) J Immunol 137:3042-3048.
Smyth et al. (1992) Tetrahedron Lett 33:4137-4140.
Strickley (2004) Pharm Res 21:201-230.
Sun et al. (2005) J Neurooncol 73:131-134.
Takahashi et al. (2002) Oncogene 21:5861-5867.
Takayama et al. (2000) Lancet 356:802-807.
Takemaru et al. (2008) Handb Exp Pharmacol 186:261-84.
Talpaz et al. (1986) N Engl J Med 314:1065-1069.
Tetsu & McCormick (1999) Nature 398:422-426.
Tibbles et al. (1996) EMBO J 15:7026-7035.
Townsend & Bodmer (1989) Ann Rev Immunol 7:601-624.
Turcotte et al. (2013) J Immunol 191:2217-2225.
Turcotte et al. (2014) Clin Cancer Res 20:331-343.
U.S. Patent Application Publication No. 2002/0119149; 2004/0202657; 2005/0277161; 2009/0117102; 2009/0226474.
U.S. Provisional Application Ser. No. 61/695,776.
U.S. Pat. Nos. 4,361,539; 4,816,567; 4,816,567; 5,225,539; 5,545,806; 5,545,807; 5,569,825; 5,625,126; 5,633,425; 5,661,016; 5,712,120; 5,861,155; 5,869,619; 5,916,771; 5,939,598; 5,968,509; 6,054,927; 6,180,370; 6,706,265; 6,750,325.
Utz et al. (1997) J Exp Med 185:843-854.
van Doom et al. (2005) J Clin Oncol 23:3886-3896.
Van Wauve (1980) J Immunol 124:2708-2718.
Verhoeyen et al. (1988) Science 239:1534-1536.
Wada et al. (1998) Hepatology 27:407-414.
Wang et al. (2007) Mol Cell Proteomics 6:1365-1379.
Wang et al. (2010) Sci Signal 3(104):ra2, including Supplemental Materials.
Watts (1997) Annu Rev Immunol 15:821-850.
Waun Ki Hong et al. Holland-Frei Cancer Medicine 10 A.D. McGraw-Hill Medical.
Weber (2002) Cancer Invest 20:208-221.
Whittaker et al. (2010) Oncogene 29:4989-5005.
Wong (1990) Transplantation 50:683-389.
Worm et al. (2004) Oncogene 23:5215-5226.
Wuttge et al. (1999) Immunol 98:273-279.
Yasumura et al. (1993) Cancer Res 53:1461-1468.
Yee et al. (2002) Proc Natl Acad Sci USA 99:16168-16173.
Yenari et al. (1998) Exp Neurol 153:223.
Yenari et al. (2001) Neurol Res 23:72.
Yewdell (2002) Mol Immunol 39:139-146.
Yoshino et al. (1994) Cancer Res 54:3387-3390.
Yost et al. (1996) Genes Dev 10:1443-1454.
Zarling et al. (2000) J Exp Med 192:1755-1762.
Zarling et al. (2006) Proc Nall Acad Sci USA 103:14889-14894.

It will be understood that various details of the presently disclosed subject matter can be changed without departing from the scope of the presently disclosed subject matter. Furthermore, the foregoing description is for the purpose of illustration only, and not for the purpose of limitation.

TABLE 17
Exemplary HCC-associated HLA-A*0201-restricted MHC-I-pP Selected for Further Immunological Testing

| | Copies/ | Found on | |
|---|---|---|---|
| MHC-I-pP | cell | other cancers | Function / Significance in cancer |
| ALDsGASLLHL | 0.1 - 0.4 | Ovarian Ca, Hematological Ca, Breast | Involved in stratified epithelial development. It is a direct transcriptional |
| (SEQ ID NO: 2) | | Ca, CRC | target of TP63. Plays a role in NF-kappa-B activation. |
| AVVsPPALHNA | 0.5 - 0.7 | Ovarian Ca, Hematological Ca. | Chromatin reader protein that plays a key role in transmission of epigenetic |
| (SEQ ID NO: 6) | | | memory across cell divisions and transcription regulation. Maintains high- |
| | | | order chromatin structure. Also acts as a regulator of p53/TP53-mediated |
| | | | transcription following phosphorylation by CK2. |
| FLDtPIAKV | 0.1 - 0.15 | CRC. | Antagonist of Wnt-signaling. |
| (SEQ ID NO: 9) | | | |
| IMDRtPEKL | 0.2 | Ovarian Ca, CRC, OEAC, Hematological | Adapter protein that couples growth factor receptors to a signaling pathways |
| (SEQ ID NO: 14) | | Ca, Melanoma, | that regulate the proliferation in breast cancer cells. When overexpressed, |
| | | Breast Ca | it confers anti-estrogen resistance in breast cancer cell lines. |
| KAFsPVRSV | 0.1 - 81 | Ovarian Ca, CRC, OEAC | Transcriptional regulator (lacking a basic DNA binding domain) including |
| (SEQ ID NO: 16) | | | cellular growth, senescence, differentiation, apoptosis, angiogenesis, and |
| | | | neoplastic transformation. |
| KIAsEIAQL | 0.3 - 0.8 | Ovarian Ca | Unknown. |
| (SEQ ID NO: 17) | | | |
| KLFPDtPLAL | 0.1 - 0.3 | Ovarian Ca, CRC, Melanoma, | May facilitate double-stranded RNA-regulated gene expression at the level |
| (SEQ ID NO: 21) | | Hematological Ca | of post-transcription. Can act as a translation inhibitory protein. |
| RLAsYLDRV | 0.4 - 1.8 | CRC | Filament reorganization. Migration and invasion of tumor cells. |
| (SEQ ID NO: 34) | | | |
| RLFsKELRC | 0.3 | Ovarian Ca, Melanoma | Transcription initiation factor of RNA Pol II. |
| (SEQ ID NO: 39) | | | |
| RLSsPLHFV | 0.3 - 1 | CRC, Melanoma, Hematological Ca | Unknown. |
| (SEQ ID NO: 43) | | | |
| RQAsIELPSMAV | 0.1 | CRC, Hematological Ca | May play a role in mediating neutrophil activation and chemotaxis. |
| (SEQ ID NO: 46) | | | Significance in cancer unclear. |
| RQDsTPGKVFL | 1.1 - 8.4 | Ovarian Ca, CRC, Melanoma, | Orphan nuclear receptor. Primarily repressor of a broad range of genes. |
| (SEQ ID NO: 48) | | Hematological Ca. | Binds to hormone response elements (HREs). Together with NR2C2, |
| | | | forms the core of the DRED complex that represses embryonic and fetal |
| | | | globin transcription. May be involved in stem cell proliferation and |
| | | | differentiation. |
| RQLsSGVSEI | 0.9 | Ovarian Ca, Melanoma, Hematological | Involved in stress resistance and actin organization. |
| (SEQ ID NO: 51) | | Ca, CRC | |
| RTFsPTYGL | 0.3 - 6.8 | Ovarian Ca, Melanoma, OEAC | Type-VI intermediate filament (IF) which plays an important cytoskeletal |
| (SEQ ID NO: 54) | | | role within the muscle cell cytoskeleton. |
| RVAsPTSGV | 0.4 - 0.8 | Ovarian Ca, | Mediates the control of various cellular processes by insulin e.g. GH-, |
| (SEQ ID NO: 57) | | CRC, | PI3K/AKT-, IGF1R-, Leptin-signaling. |
| | | Melanoma | |
| SImsPEIQL | 0.25 | Ovarian Ca | Transcriptional repressor which may play a role in development of the |
| (SEQ ID NO: 58) | | | central nervous system (CNS). |
| SMTRsPPRV | 14 | Ovarian Ca, | mRNA splicing and insulin synthesis. |
| (SEQ ID NO: 70) | | CRC, OEAC, Hematological Ca, Melanoma | |
| VMIGsPKKV | 2-3 | Ovarian Ca, | Cell migration and invasion. |
| (SEQ ID NO: 76) | | CRC, OEAC, Hematological Ca, | |
| | | Melanoma. | |
| yLQSRYYRA | 0.2 - 1.9 | Ovarian Ca | Kinase involved in transcription regulation, apoptosis and steroidogenic |
| (SEQ ID NO: 77) | | | gene expression. Phosphorylates JUN and RUNX2. |
| PmVTLsLNL | 6 | NA | Unknown. Interacts with p53 and NEDD1 and VCAM. |
| (415) | | | |
| RTHsLLLLL | 1-32 | Ovarian Ca, CRC, OEAC, Hematological | Unknown. Is secreted by colorectal cancer cell line. |
| (SEQ ID NO: | | Ca, Melanoma | |
| 428) | | | |

TABLE 18
Summary of Reactive CD8⁺ T Cell Populations to HLA-A*02-specific Phosphopeptides

| | Healthy Donors | | | | | HH Patients | | | | | |
|---|---|---|---|---|---|---|---|---|---|---|---|
| | (mean age ~ 26 years) | | | | | (mean age ~57 years) | | | | | |
| Sequence | HD1 | HD2 | HD3 | HD4 | HD5 | HH2 | HH3 | HH6 | HH8 | HH10 | HH11 |
| NLVPMVATV (SEQ ID NO: 451) | 0 | 0 | 0 | 0 | 0 | 4 | 0 | 1 | 1 | 3 | 0 |
| GLCTLVAML (SEQ ID NO: 501) | 2 | 0 | 0 | 2 | 1 | 3 | 0 | 0 | 0 | 6 | 2 |
| RTHsLLLLL (SEQ ID NO: 428) | 0 | 0 | 0 | 0 | 0 | 0 | 0 | 0 | 0 | 0 | 0 |
| SMTRsPPRV (SEQ ID NO: 70) | 0 | 0 | 0 | 0 | 0 | 0 | 0 | 0 | 0 | 0 | 0 |
| VMIGsPKKV (SEQ ID NO: 76) | 0 | 0 | 0 | 0 | 0 | 0 | 0 | 0 | 0 | 1 | 0 |
| IMDRtPEKL (SEQ ID NO: 14) | 0 | 0 | 0 | 0 | 0 | 0 | 0 | 1 | 0 | 0 | 0 |
| RVAsPTSGV (SEQ ID NO: 57) | 0 | 0 | 0 | 0 | 0 | 0 | 0 | 0 | 0 | 2 | 0 |
| RLAsYLDRV (SEQ ID NO: 34) | 0 | nd | nd | 0 | 0 | 3 | 0 | 0 | 0 | nd | nd |
| yLQSRYYRA (SEQ ID NO: 77) | 0 | nd | nd | 0 | 0 | 0 | 0 | 1 | 0 | nd | nd |
| PmVTLsLNL (SEQ ID NO: 415) | 0 | nd | nd | 0 | 0 | 0 | 0 | 0 | 0 | nd | nd |
| KAFsPVRSV (SEQ ID NO: 16) | 0 | nd | nd | 0 | 0 | 0 | 0 | 0 | 0 | nd | 0 |
| FLDtPIAKV (SEQ ID NO: 9) | 0 | nd | nd | 0 | 0 | 0 | 0 | 0 | 0 | nd | 0 |
| KIAsEIAQL (SEQ ID NO: 17) | 0 | nd | nd | 0 | 0 | 0 | 0 | 1 | 0 | nd | nd |
| RLSsPLHFV (SEQ ID NO: 43) | 0 | nd | nd | 0 | 0 | 1 | 0 | 0 | 0 | nd | 0 |
| RTFsPTYGL (SEQ ID NO: 54) | 0 | nd | nd | 0 | 0 | 0 | 0 | 0 | 0 | nd | 0 |
| SImsPEIQL (SEQ ID NO: 58) | 0 | nd | nd | 0 | 0 | 1 | 0 | 0 | 0 | nd | nd |
| ALDsGASLLHL (SEQ ID NO: 2) | 0 | nd | nd | 0 | 0 | 0 | 0 | 0 | 0 | nd | 1 |
| AVVsPPALHNA (SEQ ID NO: 6) | 0 | nd | nd | 0 | 0 | 0 | 0 | 2 | 0 | nd | nd |
| KLFPDtPLAL (SEQ ID NO: 21) | nd | nd | nd | nd | nd | 0 | 0 | nd | 0 | nd | 0 |
| RLFsKELRC (SEQ ID NO: 39) | nd | nd | nd | nd | nd | 2 | 0 | nd | 0 | nd | nd |
| RQAsIELPSMAV (SEQ ID NO: 46) | nd | nd | nd | nd | nd | 0 | 0 | nd | 0 | nd | 0 |
| RQDsTPGKVFL (SEQ ID NO: 48) | nd | nd | nd | nd | nd | 0 | 0 | nd | 0 | nd | 0 |
| RQLsSGVSEI (SEQ ID NO: 51) | nd | nd | nd | nd | nd | 0 | 0 | nd | 0 | nd | 0 |

Phospho-Ser, phospho-Thr, and phosphor-Tyr residues are indicated by s, t, and y, respectively. Oxidized methionine residues are depicted by “m”.
nd: not determined; 0: <0.25% reactive CD8⁻⁺ T cells; 1: 0.25-2.5% reactive CD8⁻⁺ T cells; 2: 2.5-5.0% reactive CD8⁻⁺ T cells; 3: 5-7.5% reactive CD8⁻⁺ T cells; 4: 7.5-10% reactive CD8⁻⁺ T cells; 5: 10-20% reactive CD8⁻⁺ T cells; 6: >20% reactive CD8⁻⁺ T cells.

TABLE 19
Characteristics of the HH Patients

| | Hereditary | Healthy |
|---|---|---|
| Characteristic | Hemochromatosis | Population |
| Mean age (range) - yr | 57 (40-70) | 26 (22-34) |
| Male sex - % | 73 | 20 |
| Genetic background - % | | |
| C282Y homozygous | 75 | — |
| C282Y/H63D compound | 25 | — |
| Steatosis - % | 45 | 0 |
| Fibrosis - % | 27 | 0 |
| Cirrhosis - % | 0 | 0 |
| HCC - % | 0 | 0 |
| Treatment | All received phlebotomy | No treatment |
| Lab values ± SEM | | |
| WBC [g/L] | 5.61 ± 3.37 | 4-11 |
| Hb [g/L] | 149.64 ± 3.09 | 135-180 |
| Plts [*10⁹/L] | 223.64 ± 14.6 | 150-450 |
| Creatinine [μmol/L] | 82.73 ± 6.22 | 60-120 |
| GPT (ALT) [U/L] | 24.36 ± 2.46 | 10-50 |
| AP [U/L] | 65.55 ± 3.59 | 40-130 |
| Bilirubin [μmol/L] | 18.09 ± 4.27 | <22 |
| INR | 1.05 ± 0.03 | 0.85-1.15 |
| Ferritin [μg/L] (range) | 99.7 ± 21.54 (26-268) | 18-360 |

TABLE 20
Characteristics of Patients from Whom Liver Tissue
was Obtained for Lymphocyte Extraction, Expansion,
and Testing for MHC-I-pP Immune Responses

| | Deceased | Liver | |
|---|---|---|---|
| | donor | cirrhosis | HCC |
| Characteristic | livers (DDL) | patients | patients |
| Mean age (range) - yr | ? | 43.5 (34-53) | 67.7 (52-82) |
| Male sex - % | ? | 60 | 82 |
| Chronic liver disease - % | | | |
| ALD/NASH | | — | 29 |
| HBV/HCV | | — | 35 |
| AIH/PBC/PSC | | 60 | 0 |
| Others | | 40 | 35 |
| Fibrosis - % | 0 | 20 | 18 |
| Cirrhosis - % | 0 | 80 | 65 |
| Child-Turcotte-Pugh- | | | |
| stadium - % | | | |
| A | — | 40 | 55 |
| B | — | 60 | 45 |
| C | — | 0 | 0 |
| BCLC - % | | | |
| A | — | — | 76 |
| B | — | — | 24 |
| C | — | — | — |
| D | — | — | — |
| Treatment - % | | | |
| Resection/Surgery | — | 0 | 41 |
| OLTx | — | 100 | 59 |
| Lab values ± SEM | | | |
| AFP [kU/L] | | 1.3 ± 0.4 | 2598 ± 4124 |
| WBC [g/L] | | 6.6 ± 6.6 | 5.9 ± 0.9 |
| Hb [g/L] | | 109.0 ± 3.1 | 135.6 ± 11.9 |
| Plts [*10⁹/L] | | 134.7 ± 29.3 | 159.2 ± 47.8 |
| Creatinine [μmol/L] | | 47.0 ± 5.4 | 74.2 ± 14.1 |
| GPT (ALT) [U/L] | | 83.0 ± 13.9 | 36.6 ± 8.1 |
| AP [U/L] | | 783.3 ± 168.8 | 152.1 ± 29.2 |
| Bilirubin [μmol/L] | | 184.0 ± 25.9 | 22.6 ± 8.8 |
| INR | | 1.2 ± 0.1 | 1.3 ± 0.1 |

TABLE 21
Initiation of TIL Cultures by ED or TF

| | | Initiation by^{a} | | | | Successful^{b} | | | |
|---|---|---|---|---|---|---|---|---|---|
| | | TF | | ED | | TF | | ED | |
| Sample ID | | Tu | Di | Tu | Di | Tu | Di | Tu | Di |
| LL4857 | TIL1 | + | − | − | + | + | | | + |
| LL4908 | TIL2 | + | + | − | + | + | − | | − |
| LL4922 | TIL3 | + | + | + | + | − | + | + | + |
| LL4959 | TIL4 | Ø | − | Ø | + | | | | + |
| LL5176 | TIL5 | + | Ø | + | Ø | − | | + | |
| LL5210 | TIL6 | + | + | + | + | + | − | − | + |
| LL5259 | TIL8 | Ø | − | Ø | + | | | | + |
| LL5437 | TIL9 | + | + | − | + | + | − | | + |
| LL5487 | TIL10 | + | Ø | + | Ø | + | | + | |
| LL5493 | TIL11 | + | + | − | + | − | − | | + |
| LLUNKN | TIL12 | + | + | − | + | − | + | | + |
| LL5549 | TIL13 | + | + | + | + | − | − | + | + |
| LL5573 | TIL14 | + | + | + | + | + | − | + | + |
| LL5721 | TIL15 | + | + | + | + | + | − | − | + |
| LL5725 | TIL16 | + | − | − | + | + | | | + |
| LL5737 | TIL17 | + | + | + | + | − | − | − | + |
| LL5975 | TIL18 | − | − | + | + | | | + | − |

TF: tissue fragments; ED: enzymatic digestion; Tu: tumor tissue; Di: distal (normal tissue)
^{a}in these columns, “+” represents that an attempt was made to establish a culture from the corresponding sample, “−” represents that an attempt was not made to establish a culture from the corresponding sample, and “Ø” represents that tissue was not available for this sample.
^{b}in these columns, “+” represents that a culture was successfully established from the corresponding sample and “−” represents that a culture was not successfully established from the corresponding sample.

TABLE 22
Classification of TIL Cultures on the Basis of
Surface Marker Expression Between Weeks 5 and 7

| | naive | EM | EMRA | CM | Tregs |
|---|---|---|---|---|---|
| | CCR7+ | CCR7− | CCD7− | CCR7+ | CD25+ |
| | CD45RA+ | CD45RA− | CD45RA+ | CD45RA− | FoxP3+ |
| TF Tu | 6.5 ± 5.5 | 37.5 ± 14.0 | 47.3 ± 29.0 | 11.8 ± 6.8 | 1.6 ± 1.3 |
| TF Di | 10.6 ± 0.0 | 58.1 ± 0.0 | 20.0 ± 0.0 | 11.3 ± 0.0 | 0.9 ± 0.0 |
| ED Tu | 5.0 ± 1.3 | 53.8 ± 15.4 | 38.1 ± 15.8 | 3.1 ± 3.3 | 6.8 ± 5.7 |
| ED Di | 3.4 ± 3.1 | 69.5 ± 21.6 | 23.3 ± 23.8 | 3.8 ± 4.2 | 2.3 ± 1.0 |
| DDLs | 6.4 ± 5.2 | 71.1 ± 3.7 | 8.4 ± 3.4 | 14.1 ± 5.8 | 5.0 ± 3.7 |
| IHLs | 22.2 ± 12.8 | 27.1 ± 12.9 | 50.0 ± 0.6 | 0.7 ± 0.5 | 24.8 ± 32.8 |
| REP | 1.64 ± 1.68 | 55.76 ± 12.89 | 1.02 ± 1.50 | 41.58 ± 13.37 | n.d. |
| PBMCs | 33.8 ± 27.8 | 28.0 ± 14.2 | 24.1 ± 9.7 | 14.1 ± 6.3 | 1.0 ± 1.2 |

Data are presented as mean ± SD.
Abbreviations: EM: effector memory T cell; EMRA: effector memory T cell expressing CD45RA; CM: central memory T cell; Tregs: regulatory T cell.

TABLE 23
Effect of Antigen-specific Expansion on
Tumor-infiltrating Lymphocyte Cultures

| | | | | Non- | | | pP- | | |
|---|---|---|---|---|---|---|---|---|---|
| | No | | | specific | | | specific | | |
| Sequence | Expansion | | | Expansion | | | Expansion | | |
| (SEQ ID NO:) | HH6 | Do2 | Do4 | HH6 | Do2 | Do4 | HH6 | Do2 | Do4 |
| NLVPMVATV | 1 | nd | 2 | 1 | 0 | 5 | 3 | 3 | 2 |
| (451) | | | | | | | | | |
| GLCTLVAML | 0 | nd | 1 | 0 | 2 | 3 | 4 | 3 | 3 |
| (501) | | | | | | | | | |
| RTHsLLLLL | 1 | nd | 2 | 1 | 0 | 0 | 0 | 0 | 0 |
| (428) | | | | | | | | | |
| SMTRsPPRV | 0 | nd | 4 | 0 | 0 | 0 | 0 | 0 | 0 |
| (70) | | | | | | | | | |
| VMIGsPKKV | 0 | nd | 0 | 0 | 0 | 0 | 0 | 1 | 0 |
| (76) | | | | | | | | | |
| IMDRtPEKL | 2 | nd | 6 | 0 | 0 | 0 | 2 | 1 | 1 |
| (14) | | | | | | | | | |
| RVAsPTSGV | 0 | nd | 3 | 0 | 0 | 0 | 0 | 0 | 2 |
| (57) | | | | | | | | | |
| RLAsYLDRV | 0 | nd | nd | 1 | 0 | 0 | 0 | 0 | 0 |
| (34) | | | | | | | | | |
| yLQSRYYRA | 1 | nd | nd | 0 | 0 | 0 | 0 | 2 | 0 |
| (77) | | | | | | | | | |
| PmVTLsLNL | 0 | nd | nd | 0 | 0 | 1 | 0 | 2 | 1 |
| (415) | | | | | | | | | |
| KAFsPVRSV | 0 | nd | nd | 0 | 0 | 0 | 0 | 0 | 0 |
| (16) | | | | | | | | | |
| FLDtPIAKV | 0 | nd | nd | 0 | 1 | 0 | 2 | 0 | 0 |
| (9) | | | | | | | | | |
| KIAsEIAQL | 1 | nd | nd | 1 | 1 | 0 | 0 | 0 | 0 |
| (17) | | | | | | | | | |
| RLSsPLHFV | 0 | nd | nd | 0 | 0 | 0 | 0 | 0 | 0 |
| (43) | | | | | | | | | |
| RTFsPTYGL | 1 | nd | nd | 0 | 0 | 0 | 3 | 0 | 0 |
| (54) | | | | | | | | | |
| SImsPEIQL | 0 | nd | nd | 0 | 0 | 0 | 2 | 0 | 0 |
| (58) | | | | | | | | | |
| ALDsGASLLHL | 2 | nd | nd | 0 | 0 | 0 | 2 | 0 | 0 |
| (2) | | | | | | | | | |
| AVVsPPALHNA | 2 | nd | nd | 0 | 0 | 0 | 2 | 0 | 0 |
| (6) | | | | | | | | | |
| KLFPDtPLAL | 0 | nd | nd | nd | nd | nd | 0 | 0 | 0 |
| (21) | | | | | | | | | |
| RLFsKELRC | 0 | nd | nd | nd | nd | nd | 0 | 0 | 0 |
| (39) | | | | | | | | | |
| RQAsIELPSMAV | 1 | nd | nd | nd | nd | nd | 0 | 0 | 0 |
| (46) | | | | | | | | | |
| RQDsTPGKVFL | 2 | nd | nd | nd | nd | nd | 1 | 0 | 0 |
| (48) | | | | | | | | | |
| RQLsSGVSEI | 2 | nd | nd | nd | nd | nd | 0 | 0 | 0 |
| (51) | | | | | | | | | |

Phospho-Ser, phospho-Thr and phosphor-Tyr residues are indicated by s, t, and y, respectively. Oxidized methionine residues are depicted by “m”.
nd: not determined; 0: <0.25% reactive CD8⁻⁺ T cells; 1: 0.25-2.5% reactive CD8⁻⁺ T cells; 2: 2.5-5.0% reactive CD8⁻⁺ T cells; 3: 5-7.5% reactive CD8⁻⁺ T cells; 4: 7.5-10% reactive CD8⁻⁺ T cells; 5: 10-20% reactive CD8⁻⁺ T cells; 6: >20% reactive CD8⁻⁺ T cells.

TABLE 24
Summary of ppCTL Responses from pP-specifically Expanded Cultures
from “Healthy” Livers, Cirrhotic Livers, and HCCs

| | | | | | | | | | HCC | | | | | |
|---|---|---|---|---|---|---|---|---|---|---|---|---|---|---|
| | Deceased Donor Liver | | | | End-stage Liver Cirrhosis | | | | Tumor | | Distal | | | |
| | Do | Do | Do | Do | IHL | IHL | IHL | IHL | HCC | HCC | HCC | HCC | HCC | HCC |
| Sequence | 2 | 3 | 4 | 6 | 11 | 12 | 13 | 15 | 1 | 9 | 1 | 4 | 9 | 12 |
| NLVPMVATV | 3 | 0 | 2 | 0 | 0 | 0 | 0 | 0 | 0 | 0 | 1 | 0 | 0 | 0 |
| (SEQ ID NO: 451) | | | | | | | | | | | | | | |
| GLCTLVAML | 3 | 0 | 3 | 0 | 0 | 0 | 0 | 0 | 0 | 0 | 0 | 0 | 0 | 0 |
| (SEQ ID NO: 501) | | | | | | | | | | | | | | |
| RTHsLLLLL | 0 | 0 | 0 | 0 | 0 | 0 | 0 | 0 | 0 | 0 | 0 | 0 | 0 | 0 |
| (SEQ ID NO: 428) | | | | | | | | | | | | | | |
| SMTRsPPRV | 1 | 0 | 0 | 0 | 0 | 0 | 0 | 6 | 0 | 0 | 0 | 0 | 0 | 0 |
| (SEQ ID NO: 70) | | | | | | | | | | | | | | |
| VMIGsPKKV | 2 | 0 | 0 | 0 | 0 | 0 | 0 | 0 | 0 | 0 | 0 | 0 | 0 | 0 |
| (SEQ ID NO: 76) | | | | | | | | | | | | | | |
| IMDRtPEKL | 2 | 0 | 1 | 0 | 0 | 0 | 0 | 0 | 0 | 0 | 0 | 0 | 0 | 0 |
| (SEQ ID NO: 14) | | | | | | | | | | | | | | |
| RVAsPTSGV | 0 | 0 | 2 | 1 | 0 | 0 | 0 | 0 | 0 | 0 | 0 | 0 | 0 | 0 |
| (SEQ ID NO: 57) | | | | | | | | | | | | | | |
| RLAsYLDRV | 0 | 0 | 0 | 0 | 0 | 0 | 0 | 0 | 0 | 0 | 0 | 0 | 0 | 0 |
| (SEQ ID NO: 34) | | | | | | | | | | | | | | |
| yLQSRYYRA | 2 | 0 | 0 | 0 | 0 | 0 | 0 | 0 | 0 | 1 | 0 | 0 | 0 | 0 |
| (SEQ ID NO: 77) | | | | | | | | | | | | | | |
| PmVTLsLNL | 2 | 0 | 2 | 1 | 0 | 0 | 0 | 0 | 0 | 0 | 0 | 0 | 0 | 0 |
| (SEQ ID NO: 415) | | | | | | | | | | | | | | |
| KAFsPVRSV | 1 | 0 | 0 | 0 | 0 | 0 | 0 | 0 | 0 | 0 | 0 | 0 | 0 | 0 |
| (SEQ ID NO: 16) | | | | | | | | | | | | | | |
| FLDtPIAKV | 0 | 0 | 0 | 0 | 0 | 0 | 0 | 0 | 0 | 0 | 0 | 0 | 0 | 0 |
| (SEQ ID NO: 9) | | | | | | | | | | | | | | |
| KIAsEIAQL | 0 | 0 | 1 | 1 | 0 | 0 | 0 | 0 | 0 | 0 | 0 | 0 | 0 | 0 |
| (SEQ ID NO: 17) | | | | | | | | | | | | | | |
| RLSsPLHFV | 0 | 0 | 0 | 0 | 0 | 0 | 0 | 0 | 0 | 0 | 0 | 0 | 0 | 0 |
| (SEQ ID NO: 43) | | | | | | | | | | | | | | |
| RTFsPTYGL | 0 | 0 | 0 | 0 | 0 | 0 | 0 | 1 | 0 | 0 | 0 | 0 | 0 | 0 |
| (SEQ ID NO: 54) | | | | | | | | | | | | | | |
| SImsPEIQL | 0 | 0 | 0 | 0 | 0 | 0 | 0 | 0 | 0 | 0 | 0 | 0 | 0 | 0 |
| (SEQ ID NO: 58) | | | | | | | | | | | | | | |
| ALDsGASLLHL | 0 | 0 | 0 | 0 | 0 | 0 | 0 | 3 | 0 | 0 | 0 | 0 | 0 | 0 |
| (SEQ ID NO: 2) | | | | | | | | | | | | | | |
| AVVsPPALHNA | 0 | 0 | 0 | 0 | 0 | 0 | 0 | 0 | 0 | 1 | 0 | 0 | 0 | 0 |
| (SEQ ID NO: 6) | | | | | | | | | | | | | | |
| KLFPDtPLAL | 0 | 0 | 0 | 0 | 0 | nd | 0 | nd | 0 | nd | 0 | 0 | nd | nd |
| (SEQ ID NO: 21) | | | | | | | | | | | | | | |
| RLFsKELRC | 1 | 0 | 0 | 0 | 0 | nd | 0 | nd | 0 | nd | 0 | 0 | nd | nd |
| (SEQ ID NO: 39) | | | | | | | | | | | | | | |
| RQAsIELPSMAV | 0 | 0 | 0 | 1 | 0 | nd | 0 | nd | 0 | nd | 0 | 0 | nd | nd |
| (SEQ ID NO: 46) | | | | | | | | | | | | | | |
| RQDsTPGKVFL | 1 | 0 | 0 | 0 | 0 | nd | 0 | nd | 0 | nd | 0 | 0 | nd | nd |
| (SEQ ID NO: 48) | | | | | | | | | | | | | | |
| RQLsSGVSEI | 0 | 0 | 0 | 0 | 0 | nd | 0 | nd | 0 | nd | 0 | 0 | nd | nd |
| (SEQ ID NO: 51) | | | | | | | | | | | | | | |

Phospho-Ser, phospho-Thr, and phosphor-Tyr residues are indicated by s, t, and y, respectively Oxidized methionine residues are depicted by “m”.
nd: not determined; 0: <0.25% reactive CD8-+ T cells; 1: 0.25-2.5% reactive CD8-+ T cells; 2: 2.5-5.0% reactive CD8-+ T cells; 3: 5-7.5% reactive CD8-+ T cells; 4: 7.5-10% reactive CD8-+ T cells; 5: 10-20% reactive CD8-+ T cells; 6: >20% reactive CD8-+ T cells.

TABLE 25
Functional Annotation Clustering of MHC-I-pP to Biological Processes (GO Term BP) using DAVID v6.7

| Cluster | | | Proteins in | Enrichment |
|---|---|---|---|---|
| rank | GO Term BP cluster | Included GO Terms | cluster | Score |
| 1 | Phosphorylation | GO:0006468; GO:0016310; GO:0006793; GO:0006796 | 22 | 3.10 |
| 2 | Negative regulation of | GO:0051253; GO:0010605; GO:0006357; GO:0045892; | 45 | 2.57 |
| | transcription | GO:0051172; GO:0010629; GO:0045934; GO:0016481; | | |
| | | GO:0009890; GO:0051252; GO:0010558; GO:0045449; | | |
| | | GO:0031327; GO:0006350; GO:0000122; GO:0006355. | | |
| 3 | Cell cycle | GO:0007049; GO:0022403; GO:0000279; GO:0022402; | 22 | 2.48 |
| | | GO:0000278; GO:0051301; GO:0000280; GO:0007067; | | |
| | | GO:0000087; GO:0048285. | | |
| 4 | Apoptosis/Cell Death | GO:0008219; GO:0016265; GO:0006915; GO:0012501 | 20 | 2.46 |
| | | GO:0042981; GO:0043067; GO:0010941 | | |
| 5 | Transcription RNA Pol II | GO:0006366; GO:0006351; GO:0032774 | 9 | 1.82 |
| 6 | Regulation of metabolic | GO:0032268; GO:0031399; GO:0044093; GO:0001932; | 17 | 1.79 |
| | processes | GO:0046328; GO:0080135; GO:0045859; GO:0070302; | | |
| | | GO:0042325; GO:0043506; GO:0043549; GO:0019220; | | |
| | | GO:0051174; GO:0051338; GO:0043408; GO:0043085; | | |
| | | GO:0010627; GO:0045860; GO:0033674; GO:0032147; | | |
| | | GO:0051347; GO:0043405 | | |
| 7 | Apoptosis/Cell Death | GO:0042981; GO:0043067; GO:0010941; GO:0043065; | 19 | 1.635 |
| | | GO:0043068; GO:0010942; GO:0006917; GO:0012502; | | |
| | | GO:0008624 | | |
| 8 | Apoptosis | GO:0042981; GO:0043067; GO:0010941; GO:0043066; | 19 | 1.54 |
| | | GO:0043069; GO:0060548; GO:0006916 | | |
| 9 | Cell cycle/mitosis | GO:0000278; GO:0051329; GO:0051325; GO:0000079 | 12 | 1.54 |
| 10 | Metabolic processes/ | GO:0009891; GO:0031328; GO:0010557; GO:0010604; | 18 | 1.51 |
| | biosythesis | GO:0045893; GO:0051254; GO:0045941; GO:0051173; | | |
| | | GO:0010628; GO:0045935; GO:0045944 | | |
| 11 | Cytoskeleton organization | GO:0007010; GO:0030036; GO:0030029 | 14 | 1.47 |
| 12 | DNA repair | GO:0006974; GO:0033554; GO:0006281; GO:0006259; | 15 | 1.45 |
| | | GO: 0006260 | | |
| 13 | Myeloid cell differentiation | GO:0045639; GO:0002763; GO:0045637; GO:0045597; | 7 | 1.42 |
| | | GO:0002761; GO:0051094 | | |
| 14 | Response to hormone | GO:0032870; GO:0010033; GO:0032869; GO:0009725; | 9 | 1.40 |
| | stimulus (e.g. insulin) | GO:0009719; GO:0032868; GO:0007169; GO:0043434 | | |
| 15 | Cell cycle/mitosis | GO:0045859; GO:0043549; GO:0051338; GO:0000280; | 11 | 1.32 |
| | | GO:0007067; GO:0000087; GO:0048285; GO:0045786; | | |
| | | GO:0006469; GO:0033673; GO:0051348; GO:0044092; | | |
| | | GO:0043086; GO:0010498; GO:0043161 | | |
| 16 | DNA binding | GO:0043388; GO:0051101; GO:0051099; GO:0051098; | 5 | 1.04 |
| | | GO:0051090 | | |
| 17 | RNA catabolic process | GO:0000956; GO:0006402; GO:0006401 | 3 | 1.00 |
| 18 | RNA splicing/processing | GO:0016071; GO:0006397; GO:0006396; GO:0008380 | 10 | 0.98 |
| 19 | Protein modification | GO:0031399; GO:0031401; GO:0032270; GO:0051247 | 11 | 0.89 |
| 20 | Protein complex assembly/ | GO:0070271; GO:0006461; GO:0043933; GO:0065003 | 12 | 0.74 |
| | biogenesis | | | |
| 21 | Chromatin organization | GO:0006325; GO:0051276; GO:0016568 | 9 | 0.56 |
| 22 | Membrane organization | GO:0016044; GO:0016192; GO:0010324; GO:0006897 | 9 | 0.56 |
| 23 | Cell motility/migration | GO:0006928; GO:0048870; GO:0051674; GO:0016477 | 9 | 0.54 |

TABLE 26
Biocarta and KEGG Signaling Pathway Mapping of Identified MHC-I-pP Revealed
that MHC-I-pP were Significantly Enriched in the MAPK Signaling Pathway

| Signaling | | | | |
|---|---|---|---|---|
| pathway | Proteins | Database | p value | Significance for cancer |
| MAPKinase | DAXX, HSPB1, JUN, | Biocarta | 0.012 | MAPK pathways are evolutionarily conserved kinases that |
| | MAP3K11, MAP3K3, | KEGG | 0.1 | link extracellular signals to the machinery that controls |
| | RPS6KA1, RAF1 | BBID | 0.074 | fundamental cellular processes such as growth, |
| | | | | proliferation, differentiation, migration and apoptosis. |
| Neurotrophin | RAPGEF1, IRS2, JUN, | KEGG | 0.017 | Neurotrophins play a major role in neuron survival, |
| | MAP3K3, RPS6KA1, | | | proliferation, differentiation and apoptosis. They |
| | RAF1 | | | function by interacting with tyrosine kinase receptors |
| | | | | which activate MAPK, PI3K and PLC-γ pathways. |

TABLE 27
Exemplary Phosphopeptides Found on >1 Sample or Cancer**

| Gene | | | | | |
|---|---|---|---|---|---|
| (UniProt | | HLA- | | | |
| Acc. No.) | Phosphopeptide | motif % | Found on | Other cancers | Function and significance for cancer |
| SRSF7 | SPRRsRSISL | B*07 | LL4857T | CRC, Mel, | Required for pre-mRNA splicing. |
| (Q16629) | (SEQ ID NO: 284) | | | BCa, HaemCa | |
| SRSF8 | SMTRsPPRV | A*02 | HepG2 | CRC, OEAC, | Involved in pre-mRNA alternative splicing. |
| (Q9BRL6) | (SEQ ID NO: 70) | | | Mel, OvCa | |
| LATS1 | KRYsGNmEY | C*07 | LL3907T | CRC, OEAC, | Negative regulator of YAP1 in the Hippo signalling pathway. |
| (O95835) | (SEQ ID NO: 242) | | LL370T | Mel | Acts as a tumor suppressor in controlling mitotic |
| | | | LL981T | | progression. Involved in the control of p53 expression. |
| | | | LL4857T | | |
| LATS2 | RRDsLQKPGL | B*27 | LL981T | CRC | Same as for LATS1. |
| (Q9NRM7) | (SEQ ID NO: 248) | | | | |
| BRD4 | REAPsPLmI | B*49 | LL3907T | HaemCa | Recognizes and binds acetylated histones and plays a key role |
| (O60885) | (SEQ ID NO: 352) | | | | in transmission of epigenetic memory across cell divisions |
| | AVVsPPALHNA | A*02 | LL981 | OvCa, | and transcription regulation. Also acts as a regulator of |
| | (SEQ ID NO: 6) | | HepG2 | HaemCa | p53/TP53-mediated transcription. |
| SIPA1L1 | REYGsTSSI | B*49 | LL3907T | — | Promotes reorganization of the actin cytoskeleton. |
| (O43166) | (SEQ ID NO: 364) | | | | |
| | RRPsYTLGM | C*07 | LL370T | Mel | |
| | (SEQ ID NO: 273) | | | | |
| SIPA1L3 | REVsPAPAV | B*49 | LL3907T | — | ? |
| (O60292) | (SEQ ID NO: 363) | | | | |
| HIPK3 | yLQSRYYRA | A*02 | LL3907T | OvCa | Kinase involved in transcription regulation, apoptosis and |
| (Q9H422) | (SEQ ID NO: 77) | | LL4857T | | steroidogenic gene expression. Phosphorylates JUN and |
| | | | LL981T | | RUNX2. |
| HIPK1 | YPLsPTKISQY | B*35 | HepG2 | OEAC | Serine/threonine-protein kinase involved in transcription |
| (Q86Z02) | (SEQ ID NO: 296) | | | | regulation and TNF-mediated cellular apoptosis. Activates |
| | | | | | nuclear MAP3K5-JNK. May be involved in anti-oxidative |
| | | | | | stress responses. Involved in the regulation of eye |
| | | | | | embryogenesis. Promotes angiogenesis. May be involved |
| | | | | | in malignant squamous cell tumor formation. |
| EPN1 | TRKtPESFL | C*06 | LL981T | CRC, OEAC | Modifies membrane curvature and facilitates the formation of |
| (Q9Y613) | (SEQ ID NO: 381) | | | | clathrin-coated invaginations. Regulates receptor-mediated |
| | RPIsPRIGAL | B*07 | LL4857T | CRC, OEAC, | endocytosis |
| | (SEQ ID NO: 141) | | | BCa, | |
| | | | | HaemCa | |
| NCOR1 | SARRtPVSY | C*02 | LL981T | — | Mediates transcriptional repression by certain nuclear |
| (O75376) | (SEQ ID NO: 529) | | | | receptors. Part of a complex which promotes histone |
| | RRPsLLSEF | B*27 | LL981N + T | CRC, OEAC | deacetylation and the formation of repressive chromatin |
| | (SEQ ID NO: 271) | | | | structures which may impede the access of basal |
| | | | | | transcription factors |
| NCOR2 | ITQGtPLKY | A*01 | LL981T | CRC, OEAC, | Transcriptional corepressor. Involved in the regulation of the |
| (Q9Y618) | (SEQ ID NO: 289) | | | Mel, HaemCa | germinal centre B-cells proliferation and survival. |
| MAP3K3 | GRsSPPPGY | B*27 | LL981T | — | Component of a MAP kinase signal transduction cascade. |
| (Q99759) | (SEQ ID NO: 232) | | | | Mediates activation of the NF-kappa-B, AP1 and DDIT3 |
| | | | | | transcriptional regulators. |
| MAP3K11 | TPRsPPLGLI | B*07 | LL4857T | Mel, BCa, | Activates the JUN N-terminal pathway. Plays a role in |
| (Q16584) | (SEQ ID NO: 217) | | | HaemCa, | mitogen-stimulated activation of BRAF. Influences |
| | | | | Mel, CRC | microtubule organization during the cell cycle. |
| GAREM | LLDPSRSYsY | A*01 | LL981T | Mel | Acts as an adapter protein that plays a role in intracellular |
| (Q9H706) | (SEQ ID NO: 290) | | | | signalling cascades triggered either EGFR and/or |
| | RSYsYPRQK | A*03 | LL4857T | Mel | cytoplasmic protein tyrosine kinases. Promotes activation |
| | (SEQ ID NO: 326) | | | | of the MAPK/ERK signalling pathway. Plays a role in the |
| | | | | | regulation of cell proliferation. |
| ZFP36L1 | GLLDsPTSI | A*02 | LL370T | OvCa | Probable regulatory protein involved in regulating the |
| (Q07352) | (SEQ ID NO: 13) | | | | response to growth factors. |
| ZFP36L2 | RRHsASNLHAL | C*06 | L981T | — | mRNA-binding protein that plays a key role in self-renewal of |
| (P47974) | (SEQ ID NO: 375) | | | | erythroid cells in response to glucocorticoids. |
| PRKAR1A | RPRAAtVV | B*07 | LL4857T | CRC | Regulatory subunit of the cAMP-dependent protein kinases |
| (P10644) | (SEQ ID NO: 153) | | | | involved in cAMP signaling in cells. Membrane association |
| PRKAR2A | SRFNRRVsV | C*06 | LL981T | — | by binding to anchoring proteins, including the MAP2 |
| (P13861) | (SEQ ID NO: 285) | | | | kinase. |
| ANAPC1 | VLLsPVPEL | A*02 | HepG2 | Mel, HaeCa, | Component of the anaphase promoting complex/cyclosome |
| (Q9H1A4) | (SEQ ID NO: 74) | | | | (APC/C), a cell cycle-regulated E3 ubiquitin ligase that |
| ANAPC7 | KRYsRALYL | C*07 | LL370T | OvCa, | controls progression through mitosis and the G1 phase of |
| (Q9UJX3) | (SEQ ID NO: 243) | | | CRC, OEAC, | the cell cycle. |
| | | | | Mel | |
| MEF2D | RLLsPQQPAL | A*02 | LL370T | BCa | Transcriptional activator mediates cellular functions in muscle |
| (Q14814) | (SEQ ID NO: 122) | | | | development, but also in neuronal differentiation and |
| | RPAsAGAmL | B*07 | LL4857T | CRC, HaemCa, | survival. Plays diverse roles in the control of cell growth, |
| | (SEQ ID NO: 125) | | | Mel | survival and apoptosis via p38 MAPK signalling. |
| AKAP13 | RRSFsLE | ? | LL370T | — | Anchors cAMP-dependent protein kinase (PKA). Augments |
| (Q12802) | (SEQ ID NO: 275) | | | | gene activation by the estrogen receptor and activates |
| | RPRsAVLL | B*07 | LL4857N + T | CRC, BCa | estrogen receptor beta by a p38 MAPK-dependent pathway. |
| | (SEQ ID NO: 169) | | | | |
| MICALL2 | KLPsPAPARK | A*03 | LL4857N + T | CRC | Regulates cell adhesion molecules transport to the plasma |
| (Q8IY33) | (SEQ ID NO: 308) | | | | membrane and actin cytoskeleton reorganization. Most |
| | RPAsPGPSL | B*07 | LL4857T | BCa | probably involved in the processes of epithelial cell |
| | (SEQ ID NO: 128) | | | | differentiation, cell spreading and neurite outgrowth. |
| MICAL3 | RPDsPTRPTL | B*07 | LL4857T | BCa | Acts by modifying actin subunits, leading to promote actin |
| (Q7RTP6) | (SEQ ID NO: 133) | | | | filament severing and prevent repolymerization. |
| BCAR3 | RPDVAKRLsL | B*07 | LL4857T | CRC | May act as an adapter protein and couples activated growth |
| (O75815) | (SEQ ID NO: 135) | | | | factor receptors to a signalling pathway that regulates the |
| (SEQ | IMDRtPEKL | A*02 | LL4857T | Mel, Haem Ca, | proliferation in breast cancer cells. When overexpressed, it |
| | ID NO: 14) | | | OvCa | confers anti-estrogen resistance in breast cancer cell lines. |
| | | | | | May also be regulated by cellular adhesion to extracellular |
| | | | | | matrix proteins. |
| TP53BP1 | SIDsPQKL | C*05 | LL4857T | Mel, CRC | Plays a key role in the response to DNA damage. May have a |
| (Q12888) | (SEQ ID NO: 388) | | | | role in checkpoint signaling during mitosis. Enhances |
| | RSDsYVEL | C*05 | LL4857T | CRC | TP53-mediated transcriptional activation. |
| | (SEQ ID NO: 385) | | | | |

**Many of these MHC-I-pP are in a central position of essential cancer-associated signaling pathways.
% Predicted.

TABLE 28
Phosphopeptides Identified in HCC and Other Cancer Types Sorted by HLA Type

| | | HLA- | | | | | | |
|---|---|---|---|---|---|---|---|---|
| SEQ ID NO: | Sequence# | Type | Leukemia | Colorectal | Melanoma | Ovarian | Breast | Esophageal |
| 286 | AEQGsPRVSY | A*0101 | | | | | | |
| 287 | GsPHYFSPFRPY | A*0101 | | | | | | |
| 288 | ISSsMHSLY | A*0101 | | ✓ | | | | |
| 289 | ITQGtPLKY | A*0101 | | ✓ | ✓ | | | ✓ |
| 290 | LLDPSRSYsY | A*0101 | | | ✓ | | | |
| 291 | SLDsPSYVLY | A*0101 | | | ✓ | | | |
| 292 | SLYDRPAsY | A*0101 | | | | | | |
| 293 | SYPsPVATSY | A*0101 | | | | | | |
| 294 | TMAsPGKDNY | A*0101 | | ✓ | ✓ | | | ✓ |
| 295 | YFsPFRPY | A*0101 | | | | | | |
| 296 | YPLsPTKISQY | A*0101 | | | | | | ✓ |
| 297 | YQRPFsPSAY | A*0101 | | | | | | |
| 1 | AIMRsPQMV | A*0201 | | | | ✓ | | |
| 2 | ALDsGASLLHL | A*0201 | | ✓ | | ✓ | | |
| 3 | ALGNtPPFL | A*0201 | | | | ✓ | | |
| 4 | ALMGsPQLV | A*0201 | | | | ✓ | | |
| 5 | ALMGsPQLVAA | A*0201 | | | | ✓ | | |
| 6 | AVVsPPALHNA | A*0201 | | | ✓ | ✓ | | |
| 7 | DLKRRsmSI | A*0201 | | ✓ | | | | |
| 8 | ELFSsPPAV | A*0201 | | | | ✓ | | |
| 9 | FLDtPIAKV | A*0201 | | ✓ | | | | |
| 10 | GIDsPSSSV | A*0201 | | | | | | |
| 11 | GLDsGFHSV | A*0201 | | | | | | |
| 12 | GLIsPVWGA | A*0201 | | | | ✓ | | |
| 13 | GLLDsPTSI | A*0201 | | | | ✓ | | |
| 14 | IMDRtPEKL | A*0201 | | | ✓ | ✓ | | |
| 404 | IQFsPPFPGA | A*0201 | | | | | | |
| 15 | KAFsPVR | A*0201 | | ✓ | | ✓ | | |
| 16 | KAFsPVRSV | A*0201 | | ✓ | | ✓ | | ✓ |
| 17 | KIAsEIAQL | A*0201 | | | | ✓ | | |
| 18 | KIGsIIFQV | A*0201 | | | | ✓ | | |
| 19 | KLAsPELERL | A*0201 | | ✓ | ✓ | ✓ | | |
| 20 | KLDsPRVTV | A*0201 | | | | ✓ | | |
| 21 | KLFPDtPLAL | A*0201 | | ✓ | ✓ | ✓ | | |
| 22 | KLIDIVsSQKV | A*0201 | | ✓ | ✓ | ✓ | | |
| 23 | KLIDRTEsL | A*0201 | | ✓ | | ✓ | | |
| 409 | KLKDRLPsI | A*0201 | | | | | | |
| 24 | KLMsDVEDV | A*0201 | | | | ✓ | | |
| 25 | KLMsPKADVKL | A*0201 | | | ✓ | ✓ | | |
| 410 | KLsGDQPAAR | A*0201 | | | | | | |
| 26 | KQDsLVINL | A*0201 | | | | ✓ | | |
| 27 | KTMsGTFLL | A*0201 | | ✓ | | ✓ | | |
| 28 | KVAsLLHQV | A*0201 | | | | ✓ | | |
| 29 | LMFsPVTSL | A*0201 | | | ✓ | ✓ | | |
| 415 | PmVTLsLNL | A*0201 | | | | | | |
| 30 | RASsLSITV | A*0201 | | | | ✓ | | |
| 31 | RLAsASRAL | A*0201 | | | | ✓ | | |
| 32 | RLAsLQSEV | A*0201 | | | | ✓ | | |
| 33 | RLAsYLDKV | A*0201 | | | | ✓ | | |
| 34 | RLAsYLDRV | A*0201 | | ✓ | | | | |
| 35 | RLDsYVR | A*0201 | | | ✓ | ✓ | | |
| 36 | RLDsYVRSL | A*0201 | | | ✓ | ✓ | | ✓ |
| 37 | RLFsKEL | A*0201 | | | | | | |
| 38 | RLFsKELR | A*0201 | | | ✓ | ✓ | | |
| 39 | RLFsKELRC | A*0201 | | | ✓ | ✓ | | |
| 40 | RLLsDLEEL | A*0201 | | | | ✓ | | |
| 41 | RLLsTDAEAV | A*0201 | | | | ✓ | | |
| 42 | RLSDtPPLL | A*0201 | | | | ✓ | | |
| 43 | RLSsPLHFV | A*0201 | | ✓ | ✓ | ✓ | | |
| 44 | RMYsFDDVL | A*0201 | | | | ✓ | | |
| 45 | RQAsIELPSM | A*0201 | ✓ | ✓ | | | | |
| 46 | RQAsIELPSMAV | A*0201 | | ✓ | | | | |
| 47 | RQAsLSISV | A*0201 | | | | ✓ | | |
| 48 | RQDsTPGKVFL | A*0201 | | ✓ | ✓ | ✓ | | |
| 49 | RQIsQDVKL | A*0201 | | ✓ | ✓ | ✓ | | ✓ |
| 50 | RQLsALHRA | A*0201 | | | | ✓ | | |
| 51 | RQLsSGVSEI | A*0201 | | ✓ | ✓ | ✓ | | |
| 52 | RSLsESYEL | A*0201 | | | | ✓ | | |
| 53 | RSLsQELVGV | A*0201 | | | | ✓ | | |
| 54 | RTFsPTYGL | A*0201 | | | ✓ | ✓ | | ✓ |
| 55 | RTLsHISEA | A*0201 | | | ✓ | ✓ | | |
| 56 | RTYsGPMNKV | A*0201 | | | | ✓ | | |
| 57 | RVAsPTSGV | A*0201 | | ✓ | ✓ | ✓ | | |
| 58 | SImsPEIQL | A*0201 | | | | ✓ | | |
| 59 | SISsMEVNV | A*0201 | | ✓ | | | | |
| 60 | SISStPPAV | A*0201 | | | | ✓ | | |
| 61 | SLFGGsVKL | A*0201 | | | | ✓ | | |
| 62 | SLFsGDEENA | A*0201 | | | | ✓ | | |
| 63 | SLFsPQNTL | A*0201 | | | | ✓ | | |
| 64 | SLFsSEESNL | A*0201 | | | | ✓ | | |
| 65 | SLFsSEESNLGA | A*0201 | | | ✓ | ✓ | | |
| 66 | SLHDIQLsL | A*0201 | | | | ✓ | | |
| 67 | SLQPRSHsV | A*0201 | | | | ✓ | | |
| 68 | SLQsLETSV | A*0201 | | | | ✓ | | |
| 69 | SMSsLSREV | A*0201 | | | | ✓ | | |
| 70 | SMTRsPPRV | A*0201 | | | ✓ | ✓ | | |
| 71 | SVKPRRTsL | A*0201 | | ✓ | | | | |
| 72 | TVFsPTLPAA | A*0201 | | | | ✓ | | |
| 443 | VLFPEsPARA | A*0201 | | | | | | |
| 73 | VLFSsPPQM | A*0201 | | | | ✓ | | |
| 444 | VLIENVAsL | A*0201 | | | | | | |
| 74 | VLLsPVPEL | A*0201 | | | ✓ | ✓ | | |
| 445 | VLSDVIPsI | A*0201 | | | | | | |
| 446 | VLVVDTPsI | A*0201 | | | | | | |
| 75 | VLYsPQMAL | A*0201 | | | | ✓ | | |
| 76 | VMIGsPKKV | A*0201 | | ✓ | ✓ | ✓ | | |
| 77 | yLQSRYYRA | A*0201 | | | | ✓ | | |
| 298 | ATYtPQAPK | A*0301 | | | ✓ | | | |
| 299 | FLIIRtVLQL | A*0301 | | | | | | |
| 300 | FRYsGKTEY | A*0301 | | | ✓ | | | |
| 301 | GIMsPLAKK | A*0301 | ✓ | ✓ | | | | |
| 402 | HTAsPTGMMK | A*0301 | | | | | | |
| 403 | HVYtPSTTK | A*0301 | | | | | | |
| 302 | IISsPLTGK | A*0301 | | | ✓ | | | |
| 303 | ILKPRRsL | A*0301 | | | | | | |
| 304 | IYQyIQSRF | A*0301 | ✓ | ✓ | ✓ | | | |
| 305 | KLPDsPALA | A*0301 | | | | | | |
| 306 | KLPDsPALAK | A*0301 | | | ✓ | | | |
| 307 | KLPDsPALAKK | A*0301 | | | ✓ | | | |
| 308 | KLPsPAPARK | A*0301 | | ✓ | | | | |
| 309 | KLRsPFLQK | A*0301 | ✓ | | ✓ | | | |
| 310 | KMPTtPVKAK | A*0301 | | ✓ | ✓ | | | |
| 311 | KRAsVFVKL | A*0301 | | | ✓ | | | |
| 312 | KTPTsPLKMK | A*0301 | ✓ | | ✓ | | | |
| 313 | KVQsLRRAL | A*0301 | | ✓ | ✓ | | | |
| 314 | MTRsPPRVSK | A*0301 | | | ✓ | | | |
| 315 | RAKsPISLK | A*0301 | ✓ | ✓ | ✓ | | | |
| 419 | RIGsPLSPK | A*0301 | | | | | | |
| 316 | RILsGVVTK | A*0301 | ✓ | ✓ | ✓ | | | |
| 317 | RIYQyIQ | A*0301 | | ✓ | ✓ | | | |
| 318 | RIYQyIQSR | A*0301 | | ✓ | ✓ | | | |
| 319 | RIYQyIQSRF | A*0301 | | ✓ | ✓ | | | |
| 320 | RLFVGsIPK | A*0301 | | | | | | |
| 321 | RLLDRSPsRSAK | A*0301 | | | ✓ | | | |
| 322 | RLSsPISKR | A*0301 | ✓ | ✓ | ✓ | | | |
| 323 | RLSsPVLHR | A*0301 | | | ✓ | | | |
| 424 | RMFsPMEEK | A*0301 | | | | | | |
| 324 | RSLsVEIVY | A*0301 | | | ✓ | | | |
| 325 | RSYsRSFSR | A*0301 | | | ✓ | | | |
| 326 | RSYsYPRQK | A*0301 | | | ✓ | | | |
| 327 | RTAsFAVRK | A*0301 | | | ✓ | | | |
| 328 | RTAsPPPPPK | A*0301 | ✓ | | ✓ | | | |
| 429 | RTNsPGFQK | A*0301 | | | | | | |
| 329 | RTRsLSSLREK | A*0301 | | ✓ | | | | |
| 432 | RTSsPLFNK | A*0301 | | | | | | |
| 433 | RTYsHGTYR | A*0301 | | | | | | |
| 330 | RVAsPTSGVK | A*0301 | | ✓ | ✓ | | | |
| 331 | RVKtPTSQSYR | A*0301 | | | ✓ | | | |
| 332 | RVLsPLIIK | A*0301 | | ✓ | ✓ | | | |
| 333 | RVRQsPLATR | A*0301 | | ✓ | ✓ | | | |
| 334 | RVYsPYNHR | A*0301 | ✓ | ✓ | ✓ | | | |
| 335 | SVKsPVTVK | A*0301 | | ✓ | ✓ | | | |
| 336 | SVRRsVLMK | A*0301 | | ✓ | ✓ | | | |
| 441 | TLLAsPMLK | A*0301 | | | | | | |
| 337 | yIQSRF | A*0301 | | | ✓ | | | |
| 416 | PYDPALGsPSR | A*24 | | | | | | |
| 389 | RYQtQPVTL | A*24 | | ✓ | | | | |
| 390 | VYTyIQSRF | A*24 | | | | | | |
| 399 | FTKsPYQEF | A*26 | | | | | | |
| 391 | RTSsFTFQN | A*31 | | ✓ | | | | |
| 78 | APDsPRAFL | B*0702 | | ✓ | | | | |
| 79 | APRKGsFSAL | B*0702 | | ✓ | ✓ | | ✓ | |
| 80 | APRNGsGVAL | B*0702 | | | | | | |
| 81 | APRRYsSSL | B*0702 | ✓ | ✓ | ✓ | | | ✓ |
| 82 | APRsPPPSRP | B*0702 | | | ✓ | | | |
| 83 | APSLFHLNtL | B*0702 | | | | | | |
| 84 | APSSARAsPLL | B*0702 | | | | | | |
| 85 | FPLDsPKTLVL | B*0702 | | | | | | |
| 86 | FPRRHsVTL | B*0702 | ✓ | ✓ | ✓ | | | |
| 87 | FRGRYRsPY | B*0702 | | ✓ | | | | |
| 88 | FRKsMVEHY | B*0702 | | | ✓ | | | |
| 89 | GPPYQRRGsL | B*0702 | | | | | | |
| 90 | GPRPGsPSAL | B*0702 | | | ✓ | | ✓ | |
| 91 | GPRSAsLL | B*0702 | ✓ | ✓ | ✓ | | | |
| 92 | GPRSAsLLSL | B*0702 | ✓ | ✓ | ✓ | | | |
| 93 | GPRSAsLLsL | B*0702 | ✓ | | | | | |
| 94 | GPRsPKAPP | B*0702 | ✓ | ✓ | ✓ | | | |
| 95 | HPKRSVsL | B*0702 | | | ✓ | | | |
| 96 | HRYsTPHAF | B*0702 | | ✓ | | | | |
| 405 | KASPKRLsL | B*0702 | | | | | | |
| 411 | KLSGLsF | B*0702 | ✓ | | | | | |
| 97 | KPAsPKFIVTL | B*0702 | ✓ | ✓ | ✓ | | | |
| 98 | KPPYRSHsL | B*0702 | ✓ | | | | | |
| 99 | KPRPLsMDL | B*0702 | | ✓ | | | | |
| 100 | KPRPPPLsP | B*0702 | | ✓ | ✓ | | | |
| 101 | KPRRFsRsL | B*0702 | ✓ | ✓ | ✓ | | | |
| 101 | KPRRFsRSL | B*0702 | ✓ | ✓ | ✓ | | | |
| 102 | KPRsPFSKI | B*0702 | | ✓ | | | | |
| 103 | KPRsPPRAL | B*0702 | ✓ | ✓ | ✓ | | ✓ | |
| 104 | KPRsPPRALVL | B*0702 | | | ✓ | | ✓ | |
| 105 | KPRsPVVEL | B*0702 | ✓ | ✓ | ✓ | | ✓ | ✓ |
| 106 | KPSsPRGSL | B*0702 | | | | | | |
| 107 | KPSsPRGSLL | B*0702 | | | | | | |
| 108 | KPVsPKSGTL | B*0702 | ✓ | | ✓ | | | |
| 109 | KPYsPLASL | B*0702 | ✓ | ✓ | ✓ | | | |
| 110 | KRAsGQAFEL | B*0702 | | | ✓ | | | |
| 111 | LPAsPRARL | B*0702 | ✓ | ✓ | ✓ | | ✓ | ✓ |
| 112 | LPIFSRLsI | B*0702 | | ✓ | ✓ | | ✓ | |
| 113 | LPKGLSAsL | B*0702 | | | | | | |
| 113 | LPKGLsASL | B*0702 | | | | | | |
| 114 | LPRGsSPSVL | B*0702 | ✓ | ✓ | ✓ | | ✓ | |
| 115 | LPRPAsPAL | B*0702 | | | | | | |
| 116 | LPRSSsMAA | B*0702 | | | | | | |
| 117 | LPRSSsMAAGL | B*0702 | | ✓ | | | | |
| 118 | MPRQPsATRL | B*0702 | ✓ | ✓ | ✓ | | | |
| 119 | QPRtPSPLVL | B*0702 | ✓ | ✓ | ✓ | | | |
| 120 | RARGIsPIVF | B*0702 | ✓ | ✓ | ✓ | | ✓ | |
| 121 | RKLsVILIL | B*0702 | | ✓ | ✓ | | | |
| 122 | RLLsPQQPAL | B*0702 | | | | | ✓ | |
| 123 | RPAFFsPSL | B*0702 | ✓ | | ✓ | | | |
| 124 | RPAKsMDSL | B*0702 | ✓ | ✓ | ✓ | | | |
| 125 | RPAsAGAmL | B*0702 | ✓ | ✓ | ✓ | | | |
| 126 | RPAsPAAKL | B*0702 | ✓ | ✓ | ✓ | | ✓ | |
| 127 | RPAsPEPEL | B*0702 | | | | | | |
| 128 | RPAsPGPSL | B*0702 | | | | | | |
| 129 | RPAsPQRAQL | B*0702 | ✓ | ✓ | ✓ | | ✓ | |
| 130 | RPAsPSLQL | B*0702 | | ✓ | | | | |
| 131 | RPAsPSLQLL | B*0702 | | ✓ | | | | |
| 132 | RPAsYKKKSML | B*0702 | | | | | | |
| 133 | RPDsPTRPTL | B*0702 | | ✓ | | | | |
| 134 | RPDsRLGKTEL | B*0702 | ✓ | ✓ | ✓ | | ✓ | |
| 135 | RPDVAKRLsL | B*0702 | | ✓ | | | | |
| 136 | RPFHGISTVsL | B*0702 | | ✓ | | | | |
| 137 | RPFsPREAL | B*0702 | ✓ | ✓ | ✓ | | ✓ | ✓ |
| 138 | RPGsRQAGL | B*0702 | | ✓ | | | | |
| 139 | RPIsPGLSY | B*0702 | | ✓ | ✓ | | ✓ | ✓ |
| 140 | RPIsPPHTY | B*0702 | | | | | | ✓ |
| 141 | RPIsPRIGAL | B*0702 | | ✓ | | | | |
| 142 | RPKLSsPAL | B*0702 | ✓ | ✓ | ✓ | | | |
| 143 | RPKsNIVLL | B*0702 | | | | | | |
| 144 | RPKsPLSKM | B*0702 | | ✓ | | | | |
| 145 | RPKsVDFDSL | B*0702 | | ✓ | ✓ | | | |
| 146 | RPKtPPVVI | B*0702 | ✓ | ✓ | ✓ | | | |
| 147 | RPLsLLLAL | B*0702 | | | | | | |
| 148 | RPLsVVYVL | B*0702 | | | | | | |
| 149 | RPMsESPHM | B*0702 | | | | | | ✓ |
| 150 | RPNsPSPTAL | B*0702 | ✓ | | ✓ | | ✓ | |
| 151 | RPPsPGPVL | B*0702 | ✓ | | ✓ | | | |
| 152 | RPQRAtSNVF | B*0702 | ✓ | ✓ | ✓ | | ✓ | |
| 153 | RPRAAtVV | B*0702 | | ✓ | | | | |
| 154 | RPRAAtVVA | B*0702 | ✓ | ✓ | | | | |
| 155 | RPRANsGGVDL | B*0702 | ✓ | ✓ | ✓ | | | |
| 156 | RPRARsVDAL | B*0702 | | ✓ | ✓ | | ✓ | ✓ |
| 157 | RPRDtRRISL | B*0702 | ✓ | | | | | |
| 158 | RPRGsESLL | B*0702 | | | | | | |
| 159 | RPRGsQSLL | B*0702 | | ✓ | | | | ✓ |
| 160 | RPRIPsPIGF | B*0702 | ✓ | | ✓ | | | |
| 161 | RPRPAsSPAL | B*0702 | | | | | | |
| 162 | RPRPHsAPSL | B*0702 | ✓ | ✓ | ✓ | | | |
| 163 | RPRPSsAHVGL | B*0702 | | ✓ | | | | |
| 164 | RPRPsSVL | B*0702 | | | | | | |
| 165 | RPRPsSVLRTL | B*0702 | | | | | | |
| 166 | RPRPVsPSSL | B*0702 | ✓ | ✓ | ✓ | | ✓ | ✓ |
| 167 | RPRPVsPSSLL | B*0702 | | | ✓ | | | |
| 168 | RPRsAVEQL | B*0702 | | ✓ | | | | |
| 169 | RPRsAVLL | B*0702 | ✓ | ✓ | ✓ | | | |
| 170 | RPRsISVEEF | B*0702 | | ✓ | ✓ | | | |
| 171 | RPRsLEVTI | B*0702 | ✓ | | | | | |
| 172 | RPRSLsSPTVTL | B*0702 | | | ✓ | | ✓ | |
| 173 | RPRsMTVSA | B*0702 | ✓ | ✓ | | | | |
| 174 | RPRsMVRSF | B*0702 | | | | | | |
| 175 | RPRsPAARL | B*0702 | ✓ | | | | | |
| 176 | RPRsPNMQDL | B*0702 | | ✓ | | | | |
| 177 | RPRsPPGGP | B*0702 | | ✓ | | | | |
| 178 | RPRsPPPRAP | B*0702 | | ✓ | ✓ | | | |
| 179 | RPRsPPSSP | B*0702 | ✓ | | ✓ | | | |
| 180 | RPRsPRENSI | B*0702 | ✓ | ✓ | ✓ | | ✓ | |
| 181 | RPRsPRPPP | B*0702 | | | ✓ | | | |
| 182 | RPRsPRQNSI | B*0702 | ✓ | ✓ | ✓ | | ✓ | ✓ |
| 183 | RPRSPsPIS | B*0702 | | | | | | |
| 184 | RPRsPTGPSNSF | B*0702 | | ✓ | ✓ | | | |
| 185 | RPRsPTGPSNSFL | B*0702 | | | ✓ | | | |
| 186 | RPRsPWGKL | B*0702 | | | | | | |
| 187 | RPRsQYNTKL | B*0702 | | | | | | |
| 188 | RPRtPLRSL | B*0702 | | | ✓ | | | |
| 189 | RPSsLPDL | B*0702 | | | ✓ | | ✓ | |
| 190 | RPSsPALYF | B*0702 | ✓ | | | | | |
| 191 | RPTsFADEL | B*0702 | | | | | | ✓ |
| 192 | RPTsRLNRL | B*0702 | ✓ | ✓ | ✓ | | ✓ | |
| 193 | RPVsPFQEL | B*0702 | ✓ | ✓ | ✓ | | ✓ | ✓ |
| 194 | RPVsPGKDI | B*0702 | ✓ | | ✓ | | ✓ | |
| 195 | RPVSPsSLL | B*0702 | | | ✓ | | | |
| 196 | RPVsTDFAQY | B*0702 | | | | | | ✓ |
| 197 | RPVtPVSDL | B*0702 | ✓ | ✓ | ✓ | | | |
| 198 | RPWsNSRGL | B*0702 | | | | | | |
| 199 | RPWsPAVSA | B*0702 | ✓ | ✓ | | | ✓ | |
| 200 | RPYsPPFFSL | B*0702 | ✓ | | ✓ | | | |
| 201 | RPYsQVNVL | B*0702 | | | | | | |
| 202 | RSRsPRPAL | B*0702 | | | ✓ | ✓ | | |
| 203 | RTRsPSPTL | B*0702 | | | ✓ | | | |
| 431 | RVRKLPsTTL | B*0702 | ✓ | | ✓ | | | |
| 204 | SPAsPKISL | B*0702 | ✓ | | ✓ | | ✓ | |
| 205 | SPFKRQLsL | B*0702 | | ✓ | ✓ | | ✓ | ✓ |
| 206 | SPFLsKRSL | B*0702 | | | | | | |
| 207 | SPGLARKRsL | B*0702 | ✓ | | | | | |
| 208 | SPKsPGLKA | B*0702 | ✓ | ✓ | ✓ | | | |
| 209 | SPRERsPAL | B*0702 | | ✓ | ✓ | | ✓ | |
| 210 | SPRGEASsL | B*0702 | | | | | | |
| 210 | SPRGEAsSL | B*0702 | | | | | | |
| 211 | SPRsPGRSL | B*0702 | ✓ | | ✓ | | ✓ | |
| 212 | SPRsPSGLR | B*0702 | | | | | | |
| 213 | SPRSPsTTYL | B*0702 | ✓ | ✓ | ✓ | | | |
| 214 | SPSsPSVRRQL | B*0702 | ✓ | | ✓ | | | |
| 215 | TPMKKHLsL | B*0702 | | | | | | |
| 216 | TPRsPPLGL | B*0702 | ✓ | ✓ | ✓ | | ✓ | ✓ |
| 217 | TPRsPPLGLI | B*0702 | ✓ | ✓ | ✓ | | ✓ | |
| 218 | VAKRLsL | B*0702 | | | | | | |
| 219 | VPRPERRsSL | B*0702 | | ✓ | | | | |
| 220 | VPRsPKHAHSSSL | B*0702 | ✓ | ✓ | | | | |
| 221 | VPTsPKSSL | B*0702 | | | | | ✓ | |
| 222 | YPDPHsPFAV | B*0702 | | | | | | |
| 223 | YPGGRRsSL | B*0702 | | ✓ | | | | |
| 224 | YPYEFsPVKM | B*0702 | | | | | | |
| 398 | DLKSSKAsL | B*08 | | | | | | |
| 438 | SsPIMRKKVSL | B*08 | | | | | | |
| 400 | GQLsPGVQF | B*1508 | | | | | | |
| 108 | KIKsFEVVF | B*1508 | | | | | | |
| 392 | RAHsEPLAL | B*1508 | ✓ | | | | | |
| 225 | FRRsPTKSSL | B*2705 | ✓ | ✓ | | | | |
| 226 | FRRsPTKSSLD | B*2705 | ✓ | ✓ | | | | |
| 227 | FRRsPTKSSLDY | B*2705 | ✓ | ✓ | | | | |
| 228 | GRKsPPPSF | B*2705 | | ✓ | | | | |
| 229 | GRLsPAYSL | B*2705 | | ✓ | | | | |
| 230 | GRLsPVPVPR | B*2705 | | ✓ | | | | |
| 231 | GRQsPSFKL | B*2705 | | | | | | |
| 232 | GRsSPPPGY | B*2705 | | | | | | |
| 233 | KRAsYILRL | B*2705 | | | | | | |
| 234 | KRFsFKKSF | B*2705 | | ✓ | | | | |
| 235 | KRFsFKKsF | B*2705 | | ✓ | ✓ | | | |
| 236 | KRFsGTVRL | B*2705 | | ✓ | ✓ | | | |
| 237 | KRKsFTSLY | B*2705 | | | | | | |
| 238 | KRLEKsPSF | B*2705 | | | | | | |
| 239 | KRLsPAPQL | B*2705 | ✓ | ✓ | ✓ | | | |
| 240 | KRmsPKPEL | B*2705 | | ✓ | ✓ | | | |
| 241 | KRWQsPVTK | B*2705 | | | | | | |
| 242 | KRYsGNmEY | B*2705 | | ✓ | ✓ | | | ✓ |
| 243 | KRYsRALYL | B*2705 | | | | | | |
| 244 | QRLsPLSAAY | B*2705 | | | | | | |
| 420 | RKLRsLEQL | B*2705 | | | | | | |
| 245 | RRAsIITKY | B*2705 | | | | | | |
| 246 | RRAsLSEIGF | B*2705 | | ✓ | | | | |
| 247 | RRDsIVAEL | B*2705 | | ✓ | | | | |
| 248 | RRDsLQKPGL | B*2705 | | ✓ | | | | |
| 249 | RRFsGTAVY | B*2705 | ✓ | ✓ | | | | ✓ |
| 250 | RRFsIATLR | B*2705 | | ✓ | | | | |
| 251 | RRFsLTTLR | B*2705 | | ✓ | | | | |
| 252 | RRFsPPRRm | B*2705 | ✓ | ✓ | | | | ✓ |
| 253 | RRFsRSDEL | B*2705 | | | | | | |
| 254 | RRFsRsPIR | B*2705 | | | | | | |
| 255 | RRFSRsPIR | B*2705 | | | ✓ | | | |
| 256 | RRFsRsPIRR | B*2705 | | | ✓ | | | |
| 257 | RRGsFEVTL | B*2705 | | ✓ | | | | ✓ |
| 258 | RRIDIsPSTF | B*2705 | | ✓ | | | | |
| 259 | RRIsDPEVF | B*2705 | ✓ | ✓ | | ✓ | | |
| 260 | RRIsDPQVF | B*2705 | | ✓ | | | | |
| 261 | RRIsQIQQL | B*2705 | | | | | | |
| 262 | RRKsQVAEL | B*2705 | ✓ | ✓ | ✓ | | | ✓ |
| 263 | RRLsADIRL | B*2705 | | ✓ | ✓ | | | |
| 264 | RRLsELLRY | B*2705 | | ✓ | | | | |
| 265 | RRLsGGSHSY | B*2705 | | | ✓ | | | |
| 266 | RRLsRKLSL | B*2705 | | | | | | |
| 267 | RRMsFQKP | B*2705 | | ✓ | | | | |
| 268 | RRmsLLSVV | B*2705 | | ✓ | ✓ | | | ✓ |
| 269 | RRNsAPVSV | B*2705 | | ✓ | | | | ✓ |
| 270 | RRPsIAPVL | B*2705 | | | | | | |
| 271 | RRPsLLSEF | B*2705 | | ✓ | | | | ✓ |
| 272 | RRPsLVHGY | B*2705 | | | ✓ | | | |
| 273 | RRPsYTLGM | B*2705 | | | ✓ | | | |
| 274 | RRRsLERLL | B*2705 | | | | | | |
| 275 | RRSFsLE | B*2705 | | | | | | |
| 276 | RRSsFLQ | B*2705 | | | | | | |
| 277 | RRSsFLQVF | B*2705 | | ✓ | ✓ | | | |
| 278 | RRSsIQSTF | B*2705 | | ✓ | | | | |
| 279 | RRSsQSWSL | B*2705 | ✓ | ✓ | ✓ | | | |
| 280 | RRVVQRSsL | B*2705 | | | ✓ | | | |
| 281 | RRYsKFFDL | B*2705 | | | | | | |
| 282 | RRYsPPIQR | B*2705 | ✓ | | ✓ | | | |
| 283 | RSRsPLEL | B*2705 | | | ✓ | | | |
| 284 | SPRRsRSISL | B*2705 | ✓ | ✓ | ✓ | ✓ | | |
| 285 | SRFNRRVsV | B*2705 | | | | | | |
| 397 | DAKKsPLAL | B*35 | | | | | | |
| 436 | SDmPRAHsF | B*37 | | | | | | |
| 338 | AENARSAsF | B*4402 | | | | | | |
| 339 | AENsPTRQQF | B*4402 | ✓ | ✓ | ✓ | | | |
| 340 | AENsSSREL | B*4402 | | | | | | |
| 341 | AtAGPRLGW | B*4402 | | | ✓ | | | |
| 342 | EELsPTAKF | B*4402 | ✓ | ✓ | | | | |
| 343 | FKtQPVTF | B*4402 | | ✓ | | | | |
| 344 | GEAsPSHII | B*4402 | | | | | | |
| 345 | GEIsPQREV | B*4402 | | | | | | |
| 346 | GETsPRTKI | B*4402 | | | | | | |
| 347 | HEKKAYsF | B*4402 | | | | | | |
| 348 | KEKsPFRET | B*4402 | | | | | | |
| 349 | KELARQIsF | B*4402 | | | | | | |
| 350 | KEmsPTRQL | B*4402 | | ✓ | ✓ | | | |
| 351 | KESsPLSSRKI | B*4402 | | | | | | |
| 352 | REAPsPLmI | B*4402 | | | | | | |
| 352 | REAPsPLmI | B*4402 | | | | | | |
| 353 | REAsPAPLA | B*4402 | | | | | | |
| 354 | REAsPRLRV | B*4402 | | | | | | |
| 355 | REAsPSRLSV | B*4402 | | | | | | |
| 356 | REIMGtPEYL | B*4402 | | | | | | |
| 357 | REKsPGRmL | B*4402 | | | | | | |
| 358 | RELARKGsL | B*4402 | | | | | | |
| 359 | RELsPLISL | B*4402 | | | | | | |
| 360 | REPsPLPEL | B*4402 | | | | | | |
| 361 | RERsPSPSF | B*4402 | | | | | | |
| 362 | RESsPTRRL | B*4402 | | | | | | |
| 363 | REVsPAPAV | B*4402 | | | | | | |
| 364 | REYGsTSSI | B*4402 | | | | | | |
| 365 | RFKtQPVTF | B*4402 | | ✓ | | | | |
| 366 | RQKsPLFQF | B*4402 | | | ✓ | | | |
| 367 | SEFKAMDsI | B*4402 | | | | | | |
| 368 | SELsPGRSV | B*4402 | | | | | | |
| 369 | TEAsPESML | B*4402 | | | | | | |
| 370 | YEGsPIKV | B*4402 | ✓ | | | | | |
| 393 | ADLsPEREV | B*49 | | | | | | |
| 437 | SFDsGSVRL | C*04 | | | | | | |
| 394 | AGDsPGSQF | C*0501 | | | | | | |
| 383 | KVDsPVIF | C*0501 | | | | | | |
| 413 | NMDsPGPML | C*0501 | | | | | | |
| 384 | RADsPVHM | C*0501 | ✓ | ✓ | | | | |
| 422 | RLLDPsSPLAL | C*0501 | | | | | | |
| 422 | RLLDPSsPLAL | C*0501 | | | | | | |
| 385 | RSDsYVEL | C*0501 | ✓ | ✓ | | | | ✓ |
| 386 | RSEsPPAEL | C*0501 | | | | | | |
| 387 | RVDsPSHGL | C*0501 | | | | ✓ | | |
| 435 | sDDEKMPDLE | C*0501 | | | | | | |
| 388 | SIDsPQKL | C*0501 | | ✓ | ✓ | | | |
| 447 | VVDsPGQEVL | C*0501 | | | | | | |
| 371 | FRFsGRTEY | C*0602 | | | | | | |
| 372 | KRAsFAKSV | C*0602 | ✓ | | ✓ | | | |
| 373 | LSSsVIREL | C*0602 | | | | | | |
| 374 | RKPsIVTKY | C*0602 | ✓ | | | | | ✓ |
| 375 | RRHsASNLHAL | C*0602 | | | | | | |
| 376 | RRLsFLVSY | C*0602 | | ✓ | ✓ | | | ✓ |
| 377 | RRLsYVLFI | C*0602 | | | | | | |
| 378 | RRPsYRKIL | C*0602 | | | ✓ | | | |
| 379 | RSAsFSRKV | C*0602 | | | | | | |
| 380 | SRSSSVLsL | C*0602 | | | | | | ✓ |
| 381 | TRKtPESFL | C*0602 | | | | | | ✓ |
| 382 | YRYsPQSFL | C*0602 | | | | | | |
| 426 | RNLsSPFIF | C*07 | | | | | | |
| 431 | RTSsFALNL | C*07 | | | | | | |
| 442 | TLMERTVsL | C*07 | | | | | | |
| 412 | KTMsPSQMIM | C*16 | | | | | | |
| 425 | RMYsPIPPSL | C*16 | | | | | | |
| 430 | RTPsDVKEL | C*16 | | | | | | |
| 448 | YARsVHEEF | C*16 | | | | | | |
| 395 | AKLsETIS | — | | | | | | |
| 396 | AsLGFVF | — | | | | | | |
| 401 | GsPHYFSPF | — | | | | | | |
| 406 | KAVsLFLcY | — | | | | | | |
| 417 | RAFsVKFEV | — | | | | | | |
| 418 | RGDGYGtF | — | | | | | | |
| 421 | RKSsIIIRM | — | | | | | | |
| 423 | RLSsLRASTSK | — | | | | | | |
| 428 | RTHsLLLLL | — | ✓ | ✓ | | ✓ | | ✓ |
| 434 | RYPsNLQLF | — | | | | | | |
| 439 | sYIEHIFEI | — | | | | | | |
| 440 | sYQKVIELF | — | | | | | | |

#Phospho- Ser, -Thr, and -Tyr residues are indicated by “s”, “t”, and “y”, respectively. A lowercase “c” in a peptide sequence indicates that in some embodiments the cysteine is present in a cysteine-cysteine disulfide bond at the surface of a cell and, in some embodiments, is presented to the immune system as such. A lowercase “m” in a peptide sequence indicates that in some embodiments the methionine is oxidized. The presence of phosphopeptides in previously analyzed samples including leukemia, colorectal cancer, melanoma, ovarian cancer, breast cancer, and esophageal cancer is indicated by ✓. White boxes indicate instances in which the phosphopeptide is unique to liver samples.

1. A composition comprising at least one peptide and an adjuvant, wherein each peptide:
(i) is 8 to 50 amino acids long; and
(ii) comprises an amino acid sequence selected from the group consisting of SEQ ID NOs: 92, 115, 160, 167, 185, 408, 4, 5, 10, 11, 15, 24, 32, 33, 37, 38, 41, 42, 52, 59, 63, 64, 66, 72, 75, 80, 83-89, 91, 95, 96, 106-108, 113, 116-117, 122, 123, 127, 128, 130-132, 146-149, 157, 158, 161, 163-165, 174, 179, 181, 186-188, 195, 198, 203, 206, 210, 212, 215, 218, 221, 222, 224, 226, 231-233, 237, 243, 245, 253, 261, 266, 270, 274, 275, 276, 281, 285-287, 292, 293, 295, 297, 299, 303-305, 317, 320, 337, 338, 340, 343-349, 351-364, 367-371, 373, 377, 379, 382, 383, 385, 386, 393-407, 409-412, 414-426, 429-436, 438-448, 502, and 509-529.

2. The composition of claim 1, wherein at least one of the peptides comprises:
(a) a substitution of a serine residue with a homo-serine residue;
(b) a phosphopeptide comprising phosphoserine, phosphothreonine, or phosphotyrosine;
(c) a phosphopeptide set forth in Tables 2-14;
(d) a phosphopeptide mimetic comprising a mimetic of phosphoserine, phosphothreonine, or phosphotyrosine; and/or
(e) a phosphopeptide mimetic of a phosphopeptide set forth in Tables 2-14, optionally wherein the phosphopeptide mimetic is resistant to dephosphorylation by a phosphatase enzyme and/or the phosphopeptide mimetic is a synthetic molecule in which a phosphorous atom is linked to a serine, threonine, or tyrosine amino acid residue through a carbon.

3-9. (canceled)

10. The composition of claim 1, wherein:
(a) the composition comprises at least 2, 3, 4, or 5 different peptides;
(b) the composition comprises at least 10 different peptides;
(c) the composition comprises at least 15 different peptides;
(d) at least one of the peptides is capable of binding to an MHC class I molecule selected from the group consisting of an HLA-A*0201 molecule, an HLA A*0101 molecule, an HLA A*0301 molecule, an HLA B*4402 molecule, an HLA B*0702 molecule, and an HLA B*2705 molecule; and/or
(e) the composition has the ability to stimulate a T cell-mediated immune response to at least one of the peptides and/or is capable of eliciting a memory T cell response to at least one of the peptides.

11-17. (canceled)

18. The composition of claim 1, further comprising at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HT-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, and TPS.

19. The composition of claim 1, wherein the adjuvant is selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof.

20. An in vitro population of dendritic cells comprising one or more of the peptides set forth in claim 1.

21. An in vitro population of CD8⁺ T cells capable of being activated upon being brought into contact with a population of dendritic cells, wherein the dendritic cells comprise one or more of the peptides set forth in claim 1.

22. An antibody or antibody-like molecule that specifically binds to a complex of an MHC class I molecule and a peptide, wherein the peptide comprises an amino acid sequence selected from the group consisting of SEQ ID NOs: 92, 115, 160, 167, 185, 408, 4, 5, 10, 11, 15, 24, 32, 33, 37, 38, 41, 42, 52, 59, 63, 64, 66, 72, 75, 80, 83-89, 91, 95, 96, 106-108, 113, 116-117, 122, 123, 127, 128, 130-132, 146-149, 157, 158, 161, 163-165, 174, 179, 181, 186-188, 195, 198, 203, 206, 210, 212, 215, 218, 221, 222, 224, 226, 231-233, 237, 243, 245, 253, 261, 266, 270, 274, 275, 276, 281, 285-287, 292, 293, 295, 297, 299, 303-305, 317, 320, 337, 338, 340, 343-349, 351-364, 367-371, 373, 377, 379, 382, 383, 385, 386, 393-407, 409-412, 414-426, 429-436, 438-448, 502, and 509-529, optionally wherein the peptide and/or the corresponding MHC class I molecule is selected from Tables 2-14.

23-24. (canceled)

25. The antibody or antibody-like molecule of claim 22, wherein the antibody or antibody-like molecule:
(a) is a member of the immunoglobulin superfamily;
(b) comprises a binding member selected from the group consisting an Fab, Fab′, F(ab′)2, Fv, and a single-chain antibody;
(c) is conjugated to a therapeutic agent selected from the group consisting of an alkylating agent, an antimetabolite, a mitotic inhibitor, a taxoid, a vinca alkaloid, and an antibiotic; and/or
(d) is a T cell receptor, optionally conjugated to a CD3 agonist.

26-28. (canceled)

29. An in vitro population of T cells transfected with a nucleic acid, optionally an mRNA, encoding a T cell receptor of claim 25.

30. A method for treating and/or preventing cancer comprising administering to a subject in need thereof a therapeutically effective dose of:
(a) a composition of claim 1;
(b) a composition comprising at least one peptide comprising an amino acid sequence as set forth in any of SEQ ID NOs: 92, 115, 160, 167, 185, 408, 4, 5, 10, 11, 15, 24, 32, 33, 37, 38, 41, 42, 52, 59, 63, 64, 66, 72, 75, 80, 83-89, 91, 95, 96, 106-108, 113, 116-117, 122, 123, 127, 128, 130-132, 146-149, 157, 158, 161, 163-165, 174, 179, 181, 186-188, 195, 198, 203, 206, 210, 212, 215, 218, 221, 222, 224, 226, 231-233, 237, 243, 245, 253, 261, 266, 270, 274, 275, 276, 281, 285-287, 292, 293, 295, 297, 299, 303-305, 317, 320, 337, 338, 340, 343-349, 351-364, 367-371, 373, 377, 379, 382, 383, 385, 386, 393-407, 409-412, 414-426, 429-436, 438-448, 502, and 509-529;
(c) an in vitro population of dendritic cells comprising one or more of the peptides set forth in claim 1; or
(d) an in vitro population of CD8⁺ T cells capable of being activated upon being brought into contact with a population of dendritic cells comprising one or more of the peptides set forth in claim 1,
optionally wherein the cancer is HCC or esophageal cancer.

31-36. (canceled)

37. A method for making a cancer vaccine, optionally a cancer vaccine for use in treating and/or preventing hepatocellular carcinoma (HCC) and/or esophageal cancer, comprising combining one or more of the peptides set forth in claim 1 with an the adjuvant selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof and a pharmaceutically acceptable carrier; and placing the composition, adjuvant, and pharmaceutical carrier into a container, optionally into a syringe.

38. A method for screening peptides for inclusion in the immunotherapy composition of claim 1 or for use in a method of using the composition of claim 1, comprising:
(a) administering the peptide to a human;
(b) determining whether the peptide is capable of inducing a peptide-specific memory T cell response in the human; and
(c) selecting the peptide for inclusion in an immunotherapy composition if the peptide elicits a memory T cell response in the human.

39. A method for determining a prognosis of a hepatocellular carcinoma (HCC) patient and/or an esophageal cancer patient, the method comprising:
(a) administering to the patient a peptide as set forth in claim 1 wherein the peptide is associated with the patient's HCC and/or esophageal cancer;
(b) determining whether the peptide is capable of inducing a peptide-specific memory T cell response in the patient; and
(c) determining that the patient has a better prognosis if the patient mounts a memory T cell response to the peptide than if the patient did not mount a memory T cell response to the peptide.

40. A kit comprising at least one peptide composition comprising at least one of the peptides set forth in claim 1 and a cytokine and/or an adjuvant.

41. The kit of claim 40, wherein:
(a) the kit comprises at least 2, 3, 4, or 5 target peptide compositions;
(b) the at least one peptide composition is one of the compositions of claim 1;
(c) the cytokine is selected from the group consisting of a transforming growth factor (TGF), optionally TGF-alpha and/or TGF-beta; insulin-like growth factor-I; insulin-like growth factor-II; erythropoietin (EPO); an osteoinductive factor; an interferon, optionally interferon-alpha, interferon-beta, and/or interferon-gamma; and a colony stimulating factor (CSF), optionally macrophage-CSF (M-CSF), granulocyte-macrophage-CSF (GM-CSF), and/or granulocyte-CSF (G-CSF);
(d) the adjuvant is selected from the group consisting of montanide ISA-51, QS-21, a tetanus helper peptide, GM-CSF, cyclophosphamide, bacillus Calmette-Guerin (BCG), corynbacterium parvum, levamisole, azimezone, isoprinisone, dinitrochlorobenezene (DNCB), a keyhole limpet hemocyanin (KLH), complete Freund's adjuvant, incomplete Freund's adjuvant, a mineral gel, aluminum hydroxide, lysolecithin, a pluronic polyol, a polyanion, an adjuvant peptide, an oil emulsion, dinitrophenol, and diphtheria toxin (DT);
(e) the cytokine is selected from the group consisting of a nerve growth factor, optionally nerve growth factor (NGF) beta; a platelet-growth factor; a transforming growth factor (TGF), optionally TGF-alpha and/or TGF-beta; insulin-like growth factor-I; insulin-like growth factor-II; erythropoietin (EPO); an osteoinductive factor; an interferon, optionally interferon-α, interferon-β, and/or interferon-γ; a colony stimulating factor (CSF), optionally macrophage-CSF (M-CSF), granulocyte-macrophage-CSF (GM-CSF), and/or granulocyte-CSF (G-CSF); an interleukin (IL), optionally IL-1, IL-1a, IL-2, IL-3, IL-4, IL-5, IL-6, IL-7, IL-8, IL-9, IL-10, IL-11, IL-12; IL-13, IL-14, IL-15, IL-16, IL-17, and/or IL-18; LIF; EPO; kit-ligand; fms-related tyrosine kinase 3 (FLT-3; also called CD135); angiostatin; thrombospondin; endostatin; tumor necrosis factor; and lymphotoxin (LT);
(f) the kit further comprises at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, and TPS;
(g) the at least one peptide composition comprises one or more peptides that specifically bind to an HLA molecule listed in Table 1 and/or that comprises an amino acid sequence at least 90% identical, optionally 100% identical, to one of the SEQ ID NOs: listed in Tables 2, 3, 5-7, and 14;
(h) the kit comprises at least two peptides, wherein the at least two peptides are in separate containers;
(i) the kit further comprising instructions related to determining whether the at least one peptide of the at least one peptide composition is capable of inducing a T cell memory response that is a T cell central memory response (Tcm) when the at least one peptide composition is administered to a patient; and/or
(j) the kit further comprises a tetanus peptide, optionally wherein the tetanus peptide:
comprises an amino acid sequence that is at least 90%, 95%, or 100% identical to SEQ ID NO: 449 or SEQ ID NO: 450;
is about or at least 10, 11, 12, 13, 14, 15, 16, 17, 18, 19, 20, 21, 22, 23, 24, or 25 natural or non-natural amino acids in length;
comprises an amino acid sequence that is at least 90% identical to a 10-25 amino acid subsequence of a wild type tetanus toxoid protein; and/or
binds to one or more MHC Class II molecules when administered to a subject.

42-56. (canceled)

57. The composition of claim 1,
(a) comprising a peptide capable of binding to an MHC class I molecule selected from the group consisting of an HLA-A*0201 molecule, an HLA A*0101 molecule, an HLA A*0301 molecule, an HLA B*4402 molecule, an HLA B*0702 molecule, and an HLA B*2705 molecule;
(b) wherein at least one of the peptides comprises an amino acid sequence selected from the group consisting of SEQ ID NOs: 92, 115, 160, 167, 185, 408; and/or
(c) wherein the composition further comprises a tetanus peptide, optionally wherein the tetanus peptide:
comprises an amino acid sequence that is at least 90%, 95%, or 100% identical to SEQ ID NO: 449 or SEQ ID NO: 450;
is about or at least 10, 11, 12, 13, 14, 15, 16, 17, 18, 19, 20, 21, 22, 23, 24, or 25 natural or non-natural amino acids in length;
comprises an amino acid sequence that is at least 90% identical to a 10-25 amino acid subsequence of a wild type tetanus toxoid protein;
binds to one or more MHC Class II molecules when administered to a subject; and/or
is modified so as to prevent formation of tetanus peptide secondary structures.

58-64. (canceled)
